SEQUENCE LISTING (137 sequences)

SEQ ID NO: 1 - protein, 534 aa, Amaranthus sp.
MVIQSITHLSPNLALPSPLSVSTKNYPVAVMGNISEREEPTSAKRVAVVGAGVSGLAAAYKLKSHGLSVTLFEADSRAGGKLKTVKKDGFIWDEGANTMTESEAEVSSLIDDLGLREKQQLPISQNKRYIARDGLPVLLPSNPAALLTSNILSAKSKLQIMLEPFLWRKHNATELSDEHVQESVGEFFERHFGKEFVDYVIDPFVAGTCGGDPQSLSMHHTFPEVWNIEKRFGSVFAGLIQSTLLSKKEKGGENASIKKPRVRGSFSFQGGMQTLVDTMCKQLGEDELKLQCEVLSLSYNQKGIPSLGNWSVSSMSNNTSEDQSYDAVVVTAPIRNVKEMKIMKFGNPFSLDFIPEVTYVPLSVMITAFKKDKVKRPLEGFGVLIPSKEQHNGLKTLGTLFSSMMFPDRAPSDMCLFTTFVGGSRNRKLANASTDELKQIVSSDLQQLLGTEDEPSFVNHLFWSNAFPLYGHNYDSVLRAIDKMEKDLPGFFYAGNHKGGLSVGKAMASGCKAAELVISYLDSHIYVKMDEKTA

SEQ ID NO: 2 - protein, 534 aa, Amaranthus sp.
MVIQSITHLSPNLALPSPLSVSTKNYPVAVMGNISEREEPTSAKRVAVVGAGVSGLAAAYKLKSHGLSVTLFEADSRAGGKLKTVKKDGFIWDEGANTMTESEAEVSSLIDDLGLREKQQLPISQNKRYIARAGLPVLLPSNPAALLTSNILSAKSKLQIMLEPFLWRKHNATELSDEHVQESVGEFFERHFGKEFVDYVIDPFVAGTCGGDPQSLSMHHTFPEVWNIEKRFGSVFAGLIQSTLLSKKEKGGENASIKKPRVRGSFSFQGGMQTLVDTMCKQLGEDELKLQCEVLSLSYNQKGIPSLGNWSVSSMSNNTSEDQSYDAVVVTAPIRNVKEMKIMKFGNPFSLDFIPEVTYVPLSVMITAFKKDKVKRPLEGFGVLIPSKEQHNGLKTLGTLFSSMMFPDRAPSDMCLFTTFVGGSRNRKLANASTDELKQIVSSDLQQLLGTEDEPSFVNHLFWSNAFPLYGHNYDSVLRAIDKMEKDLPGFFYAGNHKGGLSVGKAMASGCKAAELVISYLDSHIYVKMDEKTA

SEQ ID NO: 3 - protein, 533 aa, Amaranthus sp.
MVIQSITHLSPNLALPSPLSVSTKNYPVAVMGNISEREEPTSAKRVAVVGAGVSGLAAAYKLKSHGLSVTLFEADSRAGGKLKTVKKDGFIWDEGANTMTESEAEVSSLIDDLGLREKQQLPISQNKRYIARDGLPVLLPSNPAALLTSNILSAKSKLQIMLEPFLWRKHNATELSDEHVQESVGEFFERHFGKEFVDYVIDPFVAGTCGDPQSLSMHHTFPEVWNIEKRFGSVFAGLIQSTLLSKKEKGGENASIKKPRVRGSFSFQGGMQTLVDTMCKQLGEDELKLQCEVLSLSYNQKGIPSLGNWSVSSMSNNTSEDQSYDAVVVTAPIRNVKEMKIMKFGNPFSLDFIPEVTYVPLSVMITAFKKDKVKRPLEGFGVLIPSKEQHNGLKTLGTLFSSMMFPDRAPSDMCLFTTFVGGSRNRKLANASTDELKQIVSSDLQQLLGTEDEPSFVNHLFWSNAFPLYGHNYDCVLRAIDKMEKDLPGFFYAGNHKGGLSVGKAMASGCKAAELVISYLDSHIYVKMDEKTA

SEQ ID NO: 4 - protein, 533 aa, Amaranthus sp.
MVIQSITHLSPNLALPSPLSVSTKNYPVAVMGNISEREEPTSAKRVAVVGAGVSGLAAAYKLKSHGLSVTLFEANSRAGGKLKTVKKDGFIWDEGANTMTESEAEVSSLIDDLGLREKQQLPISQNKRYIARDGLPVLLPSNPAALLTSNILSAKSKLQIMLEPFLWRKHNATELSDEHVQESVGEFFERHFGKEFVDYVIDPFVAGTCGDPQSLSMYHTFPEVWNIEKRFGSVFAGLIQSTLLSKKEKGGENASIKKPRVRGSFSFQGGMQTLVDTMCKQLGEDELKLQCEVLSLSYNQKGIPSLGNWSVSSMSNNTSEDQSYDAVVVTAPIRNVKEMKIMKFGNPFSLDFIPEVTYVPLSVMITAFKKDKVKRPLEGFGVLIPSKEQHNGLKTLGTLFSSMMFPDRAPSDMCLFTTFVGGSRNRKLANASTDELKQIVSSDLQQLLGTEDEPSFVNHLFWSNAFPLYGHNYDSVLRAIDKMEKDLPGFFYAGNHKGGLSVGKAMASGCKAAELVISYLDSHIYVKMDEKTA

SEQ ID NO: 5 - protein, 535 aa, Amaranthus sp.
MVIQSITHLSPKLALPSPLSVSTKNYPVAVMGNISEREEPTSAKRVAVVGAGVSGLAAAYKLKSHGLNVTLFEADSRAGGKLKTVKKDGFIWDEGANTMTESEAEVSSLIDDLGLREKQQLPISQNKRYIARDGLPVLLPSNPAALLSSNILSAKSKLQIMLEPFLWRKRNATELSDEHVQESVGEFFERHFGKEFVDYVIDPFVAGTCGGDPQSLSVHHTFPDVWNVEKRFGSVFAGLIQSTLLSKKEKGGGENASIKKPRVRGSFSFHGGMQTLVDTMCKQIGEDELKLQCEVLSLSYNQKGIPSLGNWSVSSMSNNTSEDQSYDAVVVTAPIRNVKEMKIMKFGNPFSLDFIPEVTYVPLSVMITAFKKDKVKRPLEGFGVLIPSKEQHNGLKTLGTLFSSMMFPDRAPSDMCLFTTFVGGSRNRKLAKASTDELKQIVSSDLQQLLGTEDEPSFVNHLFWSNAFPLYGHNYDSVLRAIDKMEKDLPGFFYAGNHKGGLSVGKAMASGCKAAELVISYLDSHLYVKMNEKTA

SEQ ID NO: 6 - protein, 505 aa, Amaranthus sp.
MGNISERDEPTSAKRVAVVGAGVSGLAAAYKLKSHGLNVTLFEADSRAGGKLKTVKKDGFIWDEGANTMTESEAEVSSLIDDLGLREKQQLPISQNKRYIARDGLPVLLPSNPAALLTSNILSAKSKLQIMLEPFFWRKHNATELSDEHVQESVGEFFERHFGKEFVDYVIDPFVAGTCGGDPQSLSMHHTFPEVWNIEKRFGSVFAGLIQSTLLSKKEKGGGGNASIKKPRVRGSFSFHGGMQTLVDTICKQLGEDELKLQCEVLSLSYNQKGIPSLGNWSVSSMSNNTSEDQSYDAVVVTAPIRNVKEMKIMKFGNPFSLDFIPEVSYVPLSVMITAFKKDKVKRPLEGFGVLIPSKEQHNGLKTLGTLFSSMMFPDRAPSDMCLFTTFVGGSRNRKLANASTDELKQIVSSDLQQLLGTEDEPSFVNHLFWSNAFPLYGHNYDSVLRAIDKMEKDLPGFFYAGNHKGGLSVGKAMASGCKAAELVISYLDSHIYVKMDEKTA

SEQ ID NO: 7 - protein, 531 aa, Spinacia sp.
MVILPVSQLSTNLGLSLVSPTKNNPVMGNVSERNQVNQPISAKRVAVVGAGVSGLAAAYKLKSNGLNVTLFEADSRAGGKLKTVVKDGLIWDEGANTMTESDEEVTSLFDDLGIREKLQLPISQNKRYIARDGLPVLLPSNPVALLKSNILSAKSKLQIMLEPFLWKKHNGAKVSDENAQESVAEFFERHFGKEFVDYLIDPFVAGTSGGDPQSLSMRHAFPELWNIENRFGSVISGFIQSKLSSKKEKGGEKQSSNKKPRVRGSFSFQGGMQTLVDTICKEFGEDELKLQSEVLSLSYSHNGSLTSENWSVSSMSNSTIQDQPYDAVVVTAPINNVKELKIMKVENPFSLDFIPEVSCLPLSVIITTFKKTNVKRPLEGFGVLVPSNEQHNGLKTLGTLFSSMMFPDRAPSDVYLYTTFVGGSRNRELAKASTDELKQIVSSDLQQLLGTEGEPTFVNHFYWSKAFPLYGRNYDSVLRAIEKMERDLPGLFYAGNHKGGLSVGKSIASGYKAAELAISYLESNKMTEETI

SEQ ID NO: 8 - protein, 508 aa, Vitis vinifera
MAEKSDAQSHYNGSGKRVAVVGAGVSGLAAAYKLKLHGVNITLYEAEERAGGKLRSVSQHGLVWDEGANTMTESEIEVGSLLDNLRLREKQQFPISQNKRYIVRNGMPVLLPSNPIALIKSNILSAKSKFQIILEPFLWKKSDLSKVSDDHMKESVGGFFQRHFGKEVVDYLIDPFVAGTSGGDPESLSMHHTFPELWNLEKRFGSIIAGAVLSKLSAKREKRGETKGSSEKKKRQRGSFSFQGGMQTLTDTLCKELGKDKLRLKSKVLSLSYSHGEKSALENWSVAYASNPGKQSKDLSFDAVIMTAPLCNVREMKIMKKGNPFLLDFLPEVSYLPLSVIITTFKKENVKRPLEGFGVLVPSKEQQNGLKTLGTLFSSMMFPDRAPNDLYLYTTFIGGSRNRELAKASTDELKQIVTSDLRQLLGAEGEPTFVNHFYWSKAFPLFGHNYDSVLEAIDKMEKDLPGFFYAGNHKGGLSVGKAIASGCKAADLVISYLNSSSDGKMFKE

SEQ ID NO: 9 - protein, 511 aa, Ricinus communis
MSSVIKEDRNPSHVKRVAVVGAGVSGLAAAYKLKSHGLKVTVFEAEERAGGKLRSVNHDGLIWDEGANTMTESEMEVKSLIGNLGIREKQQFPISQNKRYIVRNGKPILIPTNPIALITSNILSAQSKFQIILEPFLWKKRESSETHNAYTEESVGEFFQRHFGKEVVDYLIDPFVAGTSAGDPESLSVCHSFPELWNLEKRFGSIIAGVVQAKLSTKRGKSQETKGSSVKKKQQRGSFSFFGGMQTLTDTLCKALAKDELRLESKVFSLSYNPDSKSAVENWSLSYAFKGAKHLQNSSYDAIVMTAPLCNVKEMKITKNRNIFSLNFLPEVSYMPLSVVITTFKKDNVKSPLEGFGVLVPSKEQQNGLKTLGTLFSSMMFPDRAPNDLYLYTTFVGGSRNKELAKASTDDLKQIVTSDLRQLLGAEGEPTFVNHFYWSKAFPLYGRNYDAVLEAIDTMEKDLPGFFYAGNHKGGLSVGKAIASGCKAADLVISYLESSSDDKMLKEGPSN

SEQ ID NO: 10 - protein, 504 aa, Theobroma sp.
MAAAKNKDKQTSAKRVAVVGAGVSGLAAAYKLKSHGLNVTMFEAEGRAGGKLRSVSQEGLIWDEGANTMTESEIEVRSLFDDLGIRDKQQVPIAQKKRYIVRNGVPVLIPSNPIALITSNILSAKSKFQIILEPFLWKKSDASKVSDAYNLESVGGFFQRHFGQEVVDYLIDPFVAGTSAGDPESLSMRHSFPELWDLEKRFGSIIVGAVKSKLSAKRENRGERKTSEKRKPLPGPFSFQGGMQTLTDMLCKDLSKDELKLKSKVLSLSYSHDGKSTLENWSLSYASDRDKRSQGSSFDAVVMTAPLCNVKEMKIMKGGKLFPLNFIPQVSYMPLSVIITTFKKENVKKPLEGFGVLVPSKEQQNGLKTLGTLFSSIMFPDRAPNNLYLYTTFVGGSRNKELAKASTDELKHIVTSDLRQLLGVEGEPTFLNHFYWSKAFPLYGRNYASVLKAIEKMETDLPGFFYAGNHKGGLSVGKAIASGCKAADLVISYLESSHQKLLKD

SEQ ID NO: 11 - protein, 502 aa, Glycine sp.
MASSATDDNPRSVKRVAVVGAGVSGLAAAYKLKSHGLDVTVFEAEGRAGGRLRSVSQDGLIWDEGANTMTESEIEVKGLIDALGLQEKQQFPISQHKRYIVKNGAPLLVPTNPAALLKSKLLSAQSKIHLIFEPFMWKRSDPSNVCDENSVESVGRFFERHFGKEVVDYLIDPFVGGTSAADPESLSMRHSFPELWNLEKRFGSIIAGALQSKLFAKREKTGENRTALRKNKHKRGSFSFQGGMQTLTDTLCKELGKDDLKLNEKVLTLAYGHDGSSSSQNWSITSASNQSTQDVDAVIMTAPLYNVKDIKITKRGTPFPLNFLPEVSYVPISVMITTFKKENVKRPLEGFGVLVPSKEQKNGLKTLGTLFSSMMFPDRAPSDLYLYTTFIGGTQNRELAQASTDELRKIVTSDLRKLLGAEGEPTFVNHFYWSKGFPLYGRNYGSVLQAIDKIEKDLPGFFFAGNYKGGLSVGKAIASGCKAADLVISYLNSASDNTVPDK

SEQ ID NO: 12 - protein, 502 aa, Prunus sp.
MTSLTTKEGSVKRVAVVGAGVSGLAAAYKLKSHGFDVTVFEAEGRAGGKLRSVSHDGLIWDEGANTMTESEKEVQTLLDDLGIREKQQFPISQNKRYIVRNGSPVLIPTNPIALIKSNFLSAQSKLQIILEPYLWKDKRVSDDHTEESVGGFFQRHFGEEVVDYLIDPFVAGTSAGDPESLSMRHSFPDIWNIEKRFGSVISGAIKSKLSASKGKSGETKGSVEKGKRQRGSFSFHGGMQTLTDILCNQLEKDELKLNSKVLSLSYRQGGNSASENWSVSRVADDDKHSQSLSVDALIMTAPLCNVKEMKITKRGTRFPLDFIPEVVYMPLSVIITTFKKENVKRPLEGFGVLVPSKEQKNGLKTLGTLFSSMMFPDRAPSDLYLYTTFVGGSRNKELAKASTDELKQIVTSDIRHLLGAEGEPTFVNHFYWSNAFPLYGRDYDSVIEAIENMEKNLPGFFYAGNHRGGLSVGKSIASGCKAAELVISYLESPSDEKTRHKG

SEQ ID NO: 13 - protein, 499 aa, Medicago sp.
MASSAKDDNPRSVKRVAVVGAGVSGLAAAYKLKSHGLDVTVFEAEGRAGGRLRTVSRDGLVWDEGANTMTENEIEVKGLIDALGLHEKQQYPLSQHKRYIVKNGTPVLVPANPAALLKSKLLSAQSKIQVIFEPFMWKRSDSSAVRDENSEESVSRFFERHFGKEVVDYLIDPFVGGTSAAGPESLSIRHSFPELWNLEKRFGSIIAGALQSSVFGKKDKAGETKDVPRKNKHQRGSFSFQGGMQTLTDTLCKELGKDDIKLNAKVLTLAYSHDGSSPSQNWSITCTSNRKAQDVDAVIMTAPLGNVRDIQIKKKGNPFPLNFLPEVTYLPLSVLITTFKKENVKRPLEGFGVLVPSKEQQNGFKTLGTLFSSMMFPDRAPSDMHLYTTFIGGTRNRELAQASTDELTKIVTSDLRKLLGAEGEPAFVNHFFWSKGFPLYGHNYGSVLEAIDKMEKDLPGFFYAGNHRGGLSVGRAIASGCKAADLVISYLNNASDNSV

SEQ ID NO: 14 - protein, 506 aa, Fragaria sp.
MASPSQPHNHRSVKKVAVVGAGVSGLAAAYKLKSHGFDVTVLEAEGRAGGKLRSVSYNGLIWDEGANTMTEAETEVQTLLDSLGLRDKQQFPISQNKRYVARNGMPVLLPTNPIELIKSNFLSTKSKFQILLEPYLWKKKKVSDDHTQESVAGFFQRHFGKEVVDYLIDPFVAGTSAGDPESLSMPHSFPELWNIEKRYGSVITGTIRSKVSSRKEKRGDTKGSVEKGKRQRGSFSFQGGMQTLTDTLCKQLGKHELKLNSKVLSLSYSHDGSSTSENWSLSCVANDDKHSQSSSVDAIIMTAPLCSIKEMKITRRGTIFPLDFLPEVNYMPLSVLITSFKKENIKRPLEGFGVLVPSKEQENGLKTLGTLFSSMMFPDRAPSDQYLYTTFVGGSRNKELAKASKDELKQIVTSDIRQLLGAEGEPTFVNHYYWSKAFPLYGHNYDSVIEAIEKMEKNLPGLFYAGNHRGGLSVGKAIASGCKAADLVISYLESSSDGKILQQGSS

SEQ ID NO: 15 - protein, 509 aa, Citrus sp.
MASAPGEDNQRSAKRVAVVGAGVSGLAAAYKLKSNGVNVMVFEADERAGGKLRSISKDGLIWDEGANTMTESEMEVKGLLDDLGIREKQQFPISQYKRYVVRNGVPFLIPTNPIALITSNFLSAQSKFQIILEPFLWKKSDSAKVSAEDAKESVGGFFQRHFGREVVDFLIDPFVAGTSAADPESLVMRHSFPELWNLEKRYGSVIAGAIKSKFSARKEKSAEAKGSSEKKHRQRGSFSFLGGMQTLTDALCKALGKDEVCLKSKVLSLSYSHDGKSALENWSLSSSNQDKQSQGLSFDAVIMTASLCNVKEMKITKGGNLFPLDFLPEVIYMPLSVFITAFKKENVGKPLQGFGVLVPSKEQQNGLKTLGTLFSSMMFPDRAPKDLFLYTTFVGGSRNKELAKASTDELKQIVTSDLRQLLGVEGEPTFVNHFFWSKAFPLYGRDYDSVLEAIEKMEKNLPGFFYAGNHKGGLSVGKSIASGCKAAELVISYLENSSDDKMLKEGSSK

SEQ ID NO: 16 - protein, 509 aa, Citrus sp.
MTNKWRVDFSGSAKRVAVVGAGVSGLAAAYKLKSNGVNVMVFEADERAGGKLRSISKDGLIWDEGANTMTESEMEVKGLLDDLGIREKQQFPISQYKRYVVRNGVPFLIPTNPIALITSNFLSAQSKFQIILEPFLWKKSDSAKVSAEDAKESVGGFFQRHFGREVVDFLIDPFVAGTSAADPESLVMRHSFPELWNLEKRYGSVIAGAIKSKFSARKEKSAEAKGSSEKKHRQRGSFSFLGGMQTLTDALCKALGKDEVCLKSKVLSLSYSHDGKSALENWSLSSSNQDKQSQGLSFDAVIMTASLCNVKEMKITKGGNLFPLDFLPEVIYMPLSVFITAFKKENVGKPLQGFGVLVPSKEQQNGLKTLGTLFSSMMFPDRAPKDLFLYTTFVGGSRNKELAKASTDELKQIVTSDLRQLLGVEGEPTFVNHFFWSKAFPLYGRDYDSVLEAIEKMEKNLPGFFYAGNHKGGLSVGKSIASGCKAAELVISYLENSSDDKMLKEGSSK

SEQ ID NO: 17 - protein, 502 aa, Cicer sp.
MASSAKDNNSRSVKRVAVVGAGVSGLAAAYKLKSHGLDVTVFEAEGRAGGRLRTVSRDGLVWDEGANTMTENEAEVKSLIDALGLQEKQQYPLSQHKRYIVKNGMPLLVPANPAALLKSKLLSAQSKIRVIFEPFMWKRSDSSTVCDENSEESVSRFFERHFGKEVVDYLIDPFVGGTSAADPESLSMRHSFPELWNLEKRFGSIIGGALQSNLFGKRDKTGETKDAPRKSKHQRGSFSFQGGMQTLTDTLCKELGKDNLKLNAKVLTLAYSHNGSSPSENWSITCASNLTTQDVDAVIMTAPLGNVKDIQITKRGTPFTLNFFPEVTYLPLSVLITTFKKENVKRPLEGFGVLVPSKEEQNGFKTLGTLFSSMMFPDRAPSDLHLYTTFIGGTRNRELAQASTDELKKIVTSDLRKLLGAEGEPTFVNHFYWSKGFPLYGHNYGLVLEAIDKMEKGLPGFFYAGNHRGGLSVGRAIASGCKAADLVISYLNNASDNTVADK

SEQ ID NO: 18 - protein, 502 aa, Cucumis sp.
MASPAKQDRRTSRKKVAVVGAGVSGLAAAYKLKSHGFDVTVLEADERVGGKLRSVSYKGLIWDEGANTMTESEPEVQCLLDDLGLREKQQFPISQNKRYIVRNGVPVLVPTNPIALIKSNFLSAKSKFQIILEPFLWKKYDSSKVSDDGTDESVGGFFQRHFGQEVVDYLIDPIVAGTSAGDPDSLSMSHSFPELWNIEKRFGSIFAGLVLSKLSTKKESGGVRNGTTGKSKPRRGSFSFQNGMQTLTDTLSKELGEGVLKLRSEVLSLSYNAGKYASQNWSLIYSKDKNSKDLIADAVIMTAPVCSVREMKFMKGGIPFSLNFLPEVAYMPLSVMITTFRKESVKRPLEGFGVLVPSSEQQNGLRTLGTLFSSMMFPNRASNDEYLYTTFIGGSRNRELAKSSTDELKQIVTTDLRQLLGVEGEPTFINHFYWSKAFPLYGRNYDSVVKAIETMEKNLPGFFYAGNHRDGLSVGKSIASGCKAADLVISYLESSTDQSCAE

SEQ ID NO: 19 - protein, 497 aa, Cucumis sp.
MKSSQSSRKKVAVVGAGVSGLAAAYKLKSHGFDVTVLEADERVGGKLRSVSYKGLIWDEGANTMTESEPEVQCLLDDLGLREKQQFPISQNKRYIVRNGVPVLVPTNPIALIKSNFLSAKSKFQIILEPFLWKKYDSSKVSDDGTDESVGGFFQRHFGQEVVDYLIDPIVAGTSAGDPDSLSMSHSFPELWNIEKRFGSIFAGLVLSKLSTKKESGGVRNGTTGKSKPRRGSFSFQNGMQTLTDTLSKELGEGVLKLRSEVLSLSYNAGKYASQNWSLIYSKDKNSKDLIADAVIMTAPVCSVREMKFMKGGIPFSLNFLPEVAYMPLSVMITTFRKESVKRPLEGFGVLVPSSEQQNGLRTLGTLFSSMMFPNRASNDEYLYTTFIGGSRNRELAKSSTDELKQIVTTDLRQLLGVEGEPTFINHFYWSKAFPLYGRNYDSVVKAIETMEKNLPGFFYAGNHRDGLSVGKSIASGCKAADLVISYLESSTDQSCAE

SEQ ID NO: 20 - protein, 504 aa, Nicotiana sp.
MAPSAGEDKHSSAKRVAVIGAGVSGLAAAYKLKIHGLNVTVFEAEGKAGGKLRSVSQDGLIWDEGANTMTESEGDVTFLIDSLGLREKQQFPLSQNKRYIARNGTPVLLPSNPIDLIKSNFLSTGSKLQMLLEPILWKNKKLSQVSDSHESVSGFFQRHFGKEVVDYLIDPFVAGTCGGDPDSLSMHHSFPELWNLEKRFGSVILGAIRSKLSPKNEKKQGPPKTSANKKRQRGSFSFLGGMQTLTDAICKDLREDELRLNSRVLELSCSCTEDSAIDSWSIISASPHKRQSEEESFDAVIMTAPLCDVKSMKIAKRGNPFLLNFIPEVDYVPLSVVITTFKRENVKYPLEGFGVLVPSKEQQHGLKTLGTLFSSMMFPDRAPNNVYLYTTFVGGSRNRELAKASRTELKEIVTSDLKQLLGAEGEPTYVNHLYWSKAFPLYGHNYDSVLDAIDKMEKNLPGLFYAGNHRGGLSVGKALSSGCNAADLVISYLESVSTDSKRHC

SEQ ID NO: 21 - protein, 501 aa, Solanum sp.
MAPSAGEDKQKRVAVIGAGVSGLAAAYKLKVHGLNVTVFEAEGRAGGKLRSLSQDGLIWDEGANTMTESEGDVTFLLDSLGLREKQQFPLSQNKRYIARNGTPTLIPSNPFDLFKSNFLSTGSKLQMLFEPLLWKNKKLTKVSDKHESVSGFFQRHFGKEVVDYLIDPFVAGTCGGDPDSLSMHLSFPDLWNLEKRFGSVIVGAIQSKLSPIKEKKQGPPRTSINKKRQRGSFSFLGGMQTLTDAICKNLKEDELRLNSRVLELSCSCSGDSAIDSWSIFSASPHKRQAEEESFDAVIMTAPLCDVKSMKIAKRGNPFLLNFIPEVDYVPLSVVITTFKKESVKHPLEGFGVLVPSQEQKHGLKTLGTLFSSMMFPDRAPNNVYLYTTFVGGSRNRELAKASRTELKEIVTSDLKQLLGAEGEPTYVNHLCWSKAFPLYGHNYDSVLDAIDKMEKSLPGLFYAGNHKGGLSVGKALSSGCNAADLVISYLEAVSADTKNHS

SEQ ID NO: 22 - protein, 508 aa, Arabidopsis sp.
MASGAVADHQIEAVSGKRVAVVGAGVSGLAAAYKLKSRGLNVTVFEADGRVGGKLRSVMQNGLIWDEGANTMTEAEPEVGSLLDDLGLREKQQFPISQKKRYIVRNGVPVMLPTNPIELVTSSVLSTQSKFQILLEPFLWKKKSSKVSDASAEESVSEFFQRHFGQEVVDYLIDPFVGGTSAADPDSLSMKHSFPDLWNVEKSFGSIIVGAIRTKFAAKGGKSRDTKSSPGTKKGSRGSFSFKGGMQILPDTLCKSLSHDEINLDSKVLSLSYNSGSRQENWSLSCVSHNETQRQNPHYDAVIMTAPLCNVKEMKVMKGGQPFQLNFLPEINYMPLSVLITTFTKEKVKRPLEGFGVLIPSKEQKHGFKTLGTLFSSMMFPDRSPSDVHLYTTFIGGSRNQELAKASTDELKQVVTSDLQRLLGVEGEPVSVNHYYWRKAFPLYDSSYDSVMEAIDKMENDLPGFFYAGNHRGGLSVGKSIASGCKAADLVISYLESCSNDKKPNDSL

SEQ ID NO: 23 - protein, 512 aa, Arabidopsis sp.
MESGAVGDHDTKFESISGKRVAVVGAGVSGLAAAYKLKSRGLNVTVFEADERAGGKLTSVMQNGLIWDQGANTMTEAEPEVGSLLDDLGLRDKQQFPISQKKRYIVRNGLPMMLPTNPIELVTSSVLSTQAKIQILLEPFLWKKNDSSSKVSDASAEESVSGFFQRHFGQEVVDYLIDPFVGGTSAADPDSLSMKHSFPDLWNVEKSFGSIIVGAIRTKLAAKGGKSGEAKSSPGTKRGSRRSFSFKGGMQILPDMLCKSLSHDEINLDSKVLSLSYNSGSRQENWSLSCVSHNETQRQNLHYDAVVMTAPLCNVKEMKVTKGGQPFLLNFLPEINYMPLSVLITTFTKEKVKRPLEGFGVLIPSKEKKHGFKTLGTLFSSMMFPDRCPSDLHLYTTFIGGSRNQELAKASTDELKQVVTSDLQRLLGVEGEPVSVNHYYWRKAFPLYDSSYGSVMEAIDKMEKDLPGFFYAGNHRGGLSIGKSIASGCKAADLVISYLESCSNDKKPDESL

SEQ ID NO: 24 - protein, 501 aa, Arabidopsis sp.
MASGAVADHQIEAVSGKRVAVVGAGVSGLAAAYKLKSRGLNVTVFEADGRVGGKLRSVMQNGLIWDEGANTMTEAEPEVGSLLDDLGLREKQQFPISQKKRYIVRNGVPVMLPTNPIELVTSSVLSTQSKFQILLEPFLWKKKSSKVSDASAEESVSEFFQRHFGQEVVDYLIDPFVGGTSAADPDSLSMKHSFPDLWNSFGSIIVGAIRTKFAAKGGKSRDTKSSPGTKKGSRGSFSFKGGMQILPDTLCKSLSHDEINLDSKVLSLSYNSGSRQENWSLSCVSHNETQRQNPHYDAAPLCNVKEMKVMKGGQPFQLNFLPEINYMPLSVLITTFTKEKVKRPLEGFGVLIPSKEQKHGFKTLGTLFSSMMFPDRSPSDVHLYTTFIGGSRNQELAKASTDELKQVVTSDLQRLLGVEGEPVSVNHYYWRKAFPLYDSSYDSVMEAIDKMENDLPGFFYAGNHRGGLSVGKSIASGCKAADLVISYLESCSNDKKPNDSL

SEQ ID NO: 25 - protein, 547 aa, Arabidopsis sp.
MGLIKNGTLYCRFGISWNFAAVFFSTYFRHCFRLVRDFDSELLQIAMASGAVADHQIEAVSGKRVAVVGAGVSGLAAAYKLKSRGLNVTVFEADGRVGGKLRSVMQNGLIWDEGANTMTEAEPEVGSLLDDLGLREKQQFPISQKKRYIVRNGVPVMLPTNPIELVTSSVLSTQSKFQILLEPFLWKKKSSKVSDASAEESVSEFFQRHFGQEVVDYLIDPFVGGTSAADPDSLSMKHSFPDLWNSFGSIIVGAIRTKFAAKGGKSRDTKSSPGTKKGSRGSFSFKGGMQILPDTLCKSLSHDEINLDSKVLSLSYNSGSRQENWSLSCVSHNETQRQNPHYDAAPLCNVKEMKVMKGGQPFQLNFLPEINYMPLSVLITTFTKEKVKRPLEGFGVLIPSKEQKHGFKTLGTLFSSMMFPDRSPSDVHLYTTFIGGSRNQELAKASTDELKQVVTSDLQRLLGVEGEPVSVNHYYWRKAFPLYDSSYDSVMEAIDKMENDLPGFFYAGNHRGGLSVGKSIASGCKAADLVISYLESCSNDKKPNDSL

SEQ ID NO: 26 - protein, 467 aa, Ambrosia sp.
GCAAAYKLKLHGLNVTVFEADERVGGKLRSVSQDGLIWDEGANTMTESEADVSSLIDDLGLRDKQQFPISQHKRYIVRNGKPVLIPSNPIALIRSSFLSTQSKVQILLEPFLWKKTKSSDEPESVGGFFQRHFGKEVVEYLIDPVVAGTSGGDPESLSMRHAFPELWDLERRFGSIISGAFQSMVSSRGGKRKPSGNSKRRRGSFSFFGGMQTLTDALSKEIGPHEINLQSKVLEMSYSCDDNAVGNWSIYCAPDQNKQFQQSFDAVIMTAPLNNLKEMKITKTGSPFLLNFIPEVSYLPISVIISTFKKENVKQPLEGFGVLVPAKEQENGLRTLGTLFSSMMFPDRASEDVYLYTTFVGGSRNKELAKASRDELKQIVTSDLRQLLGTEGEPKFLTHYYWSKAFPLYGRDYGSVIEAIEKMEKELPGYFYAGNHKGGLSVGKAISSGCKAAESVIAYLDSYSNQK

SEQ ID NO: 27 - protein, 542 aa, Setaria sp.
MLSSSTTTASPASSHPYRPAYPRASLRPVLAMAGSDDPRAAPARSVAVIGAGVSGLAAAYRLRKSGVNVTVFEAADRAGGKIRTNSEAGFLWDEGANTMTEGELEVSRLIDDLGLQDRQQYPNSQHKRYIVKDGAPALIPADPISLMKSSVLSTKSKLALFLEPFLYKKSNTRNSGKVSDEHLSESVGSFFERHFGREVVDYLIDPFVAGTSAGDPESLSIRHAFPALWNLERKYHSIIVGAILSKLTAKGDPVKTGSDLSGKRRNRRASFSFHGGMQSLINALHNEVGDDNVKLGTEVLSLACTFDGLPSTGGWSISVDSKDAGSKDLAKNQTFDAVIMTAPLSNVQRMKFRKGGAPFVLDFLPKVNYLPLSLMVTAFKKEDVKKPLEGFGVLIPYKEQQKHGLKTLGTLFSSMMFPDRAPDDQYLYTTFVGGSHNRDLAGAPTSILKQLVTSDLKKLLGVEGQPTFVKHIYWRNAFPLYGRDYGSVLDAIEKMEKNLPGFFYAGNNKDGLAVGNVIASGSKAAELAISYLESQTKHNNSH

SEQ ID NO: 28 - protein, 544 aa, Sorghum sp.
MLARTATVSSTSSHSHPYRPTSARSLRLRPVLAMAGSDDSRAAPARSVAVVGAGVSGLVAAYRLRKSGVNVTVFEAADRAGGKIRTNSEGGFLWDEGANTMTEGELEASRLIDDLGLQDKQQYPNSQHKRYIVKDGAPALIPSDPISLMKSSVLSTKSKIALFFEPFLYKKANTRNPGKVSDEHLSESVGSFFERHFGREVVDYLIDPFVAGTSAGDPESLSICHAFPALWNLERKYGSVVVGAILSKLTAKGDPVKTRRDSSAKRRNRRVSFSFHGGMQSLINALHNEVGDDNVKLGTEVLSLACTLDGAPAPGGWSISDDSKDASGKDLAKNQTFDAVIMTAPLSNVQRMKFTKGGAPFVLDFLPKVDYLPLSLMVTAFKKEDVKKPLEGFGVLIPYKEQQKHGLKTLGTLFSSMMFPDRAPDDQYLYTTFVGGSHNRDLAGAPTSILKQLVTSDLKKLLGVQGQPTFVKHIYWGNAFPLYGHDYNSVLEAIEKMEKNLPGFFYAGNNKDGLAVGSVIASGSKAADLAISYLESHTKHNNLH

SEQ ID NO: 29 - protein, 477 aa, Arabidopsis sp.
MASGAVADHQIEAVSGKRVAVVGAGVSGLAAAYKLKSRGLNVTVFEADGRVGGKLRSVMQNGLIWDEGANTMTEAEPEVGSLLDDLGLREKQQFPISQKKRYIVRNGVPVMKKSSKVSDASAEESVSEFFQRHFGQEVVDYLIDPFVGGTSAADPDSLSMKHSFPDLWNVEKSFGSIIVGAIRTKFAAKGGKSRDTKSSPGTKKGSRGSFSFKGGMQILPDTLCKSLSHDEINLDSKVLSLSYNSGSRQENWSLSCVSHNETQRQNPHYDAVIMTAPLCNVKEMKVMKGGQPFQLNFLPEINYMPLSVLITTFTKEKVKRPLEGFGVLIPSKEQKHGFKTLGTLFSSMMFPDRSPSDVHLYTTFIGGSRNQELAKASTDELKQVVTSDLQRLLGVEGEPVSVNHYYWRKAFPLYDSSYDSVMEAIDKMENDLPGFFYAGNHRGGLSVGKSIASGCKAADLVISYLESCSNDKKPNDS

SEQ ID NO: 30 - protein, 544 aa, Zea sp.
MLALTASASSASSHPYRHASAHTRRPRLRAVLAMAGSDDPRAAPARSVAVVGAGVSGLAAAYRLRQSGVNVTVFEAADRAGGKIRTNSEGGFVWDEGANTMTEGEWEASRLIDDLGLQDKQQYPNSQHKRYIVKDGAPALIPSDPISLMKSSVLSTKSKIALFFEPFLYKKANTRNSGKVSEEHLSESVGSFCERHFGREVVDYFVDPFVAGTSAGDPESLSIRHAFPALWNLERKYGSVIVGAILSKLAAKGDPVKTRHDSSGKRRNRRVSFSFHGGMQSLINALHNEVGDDNVKLGTEVLSLACTFDGVPALGRWSISVDSKDSGDKDLASNQTFDAVIMTAPLSNVRRMKFTKGGAPVVLDFLPKMDYLPLSLMVTAFKKDDVKKPLEGFGVLIPYKEQQKHGLKTLGTLFSSMMFPDRAPDDQYLYTTFVGGSHNRDLAGAPTSILKQLVTSDLKKLLGVEGQPTFVKHVYWGNAFPLYGHDYSSVLEAIEKMEKNLPGFFYAGNNKDGLAVGSVIASGSKAADLAISYLESHTKHNNSH

SEQ ID NO: 31 - protein, 544 aa, Zea sp.
MLALTASASSASSHPYRHASAHTRRPRLRAVLAMAGSDDPRAAPARSVAVVGAGVSGLAAAYRLRQSGVNVTVFEAADRAGGKIRTNSEGGFVWDEGANTMTEGEWEASRLIDDLGLQDKQQYPNSQHKRYIVKDGAPALIPSDPISLMKSSVLSTKSKIALFFEPFLYKKANTRNSGKVSEEHLSESVGSFCERHFGREVVDYFVDPFVAGTSAGDPESLSIRHAFPALWNLERKYGSVIVGAILSKLAAKGDPVKTRHDSSGKRRNRRVSFSFHGGMQSLINALHNEVGDDNVKLGTEVLSLACTFDGVPALGRWSISVDSKDSGDKDLASNQTFDAVIMTAPLSNVRRMKFTKGGAPVVLDFLPKMDYLPLSLMVTAFKKDDVKKPLEGFGVLIPYKEQQKHGLKTLGTLFSSMMFPDRAPDDQYLYTTFVGGSHNRDLAGAPTSILKQLVTSDLKKLLGVEGQPTFVKHVYWGNAFPLYGHDYSSVLEAIEKMEKNLPGFFYAGNSKDGLAVGSVIASGSKAADLAISYLESHTKHNNSH

SEQ ID NO: 32 - protein, 509 aa, Lemna paucicostata sp.
MEADAGKGSAGGAQILSHDSVRSVAVIGGGISGLAAAYKLKSNGFRAVVFEAEGKAGGKIRSGSQEGLIWDEGANTMAESVEAGELFDDVGIREKQQYPLSQSKRYVVRNGVPVMIPSDPISLIKSNLLSTKAKFRMFLEPFFSYRRVKPSKVSDEKLSESVGEFFQRHFGKEVVDYLIDPFVAGTSGGDPESLSMPHAFPEIWNLQEKYGSVILGAIQSKFLDKRKGDRTERATVKKRRPRGSFSFHGGMQTLIDVLCAKVGEENLELNSKVLSLACGHEGDPSFDSWSISVASNNGSQKDLLTKSFFDAVIMTAPLGNVEDMKFTKKGSPFALDFLPQVTYLPLSVLITSFKRENVKRPLEGFGVLVPSKEQEGGFKTLGTLFSSAMFPDRAPSDQYLYTTFIGGSRNRDLAGASLEELKQIVLSDLHKLLGVVGEPSFIKHVYWSKAFPLYGRDYGLVMEAIDRMERNLPGFYYAGNHRDGLSVGKAIASGFRAADLAISYINSSV

SEQ ID NO: 33 - protein, 544 aa, Lemna paucicostata sp.
MASSAAISPLHFSPAPPRRRELSHRCRVRCSIAGKPPATAANDSSATSVTGGEPVRRLRADCVIVGAGISGLCTAQALTVRPAAGRSSAPDVLVTEARDRVGGNITTVERDGYLWEEGPNSFQPSDAVLTMAVDSGLKDDLVLGDPDAPRFVLWKGKLRPVPAKPTDLPFFDLMSIGGKIRAGFGALGLRPSPPSREESVEEFVRRNLGDEVFERLIEPFCSGVYAGDPSKLSMKAAFGKVWKLEQTGGSIIGGTFKAIQERSKNPKPPRDPRLPTPKGQTVGSFSKGLAMLPNAIASRLGDKVKLSWKLSGIKKSENGGYALTYDTPEGLTSVNADCVVLTIPSYVAGDLLRPLSNEAADALTKFYYPPVAAVTISYPSDSIRSECLIDGQLKGFGQLHPRSQGVETLGTIYSSSLFPNRAPPGRVLLLNYIGGSTNTAIVSKTESELVEAVDRDLRKMLVKVNAADPAVQGVRVWPRAIPQFLIGHTDLLDAATRSLDRAGYGGLILGGNYVAGVALGRCIEGAYDTAAKVNSFLSKYARSF

SEQ ID NO: 34 - protein, 482 aa, Populus sp.
MTAAAKDDDFGSKQSKNVAVIGAGVSGLAAAYKLKSNGVKVTVFEAEGRAGGKLRSVSHHDLVWDEGANTMTESEVEVKSLLDDLGLREKQQFPIAQNKRYIVRNGMPVLIPTNPVALIKSNFLSAQSKLQIILEPFLWKKNESSKVSDADIQESVGEFFQRHFGKEVVDYLIDPFVAGTSAGDPESLSARHNFPDLWNLEKRFGSIIAGAVKAKLSAKKEKNGEKKGSSEKKKRQHGSFSFLGGMQTLTDTLCTELGKDGVKLESKVLSLSYSYDGKSTFENWSVSYASKGGKHAQASSYDAVIMTAPLCNVKEININKGRNRFSLDFLPQMSYMPLSVIITTFKKEDVKRPLEGFGVLVPSKEQQNGLKTLGTLFSSMMFPDRAPKDSYLYTTFVASTVLDCSHYYWSKAFPLYGKNYDLVLEGIERMEKNLPGFFYAGNHRGGLSVGKAIASGCKAADLVISHLNSSADDKMLKKGSQS

SEQ ID NO: 35 - protein, 569 aa, Capsella sp.
MHDAGDSHDPFEIHKHVDVELKSGLDAKTLNKGPLDKRLKIIGPEELKTRFDAELSWLLLITMASAKVADNDTKFEAVSGRRVAVVGGGVSGLAAAYKLKSKGLNVTVFEADGRAGGKLRSVMHNGLIWDEGANTMTEAEPEVGTLLDDLGLREKQQFPISQKKRYIVRNGVPVMIPTNPIDLVTSTVLSTQSKFEILLEPFLWKKNDLSSKVSDASAAESVSGFFRRHFGQEVVDYLIDPFVGGTSAADPDSLSIVFVYCLILLILISLPTIHDQMKHSFPDLWNVEKSFGSVIVGAIRTKLAAKGVQILPDMLCKGLSHDELNLDSKVLSLSYNSGSRQENWSLSCVSHNEMQRQNLHYDAVIMTAPLCNVKEMDVMKGGQPFQLNFLPEIKYMPLSVIITTFTKEKVKRPLEGFGVLIPSKEQKHGFKTLGTLFSSMMFPDRCPSDLHLYTTFIGGSRNQELAKASTDELKQVVTSDLQRLLGVEGEPEFVNHYYWRKAFPLYDSSYDSVMEAIDKMEKDLPGFFYAGNHRGGLSVGKSIASGCKAADLVISYLESCSNDKKPEDS

SEQ ID NO: 36 - protein, 544 aa, Brachypodium sp.
MLTSATASPSASTRFSSTCRPCRSDSVPARRPRPVLAMAASDDPRAAPARSVAVVGAGVSGLVAAHRLRKSGVRVTVFEADDRAGGKIRTNSDSGFLWDEGANTMTESALEASRLIDDLGLQDKQQYPNSQHKRYTVKDGAPTLIPSDPIALMKSTVLSTKSKFKLFLEPFLYEKSHTRNSQKVSDNHLSESVGSFFERHFGKEVVDYLIDPFVAGTSAGDPESLSIRHAFPGLWDLEKKYGSIIVGAILSKLTAKGDSTKKADTSSGKGRNKQASFSFHGGMQTLVEGLHKDVGDGNVKLGTQVLSLACSCDRLSASDGWSISVNSKDASSKLAAKNQLFDAVIMTAPLSNVQRMKFTKGGVPFVLDFLPKVDYLPLSLMVTAFRKEDVKRPLEGFGVLIPYKEQQKYGLKTLGTLFSSMMFPDRAPNDQHLFTTFVGGSHNRDLAAAPTAILKQLVTSDLRKLLGVEGQPTFVKHVHWKNAFPLYGHDYDLALEAIGKMENELPGFFYAGNNKDGLAVGNVIASGSKTADLVISYLESHQAR

SEQ ID NO: 37 - protein, 506 aa, Oryza sp.
MAASDDPRGGRSVAVVGAGVSGLAAAYRLRKRGVQVTVFEAADRAGGKIRTNSEGGFIWDEGANTMTESELEASRLIDDLGLQGKQQYPNSQHKRYIVKDGAPTLIPSDPIALMKSTVLSTKSKLKLFLEPFLYEKSSRRTSGKVSDEHLSESVIFLCICRDNQVVDYLIDPFVAGTSGGDPESLSIRHAFPALWNLENKYGSVIAGAILSKLSTKGDSVKTGGASPGKGRNKRVSFSFHGGMQSLIDALHNEVGDGNVKLGTEVLSLACCCDGVSSSGGWSISVDSKDAKGKDLRKNQSFDAVIMTAPLSNVQRMKFTKGGVPFVLDFLPKVDYLPLSLMVTAFKKEDVKKPLEGFGALIPYKEQQKHGLKTLGTLFSSMMFPDRAPNDQYLYTSFIGGSHNRDLAGAPTAILKQLVTSDLRKLLGVEGQPTFVKHVHWRNAFPLYGQNYDLVLEAIAKMENNLPGFFYAGNNKDGLAVGNVIASGSKAADLVISYLESCTDQDN

SEQ ID NO: 38 - protein, 494 aa, Picea sp.
MAEHIHTDQNDKRPLKSVAVVGAGISGLAAAYRLKSQGLAVTIFEADGTTGGKIKSFAQNGLIWEKGANTMTETEPEVGKLIDDLGIRGKQQFPIMQSKRYIVRDGKPQLLPSNPVAFIGSKTLSAQAKLNIFLEPILWKHKNSKEKTPNSPDIYQEESVGDFFRRHFGQEVVDYIVDPFVAGTAGADAESLSIRHMFPEIWDLEERFGSIITGAIKSSWSRKKAQRDAKHVTHGQKRQRGSFSFMGGLQTLTNALSKKLGEESLRMHCSVLSLSCNLQGNPPHNNWSVCYARNDASYKEPLKEQSFDAVVMTVTYLPMSIIITTFKKQDVKHPLEGFGILVPSKEEKNGFQTLGTLFSSNMFPDRAPTDQYLFTTFIGGNRNRKLAKSQLKDLQEVAVNDLNKILGVGSDPLSVKHIYWNEAFPLYSLDYNSVVAAIDKLGKSLPGIYFAGNYRGGLSVGKALTSGFKAADLAISDFNSKGLCTMIGTDHEVK

SEQ ID NO: 39 - protein, 404 aa, Solanum sp.
MAPSAGEDKQNCPKRVAVIGAGVSGLAAAYKLKIHGLNVTVFEAEGRAGGKLRSLSQDGLIWDEGANTMTESEGDVTFLLDSLGLREKQQFPLSQNKRYIARNGTPTLIPSNPIDLIKSNFLSTGSKLQMLFEPLLWKNNKLTKVSDEHESVSGFFQRHFGKEVVDYLIDPFVAGTCGGDPDSLSMHLSFPELWNLEKRFGSVIVGAIRSKLSPIKEKKQGPPKTSVNKKRQRGSFSFLGGMQTLTDAICKDLKEDELRLNSRVLELSCSCSGDSAIDSWSIFSASPHKRQAEEESFDAVIMTAPLCDVKSMKIAKRGNPFLLNFIPEVDYVPLSVVITTFKKESVKHPLEGFGVLVPSQEQKHGLKTLGTLFSSMMFPDRAPNNVYLYTTFVGGSRNRELAKA

SEQ ID NO: 40 - protein, 505 aa, Oryza sp.
MAASDDPRGGRSVAVVGAGVSGLAAAYRLRKRGVQVTVFEAADRAGGKIRTNSEGGFIWDEGANTMTESELEASRLIDDLGLQGKQQYPNSQHKRYIVKDGAPTLIPSDPIALMKSTVLSTKSKLKLFLEPFLYEKSSRRTSGKVSDEHLSESMFSDQREYICSVIFLCICRDNQVVDYLIDPFVAGTSGGDPESLSIRHAFPALWNLENKYGSVIAGAILSKLSTKGDSVKTGGASPGKGRNKRVSFSFHGGMQSLIDALHNEVGDGNVKLGTEVLSLACCCDGVSSSGGWSISVDSKDAKGKDLRKNQSFDAVIMTAPLSNVQRMKFTKGGVPFVLDFLPKVDYLPLSLMVTAFKKEDVKKPLEGFGALIPYKEQQKHGLKTLGTLFSSMMFPDRAPNDQYLYTSFIGGSHNRDLAGAPTAILKQLVTSDLRKLLGVEGQPTFVKHVHWRNAFPLYGQNYDLVLEAIAKMENNLPGFFYAGKSMKAPLLYKRNLRYLKHIPVD

SEQ ID NO: 41 - protein, 505 aa, Oryza sp.
MAASDDPRGGRSVAVVGAGVSGLAAAYRLRKRGVQVTVFEAADRAGGKIRTNSEGGFIWDEGANTMTESELEASRLIDDLGLQGKQQYPNSQHKRYIVKDGAPTLIPSDPIALMKSTVLSTKSKLKLFLEPFLYEKSSRRTSGKVSDEHLSESMFSDQREYICSVIFLCICRDNQVVDYLIDPFVAGTSGGDPESLSIRHAFPALWNLENKYGSVIAGAILSKLSTKGDSVKTGGASPGKGRDKRVSFSFHGGMQSLIDALHNEVGDGNVKLGTEVLSLACCCDGVSSSGGWSISVDSKDAKGKDLRKNQSFDAVIMTAPLSNVQRMKFTKGGVPFVLDFLPKVDYLPLSLMVTAFKKEDVKKPLEGFGALIPYKEQQKHGLKTLGTLFSSMMFPDRAPNDQYLYTSFIGGSHNRDLAGAPTAILKQLVTSDLRKLLGVEGQPTFVKHVHWRNAFPLYGQNYDLVLEAIAKMENNLPGFFYAGKSMKAPLLYKRNLRYLKHIPVD

SEQ ID NO: 42 - protein, 405 aa, Eutrema sp.
MAPDAVADHDKKFEALSGKRVAVVGAGVRLKSRGLNVTVFEADGRAGGKLRSVMHKGLIWDEGANTMTEAEPEVGSLLDDLGLREKQQFPISQKKRYIVRNGLPVMIPTNPIALVTSSVLSTHSKFQILLEPFLWKKNDSSSKVSDASTVESVSGFFQRHFGQEVVDYLIDPFVGGTSAADPDSLSMKHTFPDLWNIEKSFGSIIVGAIRTKFAAKGGKSGETRTSPGTKKGSRGSFSFKGGMQILPDMLCKDLSHDELNLDSKVLSLSYNSGSRQENWSLSCVSHNETLRQNLHYDAVVMTAPLCNVKEMKVVKGGQPFQLNFIPEIKYMPLSVIITTFTKEKVKRPLEGFGVLIPSKEEKHGFKTLGTLFSSMMFPDRCPSDLHLYTTFVGGSRNQELAKAST

SEQ ID NO: 43 - protein, 520 aa, Selaginella sp.
MAMAEGETAPVLGSVAVVGAGASGLAAAYRLRAAGVSVTVYEAENSIGGKLKSVSENGFIWEKGPNTMTENDPSISRMFDDLHLRDKQQFPVEQKKRYIVRNASPTMLPSNPLGFITTGLFSAQAKLKLLTEPFSWKRTKAESNEDESVGAFMERHFGDEIVDYAVDPFVAGTSGSDPSSISIRHSFPELWSLEKNYGSLFVGAIKSGFSKKKKQKLRPVEFEDEDSDFPARTRPRRGGSFSFVGGMQTLANELVSRIGKEKFKLNTFVTGLACNQQGNPSRQSWTVTGLETSGKRSKRSDKTFDAVIMTAPVDDVRAMKVVKDGKPYALDYLPTVIYEPMSVLITMFNKDSVKRALPGFGVLVPSKEQQANGFQTLGTLFSSFMFPDRAPEDQLLFTTFIGGSRNTLLASRSKEELLDITLKDLSRLIGVEGQPTAIRHVYWEKAFPRYSIGYDNVLHSIQKLESDLPGLFYAGNHRGGLAVGKTIVSGLDAAEQVLQYLQGSGGKKVFTMASLEQPVS

SEQ ID NO: 44 - protein, 514 aa, Selaginella sp.
MAMAEGETVPVLGSVAVVGAGASGLAAAYRLRAAGVSVTVYEAENSIGGKLKSVSENGFIWEKGPNTMTENDPSISRMFDDLHLRDKQQFPVEQKKRYIVRNASPTMLPSNPLGFITTGLFSAQAKLKLLTEPFSWKRTKAESNEDESVGAFMERHFGDEIVDYAVDPFVAGTSGSDPSSISIRHSFPELWSLEKNYGSLFVGAIKSGFSKKKKQKLRPVEFEDEDSDFPARTRPRRGGSFSFVGGMQTLANELVSRIGKEKFKLNTFVTGLACNQQGNPSRQSWTVTGLETSGKRSKRSDKTFDAVIMTAPVDDVRTMKVVKDGKPYALDYLPTVIYEPMSVLITMFNKDSVKRALPGFGVLVPSKEQQANGFQTLGTLFSSFMFPDRAPEDQLLFTTFIGGSRNTLLASRSKEELLDVTLKDLSRLIGVEGQPTAMRHVYWEKAFPRYSIGYDNVLNSIQKLESDLPGLFYAGNHRGGLAVGKTIVSGLDAAEQVLQYLQGSGGKKVFTMAS

SEQ ID NO: 45 - protein, 230 aa, Amaranthus sp.
MVIQSITHLSPNLALPSPLSVSTKNYPVAVMGNISEREEPTSAKRVAVVGAGVSGLAAAYKLKSHGLSVTLFEADSRAGGKLKTVKKDGFIWDEGANTMTESEAEVSSLIDDLGLREKQQLPISQNKRYIARDGLPVLLPSNPAALLTSNILSAKSKLQIMLEPFLWRKHNATELSDEHVQESVGEFFERHFGKEFVDYVIDPFVAGTCGGDPQSLSMHHTFPEVWNIEK

SEQ ID NO: 46 - protein, 229 aa, Amaranthus sp.
MVIQSITHLSPNLALPSPLSVSTKNYPVAVMGNISEREEPTSAKRVAVVGAGVSGLAAAYKLKSHGLSVTLFEADSRAGGKLKTVKKDGFIWDEGANTMTESEAEVSSLIDDLGLREKQQLPISQNKRYIARDGLPVLLPSNPAALLTSNILSAKSKLQIMLEPFLWRKHNATELSDEHVQESVGEFFERHFGKEFVDYVIDPFVAGTCGDPQSLSMHHTFPEVWNIEK

SEQ ID NO: 47 - protein, 396 aa, Zea sp.
MKSSVLSTKSKIALFFEPFLYKKANTRNSGKVSEEHLSESVGSFCERHFGREVVDYFVDPFVAGTSAGDPESLSIRHAFPALWNLERKYGSVIVGAILSKLAAKGDPVKTRHDSSGKRRNRRVSFSFHGGMQSLINALHNEVGDDNVKLGTEVLSLACTFDGVPALGRWSISVDSKDSGDKDLASNQTFDAVIMTAPLSNVRRMKFTKGGAPVVLDFLPKMDYLPLSLMVTAFKKDDVKKPLEGFGVLIPYKEQQKHGLKTLGTLFSSMMFPDRAPDDQYLYTTFVGGSHNRDLAGAPTSILKQLVTSDLKKLLGVEGQPTFVKHVYWGNAFPLYGHDYSSVLEAIEKMEKNLPGFFYAGNNKDGLAVGSVIASGSKAADLAISYLESHTKHNNSH

SEQ ID NO: 48 - protein, 414 aa, Aegilops sp.
MTESALEASRLIDDLGLEDRLQYPNSQHKRYTVKDGAPALFKLFLEPFLYEKSSTRNSKKVSDEHLRESVGSFFERHFGREVVDYLIDPFVAGTSAGDPESLSIRHAFPGLWNLEKKYGSLIVGAILSKLTAKGDSSKKGGASSGKGRSKRASFSFHGGMQTLVDALHKEVGDSNVKLGTQVLSLACNCDELSASDGWSIFVDSKDASSKELAKNQSFDAVIMTAPLSNVQRMRFTKGGAPFVLDFLPKVDYLPLSLMVTAFKKEDVKRPLEGFGVLIPFKEQQKHGLKTLGTLFSSMMFPDRAPNDQYLFTTFIGGSHNRDLAGAPTAILKQFVTSDLTKLLGVVGQPTFVKHIHWRNAFPLYGHDYDSALEAIGKMERNNKDGLAVGNVIASGSNTADLVISYLESGIKQVS

SEQ ID NO: 49 - protein, 304 aa, Genlisea sp.
GFPAKNVAVIGAGVSGLSAAYKLKLNGLNVTVFEADGRAGGKIRTSSQDSLIWDEGANTMTESEEEVGFLLDNLGLREKQQFPLSQQKRYVVKNGKPALLPSNPFALIASNILSSSSKLQIFLEPFLWKQRNSEETQAEESVGGFFQRHFGKEVVDYLVDPFVAGTSGGDPESLSMRHAFPDLWNLEKRFGSVMSGAVLSKLSAGRGASEKNKGSSTNERRKRKSFSFIGGMQTLTDALSNEIGENELKLRSKVLGLSSNNVKSSKVDSWSISYASSDGKSVSLDTGFDAVIMTAPLSDVKQMK

SEQ ID NO: 50 - protein, 265 aa, Amborella sp.
MSSKGEEKGKHEATRRKPKHQRGSFSFQGGLQTLTDKLAEELGNENVKLHSKVLSLSYGDGNGDSFSNWSVSYIKNQSDQRKLLLKQSFDAVVMTAPIRNMQEMQISKCGKPYMLDFLPNVMYLPLSIIVTTFKKENVKLPLEGFGVLVPSKEQGSGLRTLGTLFSSMMFPDRAPKDQYLYTTFVGGSRNRNLASASLDELREVVTCDLKKLLGVVGAPTFVRHVYWGDAFPLYGHNYDMVLKAIEKMEQNLPGFFYAGKFSLWC

SEQ ID NO: 51 - protein, 469 aa, Rhodothermus sp.
MASVGIIGAGIAGLTAAYELHRRGLEVTVFEATDRIGGFIQSERIDGFLVELGPQTLQRTSGDFEELLRQVDLEDACIPARPVAANRFIVRGGQPIPLPRSPRELLRTPLLSPRARLRLLAEPFIHRAHRSTEESVAKFTRRRLGPEVLDYLVEPFVAGIFAGDPEQLSVRYAFPKLFELEQQYGSLFWGLIRDRMKQRYHPAPRRSMFSFVEGLHMLPRALAERLPAHAIVRNAEVLAIRWDEKNPWTLTFRQHGRASTRFFDIIVCAVPLHRLAQLRIHPPVDRRPLSTVEHPPIALVALGFRREQVAHPLDGFGMLVPAVERDFQILGTLFSSSLFPDRAPEGHVLLTTFVGGMRHPELALLPEDRLEALVLQDLRRLLGISGAPVFRHVWRWERSIPQYRLGYDAVLACVHDVEMSRSGLFLAGNYMEGISVIDALHTGLKAARAIIQHLREEAAGGLAKLVLGD

SEQ ID NO: 52 - protein, 461 aa, Salinibacter sp.
MPNVGIIGAGISGLAAAYRLQEHGHSVRLLEASGHTGGVIRSESSEGFLVEHGPNSIRAGAAGLETLIDALDLHEDRVWANDAADTRYVVRDGRPTPLPRSVGSFLTTDLFSTRAKLRLLAEPFIGRAAAEEESVARFTERRLGPEVLNYAVAPFVGGVFAGRPDDLSVQHAFRRLAALEEESGSLLLGAIRRALTSDDGAPPDTPSGLFSFRNGLQTLPNALADTLGDRIRLNAPVHALAHDGTAWRVTVSPPDAPAHTRSFDALVCTVPLHRLAAMEIDTPVDLAPLGEVTYPPLSVLALGYGRDAIDHALDGFGMLVPPVEDTLDVLGTIFSSTLFPGRAPEGHVLLTTFVGGARAPHHATSDAAALQARVARDLDSLLGVDASPVFRRLVHWPHAIPQYELGYGTVKDTFDALEAAHPHLAFAGNYRAGVSVGDALTSGLEAADRLLETDERAAQPH

SEQ ID NO: 53 - protein, 461 aa, Salinibacter sp.
MPNVGIIGAGISGLAAAYRLQEHGHSVRVLEASGHTGGVIRSESSEGFLVEHGPNSIRAGAAGLETLIDALDLHEDRVWANDAADTRYVVRDGRPTPLPRSVGSFLTTDLFSTRAKLRLLAEPFIGRAAAEDESVARFTERRLGPEVLNYAVAPFVGGVFAGRPDDLSVQHAFRRLAALEEESGSLLLGAIRRALTSDDGAPPDTPSGLFSFRNGLQTLPNALADTLGDRIRLNAPVHALTHDGTAWRVTVSPPDAPAHTRSFDALVCTVPLHRLAAMEIDTPVDLAPLGEVTYPPLSVLALGYERDAIDHALDGFGMLVPPVEDTLDVLGTIFSSTLFPGRAPEGHVLLTTFVGGARAPHHATSDAAALQARVARDLDSLLGVDASPVFRRLVHWPHAIPQYELGYGTVKDTFDALEAAHPHLAFAGNYRAGVSVGDALTSGLEAADRLLETDERATQPH

SEQ ID NO: 54 - protein, 203 aa, Zea sp.
MTAPLSNVRRMKFTKGGAPVVLDFLPKMDYLPLSLMVTAFKKDDVKKPLEGFGVLIPYKEQQKHGLKTLGTLFSSMMFPDRAPDDQYLYTTFVGGSHNRDLAGAPTSILKQLVTSDLKKLLGVEGQPTFVKHVYWGNAFPLYGHDYSSVLEAIEKMEKNLPGFFYAGNNKDGLAVGSVIASGSKAADLAISYLESHTKHNNSH

SEQ ID NO: 55 - protein, 469 aa, Rhodothermus sp.
MASVGIIGAGIAGLAAAYELHRRGLEVTVFEATDRIGGFIQSERIDGFLVEHGPQTLQRTSGDFEELLRQVDLEDACITARPIAANRFIVRGGRPIPLPRSPRELLRTPLLSPRARLRLLAEPFIHRAHRSTEESVAKFARRRLGPEVLDYLVEPFVAGIFAGDPEQLSVRYAFPKLFELEQQYGSLFWGLIRDRMKQRYHPAPRRSMFSFVEGLHMLPRALADRLPAHAIVRNAEVLAIRWDEKNPWTLTFRQHGRASTRFFDIIVCAVPLHRLAQLRIHPPVDRRPLSTVEHPPIALVALGFRREQVAHPLDGFGMLVPAVERDFQILGTLFSSSLFPDRAPEGHVLLTTFVGGMRHPELALLPEDRLEALVLQDLRRLLGISGAPVFRHVWRWERSIPQYRLGYDAVLACVHDVEMSRSGLFLAGNYMEGISVIDALHTGLKAARAIIQHLREEAAGGLAKLVLGD

SEQ ID NO: 56 - protein, 453 aa, Caldithrix sp.
MTVAVIGAGISGLTTAYYLKQQGVDVQVFEKNNYIGGSVITEKKDGFLIDLGPNSTLETSQVLRQLIDQIGLQSQKVYASDVSNKRYVVRDGLLHALPLSPPAFIKTKLFSWKAKLQLLKEPFLPKVEVDDISLADYVRYRLGDEFLDYAINPFVAGVYAGDPEQLSAPAAFPKLYNLEQNYGSFIKGAIKGKRERKKRQEVAKDRAKMFSFLDGMQVFPQALARQLGEVIHLNCEVREVIPHGKGFKVVLEQDSGEQECFFERVVISVPTYVQAKILNSILKERAALLADVLHPPIAVVFMGFKRDDVAHALDGFGFLLPAKEKKQILGSIFSSTIFPQRAPQGKVAFTTFVGGMRNPDNALKDDEEIKELVLKDLNDLVGLHGQPVLTRIRRWPRAIPQYTLGYKKIQALFDELEQEFSGLFFAGNFRRGISVGDSVLSAFETSEKMLKEK

SEQ ID NO: 57 - protein, 467 aa, Opitutus sp.
MSTSPFNPSATASGRPPKTFAVLGAGITGLTAAHRLTQLGHKVRVFEQSDRVGGSIKTEEVDGWLIEGGPNTLLSGELAVDKLIDELGLNGERIAADPAAKNRYIVRRGRALAAPMSPPSFFASSLFSPVAKFKLLAELFARRRVRTTDVSLAEFVESHFGREFVDYALNPFVGGVYAGDPEKLSARQSFPKLWEIEQTHGSLIRGQIAAAKARKARGEPRPGIFSFKHGLHVLPEALAARLPAGAITLGASLDAIVPGDKWNVVWHDDVATHTQSFDSVVVALPAPALARLQIGTLGEKPLAALALIEHPPVSSLFLGFRREQVAHPLDGFGVLVPAVEKRSVLGVLFSSSLFPGRAPLGHVALTVMVGGTRQPQLASLPADQLLAAVRPDLTQLLGVSGDPVFVRHNFWPRAIPQYNLGHEHFIAALAAGERFHPGLFMGGQARDGIAVPACIAAGEKLAERAGQ

SEQ ID NO: 58 - protein, 455 aa, Verrucomicrobia sp.
MGNPNKTIAILGGGITGLTAAYELLKLGHKPTVFESSSRIGGAIETIRQNDFLAECGPNTLLETSPRISALISDLGLDSRKRYANPSMKNRYIIKGGKPVPMPLSPGQFVTTKLFSLGAKLNLIKEPFIAPCPSETEESLAAFVVRRLGQEFLDYAINPFVAGVYAGDPSKLSVQHAFPKLYALEQQYGSMIKGQFFGARERKKRGTVSKDKAKLVSFDQGLEVLVDGLGQKIEDTINISTVIESVEKAEEKWVVQGRKIRESFDAVITAIPTHRMTKMTFRDESDLDMSILRDIIYPPVSSVVMGFRRDQIQHSLEGFGMLVPKVERKNILGTIFSSSLFENRAPEGYVTLSTYIGGMRQPDHALLDDQEMDRLILKDLEALLGVRGEPAFINRRVWKKAIPQYTVGYGKILDRFNELEAAHSGLFFAGHYRNGISLGDSILAGLDVAQRINQQ

SEQ ID NO: 59 - protein, 462 aa, Ignavibacterium sp.
MTKTIVVIGAGISGLTTAYLLSKRGFNIRILERKSEVGGSIESIKENGFLFDRGPNSALETTPLISQLVEELNLKDELLYANKAANKRYILRNNELHALPMSPPALIKTKLFSAKAKLRLLTEPFIGRSEDGYYQSLAEFVRRRLGQEFLDYAINPFVAGVYAGKPEELSVKSAFPKLYALEEKFGGLIIGTIRSIRERKKRAEKSKQSARMLSFKSGMISLPKAIANYFADKLILSAEVISVDKTAEGFIVSYRHSGIDEAIVCDAVLSTVPSYVAGNLFSKFDKKFKVHSDEIYYPPVLVYFLAYEKKNIGQTLDGFGFLIPEKENKSFLGALWSSVIFPYRADNNFATFTLFIGGSRYPDFVKEDRNKLLEKVRKEFEQLMKIKSDPVFSAYRFWEKAIPQYNIGYIEHERFFDEFEKQNPGLFISGNFRGGISVGDCIKNAELVANKICVQFTMHNVQ

SEQ ID NO: 60 - protein, 457 aa, Coraliomargarita sp.
MEKKAIVIGAGISGLCAAIELKKSGFNVTVVEKRERAGGVIGTTARDGFRAESGSNTVMVNSQKTLDFLMEIGLKDKIVNSSPAAKKRFFARYGKPQAVPMGPLQLLTTRLFSFAGKLRMLCEPFIKPPSQDSDPSVADFTAERFGREVLDYAMNPFMAGIYGADPEKLSIKHAFPPFWNLAIKYGSVIKGAMKARREKMAAGNFFKPVMISFKSGMHTLTDALAAELGDSVKCGAKVISIDSNCDGWEVSWGTETEDVCENYDALVVAVPAPEISGLPFGGMLAAALAPLAKIQYAPVATYTMGFKRQDVSHPLDGFGVLTPKKENFSILGSLFVSTLFDDRAPDGYIALTNYVGGMRHPEFAALERGEMRKLVLEDLKKLLGVNGEPVFEELFVWKNAIAQYNVGYQEYLDIMDDIEKRIPNIALVGAYRGGVGVSSCLENGLLSAAKLAGRISD

SEQ ID NO: 61 - protein, 465 aa, Salisaeta sp.
MARIGIIGAGIAGLTAAYQLQQQGHMVRVWEARGVAGGAIRSERTSDGFLVEHGPNSLRATTPIVPRLLEDLGLERARLSAAPAATKRFIVRDGTLRPLPLSPPALLTTSLLSTRAKLRLLREPFVAAGAPTADETVASFVRRRLGPEVLAYAVDPFVAGIFAGDPHRLSLKHAFGRLYEMERTHGSLLRAALHSARTGATDDASTATDRRIFSLRDGLQMLPHALADALGEAIRYEAPVTALRQMPDGTWTVATETDATQVNALISTVPLHALGSIDWAPAVDTSPLQRVPYPPVRVVALGFRRADVAHPLDGFGMLVPSAEDQFQILGTLVSSTLFPGRAPAGHVLLTTFVGGMRHPTLGAASDAAVRKVVLNDLQALLGVQGAPVFERFIAWPRAIPQYTLNHGAAVRTLEQLEDVHPGLFFAGNYRDGISVGDAMASGEDAARRVDAHLAGADRAVAATGP

SEQ ID NO: 62 - protein, 179 aa, Ambrosia sp.
MASPTIVDNQKPAKRVAIVGAGVSGCAAAYKLKLHGLNVTVFEADERVGGKPRSVSQDGLIWDEGANTMTESEADASSLIDELGLRDKQQFPISQHKRYIVRNGKPVLIPSNPIALIRSSFLSTQSKVQILLEPFLWKKTKSSDEPESVGGFFQRHFGKEVVEYLIDPVVAGTSGGDPK

SEQ ID NO: 63 - protein, 451 aa, Melioribacter sp.
MSKKIVVLGAGISGLSTAYWLVKKGYDVTILETKNEPGGSMISRRLDNFLIDYGPNSGLETTPLIRKLVEEVNLSDKMIYANAAASKRYILKNGELIPLPMSPGSFIRTKLFSSGAKFRLMAEPFVSKSDDGYYQSIAEFVRRRLGNEFLDYAIDPFVSGVFAGDPEKLSVKSAFPKLYRLEEVYGGLIKGMIKGARERKQRNEESKQSAKMFSFLEGMQSLPNAIADKLKDNIVFSAKVLNVTGANDKQWKVTYELNGNRESITADTVISTLPAYIAAGVFGELDQKLAERLNSIYYPPVMVLYLGYNKKDIKRKLDGFGFLIPSKEKKHFLGAIWSSSIFPGRSPEDMAAFTLFVGGARSPQLFEMEKSDLIKKVLSEFHQIMNIKGEPVLIENKLWQKAIPQYNLGYIEHEKYFEVFEENHRGIYLRGNYRGGISVGDCIKNSELEIK

SEQ ID NO: 64 - protein, 472 aa, Halothiobacillus sp.
MNTHSNASEPINCPYLVIGGGISGLATAYHLSRMGKTVTVLEATSRVGGAVGSIEEDGWLRELGPNSLVQTPEMAALMSALDLVSEIIEANTVAKKRFVAKNGHPVALPGSPLELLTSPLFSMGDLWHLAREAWIKPVNKEETIAEFVRRRLGQGFLDWAVDPFVSGVYAGDPNRLSVQAAIPKIYAFEQESGSLIRGGIAKMKAAKANPAPVTLPAKGKLFSFKRGLQTLTDALATAITSSGRGNIQLDSAITNIQRLPEGGWSVKTVQGKTFHTRQLILSTPAHVSAQLLGEVDGPLAETLAAIEYPPVTSVVMGFDRSEVAHPLDGFGLLLPSKEKKRTLGVLFSSTLFPDRTPAGKVLLSAFIGGRKHPEAAQGDDQELLDRVLGDLSPLLGIKGKPEFLRVKRWQQAIPQYEIGYLELQEKISQRLTALPGLSLNGNWRGGIAVGDCLNNGNKLAERLIENTRTEES

SEQ ID NO: 65 - protein, 480 aa, Chondrus sp.
MPEPVKRNVAVLGGGPAGLSAAFRLLHDLPPGLSVKVDLYEATGRLGGAVTSACDAGFCYELGPNSMNAKHPSVADLIQEKLRLKPRMLPRSPYAKRYYFMRDGKLVPLPLSPLHFATTGLLSWRAKWHVVKEPFVARLQDVRDSHMESVASFFQRRFGREVVDYLVDPMVAGTYSGKPADLSMKHALRKVWRLEQKHGSVIGALLRGAGKTKPDPRYKPYTGKELRASFTYDKGMEVVTDALVANINDLNPRGGRLYTHGKVRTLDRDPNGAWRINGRGKYDAVISTIPTHAVKSIYTNMSALGKGFKKLNKSIKYAPVSVVVMGFDKSQVPHPLDGFGALIPTVEGRQILGINFSSSNYPQRLSDPDKVFLTVYVGGQRNPDLPFRRAKEIVDISKKELGHVLGVKGDPFFSRVKTWKRGIPQYDPQFDESLCTMARAEKSAKGFVFGGNYRDGVGLPDALRSGILSAEKTLQYLKTL

SEQ ID NO: 66 - protein, 463 aa, Rubritalea sp.
MRKHIAIIGAGVSGLSTAYHLQQAGHSVVVFESSKRAGGVIQTIREDDYLCEWGPNSLMIGDKRIEALLKSIGLDDEILEANTASKKRFIIDRDRLQALPNGPFQLLGSPIFSLKAKLRLLKEPWVKRHPANSKETLADFMERRLGPEPVAKLVNPFISGIYAGDPKRLAVEHAFPKLFQLEQDYGSLIWGMIRSKKDQKNSFKSKHKLQKRRIVSFKQGMEALPQAMAAALDEGTLIFEAKIGGISQNRNNKRWHMDWKCPNGTIGQGSFDAVILTQASHHLNDIPLDEEVLESLSKLPSIDHAPVTSMLLGFKREQIKHPLDGFGMLSTFEQKSKMLGALFTSSLFPGRAPEGHVAINVMIGGVRRPELCELPEPTLRANILDELRRLLGVDGKPSFCHIHHTSKAIPQLHTDYGIVAQQIIDCEREHPGLYLAGSYRNGIALPDRLLEGISLTEKINQSL

SEQ ID NO: 67 - protein, 457 aa, Acidobacterium sp.
MGVEVAIIGAGISGLSVAFFLKRAGAGVLVLEAEEDVGGTMRSRRFKGYLIELGPNSALETTPLFQELIAAAGLAGERVYASEAARNRYIVRGGELHPLPLTPLAFLRSRLWSWKGKLRVLAEPFHGRADREESVADFARRRVGQEFLDYAVNPFVAGIYAGDPERLSVRFAFPRLYALEAQYGGLFLGMLRGARERRRRGEAPKIAARLFSFREGMGALPRALAAALGNTVWCATRALCVERAGAAFEIAFERDGRRDTLRAERVVLATPAYAAASLLKRLAPEAARALDRIVYPPVSAVILGYPETAIGRPLDGFGFLVPEKEQRRILGTIWNSTIFPARAPQGFVTLTTFVGGMRQPELARRPNEELIALVAEELTDLLRLRGEPEFAYVSRWERAIPQYELGYGEILDALDRAEREHVGLYFCANYRGGIAVGDCVMSAHATAERILRDRARS

SEQ ID NO: 68 - protein, 441 aa, Coraliomargarita sp.
MPDTCIIGAGITGLATAWQYQRKGKDCVVLESGPQVGGAIQSILQDGYLAEEGPNSIQLNSLEIEDFLTSIPGLEAQIIEANPAAQKRYIVRKGRLRAVPMNPLQAITTQLWSIAGKLRVLREPFIKAAPPEPDQSVADFVTRRLGKELYDYAINPLVGGIYAGKPEMLSLRYGFPKLYALEQEHGGLIRGALAKMKAAKANKGPKAKKYILSFKDGLQTLPQSIANNLNEPVQTGVSIESIRQIEDMWAVRWNGQVKAFKELIVTVPAHKLPGLPFEEPIRLPAIDYPPVSVISLGYPLSAIKQPLDGFGALVPEREDRNILGVLFPSAVFDGRAPEGHGLLTVFVGGSRSPECSSPDTDQLLKTIQPDLETLLGIQDEPSFVHHKHWPMAIPQYTLGYEKVLEAITRIEQQYIGLKLAGNYRTGISLSYCLESAIASTN

SEQ ID NO: 69 - protein, 474 aa, Oscillochloris sp.
MNSYDVVVVGAGISGLSAAYTLFKRGLDVLVVEAGAEVGGVMRSIVTPEGYVLDCGPNTLASKDPRMWAEFDDLGMRDRLVVAGRAGKRRYFLKDGQPLEIPNDPIGLLRMEHVSMQAKLRVLREPFIPRATSPDESVASFFSRRIGPEVMERMIDPFVSGVYSGDPSKMSIKATFPSLWEAEQRGGSIIKGFLTAGKAKKAAGEKKAKPIGPKMRSVTFNFKGGVAEWPKTIAKVLGDQRVWKNARVTTLYPESTGWTLVVERDGQVETIEAASVIMAAPAYAAADLIAELDPAAAKGLRGIRYSSMAVVNLGYRENQVTRPVNGFGVLAPSCERRNFLGILSASTLFPPFAPAGRVLTINLMGGEINPIRPEQSDDELIARAIADNQAVIGANGAPEVVNLTRWPRAVAQYNFGHTEAMAALENLERTRPGIYFVGSYRGGVGMPKCWRNGVNMAERVATYLKTRSAVASLR

SEQ ID NO: 70 - protein, 474 aa, Opitutaceae sp.
MPVAVIGGGITGLTAAWRLAREGHPVRLFEASPRPGGLIRSERDGEWLSEAGPNSLLDNKPELAALLAELGLEDARQYAQPAAKKRFIVRRGRPLAAPSSPVSAVTTPLLSLRGKLRIFGDLFWRPRPRAEDLPLGEFAAAHFGRELADYAVDPFVSGIYAGDPQRLSARYAFPLLWELEQKHGSLIRGGIAAAKARRATRPAGPAEKPRPPRARIFSFAEGLETIPAALAERLPAGCVETGARVVRLVPPVARGGAWEVVWERKDEKDEKGASGVEGRLTERVAAVILALPGEALARLEIGANGEHPLAALAEVEYPPVASLFIGYRREQARHPLDGFGMLAPSKENRNLLGVLFSSTLFPGRAPAGHIALTVLAGGVRRPELARMELPSLMGIVRAELLELLGVSGDPVYVKHRVTPHAIPQYNLGYGRFHTVIETAETAHPGLLVGGPVRDGIAVSACVAAGEKLARRVVA

SEQ ID NO: 71 - protein, 239 aa, Amborella sp.
STKSVAVVGGGVSGLTTAYRLKSHGLNVTLYESEGTAGGRIRSIAYGGLIWEEGANTMTESEMEVKRLIDDLGLREKQQFPISQNKRFIARNGTPVLIPSNPLALFGSKLLSPHSKLRVILEPLFWRSSNRKGDISKVSDQNLQESVGDFFQRHFGQEVVDYLVDPFVAGTSAADPNSLSVQVTSMSEICQNYLQYLGWSQGPSSQLMTLTLSPSSVIDQMLSERMVTFLQVMLSPLLM

SEQ ID NO: 72 - protein, 490 aa, Opitutaceae sp.
MSASLPPPPPPPHTHTQARLPVAVIGGGITGLTAAWRLAREGHPVRLFEASPRPGGLIRSERDGEWLSEAGPNSLLDNKPELAALLAELGLEEARQYAQPAAKKRFIVRRGRPLAAPSSPVSAVTTPLLSLRGKLRIFGDLFWRPRPRAEDLPLGEFAAAHFGRELADYAVDPFVSGIYAGDPQRLSARYAFPLLWELEQKHGSLIRGGIAAAKARRATRPAGPAEKPRPPRARIFSFAEGLETIPAALAERLPAGCVETGARVVRLVPPVARGGAWEVVWERKDEKGASGVEGRLTERVAAVILALPGEALARLEIGANGEHPLAALAEVEYPPVASLFLGYRREQVRHPLDGFGMLAPSKENRNLLGVLFSSTLFPGRAPAGHIALTVLAGGVQRPELARMELPSLMGIVRAELLELLGVSGDPVYVKHRVTPHAIPQYNLGYGRFHTVIEAAETAHPGLLVGGPVRDGIAVSACVAAGEKLARRVVA

SEQ ID NO: 73 - protein, 474 aa, Chloroflexus sp.
MMAGYDSVVIGGGIAGLAAAYTLHKRGYRVLVIESTNRVGGVIQTITTPEGYILDCGPNTVGTGDARLWQELIDLGLRERITPAAPCSKRRFILINGTPVEIPTSPVGLITTRLLSWRGKLRVLAEPFINRGSTDPDESVAAFFTRRIGAEATAHLLDPFVAGVYAGDPQRLSTAAVFPSLWEAAQRSGSIVRGMLSKPKPKTQVSEPKMRSRTFTFRGGLAEWPRALAQALGAGNVWTERRVVKLQPRDSWWEVTIDGVNGPETLISRSLIIATPAFTAADLIESVDQRAAGALRGIPYAPVAVVHLGFRRDQISQELSGFGVLAPSSEQRQFLGILWTSSIFPHVAPHDHVLTTTLSGGAIRPELAERSDETLIEAAIRDHHQLLGIRGQPIFTHVTRWRTAIAQYTFGHRERIATLVQLEQRLPTIQFAGSYRDGVGVPKTWASGVQAGERIAAALAAHGTTAVSTETASG

SEQ ID NO: 74 - protein, 462 aa, Leptospirillum sp.
MAGFDCDTLVVGGGVSGLAAALTLKNRGVDVRLLESRGYLGGAIRTVREDGYLLEFGPNSLMVRPEDAIDTVLGDPELRARIVPASGLSKNRYVVKAGHLYPVPLSPWAFFRTPLLSWRGRRDILSEWKVPPRTGGPEETLSHFVRRRLGEEALDYFVDPFVKGVYASHPDLLSVEAAFPLLVRLEREHGGLLRGALKTFLKRRKRPSGSSPRGIFSFAGGMTDLVEAMGKRLGEDVGTNVDVIKYTRLEEGFRVALMYDETEYYMTSRRLILATSAPQAAELLEGDPDGPSSELKSIPYAPVTIAYAGFLREQVTHPLDGFGLLCPTVENRKVLGVIFSSSLFPGRAPEGKVLLTVFVGGMTGQKLAQAFDEDLERIVLKELTELLGVKGAPSFFRIHRWEKAIPQLILGHGETVRTIRKKLPSGLRLAGNYLDGISIARAFASGVRAAEELLSEDGGTPG

SEQ ID NO: 75 - protein, 462 aa, Leptospirillum sp.
MAGFDCDTLVVGGGVSGLAAALTLKNRGVDVRLLESRGYLGGAIRTVREDGYLLEFGPNSLMVRPEDAIDTVLGDPELRARIVPASGLSKNRYVVKAGHLYPVPLSPWAFFRTPLLSWRGRRDILSEWKVPPRTGGPEETLSHFVRRRLGEEALDYFVDPFVKGVYASHPDLLSVEAAFPLLVRLEREHGGLLRGALKTFLKRRKRPSGSSPRGIFSFAGGMTDLVEAMGKRLGEDVGTNVDVIKYTRLEEGFRVALMYDETEYYMTSRRLILATSAPQAAELLEGDPDGPSSELKSIPYAPVTIAYAGFLREQVTHPLDGFGLLCPTVENRKVLGVIFSSSLFPGRAPEGKVLLTVFVGGMTGQKLAQAFDEDLERIVLKELTELLGVKGAPSFFRIHRWEKAIPQLILGHRETVRTIRKKLPSGLRLAGNYLDGISIARAFASGVRAAEELLSEDGGTPG

SEQ ID NO: 76 - protein, 446 aa, Verrucomicrobia sp.
MNTKKVCILGAGLSGISLGLSHENKGNQVTIFEKDLRVGGVLQSIKSEGFLMDYGANTLSIRTKKTVDFLKQYEILEHAMDANQESSKRFIIRKNRIISLPQGPLSFLCSSFLSPVGKLRLCLEPFIGRKKDNGSDETMAEFVERRLGREVLDYGVNPFIGGVYAARPESLILKYAFPSLHDTELTFGSIFWGMIRGGAQPSEKISKSRLISFREGMQELPNRLAARMHNPPVLGCEIKKIEFKDDVQWCVQGEKHDGKIQKEVFDQIICTLPSHALDKIEWVGINSSHLLETLTRAYHPPLALVFQGYQQRQIKHPLDGFGFLVPEKERRKILGTLFSSTLFQNRAPENSVLLTTYIGGERNPELCDLPQNEILGHAFRENQDLLGIEGKPIFEHLKLWPKSIPIPDHTLEDRKKAASTLTLENKGLQILGAHINGAPLPNCMVL

SEQ ID NO: 77 - protein, 475 aa, Chloroflexus sp.
MMMANYDSVVIGGGIGGLAAAYTLYKRGYRVLVIEAANRVGGVIHSITTPEGFTLDCGPNTIGTNDVRLWQELIDLGLRDRIRPAARCGRRRYILINGTPIEIPSSPVGLITTRLLSWRGKLRVLGEPFVNIGTPTGEESVAAFFSRRIGHEAVAHLLDPFVAGVYAGDPNQLSAAAVFPSLWEAVQRGGSIVRGMLRRPKQKTLISEPKMRSRTFSFQGGLADWPRAIARALGTGNVWTGRRAVGLRDLGTYWEVTVDGTGRLETITTRSVIIATPAYVAAELVEALDPAAASALRSIPYAPVSVVHLGFRRDQLSHELNGFGVLAPSSERRQFLGILWASSLFPHVAPPDRVLTITLSGGAIRPEVAEQSEEALIESAIRDNQEVLGIRGQPLLTHVTRWHHAIAQYTLGHRERIATLERLEQRRPTLQLTGSYRGGIGIPKTWASGVGAGERIAAALDAQGTTADTLEQARG

SEQ ID NO: 78 - protein, 456 aa, Desulfurobacterium sp.
MKVAVIGAGISGLSVAFYLKKGGAEVKVFEKEKTVGGKMKTIHEDGYIIETGPNGFLDGKPYTLNLVKELGIESKLYRSSDKARKRFIYTNGRLVRLPESPIAFLASYLLSWKGKLRLVGEFLVPPKKEDIDESLSEFAKRRIGEEALEKLLDPMVAGIFAGDPDRLSLKAAFPAIYYLEKQYGGLIKGLIAKMKEAKKSGKKSGPAGPGGVLTSFKGGVKDLIDSLSEFLGDSIETEVEILGLDRIEKGWKVKYKKENEVFEETFDAIVFSTPAYITAKLLNDLNLELSKLLSEIEYSPISVVALGFEKKGLGHDLDGFGFLVPRSEKRKILGALWDSSVFPNRAPSGKALIRVMIGGARQPELALLPDEELVNIALKELRRIMKIRHYPEKIKVFKHEKGIPHYTVGHAERVEKIFRLISKYPGLYLCNNAYTGVGVNDCTKAAEEVARRILDG

SEQ ID NO: 79 - protein, 455 aa, Desulfurobacterium sp.
MKKVAVVGAGVSGLSTAFYIEKFGGDNVSVTIFEKENVPGGKMLTVQKDGFIIETGPNGFLDNKPYTLDLVKDLKIEDRLYRSSDKARKRFVFVNGKLVRLPENPIAFLSSYVMSFKGKLRLAAEYFIPPKKDDSDESLSSFVKRRIGKEALEKLIDPMAAGIFAGDPDKLSVKAAFPAVWHLEKKYGGLIKGLLAMKKEKKDATAGPGGVLTSFKGGVKDLIDALVANIKGEIHPGTTVKKLIPENGKWKIVYEKDDEIFEESFDKVVLSTPSYVAAALVKNFDEKLAEKLYEIEYSPIAVIAFGFIKKGLGHHLDGFGFLVPRSEGRKILGVLWDSSVFPNRAPEGKALIRAMVGGARQPHLALAGEEEIARMTYKDIKRIMKIRHRPIMTAVFKHPKGIPHYTVGHVEKVNEIFKLASNHGGLFLNSNAYRGVGVNDCVYNSLKTAEMVTSE

SEQ ID NO: 80 - protein, 484 aa, Arthrospira sp.
MTNLVDSLIVGAGISGLSLAYSLNREKSVREPLKVLVTESQNRVGGNITTGRADDFLWEEGPNSFAPTPELLGLAVDLGLKEELIFADRKLPRYVYWNLMLHPVPMNPPALLSSELISARGKLRAALGAIGFVPPPVGAHLSQQGGEETITQFFDRHLGSEVLERLVQPFVSGVYAGDPQQLAVRSAFSRIVAAEEAGGGLLPGFVRSRLNKKAPVSTPDPNIPKTRPGELGSFRYGLQTLPETLASKLGDRVKLNWTIDRFYPTDHQTYIAEFSTPDGPQQVEARTLALMTPAHVSARLLQPLHSPIATALSQIPYPPVACVVLAYPKSALKQQLKGFGNLIPRHQGIRTLGTIWTSSLFPGRAPESWQVLSNYIGGATDPEIGEMDDDQIVAAVHQDLRQILLAEDVPPKVLAVHLWRRAIPQYTLGHQNRLNCIDAGLRSLPGLYLCSNYIDGVSVGDCVRRGQQWASKIQSHLHDCQTAN

SEQ ID NO: 81 - protein, 462 aa, Leptospirillum sp.
MAGFDCDTLVVGGGISGLAAALTLKNRGVDVRLLESRGYLGGAVRTVREEGYLLEFGPNSLMVRPDDAIDAVLGDPELRARIVAASGLSKNRYVVKSGNLYPVPLSPWAFIRTPLLSWRGRRDILSEWKVPPRTGGPEETLSHFVRRRLGEEALDYFVDPFVKGVYASHPDLLSVEAAFPLLVRLEREHGGLLRGALKTFLKRRKRPSGSAPRGIFSFTGGMGDLVEAMGKRLGEDVGTNVDVIKYTRLEEGFRVALMYDETEYYMTSRRLILATSAPQAAELLEGDPEGPSGELKAIPYAPVTIAYAGFLREQITHPLDGFGLLCPTAENRKVLGVIFSSSLFPGRAPDGKVLLTVFVGGMTGQKLAQTFDEDLERIVLRELTELLGVKGAPAFFRIHRWEKAIPQFILGHGETVRTIRKKLPSGLRLAGNYLDGISIARAFGSGVRAAEELLSEDGGTPG

SEQ ID NO: 82 - protein, 458 aa, Verrucomicrobia sp.
MQDAIILGGGISGLTAGYLAQKQGQDISVIEKGKIPGGPISSFREEGYLVERGPNSLLLPDPWVETFIEELGLRDQLQETNPIASKRYIVKNGRPEAVPSSPLQAVFTPLFSLRGKFGFLLEPFRKKISDRAGSRETVASFVKRRMGLDFLDYAIDPFVSGVYAGDPNRLILEHAFPLMRGFERDSGSIIRGAIKHKKKQKREGTAYKKRSISFKDGLGILPQTIARKLGNRLWLGSEVVAVNRVEDHWQVTWKREGENFEGFAKNLLVCLPSHAIKRIAWSERIAAPLRSSPNLEYPAVHSLALGFRREQIAHALDGFGVLVPSKEPPTILGALFSSSLYEGRAPDGHCLLTVMLGGIRHPELAALPQDRLLELALRDLRALLGLKGDPSFYRCTSWPRAIPQYTRDFGPWRDTLKSLAEEFPGLHFGGNSVDGIAMGASILSGKRLAECLDKDIDV

SEQ ID NO: 83 - protein, 446 aa, Verrucomicrobia sp.
MNTKKVCILGAGLSGISLGLSHENKGNQVTIFEKDLRVGGVLQSIKSEGFLMDYGGNTLSIRTKKTVDFLKQYEILEHAMDANQESSKRFIIRKNRIISLPQGPLSFLFSSFLSPVGKLRLCLEPFIGRKKDNGSDETMAEFVERRLGREVLDYGVNPFIGGVYAARPESLILKYAFPSLHDTELTFGSIFWGMIRGGTQPSEKISKSRLISFREGMQELPNRLATRMHNPPVLGCEIKKIEFKDDVQWCVQGETRDGKIQKEVFDQVICTLPSHALDKIEWVGINSSHLLETLTRAYHPPLALAFQGYQQRQIKHPLDGFGFLVPEKERRKILGTLFSSTLFQNRAPENSVLLTTFIGGERNPELCDLPQNEILGHAFRENQDLLGIEGNPIFEHLKLWPKSIPIPDHTLEDRKKAASTLTLENKGLQILGAHINGAPLPNCMVL

SEQ ID NO: 84 - protein, 485 aa, Synechococcus sp.
MNPATPEPLNAEVVVIGAGISGLTLAWRLQQGLSARGGSPQAVLLAEASSRVGGCISTQSKDGYRWEEGPNSFTPTPALLNLIAEVGLTDQLVLADAKLPRYIYWEGALLPVPLSPAAALGSRLLSVGGKLRALQGLLGFVPPPPGHEETVRQFFRRQLGSEVAERLVEPFTSGVYAGDPDQLSAVAAFPRVAGLEERYGSLFAGALQALRQRPQPSPAAIQPPPKRGQLGNLRQGLQQLPEALAQKLGDSLRLGWRALQLKRAGELYWVGFETPEGSRWVAARQVVLALPAYEAAALLQELNPPASQLLAEILYPPVAVVALAYPQEALPQPLRGFGHLIPRSQGLRTLGTIWASCLFPERAPQGYHSFLSFLGGATDAALARRRGIPPIPALSPEERAQIAHAELSQVLLTRRAEPVYLGERLWPRAIPQYTLGHRQRIAQVQAHLASQTPGIWVCANYLDGVALGDCVRRAEALAQQLLSQV

SEQ ID NO: 85 - protein, 443 aa, Hymenobacter norwichensis
MTIAILGGGISGLVVAWQLQKAGVAYDLFEAETTPGGCLRSVSHPDGYLVETGPNSLQLSDELLELITELGLVDEIQDAAAVSKNRYVLRNGRYQQLPSTPPALLTNGFFSWKAKFNILRELRRPAAPLNPTETVDAFFRRRFGPEIVDYAVNPFISGIYAGDPAQLLIHKTFSKVAALEQQYGSVLRGLAKTGGGAGRRRIISLQGGIQKLTDTLAAKLTHHHVRQRVLALHRTTKGGYQVQTSAGSNGGFSYDAVVLALPTFAAAPLLAPLFPEAAAALAAVHYPPMAAVYTAYRREDVGHPLDGFGALHPKVEQPYAAGSIWTSSIFPNRVPDGQVLFTTFVGGAQYEANAQQSETAQKAAVHEELSRFYDIKAAQPLWQYRYLWDKAIPQYDQRIMAAHTTTDALQAQGIWSAANWRGGVGVPDCIRHARHVADQLTGK

SEQ ID NO: 86 - protein, 448 aa, Pontibacter sp.
MRVAIIGAGISGLSLAYYLQRQGVAYDLFEAGSEVGGNMRTLRKNGYTFELGPNTLQMNEELLQLITELKLETELLPLTPRNNKRYVLHDGKLYPVPASPKSFLANDLFSNEEKHRILQERKQPPAEVENETVSDFFERRFGVKQMDYLAAPMISGLYGGDPRQLLVNKAHPELKELETQYGSVLEGMVQKKKRGVFQRAFSFRNGMSTLPHAIADKLISLHLDHKVEFITRIKGKFIISCASNGDHDNEEYDKLVLALPAHQAAELIEFTYPGMSAALQNINYPPMAVVHTVYNRAEVGHPLQGFGALHPWEEQSFTSGSIWTSSLFEGRCRSHEVLITSYVGGTRFAEHAQLEERSLLEQVHQELCQTYQIKALAPVYQHLHLWQHALPQFDLYIEDAHHMAEVLEQDGLFISANWYAGVSVPDCVREAKAIAAKINTRAASRSIA

SEQ ID NO: 87 - protein, 463 aa, Leptospirillum sp.
MATEDVETLVIGGGIAGLACASHLKLAGRTVRLVERNDYLGGVIRTLSDSGYRIETGPNSLLVRTEEPLLKYLSRPEIAARIQLAGRMGKKRFILKDGHPVALPMSLSEGIFTQILSLPAKVRLLKEPFIPPAGGVDGVDPEKETVADFVRRRLGNEFLESLIDPFVKGVYASDPHLLSMADTFPRLVQMEKSYGSLIKGGLALARQKKAPAPAFAREILSFSEGMGTLPESLANILDDDAGTNAEVIGCAPSESGFRTALLFEEETYYIRSKHLVLALPAAQTAELIEPMAPGIPSLLGQIPYAPIAVVYLGYPRDRISHPLDGFGLLVPSRERRKILGALFSSSLFPGRSPDGHVLLTVFVGGMTQPKLAQAFDEDLLPMVTKEIGSMLGVLGAPSYVRIQRWAGAIPQSVPGHGERIRSIESALPSGLHLAGSYLSGVSVSQTFSSGIRAAEKILAQSPG

SEQ ID NO: 88 - protein, 451 aa, Prevotella sp.
MERQRKIIVIGAGLTGLTCAAHLRHKGQDVEVLEATERIGGLMQTEDFDGFIMEQGPSTGTIKYPEVAELFDMLGDKCTLEVAQSSAKCRLIWKDGHFCALPSGLWSAVTTPLFTLKDKFRILGEPWRRKGTDPNESVGALAERRLGRSFVDYAVDPFLSGVYAGDPYKLPTRLALPKLYNLEQHYGSFIKGAMALAKAPKSDRDKRATKEVFSTRGGFRSLVSALAKTIGDDRITTGCAHLHVEPLGDKWKLSWGENTIIADQVVTTCPAYSLPDLLTFLPKEQLDDLSNLYYAPVIEIGVGMKHTGNVHWNAFGGLVPSKEQQKVLGILMPSACFVGRSPEEGATYAFFIGGARHPEYLDKTDEELRELVNTSLHTMLGYPKGTQADAIRIYRHSHAIPQYMTETDSRLRAIDTVEHTYPSLHIIGNLKDGIGMGDRIKQAVDLAERIG

SEQ ID NO: 89 - protein, 436 aa, Flexithrix sp.
MVGIIGAGISGLSLAYHLQKLGKAYCLFEAGDKPGGNICSVKKEGFLLEKGPNSLLADSEIIDLVKELGLEDQMIIPETVSKKRYIFKNGKYQQLPSSPPGLIFNNFFSWHAKFSIYKELNNKSTSPQNETVADFFERRFCKEIVDQAVNPFISGIYAGDPEKLIMEKTFPAFLENEQKFGSVIRGFIKNKKNTQRKLTFSFKEGLGILADKLAENLSVNYNSPIKEIRQEAGGFLLITENGETKVTALVFSIPAYAAGKLIKDISPESAQAWEGVNYPPICVVHTAFRRKDLGFDFNGFGGLNPKKEDLFTAGSIWNSSLFENRSPKDQFLITSFVGGAQSLDNFQLTDEEIKAKVTDELQQNFKIKGSPTHQEITRWEKSIPQYDIDIFPAHQAIENLQREGIYVCSNWEGGVSVPDCIKKGKQLAERIKENKF

SEQ ID NO: 90 - protein, 469 aa, Geobacter sp.
MKKVIVVGGGISGLATAFELRNKGAEAGIELDVTLLEKEERVGGKIWSIKEEGYLCEWGPNGFLDSKPQTLDLCRDLGASERLLRSNDNARKRFIYTGGVLNRLPENGPTFLKSSLISWPGKLRLAMEPFISKRTDGTDETLASFGRRRLGEEALQKLISPMVSGIFAGDPETMSLRSCFPRIAELEDEYGSLIKAMIKLAKKKKQEAAQGKAVSSAAGPGGVLTSFRWGIQELTDILAEQLGSATVVTGQPVTGLTRGSSVPWRLKTPTVDIDADVVILASPAHATAGIVSGVDAAMAQVLGEIPYASMTVVCFGFERERIAYDLNGFGYLIPKDEGMNTLGTLWDSSIFENRAPEGKVLLRSMLGGACFPEYVKLSDAEVMQRVKADLKATMGITADPSFIRIFRHPQAIPQYTVGHGKRLAALQERSSALPGLFLTGNSYRGIGLNDCATAANRTTDEVVAYLKGR

SEQ ID NO: 91 - protein, 498 aa, Synechococcus sp.
MQAEVVVVGAGISGLTLALRLQQGLSPKDESTQPLLLAEASSRVGGCISTQSKDSYRWEEGPNSFTPVPALLNLIAEVGLAEHLVLADAKLPRYIYWEKELLPVPLSPSAAIGSRLLSVGGKLRALRGLLGFVAPPPGGEETVRQFFRRQLGSEVVERLVEPFTSGVYAGDPDQLSALAAFPRIAGLEERYGSLFAGAVQALRSRYRYATLPRTRHQDSANSPIQPPPKRGQLGNLRQGLQQLPEAIAQKLGSALRLGWRAVHLKRDETGYRVGFVIHDSGAEHTAPEEIHWVAAQQVVLTLPAYAAATLLQDLNPQASRLLREIPYPPVAVVALAYPEEALPQPLRGFGHLIPRSQGLRTLGTIWASSLFPERAPQGYHCLISFIGGATDAAFARQKGIPPITALSPDERAQIVHAELSQILLTRPVEPIRLGERLWPQAIPQYTLGHRQRIAQLQASLADQTPGVWVCANYLDGVALGDCVRRAEALAQQILSVRR

SEQ ID NO: 92 - protein, 475 aa, Crinalium sp.
MVDTLIIGAGISGLSLAYALHQDGRKVLLCERQERVGGNITTGKAGGFLWEEGPTSFTPTPALLKLAVDVGLREELVLADHRLPRFVYWKGQLLPVPMSPPSAVTSKLLSLSGKFRALVGALGFIPPAIGNHLSQQGGEETVAQFFKRHLGTEVAERLVAPFVSGVYAGDVHQLSARSAFRRIAQLENVGGGLVSGAILSRKQRQQQKPPTDPSLPTVRRGELGSFKEGLQSLPKAIASHLGENIKLNWTLTELRQTANQTYIAEFSTPEGSQQVEARTVVLTTPAYVTAELLHNLAPNASIALKEIPYPSVACVVLAYPDDALKFPLKGFGNLIPRGQGIRTLGTIWSSSLFPGRAPQGWQMLTNFIGGATDPEVGNLDNEQLVQAVHKDLQRVLLKKDVPPKAIAVHLWKRAIPQYTLGHHLRLAQINQDLAQLPGLYLCSNYTDGVSLGDCVQRAYDQLPIINKQLSIINDN

SEQ ID NO: 93 - protein, 486 aa, Planctomyces sp.
MNQSAPAKRIAVVGGGITGLSAAFHLQELAQEKNQPVEVTLFESQPEAGGWIGTISQDGYRIDTGADMFITNKPAAIELCQRLGLEDQLISTNQQYRGALILKDGTPVPVPLGFELMTPSRILPMLKTPLLSPIGKLRMGLEYFLPRRTSESGLDADDESLAQFVTRRFGREALTRLIQPLVAGIYTSDPEKLSLRATLPRFLDMERDHRSLIKAIRKQKKQTKSADATGARYGLFAAFKEGMQTLIRTLADRVSSTGTILYEHRVTHVAAADSGYDLTIESTVETQTQHFDAVLLTTAAPQAGQMLEAYAPVLSGLLKQIEYASTAIQVSVYRQENIKHPLHAFGLVIPAAEQRKIFAVAFASRKFPGRAPEGCVQLRTFVGGAMQPELLEHSDDELNAIVNQELADILGVSGEPIFSKLLRHNQSMPQYHLGHLQLVERIEQSAATLAGLELAGNAYRGVGIPDCIHSAEQAAERLLVDLTARV

SEQ ID NO: 94 - protein, 471 aa, Geobacter sp.
MKKAIVVGGGISGLASAYLLREKAKNSGMELEITIVEKEDRTGGKIRSIKEDGYLCEWGPNGFLDSKPQTLDLCRELKVDSQLLRSNDNARKRFIYSGGVLNRLPENGPSFLKSRLISWPGKLRLALEPTPFIAKAPEGVDETLAAFGRRRLGDEALRKLIAPMVSGIFAGDPETMSLVSCFPRIAELEREYGGLVKAMVKLAKKKKQEIAEGKQVASAAGPGGVLTSFRDGIQTLTDILNERLGKDMLVIGAEVTGVSRGNSTPYRVQTGGRELDADIVVLATPAYATAQALEGIDGGMSATLNQIPYATMTVVCFGYEQEKVAHDLNGFGYLIPKAEGMNILGTLWDSSIFENRAPEGKVLLRSMMGGACFPEYIRLSDAEVVQKVRDNLKTIMGIKEAPEFVRIFRHEKAIPQYTVGHGRRLAALEEQAKSHPGLFLSGNSYRGIGLNDCVAAANRTADEVVAFLQSR

SEQ ID NO: 95 - protein, 446 aa, Acidithiobacillus sp.
MEDVIIIGAGISGLATAYFLRKQGWSPLLLEAAAKPGGNLQSRQEEGYLRDMGPNSLMLKGHIVPEWLRELRLEEDIVEANPLAKRRYILNRHRQPVALGPGVLFGGGLLSWRGRLRLLGEPFRSPRRMQDSEESVADFVRRRLGEEALTWLVDPFISGVFAGNPARLSVQATLPRLIALEQDGGSLLRGALRARNKKSPDTPKTRLISFREGLQTLPLRVASALGDALRCNTPVEQLGNSDGSWQVSSGSQTWQSKRLILALPAGAAARLLAPTDAALAHELDAIPYPAVGSLSIGFQRMQVQHPLDGFGILIPRVMGLETLGILFSSTLFPGRAPADQVLLTAFIGGSQNDISGRDDDDLLATALREICPLLGISGKPVFSRCQTWPKAIPQYEIGHLDRIKRIDALSARHPGLYFRANWREGVALGDCMEEAYRFSQDVGWQR

SEQ ID NO: 96 - protein, 454 aa, Prevotella sp.
MEERKIVVVGAGLTGLTCAAYLRRKGQDVVVLEAADRIGGLMQTEEVDGFVMEQGPSTGTIKYPEVAELFDMLGDDCTLEVAQSSAKCRLIWKDGRFHALPSGLWSAITTPLFTLKDKFRILGEPWRKKGIDPNESVGSLAERRLGRSFVDYAVDPFLSGVYAGDPYQLPTRLALPKLYDLEQRYGSFIKGAMALAKQPKTDREKRATKAVFSTRGGFRSLVSALGRVIGDERIRTNCKELSIEPLGDKWKLSWGENTIIAEQVITTCPAYALPKLLNFLPKEQLDDLSNLYYAPVIEIGVGMKNTGDVHWNAFGGLVPSKEKQNVLGVLMPSACFQGRSPKEGANYACFIGGACHPEYINKTDEELIGLVNTSLHTMLGYPKGTCADVIRIYRHSHAIPQYMPETDARLRTIDAVELAYPGLHIIGNLKDGIGMGDRIKQAVDMAEKISLSVS

SEQ ID NO: 97 - protein, 458 aa, Thermovibrio sp.
MRVCVIGGGVSGLSTAFYLKRGGAQVKLLERENYPGGKARTYYEKGYIVESGPNGFLDGKPDTLELVKLLGAEKLLYRSSDKARKRFIYKNGRLVRLPENPIAFLSSYLLSWKGKVRVLGELLVPPSEKEDETLAEFVRRRLGKEALDYLLDPMVAGIFAGDPERMSLKAAFPTIYRLEREYGGLIRGLIAKAKEAKKKGAKSSGPAGPGGVLTSFVKGMSQFTQLLAQELGESFTPEAQVKTLEKKKDKWLVTYTLRGKEKSEEFDAVVLSLPAYAAAQVLKETSRELSELLASIEYSPISVVALGFEKRGLGHNLDGFGFLVPKVEGRKILGALWDSSVFPNRAPEGKALIRVMIGGARQPELALKSEEELTEIALKELKRIMKIRHYPEMVKVFRHEKGIPHYTIGHAEKVERIFKLGRELGNLFFCNNAYKGVGINDCTKSARETAEEVLNSLC

SEQ ID NO: 98 - protein, 536 aa, Brassica sp.
MDLSLLRPQPFLSPFSNPFPRSRPYKPLNLRCSVSGGSVVGSSTIEGGGGGKTVTADCVIVGGGISGLCIAQALVTKHPDAAKNVMVTEAKDRVGGNIITREEQGFLWEEGPNSFQPSDPMLTMVVDSGLKDDLVLGDPTAPRFVLWNGKLRPVPSKLTDLPFFDLMSIGGKIRAGFGAIGIRPSPPGREESVEEFVRRNLGDEVFERLIEPFCSGVYAGDPAKLSMKAAFGKVWKLEENGGSIIGGAFKAIQAKNKAPKTTRDPRLPKPKGQTVGSFRKGLTMLPEAISARLGDKVKVSWKLSSITKLASGEYSLTYETPEGIVTVQSKSVVMTVPSHVASSLLRPLSDSAAEALSKLYYPPVAAVSISYAKEAIRSECLIDGELKGFGQLHPRTQKVETLGTIYSSSLFPNRAPPGRVLLLNYIGGATNTGILSKSEGELVEAVDRDLRKMLIKPSSTDPLVLGVKVWPQAIPQFLIGHIDLVDAAKASLSSSGHEGLFLGGNYVAGVALGRCVEGAYETATQVNDFMSRYAYK

SEQ ID NO: 99 - protein, 521 aa, Brassica sp.
MASNAVADHDKPVSGKRVAVVGAGVSGLAAAYKLKSKGVNVTVFEADGRVGGKLRSVMHNGLIWDEGANTMTEAEPEVGSLLDDLGLREKQQFVSTFHALSIMFQPLSQKKRYIVRNGLPVMIPTNPIALVTSSVLSTQSKFQILLEPFLWKKNDSSSKVSDASVVESVSGFFQRHFGQEVVDYLIDPFMGGTSAADPESLSMKHSFPDLWNIEKSFGSIIVGAIRSKFAAKGSKNGETKSSTGTKKGSRGSFSFKGGMQILPDMLCKDLSREDLNLDSKVLSLSYNTGPREENWSLSCVSHNETQRQNLHYDAVVMTAPLCNVKEMKVMKGGEPFKLNFLPEIKYMPLSVIITTFTKEKVKRPLEGFGVLIPSIEQKHGFKTLGTLFSSMMFPDRCPSDLHLYTTFIGGSRNQELAKASTDELKQVVTSDLQRLLGIEGEPVFVNHVYWNKAFPLYDRSYDSVMEAIDKMEKDLPGFFYAGNHRGGLSVGKSIASGCKAADLVISYLESCSNDNKPEDSL

SEQ ID NO: 100 - protein, 539 aa, Gossypium sp.
MTALIDLSLLRSSPSVSPFSIPHHQHPPRFRKPFKLRCSLAEGPTISSSKIDGGESSIADCVIVGGGISGLCIAQALATKHRDVASNVIVTEARDRVGGNITTVERDGYLWEEGPNSFQPSDPILTMAVDSGLKDDLVLGDPNAPRFVLWEGKLRPVPSKPTDLPFFDLMSIAGKLRAGFGAIGIRPPPPGYEESVEEFVRRNLGAEVFERFIEPFCSGVYAGDPSKLSMKAAFGRVWKLEEIGGSIIGGTFKTIQERNKTPKPPRDPRLPKPKGQTVGSFRKGLTMLPEAIANSLGSNVKLSWKLSSITKLGNGGYNLQFETPEGMVSLQSRSVVMTIPSHVASNLLHPLSAAAADALSQFYYPPVASVTVSYPKEAIRKECLIDGELKGFGQLHPRSQGIETLGTIYSSSLFPNRAPSGRVLLLNYIGGATNTGILSKTEGELVEAVDRDLRKMLINPNAKDPLVLGVRVWPKAIPQFLVGHLDLLDSAKMALRDSGFHGLFLGGNYVSGVALGRCVEGAYEVAAEVKEFLSQYAYK

SEQ ID NO: 101 - protein, 503 aa, Gossypium sp.
MASTENKDDHSSAKRVAVIGAGVSGLAAAYKLKSQGLHVTVFESEGRAGGKLRSVSREGLIWDEGANTMTESEIEVRSLFDDLGIQDKEQVPIAQNKRYIVRNGVPVLIPSNPLALFTSSILSAKSKFQIILEPFLWRKSEASKVSDAYNQESVGGFFQRHFGQEVVDYLVDPFVAGTSAGDPESLSMCHSFPELWDLEQRFGSIIVGAVKSKFSAKRTNREETKNSVKRKALRGSFSFQGGMQTLADMLCKDLSKDELKLKSKVLSLSYSHEGKSTSENWSLSYASDRDKRSQGSSFDAVIMTAPVCNVKEMKITKGGNVFPLNFIPEVSYMPLSVIITAFKKENVKKPLEGFGVLIPSKEQQNGLKTLGTLFSSVMFPDRAPNNLYLYTTFVGGNRNKELAKASTDELKHIVTSDLQQLLGVEGEPTFFNHFYWSKAFPLYGRNYASVLEAIEKIERDLPGFFYAGNHKGGLSVGKSIASGCKAADNVITYLESSHDKLLK

SEQ ID NO: 102 - protein, 535 aa, Conyza sp.
MTSLTNFTPLKLTNPNYLTTTTTYNHRKLSNFRFRCSIARDSPTAPSISGDSSSRPLLDCVVVGAGISGLCIAQALSTKHGGDVVVTEARERVGGNISTVERDGYLWEEGPNSFQPSDPMLTMVVDSGLKDDLVLGDPTAPRFVLWDGDLKPVPSSPSDLPTFDLMSLGGKLRAGFGALGIRPPPPDREESVEEFVRRNLGDEVFERLIEPFCSGVYAGDPSKLSMKAAFGKVWKLEQNGGSIVGGAFKAIQAKNKSTKPPRDPRLPTPKGQTVGSFRKGQAMLPNAISKGLGSRVKLSWELVGITKSENRGYSLTYRTPDGLESLQTKTVVMTVPSYVASDLLRPLSVEAADALSKFYYPPVAAVSVSYPKEAIRADRLIDGQLKGFGQLHPRSQGVETLGTIYSSSLFPNRAPPGRVLLLNYIGGATNPGILSKTESQIVEAVDRDLRKMLINPKAGEPLTLGVKVWPRAIPQFLIGHYDILEAAKCALSLAGYRGMFLGGNYVSGVALGRCVENAYEVAADVSNFLSRGVYK

SEQ ID NO: 103 - protein, 498 aa, Conyza sp.
MASPTTTTDDNKEKAPAKRVAVVGAGVSGLAAAYKLKLHGINVTVFEAGEIAGGKLRSISQNGLIWDEGANTMTESEPDVSRLLDDLGLRDKQQSPLSQHKRYIVRNGKPVLVPSNPIALIQSSLLSTQSKLQILLEPFSWKKKNSSDTQESVGAFFQRHFGKEVVEYLINPVVAGTSGGDPESLSMRYSFPELWDLERRFGSLISGAFQSMISSRGRKKSPSGSSKRRRGSFSFLGGMQTLTNALSKEVGQHELNLQSKVLEMSYSCDDNTTGNWSIYCAPDQNKQLNQQPFDAVIMTAPLGNVKEMKITKTGSPFLLNFIPELSYMPVSVIISTFKKENVKRPLEGFGMLVPAKEQENGLKTLGTLFSSMMFPDRAPEDLYLYTTFVGGSRNKELANASRDELKQIVTSDLRQLLGAEGEPQFLTHYYWSKAYPLYGRDYGLVMEAIEKMERELPGYFYAGNHKGGLAVGKAISSGCKAAESVISYLDSYSDEKRC

SEQ ID NO: 104 - protein, 565 aa, Kochia sp.
MSAMASPSIIPQSFLQRSPTSLQSRSNYSKNHIIISISTPCSHGKNQRRFLRKTTHFRSIHCSTISTSTPTSSSNPGTLGEGGLLDCVIVGGGISGLCIAQALSTKYSSLSTNFIVTEAKDRVGGNITTKEDDGYIWEEGPNSFQPSDAVLTMAVDCGLKDELVFGDPKAPRFVLWNGKLRRVPSKLTDLPFFDLMSFPGKIRAGLGALGFRPSPPGREESVEDFVRRNLGDEVFERLIEPFCSGVYAGDPAKLSMKAAFGRVWVLEQMGGNIIGGALKTIQEKKNKPKPPRDPRLPKPKGQTVGSFRKGLIMLPNAISARLGSKVKLSWTLASISKTHNGEYNLIYDTPDGPVSVRTKSIVMTIPSYVASSLLRPFSDAAADSLSKFHYPPVAAVSLSYPEEAIRPECLIDGKLQGFGQLHPRSQGVETLGSIYSSSLFPGRAPPGRTMILSFIGGATNPGIVDKTQDELAKTVDKDLRRILINPSAKDPRVLGVKVWPQAIPQFLIGHFDLLDAAKAALTDAGCKGLFLGGNYVSGVALGRCIEGAYESAAEVVDFLSQYSDK

SEQ ID NO: 105 - protein, 531 aa, Lolium sp.
MVGATMATATVTAALPLRLRVPARSRRGQTRCAVASDATEAPAVPSARLSADCVIVGGGISGLCTAQALATKYGVTDLLVTEARARAGGNITTVERPDEGYLWEEGPNSFQPSDPVLTMAVDSGLKDDLVFGDPNAPRFVLWQGKLRPVPSKPGDLPFFDLMSIPGKLRAGLGALGIRPPPPGREESVEEFVRRNLGAEVFERLIEPFCSGVYAGDPSKLSMRAAFGKVWRLEENGGSIIGGTIKAIQDRGKNPKPPRDPRLPTPKGQTVASFRKGLAMLPNAIASRLGSKVKLSWKLTGITKSDNQGYVLAYETPEGVVSVQAKSVIMTIPSYIASEILRPLSSDAADGLSKFYYPPVAAVTVSYPTEAIRKECLIDGELQGFGQLHPRSQGVETLGTIYSSSLFPNRAPAGRVLLLNYIGGATNTGIVSKTESDLVEAVDRDLRKMLINPTAPDPLALGVRVWPQAIPQFLIGHLDRLDAAKSALARGGCSGLFLGGNYVAGVALGRCIEGAYESASEVSDFLTKYAYK

SEQ ID NO: 106 - protein, 509 aa, Lolium sp.
MAASDDPRAAPARSVAVVGAGVSGLVAAYRLRKSGVRVTVFEADDRAGGKIRTNSDGGFLWDEGANTMTESALEASRLIDDLGLEGRLQYPNSQHKRYTVKDGAPALIPSDPIALMKSSLLSTKSKFKLFLEPFLYEKSSTNNSKKVSDEHIRESVGSFFERHFGKEVVDYLIDPFVAGTSAGDPESLSIRHAFPALWNLEKKYGSIIAGAILSKLTAKGDSTKKGSAVSGKGRNKRVSFSFHGGMQTLVDALHKEIGDGNVKLATQVLSLACSCDGLSASNGWSIFVDSKDASNRELAKNQPFDAVIMTAPLSNVQRMKFTKGGAPFVLDFLPKVDYLPLSLMVTAFKKEDVKRPLEGFGVLVPYKEQQKHGLKTLGTLFSSMMFPDRAPNDQHLFTTFVGGSHNRDLAGAPTAILKQLVTSDLRKLLGVEGQPTFVRHIHWKNAFPLYGHDYDSALEAIGKMESDLPGFFYAGNNKDGLAVGNVIASGSKTADLVISYLELGIKRDN

SEQ ID NO: 107 - protein, 539 aa, Gossypium hirsutum
MTALIDLSLLRSSPSVSPFSIPHHQLPPRSRKPFKLRCSLAEGPTISSSKIDGGESSIADCVVVGGGISGLCIAQALATKHRDVASNVIVTEARDRVGGNITTVERDGYLWEEGPNSFQPSDPILTMAVDSGLKDDLVLGDPNAPRFVLWEGKLRPVPSKPTDLPFFDLMSIAGKLRAGFGAIGIRPPPPGYEESVEEFVRRNLGAEVFERFIEPFCSGVYAGDPSKLSMKAAFGRVWKLEEIGGSIIGGTFKTIQERNKTPKPPRDPRLPKPKGQTVGSFRKGLTMLPEAIANSLGSNVKLSWKLSSITKLGNGGYNLTFETPEGMVSLQSRSVVMTIPSHVASNLLHPLSAAAADALSQFYYPPVASVTVSYPKEAIRKECLIDGELKGFGQLHPRSQGIETLGTIYSSSLFPNRAPSGRVLLLNYIGGATNTGILSKTEGELVEAVDRDLRKMLINPNAKDPLVLGVRVWPKAIPQFLVGHLDLLDTAKMALRDSGFHGLFLGGNYVSGVALGRCVEGAYEVAAEVKEFLSQYAYK

SEQ ID NO: 108 - protein, 560 aa, Beta vulgaris
MKSMALSNCIPQTQCMPLHSSGHYRGNCIMLSIPCSLIGRRGYYSHKKRRMSMSCSTSSGSKSAVKEAGSGSGSGAGGLLDCVIVGGGISGLCIAQALCTKQSSLSPNFIVTEAKDRVGGNIVTVEADGYIWEEGPNSFQPSDAVLTMAVDSGLKDELVLGDPNAPRFVLWNDKLRPVPSSLTDLPFFDLMTIPGKIRAALGALGFRPSPPPHEESVEHFVRRNLGDEVFERLIEPFCSGVYAGDPAKLSMKAAFGKVWKLEQKGGSIIGGTLKAIQERGSNPKPPRDQRLPKPKGQTVGSFRKGLVMLPTAISARLGSRVKLSWTLSSIVKSLNGEYSLTYDTPDGLVSVRTKSVVMTVPSYVASRLLRPLSDSAADSLSKFYYPPVAAVSLSYPKEAIRSECLINGELQGFGQLHPRSQGVETLGTIYSSSLFPGRAPPGRILILSYIGGAKNPGILNKSKDELAETVDKDLRRMLINPDAKLPRVLGVRVWPQAIPQFSIGHFDLLDAAKAALTDTGVKGLFLGGNYVSGVALGRCIEGAYESAAEVVDFLSQYSDK

SEQ ID NO: 109 - protein, 530 aa, Hordeum vulgare
MAGAGATMATATAPPLRGRVTRRPHGVRPRCAAAGSATETPAAPGVRLSADCVIVGAGISGLCTAQALATRHGVGDLLVTEARDRPGGNITTVERPDEGYLWEEGPNSFQPSDPVLTMAVDSGLKDDLVFGDPNAPRFVLWEGKLRPVPSKPGDLPFFSLMSVPGKLRAGLGALGIRPPPPGREESVEEFVRRNLGAEVFERLIEPFCSGVYAGDPSKLSMKAAFGKVWRLEEIGGSIIGGTIKAIQDKGKNPKPPRDPRLPAPKGQTVASFRKGLAMLPNAIASRLGSKVKLSWKLTSITKADNQGYVLGYETPEGLVSVQAKSVIMTIPSYVASDILRPLSIDAADALSKFYYPPVAAVTVSYPKEAIRKECLIDGELQGFGQLHPRSQGVETLGTIYSSSLFPNRAPAGRVLLLNYIGGSTNTGIVSKTESDLVEAVDRDLRKMLINPRAADPLALGVRVWPQAIPQFLIGHLDRLAAAKSALGRGGYDGLFLGGNYVAGVALGRCIEGAYESASQVSDFLTKYAYK

SEQ ID NO: 110 - protein, 554 aa, Hordeum vulgare
MLTSATAPPSSSSCSSHAPARFASPSRPRRSASASARGRGRRVRPVLAMAASDDPRARSVAVVGAGVSGLVAAYMLRKSGVRVTVFEAEDRAGGKIRTNSDGGFLWDEGANTMTESALEASRLIDDLGLQDRLQYPNSQHKRYTVKDGAPALIPSDPIALMKSTVLSTKSKFKLFLEPFLYEKSSTRNSKKVSDEHLRESVGSFFERHFGKEVVDYLIDPFVAGTSAGDPESLSIRHAFPGLWNLEKKYGSLIVGAILSKLTAKGGSAKKGGASSGKGRNKRASFSFHGGMQTLVDALHKEVGDTNVKLGTQVLSLACNCDGLSASDGWSIFVDSKDASSKELARNQSFDAVIMTAPLSNVQRMKFTKGGRPFVLDFLPKVDYLPLSLMVTAFKKEDVKRPLEGFGVLIPFKEQQKHGLKTLGTLFSSMMFPDRAPNDQHLFTTFIGGSHNRDLAGAPTAILKQFVTSDLTKLLGVEGQPTFVKHIHWRNAFPLYGHDYDLALEAIGKMEGDLPGFFYAGNNKDGLAVGNVIASGSNTADLVISYLESGIKHVN

SEQ ID NO: 111 - protein, 531 aa, Triticum aestivum
MAGATMATATVAAASPLRGRVTGRPHRVRPRCATASSATETPAAPGVRLSAECVIVGAGISGLCTAQALATRYGVSDLLVTEARDRPGGNITTVERPDEGYLWEEGPNSFQPSDPVLTMAVDSGLKDDLVFGDPNAPRFVLWEGKLRPVPSKPGDLPFFSLMSIPGKLRAGLGALGIRPPPPGREESVEEFVRRNLGAEVFERLIEPFCSGVYAGDPSKLSMKAAFGKVWRLEEIGGSIIGGTIKAIQDKGKNPKPPRDPRLPAPKGQTVASFRKGLAMLPNAIASRLGSKVKLSWKLTSITKADNQGYVLGYETPEGLVSVQAKSVIMTIPSYVASDILRPLSIDAADALSKFYYPPVAAVTVSYPKEAIRKECLIDGELQGFGQLHPRSQGVETLGTIYSSSLFPNRAPAGRVLLLNYIGGSTNTGIVSKTESDLVGAVDRDLRKMLINPRAADPLALGVRVWPQAIPQFLIGHLDRLAAAKSALGQGGYDGLFLGGNYVAGVALGRCIEGAYESASQVSDFLTKYAYK

SEQ ID NO: 112 - protein, 501 aa, Solanum lycopersicum; identical to SEQ ID NO: 21

SEQ ID NO: 113 - protein, 546 aa, Triticum aestivum
MGIHPAALTFPRATEMAGATMATATVAAASPLRGRVTGRPHRVRPRCATASSATETPAAPGVRLSAECVIVGAGISGLCTAQALATRYGVSDLLVTEARDRPGGNITTVERPDEGYLWEEGPNSFQPSDPVLTMAVDSGLKDDLVFGDPNAPRFVLWEGKLRPVPSKPGDLPFFSLMSIPGKLRAGLGALGIRPPPPGREESVEEFVRRNLGAEVFERLIEPFCSGVYAGDPSKLSMKAAFGKVWRLEEIGGSIIGGTIKAIQDKGKNPKPPRDPRLPAPKGQTVASFRKGLAMLPNAIASRLGSKVKLSWKLTSITKADNQGYVLGYETPEGLVSVQAKSVIMTIPSYVASDILRPLSIDAADALSKFYYPPVAAVTVSYPKEAIRKECLIDGELQGFGQLHPRSQGVETLGTIYSSSLFPNRAPAGRVLLLNYIGGSTNTGIVSKTESDLVGAVDRDLRKMLINPRAADPLALGVRVWPQAIPQFLIGHLDRLAAAKSALGQGGYDGLFLGGNYVAGVALGRCIEGAYESASQVSDFLTKYAYK

SEQ ID NO: 114 - protein, 539 aa, Gossypium hirsutum
  UNSURE 137
  UNSURE 156
  UNSURE 168
MTALIDLSLLRSSPSVSPFSIPHHQLPPRSRKPFRLRCSVAEGPTISSSKIDGGESSIADCVVVGGGISGLCIAQALATKHRDVASNVIVTEARDRVGGNITTVERDGYLWEEGPNSFQPSDPILTMAVDSGLKDDXVLGDPNAPRFVLWEGKLRXVPSKPTDLPFFXLMSIAGKLRAGFGAIGIRPPPPGYEESVEEFVRRNLGAEVFERFIEPFCSGVYAGDPSKLSMKAAFGRVWKLEEIGGSIIGGTFKTIQERNKTPKPPRDPRLPKPKGQTVGSFRKGLTMLPEAIANSLGSNVKLSWKLSSITKLGNGGYNLTFETPEGMVSLQSRSVVMTIPSHVASNLLHPLSAAAADALSQFYYPPVASVTVSYPKEAIRKECLIDGELKGFGQLHPRSQGIETLGTIYSSSLFPNRAPSGRVLLLNYIGGATNTGILSKTEGELVEAVDRDLRKMLINPNAKDPLVLGVRVWPKAIPQFLVGHLDLLDSAKMALRDSGFHGLFLGGNYVSGVALGRCVEGAYEVAAEVKEFLSQYAYK

SEQ ID NO: 115 - protein, 503 aa, Gossypium hirsutum
MASTENKDDHSSAKRVAVIGAGVSGLAAAYKLKSQGLHVTVFESEGRAGGKLRSVSRDGLIWDEGANTMTESEIEVRSLFDDLGIQDKQQVPIAQNKRYIMRNGVPVLIPSNPLSLFTSSILSAKSKFQIILEPFLWRNSEASKVSDAYNQESVGGFFQRHFGQEVVDYLVDPFVAGTSAGDPESLSMCHSFPGLWDLEQRFGSIIVGAVKSKFSAKRTNREETKNSVKRKALRGSFSFKGGMQTLADMLCKDLSKDELKLKSKVLSLSYSHEGKSTSENWSLSYASDRDKRSQGSSFDAVIMTAPVCNVKEMKITKGGNVFPLNFIPEVSYMPLSVIITAFKKENVKKPLEGFGVLIPSKEQQNGLKTLGTLFSSVMFPDRAPNNLYLYTTFVGGNRNEKLAKASTDELKHIVTSDLQQLLGVEGEPTFFNHFYWSKAFPLYGRNYASVLEGIEKMERDLPGFFYAGNHKGGLSVGKSIASGCKAADNVITYLESSHDKLLK

SEQ ID NO: 116 - protein, 560 aa, Beta vulgaris
MKSMALSNCIPQTQCMPLHSSGHYRGNCIMLSIPCSLIGRRGYYSHKKRRMSMSCSTSSGSKSAVKEAGSGSGSGAGGLLDCVIVGGGISGLCIAQALCTKQSSLSPNFIVTEAKDRVGGNIVTVEADGYIWEEGPNSFQPSDAVLTMAVDSGLKDELVLGDPNAPRFVLWNDKLRPVPSSLTNLPFFDLMTIPGKIRAALGALGFRPSPPPHEESVEHFVRRNLGDEVFERLIEPFCSGVYAGDPAKLSMKAAFGKVWKLEQKGGSIIGGTLKAIQERGSNPKPPRDQRLPKPKGQTVGSFRKGLVMLPTAISARLGSRVKLSWTLSSIVKSLNGEYSLTYDTPDGLVSVRTKSVVMTVPSYVASRLLRPLSDSAADSLSKFYYPPVAAVSLSYPKEAIRSECLINGELQGFGQLHPRSQGVETLGTIYSSSLFPGRAPPGRILILSYIGGAKNPGILNKSKDELAETVDKDLRRMLINPDAKLPRVLGVRVWPQAIPQFSIGHFDLLDAAKAALTDTGVKGLFLGGNYVSGVALGRCIEGAYESAAEVVDFLSQYSDK

SEQ ID NO: 117 - protein, 508 aa, Brassica napus
MASNAVADHDKLSGKRVAVVGAGVSGLAAAYKLKSKGVNVTVFEADGRVGGKLRSVMHNGLIWDEGANTMTEAEPEVGSLLDDLGLREKQQFPLSQKKRYIVRNGLPVMIPTNPIALVTSSVLSTQSKFQILLEPFLWKKNDSSSKVSDASVVESVSGFFQRHFGQEVVDYLIDPFMGGTSAADPESLSMKHSFPDLWNIEKSFGSIIVGAIRSKFAAKGSKNGETKSSTGTKKGSRGSFSFKGGMQILPEMLCKDLSRDELNLDSKVLSLSYNAGPRQENWSLSCVSHNEAQGQNLHYDAVIMTAPLCNVKEMKVMKGGEPFKLNFLPEIKYMPLSVIITTFTKEKVKRPLEGFGVLIPTKEQKHGFKTLGTLFSSMMFPDRCPSDLHLYTTFIGGSRNQELAKASTDELKQVATSDLQRLLGVEGEPVFVNHVYWNKAFPLYDRSYDSVMEAIDKMEKDLPGFFYAGNHRGGLSVGKSIASGCKAADLVISYLESCSNDKKSEDSL

SEQ ID NO: 118 - DNA, 1638 bp, Triticum aestivum
  source 1..1638: mol_type = genomic DNA
atggggatccaccccgccgcactgacattcccccgagcgacagaaatggccggcgcaacaatggccaccgccaccgtcgcggccgcgtcgccgctccgcggcagggtcaccgggcgcccacaccgcgtccgcccgcgttgcgctaccgcgagcagcgcgaccgagactccggcggcgcccggcgtgcggctgtccgcggaatgcgtcattgtgggcgccggcatcagcggcctctgcaccgcgcaggcgctggccacccgatacggcgtcagcgacctgctcgtcacggaggcccgcgaccgcccgggcggcaacatcaccaccgtcgagcgtcccgacgaggggtacctgtgggaggagggacccaacagcttccagccctccgacccggtcctcaccatggccgtggacagcgggctcaaggatgacttggtgttcggggaccccaacgcgccccggttcgtgctgtgggaggggaagctgaggccggtgccgtcgaagccaggcgacctgcctttcttcagcctcatgagtatccctgggaagctcagggccggccttggcgcgctcggcattcgcccacctcctccagggcgcgaggagtcggtggaggagtttgtgcgccgcaacctcggtgccgaggtctttgagcgcctcatcgagcctttctgctcaggtgtatatgctggtgatccttcgaagcttagtatgaaggctgcatttgggaaggtctggaggttggaggagattggaggtagtattattggtggaaccatcaaggcgattcaggataaagggaagaaccccaaaccgccaagggatccccgacttccggcaccaaagggacagacggtggcatctttcaggaagggtctagccatgctcccgaatgccatcgcatctaggctgggtagtaaagtcaagctgtcatggaagcttacgagcattacaaaggcggacaaccaaggatatgtattaggttatgaaacaccagaaggacttgtttcagtgcaggctaaaagtgttatcatgaccatcccgtcatatgttgctagtgatatcttgcgcccactttcaattgatgcagcagatgcactctcaaaattctattatccgccagttgctgctgtaactgtttcatatccaaaagaagctattagaaaagaatgcttaattgatggggagctccagggtttcggccagttgcatccacgtagccaaggagtcgagactttagggacaatatatagctcttctctctttcctaatcgtgctcctgctggaagagtgttacttctgaactatatcgggggttctacaaatacagggatcgtctccaagactgagagtgacttagtaggagccgttgaccgtgacctcagaaaaatgttgataaaccctagagcagcagaccctttagcattaggggttcgagtgtggccacaagcaataccacagtttttgattgggcaccttgatcgccttgctgctgcaaaatctgcactgggccaaggcggctacgacgggmtgttcctaggaggaaactacgtmgcaggagttgccttgggccgatgcatcgagggtgcgtacgagagtgcctcacaagtatctgacttcttgaccaagtatgcctacaag

SEQ ID NO: 119 - DNA, 1617 bp, Gossypium hirsutum
  misc_feature 411: note = /note="n is a, c, g, or t"
  misc_feature 447: note = /note="n is a, c, g, or t"
  misc_feature 467: note = /note="n is a, c, g, or t"
  misc_feature 504: note = /note="n is a, c, g, or t"
  misc_feature 564: note = /note="n is a, c, g, or t"
  misc_feature 918: note = /note="n is a, c, g, or t"
  misc_feature 939: note = /note="n is a, c, g, or t"
  misc_feature 1008: note = /note="n is a, c, g, or t"
  source 1..1617: mol_type = genomic DNA
atgacggctctaatcgacctttctcttctccgctcctcgccctccgtttcccctttctccataccccaccaccagcttccgccccgctctcgtaaacctttcaggctccgatgctccgtcgccgagggtcccacgatttcctcatctaaaatcgacgggggagaatcatccatcgcggattgcgtcgtcgttggaggtggtatcagtggactttgcattgctcaagctctcgccaccaagcaccgtgacgtcgcttccaatgtgattgtgacggaagccagagaccgtgttggtggcaacatcactaccgttgagagagatggatatctgtgggaagaaggccccaacagttttcagccctccgatcctattctaaccatggccgtggatagtggattgaaggacgatttngttttaggtgaccctaatgcaccgcgatttgtactntgggagggaaaactaaggcntgtgccctccaagccaaccgacttgccgttttttganttgatgagcattgctggaaaacttagggctgggttcggggctattggcattcggcctccncctccgggttatgaagaatcggtggaggagtttgtgcgccgtaatcttggtgctgaggtttttgaacgctttattgaaccattttgttcaggtgtttatgcaggggatccttcaaaattaagcatgaaagcagcatttggaagagtatggaagctagaagagattggtggcagcatcattggtggcactttcaagacaatccaggagagaaataagacacctaagccacccagagacccgcgtctgccaaaaccgaagggccaaacagttggatcttttaggaagggacttaccatgctgcctgaggcaattgctaacagtttgggtagcaatgtaaaattatcttggaagctntccagtattaccaaattgggnaatggagggtataacttgacatttgaaacacctgaaggaatggtatctcttcagagtagaagtgttgtnatgaccattccatcccatgttgccagtaacttgttgcatcctctctcggctgctgctgcagatgcattatcccaattttattatcctccagttgcatcagtcacagtctcctatccaaaagaagccattcgaaaagaatgtttgattgatggtgaacttaaggggtttggccagttgcacccacgcagccaaggaattgaaactttagggacgatatacagttcatcacttttccccaatcgagctccatctggcagggtgttgctcttgaactacataggaggagctaccaacactggaattttgtccaagactgaaggggaacttgtagaagcagttgatcgtgatttgagaaaaatgcttataaatcctaatgcaaaggatcctcttgttttgggtgtaagagtatggccaaaagccattccacagttcttggttggtcatttggatctccttgatagtgcaaaaatggctctcagggattctgggtttcatggactgtttcttgggggcaactatgtatctggtgtggcattaggacggtgtgtggaaggtgcttacgaggttgcagctgaagtgaaggaattcctgtcacaatatgcatacaaa

SEQ ID NO: 120 - DNA, 1509 bp, Gossypium hirsutum
  misc_feature 630: note = /note="n is a, c, g, or t"
  source 1..1509: mol_type = genomic DNA
atggcgtcaactgaaaacaaagatgaccactcttctgctaaaagagtagctgtcattggcgctggtgttagtggactcgcggcagcttacaagttgaaatcacaaggtttacatgttacggtatttgaatctgaaggaagagctggagggaagttaagaagtgtttcgagggatggtttgatatgggatgaaggagcaaatacaatgactgaaagtgagattgaagtgagaagtttgtttgatgatcttggtattcaagataagcaacaagttccaattgcacagaacaagcggtatatcatgagaaatggcgtgcctgtattgatcccctcaaatcctctctcattattcacaagcagcattctttcagcaaaatcgaagtttcagattattttggagccttttctgtggagaaacagtgaagcctcaaaagtatctgatgcttataatcaggaaagtgtgggaggattttttcagcgccattttgggcaggaggttgtggattaccttgttgatccctttgttgctggcacaagtgctggagatcctgaatctctatctatgtgccattcctttccggggctatgggatcttgagcaaaggtttggctctatcattgttggagcagtnaaatctaaattctctgccaaaaggacaaatcgtgaagaaacaaaaaattcagtgaaaagaaaggctctacgtggctcattttccttcaagggtggaatgcagacacttgctgatatgttgtgcaaagatctttccaaagatgagcttaaactgaaatcaaaggttttgtcattatcttacagtcatgaggggaagtctacatcagagaactggtctctctcttatgcttctgatcgagacaagcgctcacaaggctcatcatttgatgctgtaataatgacggctccggtgtgcaatgttaaagaaatgaaaattactaaaggaggaaatgtctttccactgaacttcatccctgaggtgagttatatgccactatccgtcataattactgcttttaagaaggagaatgtcaagaaacccctagaaggttttggagttcttataccttcaaaggagcagcaaaatggtttaaaaactctcggtacacttttttcatctgtgatgtttcctgatcgtgcacctaataatttgtatctctatacaacctttgttggaggaaatcgaaatgagaagctggcaaaagcctcaacagatgaattgaagcatattgttacttccgaccttcagcagttgttgggagtggagggagaaccgacattcttcaatcatttctattggagcaaggcatttcccttgtatggccgtaactatgcttcggtcttggaaggcattgaaaagatggagagagatctccctggattcttctatgcaggtaaccacaaagggggattatcggtgggcaaatcgattgcttctggttgcaaagcagcagataatgtaattacatatttggaatcttcacatgacaagctgctgaaa

SEQ ID NO: 121 - DNA, 1680 bp, Beta vulgaris
  source 1..1680: mol_type = genomic DNA
atgaaatcaatggcgttatcaaactgcattccacagacacagtgcatgccattgcacagcagcgggcattacaggggcaattgtatcatgttgtcaattccatgtagtttaattggaagacgaggttattattcacataagaagaggaggatgagcatgagttgcagcacaagctcaggctcaaagtcagcggttaaagaagcaggatcaggatcaggatcaggagcaggaggattgctagactgcgtaatcgttggaggtggaattagcgggctttgcatcgcgcaggctctttgtacaaaacagtcctctttatccccaaattttatagtgacagaggccaaagacagagttggcggcaacatcgtcactgtggaggccgatggctatatctgggaggagggacccaatagcttccagccttccgacgcggtgctcaccatggcggtcgacagtggcttgaaagatgagttggtgctcggagatcccaatgctcctcgctttgtgctgtggaatgacaaattaaggcccgtaccttccagtctcaccaacctccctttcttcgacctcatgaccattcccggcaagattagggctgctcttggtgctctcggatttcgcccttctcctccacctcatgaggaatctgttgaacactttgtgcgtcgtaatctcggagatgaggtctttgaacgcttgattgaacccttttgttcaggtgtgtatgccggtgatcctgccaagctgagtatgaaagctgcttttgggaaggtctggaagttggagcaaaagggtggcagcataattggtggcactctcaaagctatacaggaaagagggagtaatcctaagccgccccgtgaccagcgcctccctaaaccaaagggtcagactgttggatcctttagaaagggactcgttatgttgcctaccgccatttctgctcgacttggcagtagagtgaaactatcttggaccctttctagtatcgtaaagtcactcaatggagaatatagtctgacttatgataccccagatggcttggtttctgtaagaaccaaaagtgttgtgatgactgtcccatcatatgttgcaagtaggcttcttcgtccactttcagactctgctgcagattctctttcaaaattttactatccaccagttgcagcagtgtcactttcctatcctaaagaagcgatcagatcagaatgcttgattaatggtgaacttcaaggtttcgggcaactacatccccgcagtcagggtgtggaaaccttgggaacaatttatagttcgtctcttttccctggtcgagcaccacctggtaggatcttgatcttgagctacatcggaggtgctaaaaatcctggcatattaaacaagtcgaaagatgaacttgccgagacagttgacaaggacctgagaagaatgcttataaatcctgatgcaaaacttcctcgtgtactgggtgtgagagtatggcctcaagcaataccccagttttctattgggcactttgatctgctcgatgctgcaaaagctgctctgacagatacaggggtcaaaggactgtttcttggtggcaactatgtttcaggtgttgccttggggcggtgtatagagggtgcttatgagtctgcagctgaggtagtagatttcctctcacagtactcagacaaa

SEQ ID NO: 122 - DNA, 1593 bp, Hordeum vulgare
  source 1..1593: mol_type = genomic DNA
atggccggtgcaggtgcaaccatggccaccgccaccgcgccgccgctccgcggcagggtcacccggcgcccgcacggcgtccgcccgcgttgcgcggccgcgggcagcgcgaccgagacccccgcggcgcccggcgtgcgtctgtccgcggactgcgtcatcgtgggcgccggcatcagcggcctctgcaccgcgcaggcgctggccacccggcacggcgtcggcgacctgctcgtcacggaggcccgcgaccgcccgggcggcaacatcaccaccgtcgagcgccccgacgaggggtacctctgggaggagggccccaacagcttccagccctccgaccccgtcctcaccatggccgtggacagcgggctcaaggatgacctggtgttcggggaccccaacgcgccccggttcgtgctgtgggaggggaagctgaggccggtgccgtccaagccaggcgacctgccgttcttcagcctcatgagcgtccccgggaagctcagggccggcctcggcgccctcggcattcgcccgcctcctccagggcgcgaggagtcggtggaggagtttgtgcgccgcaacctcggtgccgaggtctttgagcgccttatcgaacctttctgctcaggtgtgtatgctggtgatccttcgaagctcagtatgaaggctgcatttgggaaggtttggaggttggaggagattgggggtagtattattggtggaaccatcaaggcaattcaggataaagggaagaaccccaaaccgccaagggatccccgacttccggcaccaaagggacagacggtggcatctttcaggaagggtctggccatgctcccgaatgccatcgcatctaggttgggtagtaaagtcaagctgtcatggaagcttacgagcattacaaaggcggacaaccaaggatatgtattaggttatgaaacaccagaaggacttgtttcagtgcaggctaaaagtgttatcatgaccatcccgtcatatgttgctagtgatatcttacgcccactttcaattgatgcagcagatgcactctcaaaattctattatccgccagttgctgctgtaactgtttcatatccaaaagaagctattagaaaagaatgcttaattgatggggagctccagggtttcggccagctgcatccacgtagccaaggagtcgagactttagggacaatatatagctcttctctctttcccaatcgtgctcctgctggaagagtgttacttctgaactatatcgggggttctacaaatacagggatcgtctccaagaccgagagtgacttagtagaagctgttgatcgtgatctcagaaaaatgttgataaaccctagagcagcagaccctttagcattaggggtcagagtgtggccacaagcaataccacagtttttgattggacaccttgatcgccttgctgctgcaaaatctgcactgggccgaggcgggtacgacgggttattcctaggaggaaactacgtagcaggagttgccttgggccgatgcatcgagggtgcgtacgagagtgcctcacaagtatctgacttcttgaccaagtatgcctacaagtga

SEQ ID NO: 123 - DNA, 1665 bp, Hordeum vulgare
  source 1..1665: mol_type = genomic DNA
atgctcacttccgccaccgctcccccctcctcctcctcctgctcgtcccacgctcccgcccgcttcgcctccccgtcccggccccgtcgctccgcgtccgcgtccgcgcgcgggcgagggcgccgcgtccgccccgtgctcgccatggccgcctccgacgacccccgcgccaggtcggtcgccgtcgtcggcgccggcgtcagtgggctcgtggcggcgtacatgctgaggaagagcggcgtgcgggtcaccgtgttcgaggcggaagaccgcgcgggagggaagatacggaccaactccgacggcggattcctctgggacgaaggagccaacaccatgacagaaagtgcactggaggctagtaggctaatcgacgatcttggtcttcaggacagactgcagtatcctaactcccagcacaagcgttacaccgttaaggatggggcgccagcactgattccttcagatcccattgcgctaatgaaaagcactgttctttctacgaaatcaaagttcaagttatttctggaaccatttctctatgaaaaatctagcacaaggaactccaaaaaagtgtctgacgagcatttgcgcgagagcgttgggagtttttttgaacgccattttgggaaagaggttgttgactatcttattgatccatttgtagctggaacaagtgcaggagatcccgagtcattatctattcgtcatgcatttccggggttatggaatttagaaaaaaagtatggttctctcatcgttggtgccatcttgtcaaaactaacagctaaaggtggttcagcaaagaaaggaggcgcttcgtcaggaaaaggaaggaacaagcgggcctcattttcatttcatggtggcatgcagacactagtagacgcacttcacaaggaagttggagatactaatgtgaagcttggaacacaagtattgtcattggcgtgtaactgtgatggactctctgcatcagatgggtggtcaatttttgttgattcaaaggatgctagtagtaaggagcttgcaaggaaccaatcctttgatgctgttataatgacagctccactgtccaatgtccagaggatgaagttcacaaaaggcggacgtccctttgtgctagactttcttcctaaggtggattatctgccgttgtccctcatggtaacagcatttaagaaggaagacgtcaaaagacccctcgaaggatttggggtcttaataccctttaaggaacaacaaaaacatggtttgaaaacgctcggaactctcttctcctctatgatgttcccagatcgagctcctaatgaccagcacttgtttacaacattcattgggggaagccacaatagagatctcgctggagctccaacggctatcttgaaacaatttgtgacatctgaccttacaaagctactgggggtagaggggcagccaacttttgtgaaacatatacattggagaaatgcttttcctttgtatggccatgattatgatttggcactggaagctataggaaagatggaaggtgatctcccagggttcttctatgcaggaaataacaaggatgggttggctgttggaaatgtcatagcttcaggaagtaacactgcagaccttgtgatctcataccttgagtcaggcatcaagcatgttaattga

SEQ ID NO: 124 - DNA, 1593 bp, Triticum aestivum
  source 1..1593: mol_type = genomic DNA
atggccggcgcaacaatggccaccgccaccgtcgcggccgcgtcgccgctccgcggcagggtcaccgggcgcccacaccgcgtccgcccgcgttgcgctaccgcgagcagcgcgaccgagactccggcggcgcccggcgtgcggctgtccgcggaatgcgtcattgtgggcgccggcatcagcggcctctgcaccgcgcaggcgctggccacccgatacggcgtcagcgacctgctcgtcacggaggcccgcgaccgcccgggcggcaacatcaccaccgtcgagcgtcccgacgaggggtacctgtgggaggagggacccaacagcttccagccctccgacccggtcctcaccatggccgtggacagcgggctcaaggatgacttggtgttcggggaccccaacgcgccccggttcgtgctgtgggaggggaagctgaggccggtgccgtcgaagccaggcgacctgcctttcttcagcctcatgagtatccctgggaagctcagggccggccttggcgcgctcggcattcgcccacctcctccagggcgcgaggagtcggtggaggagtttgtgcgccgcaacctcggtgccgaggtctttgagcgcctcatcgagcctttctgctcaggtgtatatgctggtgatccttcgaagcttagtatgaaggctgcatttgggaaggtctggaggttggaggagattggaggtagtattattggtggaaccatcaaggcgattcaggataaagggaagaaccccaaaccgccaagggatccccgacttccggcaccaaagggacagacggtggcatctttcaggaagggtctagccatgctcccgaatgccatcgcatctaggctgggtagtaaagtcaagctgtcatggaagcttacgagcattacaaaggcggacaaccaaggatatgtattaggttatgaaacaccagaaggacttgtttcagtgcaggctaaaagtgttatcatgaccatcccgtcatatgttgctagtgatatcttgcgcccactttcaattgatgcagcagatgcactctcaaaattctattatccgccagttgctgctgtaactgtttcatatccaaaagaagctattagaaaagaatgcttaattgatggggagctccagggtttcggccagttgcatccacgtagccaaggagtcgagactttagggacaatatatagctcttctctctttcctaatcgtgctcctgctggaagagtgttacttctgaactatatcgggggttctacaaatacagggatcgtctccaagactgagagtgacttagtaggagccgttgaccgtgacctcagaaaaatgttgataaaccctagagcagcagaccctttagcattaggggttcgagtgtggccacaagcaataccacagtttttgattgggcaccttgatcgccttgctgctgcaaaatctgcactgggccaaggcggctacgacgggttgttcctaggaggaaactacgtcgcaggagttgccttgggccgatgcatcgagggtgcgtacgagagtgcctcacaagtatctgacttcttgaccaagtatgcctacaag

SEQ ID NO: 125 - DNA, 1506 bp, Solanum lycopersicum
  source 1..1506: mol_type = genomic DNA
atggctccatctgccggagaagataaacaaaagagagttgcagtcattggtgctggcgtcagtggacttgctgcagcatacaagttgaaagttcatggcttgaatgtcacagtatttgaagcagaagggagagctggagggaagttacgaagcctgagtcaagatggcctaatatgggatgaaggcgcaaatactatgactgaaagtgaaggtgatgtcacatttttgcttgattcgcttggactccgagaaaaacaacaattcccactttcacagaacaagcgctacattgccagaaatggtactcctactctgataccttcaaatccatttgacctattcaaaagcaattttctttccactggatcaaagcttcagatgcttttcgagccacttttgtggaagaataaaaagcttacaaaggtgtctgacaaacacgaaagtgtcagtggattcttccagcgtcattttggaaaggaggttgtcgactatctaattgacccttttgttgctggaacatgtggtggtgatcctgattcgctttcaatgcacctttcatttccagacttgtggaatttagagaaaaggtttggttcagtcatagttggggcaattcaatctaagttatctcctataaaggaaaagaaacaagggccacccagaacttcaataaataagaagcgccagcgggggtccttttcatttttgggcggaatgcaaacacttactgacgcaatatgcaaaaatctcaaagaagatgaacttaggctaaactctagagttctggaattatcttgtagctgtagtggggactctgcgatagatagctggtcaattttttctgcctctccacacaagcggcaagcagaagaagaatcatttgatgctgtaattatgacggcccctctctgtgacgttaagagtatgaagattgctaagagaggaaatccatttctgctcaactttattcctgaggtcgattatgtaccactatctgttgttataaccacatttaagaaggagagtgtaaagcatccccttgagggttttggagtgcttgtaccctcccaggagcaaaaacatggtctgaagacactaggcaccctcttctcttctatgatgtttccagatcgtgcacccaacaatgtctatctctatactacatttgttggtggaagccgaaatagagaactcgcgaaagcctcgaggactgagctgaaagagatagtaacttctgaccttaagcagttgttgggtgctgagggagagccaacatatgtgaatcatttatgctggagtaaagcatttccattgtacgggcataactatgattcagtcctagacgcaattgacaaaatggagaaaagccttcctggattattctatgcaggtaaccacaaggggggattgtcagttggcaaagcattatcttctggatgcaatgcagcagatcttgttatatcatatcttgaagcggtttcagctgacaccaaaaaccatagctga

SEQ ID NO: 126 - DNA, 1677 bp, Solanum lycopersicum
  source 1..1677: mol_type = genomic DNA
atgacaacaacggccgtcgtcaaccatcctagcattttcactcaccggtcgccgctgccgtcgccgtcctcctcctcatcctcatcgccgtcatttttatttttaaatcgtacgaattttattccatacttttccacctccaagcgcagtagtgtcaattgcaatggctggagaacacggtgttccgttgcgaagaattatacagttcctccctcagaagttgacggtaatcagttaccggagctggattgtgtggtagtcggagcaggaattagtggtctctgcattgctaaggtgatatcggctaattatcccaatttgatggtgacggaggcgagggatcgtgccggtggaaacataacgacggtggaaagagatggatacttatgggaagaaggtcctaacagtttccagccttcggatcctatgttgactatggctgtagattgtggattgaaggatgatttggtgttgggagatcctgatgcgcctcgctttgtcttgtggaaggataaactaaggcctgttcccggcaagctcactgatcttcccttctttgatttgatgagtattcctggcaagctcagagctggttttggtgccattggccttcgcccttcacctccaggttatgaggaatcagttgagcagttcgtgcgtcgtaatcttggtgctgaagtctttgaacgtttgattgaaccattttgttctggtgtttatgctggcgacccatcaaaattgagtatgaaagcagcatttgggaaagtgtggaagctagaacaaactggtggtagcattattgggggaacctttaaggcaataaaggagagatccagtaaccctaaaccgcctcgtgatccgcgtttaccaacaccaaaaggacaaactgttggttcatttaggaagggtctgagaatgctgccagatgcaatttgtgaaagactgggaagcaaagtgaaactatcatggaagctttctagcattacaaagtcagataaaggaggatatctcttgacatacgagacaccagaaggagtagtttctctgcgaagtcgaagcattgtcatgactgttccatcctatgtagcaagcaacatattacgccctctttcggtcgccgcagcagatgcactttcaagtttctactatcccccagttgcagcagtgacaatttcatatcctcaagaggctattcgtgatgagcgtctggttgatggtgaactaaagggatttgggcagttgcatccacgttcacagggagtggaaacactaggaacaatatatagttcatcactcttccctaaccgtgctccaaatggccgggtgctactcttgaactacattggaggagcaacaaatactgaaattgtgtctaagacagagagccaacttgtggaagcagttgaccgtgacctcagaaagatgcttataaaacccaaagcacaagatccctttgttacgggtgtgcgagtatggccacaagctatcccacagtttttggtcggacatctggatacactaggtactgctaaagctgctctaagtgataatgggcttgacgggctattccttgggggtaattatgtgtctggtgtagcattgggaaggtgtgttgaaggtgcttatgaaattgcatctgaagtaactgggtttctgtctcagtatgcatacaaatga

SEQ ID NO: 127 - DNA, 1620 bp, Gossypium hirsutum
  source 1..1620: mol_type = genomic DNA
atgacggctctaatcgacctttctcttctccgctcctcgccctccgtttcccctttctccataccccaccaccagcttccgccccgctctcgtaaacctttcaagctccgatgctccctcgccgagggtcccacgatttcctcatctaaaatcgacgggggagaatcatccatcgcggattgcgtcgtcgttggaggtggtatcagtggactttgcattgctcaagctctcgccaccaagcaccgtgacgtcgcttccaatgtgattgtgacggaagccagagaccgtgttggtggcaacatcactaccgttgagagagatggatatctgtgggaagaaggacccaacagttttcagccctccgatcctattctaaccatggccgtggatagtggattgaaggacgatttagttttaggtgaccctaatgcaccgcgatttgtactgtgggagggaaaactaaggcctgtgccctccaagccaaccgacttgccgttttttgacttgatgagcattgctggaaaacttagggctgggttcggggctattggcattcggcctccccctccgggttatgaagaatcggtggaggagtttgtgcgccgtaatcttggtgctgaggtttttgaacgctttattgaaccattttgttcaggtgtttatgcaggggatccttcaaaattaagcatgaaagcagcatttggaagagtatggaagctagaagagattggtggcagcatcattggtggcactttcaagacaatccaggagagaaataagacacctaagccacccagagacccgcgtctgccaaaaccgaagggccaaacagttggatcttttaggaagggacttaccatgctgcctgaggcaattgctaacagtttgggtagcaatgtaaaattatcttggaagctctccagtattaccaaattgggtaatggagggtataacttgacatttgaaacacctgaaggaatggtatctcttcagagtagaagtgttgtcatgaccattccatcccatgttgccagtaacttgttgcatcctctctcggctgctgctgcagatgcattatcccaattttattatcctccagttgcatcagtcacagtctcctatccaaaagaagccattcgaaaagaatgtttgattgatggtgaacttaaggggtttggccagttgcacccacgcagccaaggaatcgaaactttagggacgatatacagttcatcacttttccccaatcgagctccatctggcagggtgttgctcttgaactacataggaggagctaccaacactggaattttgtccaagactgaaggggaacttgtagaagcagttgatcgtgatttgagaaaaatgcttataaatcctaatgcaaaggatcctcttgttttgggtgtaagagtatggccaaaagccattccacagttcttggttggtcatctggatctccttgatactgcaaaaatggctctcagggattctgggtttcatggactgtttcttgggggcaactatgtatctggtgtggcattaggacggtgtgtggaaggtgcttacgaggttgcagctgaagtgaaggaattcctgtcacaatatgcatacaaataa

SEQ ID NO: 128 - DNA, 1683 bp, Beta vulgaris
  source 1..1683: mol_type = genomic DNA
atgaaatcaatggcgttatcaaactgcattccacagacacagtgcatgccattgcacagcagcgggcattacaggggcaattgtatcatgttgtcaattccatgtagtttaattggaagacgaggttattattcacataagaagaggaggatgagcatgagttgcagcacaagctcaggctcaaagtcagcggttaaagaagcaggatcaggatcaggatcaggagcaggaggattgctagactgcgtaatcgttggaggtggaattagcgggctttgcatcgcgcaggctctttgtacaaaacagtcctctttatccccaaattttatagtgacagaggccaaagacagagttggcggcaacatcgtcactgtggaggccgatggctatatctgggaggagggacccaatagcttccagccttccgacgcggtgctcaccatggcggtcgacagtggcttgaaagatgagttggtgctcggagatcccaatgctcctcgctttgtgctgtggaatgacaaattaaggcccgtaccttccagtctcaccgacctccctttcttcgacctcatgaccattcccggcaagattagggctgctcttggtgctctcggatttcgcccttctcctccacctcatgaggaatctgttgaacactttgtgcgtcgtaatctcggagatgaggtctttgaacgcttgattgaacccttttgttcaggtgtgtatgccggtgatcctgccaagctgagtatgaaagctgcttttgggaaggtctggaagttggagcaaaagggtggcagcataattggtggcactctcaaagctatacaggaaagagggagtaatcctaagccgccccgtgaccagcgcctccctaaaccaaagggtcagactgttggatcctttagaaagggactcgttatgttgcctaccgccatttctgctcgacttggcagtagagtgaaactatcttggaccctttctagtatcgtaaagtcactcaatggagaatatagtctgacttatgataccccagatggcttggtttctgtaagaaccaaaagtgttgtgatgactgtcccatcatatgttgcaagtaggcttcttcgtccactttcagactctgctgcagattctctttcaaaattttactatccaccagttgcagcagtgtcactttcctatcctaaagaagcgatcagatcagaatgcttgattaatggtgaacttcaaggtttcgggcaactacatccccgcagtcagggtgtggaaaccttgggaacaatttatagttcgtctcttttccctggtcgagcaccacctggtaggatcttgatcttgagctacatcggaggtgctaaaaatcctggcatattaaacaagtcgaaagatgaacttgccgagacagttgacaaggacctgagaagaatgcttataaatcctgatgcaaaacttcctcgtgtactgggtgtgagagtatggcctcaagcaataccccagttttctattgggcactttgatctgctcgatgctgcaaaagctgctctgacagatacaggggtcaaaggactgtttcttggtggcaactatgtttcaggtgttgccttggggcggtgtatagagggtgcttatgagtctgcagctgaggtagtagatttcctctcacagtactcagacaaatag

SEQ ID NO: 129 - protein, 547 aa, Alopecurus myosuroides
MLTSATTPSSSSASSRASTRFASSSRPRRTAYARGRRLRPVLAMAASDDPRARSVAVVGAGISGLVAAYRLSKSGVRVTVFEADDRAGGKIRTNSDSGFLWDEGANTMTESALEASRLIDDLGLEDRLQYPNSQHKRYTVKDGAPALIPSDPIALMKSSLLSTKSKFKLFLEPFLYDKSSTKSSKKVSDEHISESVGSFFERHFGKEVVDYLIDPFVAGTSAGDPESLSIRHAFPGLWNLEKKYGSIIVGAIMSKLTAKGDKKGSAVSGKGRNKRASFSFHGGMQTLVDALHKEVGDGNVKLGAQVLSLACICDGLSASDGWSISVDSKDASNKELTKNHSFDAVIMTAPLSNVQRMKFTKGGAPFVLDFLPKVDYLPLSLMVTAFKKEDVKRPLEGFGVLIPYKEQQKHGLKTLGTLFSSMMFPDRAPNDQHLFTTFVGGSHNRDLAGAPTSILKQLVTSDLGKLLGVEGQPTFVKHIHWRNAFPLYGHDYDSALEAIGKMESDLPGFFYAGNNKDGLAVGNVIASGSKTADLVISYLESGIKQDN

SEQ ID NO: 130 - DNA, 1641 bp, Alopecurus myosuroides
  source 1..1641: mol_type = genomic DNA
atgctcacttccgccaccaccccctcctcctcctccgcttcgtcccgcgcgtccacccgcttcgcctcctcgtcccgtcctcgtcgcaccgcctacgcgcgcgggcgccggcttcgccccgtgctcgccatggccgcctccgacgacccacgcgccaggtcggtcgccgttgtaggcgccggcatcagtgggctcgtggcggcgtacagactgagcaagagcggcgtgcgggtcacggtgttcgaggcggacgaccgggcaggagggaagatacggaccaactccgacagcggattcctctgggacgaaggagccaacaccatgacagaaagtgcgctggaggcgagtagactaatcgatgatcttggtcttgaggacaggctgcagtatcctaactcccagcacaagcggtacactgttaaagatggagcgccagcactgatcccctcagatcccattgcgctgatgaaaagcagtcttctttctacgaaatcaaagttcaagttatttttggaaccatttctctacgacaagtctagcacaaagagctccaagaaagtgtctgatgagcatataagtgagagtgttgggagcttctttgaacgccactttggaaaagaagttgttgactatcttattgatccatttgtagctggaacaagtgcaggagatcccgagtcattatctatccgtcatgcatttccagggttatggaatttagaaaagaagtatggttctatcattgttggtgccatcatgtctaaactaacagctaaaggtgataagaaaggaagcgctgtatcaggaaaaggaaggaataagcgggcgtcattttcatttcatggtggtatgcagacactagtagatgcacttcacaaagaagttggagatggtaatgtgaaacttggagcacaagtgttgtcattggcatgtatctgtgatgggctctctgcatcagatgggtggtcaatttctgttgattcaaaagatgctagtaacaaggagctaacaaagaaccattcctttgatgctgttatcatgacagctccactgtctaatgtccagaggatgaagtttacaaaaggtggagctccatttgtgctagactttcttcctaaggtggattatctgccgttgtccctcatggtaacagcttttaagaaggaagatgtcaaaagacctctggaaggatttggggtgttgataccctacaaggaacagcaaaagcatggtctgaaaacccttggaactctcttctcctctatgatgtttccagatcgagctcctaatgaccaacacttatttacaacattcgttgggggaagccacaacagggatcttgctggagctccaacgtctatcttaaaacaacttgtgacctctgaccttggaaagctcctgggtgtagagggacagccaacttttgtgaaacatatacattggagaaatgcttttcctttatatggccatgattatgattcggcattggaagctataggaaagatggagagtgatcttccagggttcttctatgcaggaaataacaaggacgggttggctgttggaaatgttatagcttcaggaagtaagactgctgatctggtgatctcgtatcttgagtcaggcatcaagcaagataat

SEQ ID NO: 131 - protein, 7 aa, Amaranthus sp.
  VARIANT 3: note = / replace = "Ser"
GTCGGDP

SEQ ID NO: 132 - protein, 7 aa, Amaranthus sp.
  VARIANT 1: note = / replace = "Ser" or "Cys"
  VARIANT 4: note = / replace = "Asn"
  VARIANT 5: note = / replace = any amino acid
  VARIANT 6: note = / replace = any amino acid
APSDMCL

SEQ ID NO: 133 - protein, 7 aa, Amaranthus sp.
  VARIANT 1: note = / replace = "Gln"
  VARIANT 2: note = / replace = "Asp"
  VARIANT 6: note = / replace = "Leu"
REKQQLP

SEQ ID NO: 134 - protein, 6 aa, Amaranthus sp.
  VARIANT 2: note = / replace = "Val"
LIPSKE

SEQ ID NO: 135 - protein, 7 aa, Amaranthus sp.
  VARIANT 3: note = / replace = "Lys" or "His"
SQNKRYI

SEQ ID NO: 136 - protein, 8 aa, Amaranthus sp.
TLGTLFSS

SEQ ID NO: 137 - protein, 7 aa, Amaranthus sp.
  VARIANT 1: note = / replace = "Tyr"
  VARIANT 5: note = / replace = "Ile"
FTTFVGG

(19) United States
(12) Patent Application Publication
(10) Pub. No.: US 2024/0254502 A1
(43) Pub. Date: Aug. 1, 2024

(54) PLANTS HAVING INCREASED TOLERANCE TO HERBICIDES

(71) Applicant: Basf Agro B.V., Arnhem (NL)

(72) Inventors: Raphael Aponte, Mannheim (DE); Stefan Tresch, Ludwigshafen (DE); Dario Massa, Limburgerhof (DE); Matthias Witschel, Ludwigshafen (DE); Tobias Seiser, Ludwigshafen (DE); Jill Marie Paulik, Durham, NC (US)

(21) Appl. No.: 18/419,299

(22) Filed: Jan. 22, 2024

Related U.S. Application Data
(63) Continuation of application No. 16/468,036, filed on Jun. 10, 2019, now Pat. No. 11,879,132.

(30) Foreign Application Priority Data
Dec. 20, 2016 (EP) 16205383.9

(51) Int. Cl. C12N 15/82; C07K 14/195; A01H 6/20; A01H 6/46; A01H 6/54

(52) U.S. Cl. CPC C12N 15/8274; C07K 14/195; A01H 6/20; A01H 6/4684; A01H 6/542

(57) ABSTRACT

The present invention refers to a plant or plant part comprising a polynucleotide encoding a mutated PPO polypeptide, the expression of said polynucleotide confers to the plant or plant part tolerance to herbicides.

This application is a continuation of U.S. patent application Ser. No. 16/468,036, which is a U.S. National Stage application of International Application No. PCT/EP2017/083244, filed Dec. 18, 2017, which claims priority to European Patent Application No. 16205383.9, filed on Dec. 20, 2016. The entire contents of the aforementioned applications are hereby incorporated herein by reference in their entirety.

### INCORPORATION BY REFERENCE OF MATERIAL SUBMITTED ELECTRONICALLY

The Sequence Listing, which is a part of the present disclosure, is submitted concurrently with the specification as an XML file. The name of the file containing the Sequence Listing is “78939A_Seqlisting.xml”, which was created on Dec. 8, 2023 and is 202,681 bytes in size. The subject matter of the Sequence Listing is incorporated herein in its entirety by reference.

### FIELD OF THE INVENTION

The present invention relates in general to methods for conferring on plants agricultural level tolerance to a herbicide. Particularly, the invention refers to plants having an increased tolerance to PPO-inhibiting herbicides. More specifically, the present invention relates to methods and plants obtained by mutagenesis and cross-breeding and transformation that have an increased tolerance to PPO-inhibiting herbicides.

### BACKGROUND OF THE INVENTION

Herbicides that inhibit protoporphyrinogen oxidase (hereinafter referred to as Protox or PPO; EC:1.3.3.4), a key enzyme in the biosynthesis of protoporphyrin IX, have been used for selective weed control since the 1960s. PPO catalyzes the last common step in chlorophyll and heme biosynthesis which is the oxidation of protoporphyrinogen IX to protoporphyrin IX. (Matringe et al. 1989. Biochem. 1. 260: 231). PPO-inhibiting herbicides include many different structural classes of molecules (Duke et al. 1991. Weed Sci. 39: 465; Nandihalli et al. 1992. Pesticide Biochem. Physiol. 43: 193; Matringe et al. 1989. FEBS Lett. 245: 35; Yanase and Andoh. 1989. Pesticide Biochem. Physiol. 35: 70). These herbicidal compounds include the diphenylethers (e.g. lactofen, (+−)-2-ethoxy-1-methyl-2-oxoethyl 5-{2-chloro-4-(trifluoromethyl)phenoxy}-2-nitrobenzoate; acifluorfen, 5-{2-chloro-4-(trifluoromethyl)phenoxy}-2-nitrobenzoic acid; its methyl ester; or oxyfluorfen, 2-chloro-1-(3-ethoxy-4-nitrophenoxy)-4-(trifluorobenzene)}, oxidiazoles, (e.g. oxidiazon, 3-{2,4-dichloro-5-(1-methylethoxy)phenyl}-5-(1,1-dimethylethyl)-1,3,4-oxadiazol-2-(3H)-one), cyclic imides (e.g. S-23142, N-(4-chloro-2-fluoro-5-propargyloxyphenyl)-3,4,5,6-tetrahydrophthalimide; chlorophthalim, N-(4-chlorophenyl)-3,4,5,6-tetrahydrophthalimide), phenyl pyrazoles (e.g. TNPP-ethyl, ethyl 2-{1-(2,3,4-trichlorophenyl)-4-nitropyrazolyl-5-oxy}propionate; M&B 39279), pyridine derivatives (e.g. LS 82-556), and phenopylate and its O-phenylpyrrolidino- and piperidinocarbamate analogs. Many of these compounds competitively inhibit the normal reaction catalyzed by the enzyme, apparently acting as substrate analogs.

Application of PPO-inhibiting herbicides results in the accumulation of protoporphyrinogen IX in the chloroplast and mitochondria, which is believed to leak into the cytosol where it is oxidized by a peroxidase. When exposed to light, protoporphyrin IX causes formation of singlet oxygen in the cytosol and the formation of other reactive oxygen species, which can cause lipid peroxidation and membrane disruption leading to rapid cell death (Lee et al. 1993. Plant Physiol. 102: 881).

Not all PPO enzymes are sensitive to herbicides which inhibit plant PPO enzymes. Both the Escherichia coli and Bacillus subtilis PPO enzymes (Sasarmen et al. 1993. Can. J. Microbiol. 39: 1155; Dailey et al. 1994. J. Biol. Chem. 269: 813) are resistant to these herbicidal inhibitors. Mutants of the unicellular alga Chlamydomonas reinhardtii resistant to the phenylimide herbicide S-23142 have been reported (Kataoka et al. 1990. J. Pesticide Sci. 15: 449; Shibata et al. 1992. In Research in Photosynthesis, Vol. III, N. Murata, ed. Kluwer:Netherlands. pp. 567-70). At least one of these mutants appears to have an altered PPO activity that is resistant not only to the herbicidal inhibitor on which the mutant was selected, but also to other classes of protox inhibitors (Oshio et al. 1993. Z. Naturforsch. 48c: 339; Sato et al. 1994. In ACS Symposium on Porphyric Pesticides, S. Duke, ed. ACS Press: Washington, D.C.). A mutant tobacco cell line has also been reported that is resistant to the inhibitor S-21432 (Che et al. 1993. Z. Naturforsch. 48c: 350). Auxotrophic E. coli mutants have been used to confirm the herbicide resistance of cloned plant PPO-inhibting herbicides.

Three main strategies are available for making plants tolerant to herbicides, i.e. (1) detoxifying the herbicide with an enzyme which transforms the herbicide, or its active metabolite, into non-toxic products, such as, for example, the enzymes for tolerance to bromoxynil or to basta (EP242236, EP337899); (2) mutating the target enzyme into a functional enzyme which is less sensitive to the herbicide, or to its active metabolite, such as, for example, the enzymes for tolerance to glyphosate (EP293356, Padgette S. R. et al., J. Biol. Chem., 266, 33, 1991); or (3) overexpressing the sensitive enzyme so as to produce quantities of the target enzyme in the plant which are sufficient in relation to the herbicide, in view of the kinetic constants of this enzyme, so as to have enough of the functional enzyme available despite the presence of its inhibitor. The third strategy was described for successfully obtaining plants which were tolerant to PPO inhibitors (see e.g. U.S. Pat. No. 5,767,373 or U.S. Pat. No. 5,939,602, and patent family members thereof.). In addition, US 2010/0100988 and WO 2007/024739 discloses nucleotide sequences encoding amino acid sequences having enzymatic activity such that the amino acid sequences are resistant to PPO inhibitor herbicidal chemicals, in particular 3-phenyluracil inhibitor specific PPO mutants.

WO 2012/080975 discloses plants the tolerance of which to a PPO-inhibiting herbicide. In particular, WO 2012/080975 discloses that the introduction of nucleic acids which code for a mutated PPO of an Amaranthus type II PPO in which the Arginine at position 128 had been replaced by a leucine, alanine, or valine, and the phenylalanine at position 420 had been replaced by a methionine, cysteine, isoleucine, leucine, or threonine, confers increased tolerance/resistance to a benzoxazinone-derivative herbicide. WO 2013/189984 describes that the introduction of nucleic acids which code for a mutated PPO in which the leucine corresponding to position 397, and the phenylalanine corresponding to position 420 of an Amaranthus type II PPO are replaced confers increased tolerance/resistance to PPO inhibiting herbicides. WO2015/022636 describes novel PPO mutants in which which the arginine corresponding to position 128, and the phenylalanine corresponding to position 420 of an Amaranthus type II PPO are replaced by amino acids which are different from those disclosed by WO 2012/080975.

WO2015/022640 discloses novel PPO enzymes and mutants thereof derived from Alopecurus myosuroides. WO2015/092706 decribes that the transfer of mutations from Amaranthus (as decribed above) into crop PPO sequences show an increased tolerance effect in plant. U.S. Pat. No. 7,671,254 describes mutated PPO of an Amaranthus type II PPO in which the glycine at positions 210 and/or 211 are deleted. In contrast to positions corresponding to 128, 397, and 420, said positions 210 and/or 211 are known to lie outside the catalytic site of PPO (“non-active sites”). The inventors of the present invention have now surprisingly found out that a substitution of amino acids at Gly210/211, or at other non-active sites, in particular when combined with a substitution at positions at active sites, i.e. corresponding to positions 128, 397, and/or 420 of the Amaranthus type II PPO increase herbicide tolerance.

### SUMMARY OF THE INVENTION

Accordingly, in one aspect, the present invention provides a plant or plant part comprising a polynucleotide encoding a mutated PPO polypeptide, the expression of said polynucleotide confers to the plant or plant part tolerance to herbicides.

In a preferred embodiment, said mutated PPO polypeptide comprises one or more of the following motifs.

i) Motif 1:
(SEQ ID NO: 131)
GT[C/S]GGDP

Wherein the glycine at position 4, and/or 5 within said motif of the corresponding wildt e sequence is substituted by any other amino acid

ii) Motif 2:
(SEQ ID NO: 132)
[A/S/C]PS[D/N][X][X]L

Wherein the serine at position 3 within said motif of the corresponding wildtype sequence is substituted by any other amino acid

iii) Motif 3:
(SEQ ID NO: 133)
[R/Q][E/D]KQQ[L/Y]P

Wherein the glutamine at position 4 within said motif of the corresponding wildt e sequence is substituted b any other amino acid

iv) Motif 4:
(SEQ ID NO: 134)
L[I/V]PSKE

wherein the serine at position 4 within said motif of the corresponding wildtype sequence is substituted by any other amino acid.

Preferably, said mutated PPO polypeptide in addition comprises one or more of the following motifs
a. Motif 5: SQ[N/K/H]KRYI (SEQ ID NO: 135), wherein the Arg at position 5 within said motif is substituted by any other amino acid;
b. Motif 6: TLGTLFSS (SEQ ID NO: 136), wherein the Leu at position 2 within said motif is substituted by by any other amino acid;
c. Motif 7: [F/Y]TTF[V/I]GG (SEQ ID NO: 137), wherein the Phe at position 4 within said motif is substituted by by any other amino acid.

In some aspects, the present invention provides a seed capable of germination into a plant comprising in at least some of its cells a polynucleotide operably linked to a promoter operable in plant cells, the promoter capable of expressing a mutated PPO polypeptide encoded by the polynucleotide of the present invention, the expression of the mutated PPO polypeptide conferring to the plant tolerance to herbicides.

In one aspect, the present invention provides a plant cell capable of regenerating a plant comprising in at least some of its cells a polynucleotide operably linked to a promoter operable in plant cells, the promoter capable of expressing a mutated PPO polypeptide encoded by the polynucleotide of the present invention, the expression of the mutated PPO polypeptide conferring to the plant tolerance to herbicides, wherein the plant cell comprises the polynucleotide operably linked to a promoter.

In another aspect, the present invention provides a plant cell comprising a polynucleotide operably linked to a promoter operable in a cell, the promoter capable of expressing a mutated PPO polypeptide encoded by the polynucleotide of the present invention, the expression of the mutated PPO polypeptide conferring to the plant tolerance to herbicides.

In other aspects, the present invention provides a plant product prepared from a plant or plant part comprising in at least some of its cells a polynucleotide operably linked to a promoter operable in plant cells, the promoter capable of expressing a mutated PPO polypeptide encoded by the polynucleotide of the present invention, the expression of the mutated PPO polypeptide conferring to the plant tolerance to herbicides.

In some aspects, the present invention provides a progeny or descendant plant derived from a plant comprising in at least some of its cells a polynucleotide operably linked to a promoter operable in plant cells, the promoter capable of expressing a mutated PPO polypeptide encoded by the polynucleotide of the present invention, wherein the progeny or descendant plant comprises in at least some of its cells the recombinant polynucleotide operably linked to the promoter, the expression of the mutated PPO polypeptide conferring to the progeny or descendant plant tolerance to the herbicides.

In other aspects, the present invention provides a method for controlling weeds at a locus for growth of a plant, the method comprising: (a) applying an herbicide composition comprising herbicides to the locus; and (b) planting a seed at the locus, wherein the seed is capable of producing a plant that comprises in at least some of its cells a polynucleotide operably linked to a promoter operable in plant cells, the promoter capable of expressing a mutated PPO polypeptide encoded by the polynucleotide of the present invention, the expression of the mutated PPO polypeptide conferring to the plant tolerance to herbicides.

In some aspects, the present invention provides a method for controlling weeds at a locus for growth of a plant, the method comprising: applying an herbicidal composition comprising herbicides to the locus; wherein said locus is: (a) a locus that contains: a plant or a seed capable of producing said plant; or (b) a locus that is to be after said applying is made to contain the plant or the seed; wherein the plant or the seed comprises in at least some of its cells a polynucleotide operably linked to a promoter operable in plant cells, the promoter capable of expressing a mutated PPO polypeptide encoded by the polynucleotide of the present invention, the expression of the mutated PPO polypeptide conferring to the plant tolerance to herbicides.

In one aspect, step (a) occurs before, after, or concurrently with step (b).

In other aspects, the present invention provides a method of producing a plant having tolerance to herbicides, the method comprising regenerating a plant from a plant cell transformed with a polynucleotide operably linked to a promoter operable in plant cells, the promoter capable of expressing a mutated PPO polypeptide encoded by the polynucleotide of the present invention, the expression of the mutated PPO polypeptide conferring to the plant tolerance to herbicides.

In one aspect, the present invention provides a method of producing a progeny plant having tolerance to herbicides, the method comprising: crossing a first herbicide-tolerant plant with a second plant to produce a herbicide-tolerant progeny plant, wherein the first plant and the progeny plant comprise in at least some of their cells a polynucleotide operably linked to a promoter operable in plant cells, the promoter capable of expressing a mutated PPO polypeptide encoded by the polynucleotide of the present invention, the expression of the mutated PPO polypeptide conferring to the plant tolerance to herbicides.

In addition, the present invention refers to a method for identifying a herbicide by using a mutated PPO of the present invention.

Said method comprises the steps of:
a) generating a transgenic cell or plant comprising a nucleic acid encoding a mutated PPO of the present invention, wherein the mutated PPO of the present invention is expressed;
b) applying a herbicide to the transgenic cell or plant of a) and to a control cell or plant of the same variety;
c) determining the growth or the viability of the transgenic cell or plant and the control cell or plant after application of said test compound, and
d) selecting test compounds which confer reduced growth to the control cell or plant as compared to the growth of the transgenic cell or plant.

Another object refers to a method of identifying a nucleotide sequence encoding a mutated PPO of the present invention which is resistant or tolerant to a herbicide, the method comprising:
a) generating a library of mutated PPO-encoding nucleic acids,
b) screening a population of the resulting mutated PPO-encoding nucleic acids by expressing each of said nucleic acids in a cell or plant and treating said cell or plant with a herbicide,
c) comparing the herbicide-tolerance levels provided by said population of mutated PPO encoding nucleic acids with the herbicide-tolerance level provided by a control PPO-encoding nucleic acid,
d) selecting at least one mutated PPO-encoding nucleic acid that provides a significantly increased level of tolerance to a herbicide as compared to that provided by the control PPO-encoding nucleic acid.

In a preferred embodiment, the mutated PPO-encoding nucleic acid selected in step d) provides at least 2-fold as much tolerance to a herbicide as compared to that provided by the control PPO-encoding nucleic acid.

The resistance or tolerance can be determined by generating a transgenic plant comprising a nucleic acid sequence of the library of step a) and comparing said transgenic plant with a control plant.

Another embodiment refers to an isolated and/or recombinantly produced and/or synthetic nucleic acid molecule comprising a nucleic acid molecule encoding a mutated PPO polypeptide selected from the group consisting of:
(a) a nucleic acid molecule encoding a mutated PPO polypeptide comprising the sequence of—SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129, or a variant, paralogue, orthologue or homolog thereof;
(b) a nucleic acid molecule, which, as a result of the degeneracy of the genetic code, can be derived from a nucleic acid molecule encoding a mutated PPO polypeptide comprising the sequence of—SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129, or a variant, paralogue, orthologue or homolog thereof, and confers increased herbicide tolerance or resistance, as compared to a corresponding, e.g. non-transformed, wild type plant cell, a plant or a part thereof;
(c) a nucleic acid molecule encoding a mutated PPO polypeptide having 30% or more identity, preferably at least 40%, 50%, 60%, 70%, 75%, 80%, 85%, 90%, 95%, 96%, 97%, 98%, 99%, 99.5% or more, with the amino acid sequence of the PPO polypeptide sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129, and confers increased herbicide tolerance or resistance, as compared to a corresponding, e.g. non-transformed, wild type plant cell, a plant or a part thereof;
(d) nucleic acid molecule which hybridizes with a nucleic acid molecule of (a), (b), or (c), under stringent hybridization conditions and confers increased herbicide tolerance or resistance, as compared to a corresponding, e.g. non-transformed, wild type plant cell, a plant or a part thereof;
wherein the amino acid sequence of the encoded mutated PPO polypeptide differs from the wildtype amino acid sequence of a PPO polypeptide at one or more positions corresponding to positions 32, 53, 57, 61, 63, 64, 65, 67, 71, 76, 82, 83, 85, 86, 87, 88, 91, 103, 104, 106, 108, 116, 119, 126, 127, 129, 139, 159, 210, 211, 224, 245, 246, 248, 249, 252, 253, 254, 255, 257, 259, 260, 262, 264, 286, 291, 305, 308, 309, 323, 335, 343, 345, 358, 372, 373, 387, 391, 392, 400, 412, 414, 415, 425, 428, 431, 433, 434, 435, 436, 447, 451, 453, 464, 466, 482 of SEQ ID NO: 1 or 2, wherein said difference refers to a substitution of the amino acid at that positions by any other amino acid.

Another embodiment refers to an isolated and/or recombinantly produced and/or synthetic nucleic acid molecule comprising a nucleic acid molecule encoding a mutated PPO polypeptide selected from the group consisting of:
(a) a nucleic acid molecule encoding a mutated PPO polypeptide comprising the sequence of—SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129, or a variant, paralogue, orthologue or homolog thereof;
(b) a nucleic acid molecule, which, as a result of the degeneracy of the genetic code, can be derived from a nucleic acid molecule encoding a mutated PPO polypeptide comprising the sequence of—SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129, or a variant, paralogue, orthologue or homolog thereof, and confers increased herbicide tolerance or resistance, as compared to a corresponding, e.g. non-transformed, wild type plant cell, a plant or a part thereof;
(c) a nucleic acid molecule encoding a mutated PPO polypeptide having 30% or more identity, preferably at least 40%, 50%, 60%, 70%, 75%, 80%, 85%, 90%, 95%, 96%, 97%, 98%, 99%, 99.5% or more, with the amino acid sequence of the PPO polypeptide sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129, and confers increased herbicide tolerance or resistance, as compared to a corresponding, e.g. non-transformed, wild type plant cell, a plant or a part thereof;
(d) nucleic acid molecule which hybridizes with a nucleic acid molecule of (a), (b), or (c), under stringent hybridization conditions and confers increased herbicide tolerance or resistance, as compared to a corresponding, e.g. non-transformed, wild type plant cell, a plant or a part thereof;
wherein the amino acid sequence of the encoded mutated PPO polypeptide differs from the wildtype amino acid sequence of a PPO polypeptide at one or more positions corresponding to positions 40, 250, 391, 520, 521, 522, 523, 524, 525, 526, 527, 528, 529, 530, 531, 532, 533, 534 of SEQ ID NO: 1 or 2, wherein said difference refers to a deletion or an insertion of the amino acid at that positions.

Preferably, the difference corresponding to position 391 of SEQ ID NO:1 referes to an insertion, and the difference corresponding to positions 40, 250, 520, 521, 522, 523, 524, 525, 526, 527, 528, 529, 530, 531, 532, 533, 534 of SEQ ID NO: 1 or 2 refers to a deletion.

Another object refers to an expression cassette comprising the nucleic acid molecule of the present invention and a promoter operable in plant cells.

Another object refers to an isolated, recombinant and/or chemically synthesized mutated PPO polypeptide encoded by the nucleic acid molecule as claimed in any of claims 17 to 25 or a polypeptide having at least 80%, 90%, 95%, 98%, 99% identity to the sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129, or a variant, paralogue, orthologue or homolog thereof, wherein the amino acid sequence of the mutated PPO polypeptide differs from the wildtype amino acid sequence of a PPO polypeptide at one or more positions corresponding to positions 32, 53, 57, 61, 63, 64, 65, 67, 71, 76, 82, 83, 85, 86, 87, 88, 91, 103, 104, 106, 108, 116, 119, 126, 127, 129, 139, 159, 210, 211, 224, 245, 246, 248, 249, 252, 253, 254, 255, 257, 259, 260, 262, 264, 286, 291, 305, 308, 309, 323, 335, 343, 345, 358, 372, 373, 387, 391, 392, 400, 412, 414, 415, 425, 428, 431, 433, 434, 435, 436, 447, 451, 453, 464, 466, 482 of SEQ ID NO: 1 or 2, wherein said difference refers to a substitution of the amino acid at that positions by any other amino acid.

Another object refers to an isolated, recombinant and/or chemically synthesized mutated PPO polypeptide encoded by the nucleic acid molecule as claimed in any of claims 17 to 25 or a polypeptide having at least 80%, 90%, 95%, 98%, 99% identity to the sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129, or a variant, paralogue, orthologue or homolog thereof, wherein the amino acid sequence of the mutated PPO polypeptide differs from the wildtype amino acid sequence of a PPO polypeptide at one or more positions corresponding to positions 40, 250, 391, 520, 521, 522, 523, 524, 525, 526, 527, 528, 529, 530, 531, 532, 533, 534 of SEQ ID NO: 1 or 2, wherein said difference refers to a deletion or an insertion of the amino acid at that positions.

In still further aspects, the present invention provides a plant or plant part comprising in at least some of its cells a polynucleotide operably linked to a promoter operable in plant cells, the promoter capable of expressing a mutated PPO polypeptide encoded by the polynucleotide, the expression of the mutated PPO polypeptide conferring to the plant tolerance to herbicides, wherein the plant or plant part further exhibits a second or third herbicide-tolerant trait.

In another embodiment, the invention refers to a plant cell transformed by and expressing a mutated PPO nucleic acid according to the present invention or a plant which has been mutated to obtain a plant expressing, preferably over-expressing a mutated PPO nucleic acid according to the present invention, wherein expression of said nucleic acid in the plant cell results in increased resistance or tolerance to a herbicide as compared to a wild type variety of the plant cell In another embodiment, the invention refers to a plant comprising a plant cell according to the present invention, wherein expression of the nucleic acid in the plant results in the plant's increased resistance to herbicide as compared to a wild type variety of the plant.

The plants of the present invention can be transgenic or non-transgenic.

Preferably, the expression of the nucleic acid of the invention in the plant results in the plant's increased resistance to herbicides as compared to a wild type variety of the plant.

In another embodiment, the invention refers to a seed produced by a transgenic plant comprising a plant cell of the present invention, wherein the seed is true breeding for an increased resistance to a herbicide as compared to a wild type variety of the seed.

In another embodiment, the invention refers to a method of producing a transgenic plant cell with an increased resistance to a herbicide as compared to a wild type variety of the plant cell comprising, transforming the plant cell with an expression cassette comprising a polynucleotide operably linked to a promoter operable in plant cells, the promoter capable of expressing a mutated PPO polypeptide encoded by the polynucleotide.

In another embodiment, the invention refers to a method of producing a transgenic plant comprising, (a) transforming a plant cell with an expression cassette comprising a polynucleotide operably linked to a promoter operable in plant cells, the promoter capable of expressing a mutated PPO polypeptide encoded by the polynucleotide, and (b) generating a plant with an increased resistance to herbicide from the plant cell.

Preferably, the expression cassette further comprises a transcription initiation regulatory region and a translation initiation regulatory region that are functional in the plant.

### BRIEF DESCRIPTION OF THE DRAWINGS

FIG. 1 shows a germination assay using transgenic Arabidopsis plants (SEQ ID NO:1 mutated AMATU_PPO2_G211A_F420M). Plants were treated with the indicated concentrations of Saflufenacil. Pictures were taken 14 days after treatment.

FIG. 2 shows T1 Tolerance Spray using transgenic Arabidopsis plants (SEQ ID NO:1 mutated AMATU_PPO2_G211A_F420M). Pictures were taken 8 days after treatment (days after spraying). Top at bottom contain 2 wild type plants, upper 5 pots contain independent transgenic events (T1 plants, selected by confirming presence of resistance gene AHAS).

FIG. 3 shows Transgenic Arabidopsis plants sprayed post in the greenhouse with the indicated amount of Saflufenacil+1% (v/v) MSO. Plants harbor the SEQ ID NO:1 mutated traits: A) AMATU_PPO2_R128A_G210S_F420M, B) AMATU_PPO2_R128A_S387L_F420M, C) AMATU_PPO2_G211A_L397Q_F420M, D) AMATU_PPO2_R128A_F420M E) AMATU_PPO2_F420V F) AMATU_PP02_L397Q_F420M G) Wild-type (non-transgenic). Evaluation performed 6 Days After Treatment (DAT).

FIG. 4 shows transgenic Arabidopsis plants sprayed post in the greenhouse with the indicated amount of Trifludimoxazine+1% (v/v) MSO. Plants harbor the SEQ ID NO:1 mutated traits: A) AMATU_PPO2_R128A_G210S_F420M, B) AMATU_PPO2_R128A_S387L_F420M, C) AMATU_PPO2_G211A_F420V, D) AMATU_PPO2_G211A_L397Q_F420M, E) AMATU_PPO2_R128A_F420M F) AMATU_PPO2_F420V G) AMATU_PPO2_L397Q_F420M H) Wild-type (non-transgenic). Evaluation performed 6 Days After Treatment (DAT).

FIG. 5 shows transgenic Arabidopsis plants sprayed post in the greenhouse with the indicated amount of Saflufenacil+Trifludimoxazine+1% (v/v) MSO. Plants harbor the SEQ ID NO:1 mutated traits: A) AMATU_PPO2_R128A_G210S_F420M, B) AMATU_PPO2_R128A_S387L_F420M, C) AMATU_PPO2_G211A_F420V, D) AMATU_PPO2_G211A_L397Q_F420M, E) AMATU_PPO2_R128A_F420M F) AMATU_PPO2_F420V G) AMATU_PPO2_L397Q_F420M H) Wild-type (non-transgenic). Evaluation performed 6 Days After Treatment (DAT).

FIG. 6 shows transgenic Arabidopsis plants sprayed post in the greenhouse with the indicated amount of Uracilpyridine 2+1% (v/v) MSO. Plants harbor the SEQ ID NO:1 mutated traits: A) AMATU_PPO2_R128A_G210S_F420M, B) AMATU_PPO2_R128A_S387L_F420M, C) AMATU_PPO2_G211A_F420V, D) AMATU_PPO2_G211A_L397Q_F420M, E) AMATU_PPO2_R128A_F420M F) AMATU_PPO2_F420V G) AMATU_PPO2_L397Q_F420M H) Wild-type (non-transgenic). Evaluation performed 6 Days After Treatment (DAT).

FIG. 7 shows transgenic Arabidopsis plants sprayed post in the greenhouse with the indicated amount of Uracilpyridine 4+1% (v/v) MSO. Plants harbor the SEQ ID NO:1 mutated traits: A) AMATU_PPO2_R128A_G210S_F420M, B) AMATU_PPO2_R128A_S387L_F420M, C) AMATU_PPO2_G211A_F420V, D) AMATU_PPO2_G211A_L397Q_F420M, E) AMATU_PPO2_R128A_F420M F) AMATU_PPO2_F420V G) AMATU_PPO2_L397Q_F420M H) Wild-type (non-transgenic). Evaluation performed 6 Days After Treatment (DAT).

FIG. 8 shows transgenic Arabidopsis plants sprayed post in the greenhouse with the indicated amount of Uracilpyridine 1+1% (v/v) MSO. Plants harbor the SEQ ID NO:1 mutated traits: A) AMATU_PPO2_R128A_G210S_F420M, B) AMATU_PPO2_R128A_S387L_F420M, C) AMATU_PPO2_G211A_L397Q_F420M, D) AMATU_PPO2_R128A_F420M E) AMATU_PPO2_F420V F) AMATU_PP02_L397Q_F420M G) Wild-type (non-transgenic). Evaluation performed 6 Days After Treatment (DAT).

FIG. 9 shows transgenic Arabidopsis plants sprayed post in the greenhouse with the indicated amount of Sulfentrazone+1% (v/v) MSO. Plants harbor the SEQ ID NO:1 mutated traits: A) AMATU_PPO2_R128A_G210S_F420M, B) AMATU_PPO2_R128A_S387L_F420M, C) AMATU_PPO2_G211A_L397Q_F420M, D) AMATU_PPO2_R128A_F420M E) AMATU_PPO2_F420V F) AMATU_PP02_L397Q_F420M G) Wild-type (non-transgenic). Evaluation performed 6 Days After Treatment (DAT).

FIG. 10 shows transgenic Arabidopsis plants sprayed post in the greenhouse with the indicated amount of Flumioxazin+1% (v/v) MSO. Plants harbor the SEQ ID NO:1 mutated traits: A) AMATU_PPO2_R128A_G210S_F420M, B) AMATU_PPO2_R128A_S387L_F420M, C) AMATU_PPO2_R128A_F420M D) AMATU_PPO2_F420V E) AMATU_PPO2_L397Q_F420M F) Wild-type (non-transgenic). Evaluation performed 6 Days After Treatment (DAT).

FIG. 11 shows transgenic Arabidopsis plants harboring SEQ ID NO:1 mutated AMATU_PPO2_G211A_F420M construct, sprayed post in the greenhouse with the indicated amount of A) Saflufenacil+Trifludimoxazine, B) Tiafenacil, C) Uracilpyridine 6, D) Pyraflufen-ethyl, E) Uracilpyridine 7, with +1% (v/v) MSO. Evaluation performed 14 Days After Treatment (DAT) and is shown as injury (%) relative to non-transgenic treated plants.

FIG. 12 shows examples of transgenic Zea mays harboring AmtuPPX2L variants after pre-emergent treatment with Saflufenacil.

FIG. 13 shows tolerance of transgenic Glycine max harboring AmtuPPX2L variants after treatment with a mixture of Saflufenacil and Trifludimoxazine. 0/0 denotes no active ingredient, 50/25 denotes 50 grams of active ingredient per hectare (AI/Ha) of Saflufenacil and 25 grams AI/Ha of Trifludimoxazine, 100/50 denotes 100 g AI/Ha Saflufenacil and 50 g AI/Ha Trifludimoxazine and 200/100 denotes 200 g AI/Ha Saflufenacil and 100 g AI/Ha Trifludimoxazine. Wild type untransformed is represented by the soy (Glycine max) cultivar Jake (cv. Jake).

FIG. 14 shows examples of herbicide tolerance of transgenic Glycine max harboring AmtuPPX2L variants after treatment with a mixture of Saflufenacil and Trifludimoxazine. 0/0 denotes no active ingredient, 25/50 denotes 50 grams of active ingredient per hectare (AI/Ha) of Saflufenacil and 25 grams AI/Ha of Trifludimoxazine, 50/100 denotes 100 g AI/Ha Saflufenacil and 50 g AI/Ha Trifludimoxazine and 100/200 denotes 200 g AI/Ha Saflufenacil and 100 g AI/Ha Trifludimoxazine. Wild type untransformed is represented by the soy (Glycine max) cultivar Jake (cv. Jake).

LEGEND TO SEQUENCE LISTING

| SEQ ID | Organism |
|---|---|
| 1 | Amaranthus tuberculatus |
| 2 | Amaranthus tuberculatus |
| 3 | Amaranthus tuberculatus |
| 4 | Amaranthus tuberculatus |
| 5 | Amaranthus hypochondriacus |
| 6 | Amaranthus tuberculatus |
| 7 | Spinacia oleracea |
| 8 | Vitis vinifera |
| 9 | Ricinus communis |
| 10 | Theobroma cacao |
| 11 | Glycine max |
| 12 | Prunus persica |
| 13 | Medicago truncatula |
| 14 | Fragaria vesca subsp. vesca |
| 15 | Citrus clementina |
| 16 | Citrus clementina |
| 17 | Cicer arietinum |
| 18 | Cucumis sativus |
| 19 | Cucumis sativus |
| 20 | Nicotiana tabacum |
| 21 | Solanum lycopersicum |
| 22 | Arabidopsis thaliana |
| 23 | Arabidopsis lyrata subsp. lyrata |
| 24 | Arabidopsis thaliana |
| 25 | Arabidopsis thaliana |
| 26 | Ambrosia artemisiifolia |
| 27 | Setaria italica |
| 28 | Sorghum bicolor |
| 29 | Arabidopsis thaliana |
| 30 | Zea mays |
| 31 | Zea mays |
| 32 | LEMPA |
| 33 | LEMPA |
| 34 | Populus trichocarpa |
| 35 | Capsella rubella |
| 36 | Brachypodium distachyon |
| 37 | Oryza sativa Japonica Group |
| 38 | Picea sitchensis |
| 39 | Solarium tuberosum |
| 40 | Oryza sativa Indica Group |
| 41 | Oryza sativa Japonica Group |
| 42 | Eutrema salsugineum |
| 43 | Selaginella moellendorffii |
| 44 | Selaginella moellendorffii |
| 45 | Amaranthus tuberculatus |
| 46 | Amaranthus tuberculatus |
| 47 | Zea mays |
| 48 | Aegilops tauschii |
| 49 | Genlisea aurea |
| 50 | Amborella trichopoda |
| 51 | Rhodothermus marinus |
| 52 | Salinibacter ruber |
| 53 | Salinibacter ruber M8 |
| 54 | Zea mays |
| 55 | Rhodothermus marinus |
| 56 | Caldithrix abyssi |
| 57 | Opitutus terrae PB90-1 |
| 58 | Verrucomicrobia bacterium |
| 59 | Ignavibacterium album |
| 60 | Coraliomargarita sp. CAG:312 |
| 61 | Salisaeta longa |
| 62 | Ambrosia artemisiifolia |
| 63 | Melioribacter roseus P3M-2 |
| 64 | Halothiobacillus neapolitanus c2 |
| 65 | Chondrus crispus |
| 66 | Rubritalea marina |
| 67 | Acidobacteria bacterium |
| 68 | Coraliomargarita akajimensis DSM 45221 |
| 69 | Oscillochloris trichoides DG6 |
| 70 | Opitutaceae bacterium TAV1 |
| 71 | Amborella trichopoda |
| 72 | Opitutaceae bacterium TAV5 |
| 73 | Chloroflexus sp. Y-400-fl |
| 74 | Leptospirillum sp. Group II ‘5-way CG’ |
| 75 | Leptospirillum ferriphilum ML-04 |
| 76 | Verrucomicrobia bacterium SCGC AAA300-O17 |
| 77 | Chloroflexus aggregans DSM 9485 |
| 78 | Desulfurobacterium thermolithotrophum |
| 79 | Desulfurobacterium sp. TC5-1 |
| 80 | Arthrospira platensis C1 |
| 81 | Leptospirillum sp. Group II ‘C75’ |
| 82 | Verrucomicrobiae bacterium DG1235 |
| 83 | Verrucomicrobia bacterium SCGC AAA300-K03 |
| 84 | Synechococcus sp. JA-3-3Ab |
| 85 | Hymenobacter norwichensis |
| 86 | Pontibacter sp. BAB1700 |
| 87 | Leptospirillum ferrodiazotrophum |
| 88 | Prevotella histicola F0411 |
| 89 | Flexithrix dorotheae |
| 90 | Geobacter metallireducens GS-15 |
| 91 | Synechococcus sp. JA-2-3B′a(2-13) |
| 92 | Crinalium epipsammum PCC 9333 |
| 93 | Planctomyces maris |
| 94 | Geobacter uraniireducens Rf4 |
| 95 | Acidithiobacillus ferrivorans |
| 96 | Prevotella melaninogenica |
| 97 | Thermovibrio ammonificans |
| 98 | Brassica_rapa |
| 99 | Brassica_rapa |
| 100 | Gossypium |
| 101 | Gossypium |
| 102 | Conyza_canadensis |
| 103 | Conyza_canadensis |
| 104 | Kochia_scobaria |
| 105 | Lolium_rigidum |
| 106 | Lolium_rigidum |
| 107 | Gossypium hirsutum PPO1 |
| 108 | Beta vulgaris PPO1 |
| 109 | Hordeum vulgare PPO1 |
| 110 | Hordeum vulgare PPO2 |
| 111 | Triticum aestivum PPO1 |
| 112 | Solarium lycopersicum PPO2 |
| 113 | Triticum aestivum PPO1_v2 |
| 114 | Gossypium hirsutum PPO1_v2 |
| 115 | Gossypium hirsutum PPO2 |
| 116 | Beta vulgaris PPO1_v2 |
| 117 | Brassica napus_PPO2 |
| 129 | Alopecurus myosuroides |

### DETAILED DESCRIPTION

The articles “a” and “an” are used herein to refer to one or more than one (i.e., to at least one) of the grammatical object of the article. By way of example, “an element” means one or more elements.

As used herein, the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

The term “control of undesired vegetation or weeds” is to be understood as meaning the killing of weeds and/or otherwise retarding or inhibiting the normal growth of the weeds. Weeds, in the broadest sense, are understood as meaning all those plants which grow in locations where they are undesired. The weeds of the present invention include, for example, dicotyledonous and monocotyledonous weeds. Dicotyledonous weeds include, but are not limited to, weeds of the genera: Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, and Taraxacum. Monocotyledonous weeds include, but are not limited to, weeds of of the genera: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, and Apera. In addition, the weeds of the present invention can include, for example, crop plants that are growing in an undesired location. For example, a volunteer maize plant that is in a field that predominantly comprises soybean plants can be considered a weed, if the maize plant is undesired in the field of soybean plants.

The term “plant” is used in its broadest sense as it pertains to organic material and is intended to encompass eukaryotic organisms that are members of the Kingdom Plantae, examples of which include but are not limited to vascular plants, vegetables, grains, flowers, trees, herbs, bushes, grasses, vines, ferns, mosses, fungi and algae, etc, as well as clones, offsets, and parts of plants used for asexual propagation (e.g. cuttings, pipings, shoots, rhizomes, underground stems, clumps, crowns, bulbs, corms, tubers, rhizomes, plants/tissues produced in tissue culture, etc.). The term “plant” further encompasses whole plants, ancestors and progeny of the plants and plant parts, including seeds, shoots, stems, leaves, roots (including tubers), flowers, florets, fruits, pedicles, peduncles, stamen, anther, stigma, style, ovary, petal, sepal, carpel, root tip, root cap, root hair, leaf hair, seed hair, pollen grain, microspore, cotyledon, hypocotyl, epicotyl, xylem, phloem, parenchyma, endosperm, a companion cell, a guard cell, and any other known organs, tissues, and cells of a plant, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amaranth, artichoke, asparagus, broccoli, Brussels sprouts, cabbage, canola, carrot, cauliflower, celery, collard greens, flax, kale, lentil, oilseed rape, okra, onion, potato, rice, soybean, strawberry, sugar beet, sugar cane, sunflower, tomato, squash, tea and algae, amongst others. According to a preferred embodiment of the present invention, the plant is a crop plant. Examples of crop plants include inter alia soybean, sunflower, canola, alfalfa, rapeseed, cotton, tomato, potato or tobacco. Further preferebly, the plant is a monocotyledonous plant, such as sugarcane. Further preferably, the plant is a cereal, such as rice, maize, wheat, barley, millet, rye, sorghum or oats.

Generally, the term “herbicide” is used herein to mean an active ingredient that kills, controls or otherwise adversely modifies the growth of plants. The preferred amount or concentration of the herbicide is an “effective amount” or “effective concentration.” By “effective amount” and “effective concentration” is intended an amount and concentration, respectively, that is sufficient to kill or inhibit the growth of a similar, wild-type, plant, plant tissue, plant cell, or host cell, but that said amount does not kill or inhibit as severely the growth of the herbicide-resistant plants, plant tissues, plant cells, and host cells of the present invention. Typically, the effective amount of a herbicide is an amount that is routinely used in agricultural production systems to kill weeds of interest. Such an amount is known to those of ordinary skill in the art. Herbicidal activity is exhibited by herbicides useful for the the present invention when they are applied directly to the plant or to the locus of the plant at any stage of growth or before planting or emergence. The effect observed depends upon the plant species to be controlled, the stage of growth of the plant, the application parameters of dilution and spray drop size, the particle size of solid components, the environmental conditions at the time of use, the specific compound employed, the specific adjuvants and carriers employed, the soil type, and the like, as well as the amount of chemical applied. These and other factors can be adjusted as is known in the art to promote non-selective or selective herbicidal action. Generally, the herbicide treatments can be applied PPI (Pre Plant Incorporated), PPSA (Post plarIt surface applied), PRE- or POST-emergent. Postemergent treatment typically occurs to relatively immature undesirable vegetation to achieve the maximum control of weeds.

By a “herbicide-tolerant” or “herbicide-resistant” plant, it is intended that a plant that is tolerant or resistant to at least one herbicide at a level that would normally kill, or inhibit the growth of, a normal or wildtype plant. Levels of herbicide that normally inhibit growth of a non-tolerant plant are known and readily determined by those skilled in the art. Examples include the amounts recommended by manufacturers for application. The maximum rate is an example of an amount of herbicide that would normally inhibit growth of a non-tolerant plant. For the present invention, the terms “herbicide-tolerant” and “herbicide-resistant” are used interchangeably and are intended to have an equivalent meaning and an equivalent scope. Similarly, the terms “herbicide-tolerance” and “herbicide-resistance” are used interchangeably and are intended to have an equivalent meaning and an equivalent scope. Similarly, the terms “tolerant” and “resistant” are used interchangeably and are intended to have an equivalent meaning and an equivalent scope. As used herein, in regard to an herbicidal composition useful in various embodiments hereof, terms such as herbicides, and the like, refer to those agronomically acceptable herbicide active ingredients (A.I.) recognized in the art. Similarly, terms such as fungicide, nematicide, pesticide, and the like, refer to other agronomically acceptable active ingredients recognized in the art.

When used in reference to a particular mutant enzyme or polypeptide, terms such as herbicide-tolerant and herbicide-tolerance refer to the ability of such enzyme or polypeptide to perform its physiological activity in the presence of an amount of an herbicide A.I. that would normally inactivate or inhibit the activity of the wild-type (non-mutant) version of said enzyme or polypeptide. Furthermore, the PPO activity of such a herbicide-tolerant or herbicide-resistant mutated PPO protein may be referred to herein as “herbicide-tolerant” or “herbicide-resistant” PPO activity.

As used herein, “recombinant,” when referring to nucleic acid or polypeptide, indicates that such material has been altered as a result of human application of a recombinant technique, such as by polynucleotide restriction and ligation, by polynucleotide overlap-extension, or by genomic insertion or transformation. A gene sequence open reading frame is recombinant if that nucleotide sequence has been removed from its natural context and cloned into any type of artificial nucleic acid vector. The term recombinant also can refer to an organism having a recombinant material, e.g., a plant that comprises a recombinant nucleic acid can be considered a recombinant plant.

The term “transgenic plant” refers to a plant that comprises a heterologous polynucleotide. Preferably, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to refer to any cell, cell line, callus, tissue, plant part or plant, the genotype of which has been so altered by the presence of heterologous nucleic acid including those transgenic organisms or cells initially so altered, as well as those created by crosses or asexual propagation from the initial transgenic organism or cell. In some embodiments, a “recombinant” organism is a “transgenic” organism. The term “transgenic” as used herein is not intended to encompass the alteration of the genome (chromosomal or extra-chromosomal) by conventional plant breeding methods (e.g., crosses) or by naturally occurring events such as, e.g., self-fertilization, random cross-fertilization, non-recombinant viral infection, non-recombinant bacterial transformation, non-recombinant transposition, or spontaneous mutation.

As used herein, “mutagenized” refers to an organism or DNA thereof having alteration(s) in the biomolecular sequence of its native genetic material as compared to the sequence of the genetic material of a corresponding wild-type organism or DNA, wherein the alteration(s) in genetic material were induced and/or selected by human action. Examples of human action that can be used to produce a mutagenized organism or DNA include, but are not limited to, treatment with a chemical mutagen such as EMS and subsequent selection with herbicide(s); or by treatment of plant cells with x-rays and subsequent selection with herbicide(s). Any method known in the art can be used to induce mutations. Methods of inducing mutations can induce mutations in random positions in the genetic material or can induce mutations in specific locations in the genetic material (i.e., can be directed mutagenesis techniques), such as by use of a genoplasty technique.

As used herein, a “genetically modified organism” (GMO) is an organism whose genetic characteristics contain alteration(s) that were produced by human effort causing transfection that results in transformation of a target organism with genetic material from another or “source” organism, or with synthetic or modified-native genetic material, or an organism that is a descendant thereof that retains the inserted genetic material. The source organism can be of a different type of organism (e.g., a GMO plant can contain bacterial genetic material) or from the same type of organism (e.g., a GMO plant can contain genetic material from another plant). As used herein in regard to plants and other organisms, “recombinant,” “transgenic,” and “GMO” are considered synonyms and indicate the presence of genetic material from a different source; in contrast, “mutagenized” is used to refer to a plant or other organism, or the DNA thereof, in which no such transgenic material is present, but in which the native genetic material has become mutated so as to differ from a corresponding wild-type organism or DNA.

As used herein, “wild-type” or “corresponding wild-type plant” means the typical form of an organism or its genetic material, as it normally occurs, as distinguished from, e.g., mutagenized and/or recombinant forms. Similarly, by “control cell” or “similar, wild-type, plant, plant tissue, plant cell or host cell” is intended a plant, plant tissue, plant cell, or host cell, respectively, that lacks the herbicide-resistance characteristics and/or particular polynucleotide of the invention that are disclosed herein. The use of the term “wild-type” is not, therefore, intended to imply that a plant, plant tissue, plant cell, or other host cell lacks recombinant DNA in its genome, and/or does not possess herbicide-resistant characteristics that are different from those disclosed herein.

As used herein, “descendant” refers to any generation plant. In some embodiments, a descendant is a first, second, third, fourth, fifth, sixth, seventh, eight, ninth, or tenth generation plant.

As used herein, “progeny” refers to a first generation plant.

The term “seed” comprises seeds of all types, such as, for example, true seeds, caryopses, achenes, fruits, tubers, seedlings and similar forms. In the context of Brassica and Sinapis species, “seed” refers to true seed(s) unless otherwise specified. For example, the seed can be seed of transgenic plants or plants obtained by traditional breeding methods. Examples of traditional breeding methods can include cross-breeding, selfing, back-crossing, embryo rescue, in-crossing, out-crossing, inbreeding, selection, asexual propagation, and other traditional techniques as are known in the art.

Although exemplified with reference to specific plants or plant varieties and their hybrids, in various embodiments, the presently described methods using herbicides can be employed with a variety of commercially valuable plants. Herbicide-tolerant plant lines described as useful herein can be employed in weed control methods either directly or indirectly, i. e. either as crops for herbicide treatment or as herbicide-tolerance trait donor lines for development, to produce other varietal and/or hybrid crops containing such trait or traits. All such resulting variety or hybrids crops, containing the ancestral herbicide-tolerance trait or traits can be referred to herein as progeny or descendant of the ancestral, herbicide-tolerant line(s). Such resulting plants can be said to retain the “herbicide tolerance characteristic(s)” of the ancestral plant, i.e. meaning that they possess and express the ancestral genetic molecular components responsible for the trait.

In one aspect, the present invention provides a plant or plant part comprising a polynucleotide encoding a mutated PPO polypeptide, the expression of said polynucleotide confers to the plant or plant part tolerance to herbicides.

In a preferred embodiment, the plant has been previously produced by a process comprising recombinantly preparing a plant by introducing and over-expressing a mutated PPO transgene according to the present invention, as described in greater detail hereinfter.

In another embodiment, the polynucleotide encoding the mutated PPO polypeptide polypeptide comprises the nucleic acid sequence set forth in SEQ ID NO: 118, 119, 120, 121, 122, 123, 124, 125, 126, 127, 128, or 130, or a variant or derivative thereof.

In other embodiments, the mutated PPO polypeptide according to the present invention is a functional variant having, over the full-length of the variant, at least about 80%, illustratively, at least about 80%, 90%, 95%, 98%, 99% or more amino acid sequence identity to SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129.

In another embodiment, the mutated PPO polypeptide for use according to the present invention is a functional fragment of a polypeptide having the amino acid sequence set forth in SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129.

It is recognized that the PPO polynucleotide molecules and PPO polypeptides of the invention encompass polynucleotide molecules and polypeptides comprising a nucleotide or an amino acid sequence that is sufficiently identical to nucleotide sequence set forth in SEQ ID NOs: 118, 119, 120, 121, 122, 123, 124, 125, 126, 127, 128, or 130, or to the amino acid sequence set forth in SEQ ID Nos: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129. The term “sufficiently identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity.

Generally, “sequence identity” refers to the extent to which two optimally aligned DNA or amino acid sequences are invariant throughout a window of alignment of components, e.g., nucleotides or amino acids. An “identity fraction” for aligned segments of a test sequence and a reference sequence is the number of identical components that are shared by the two aligned sequences divided by the total number of components in reference sequence segment, i.e., the entire reference sequence or a smaller defined part of the reference sequence. “Percent identity” is the identity fraction times 100. Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of these algorithms such as GAP, BESTFIT, FASTA, and TFASTA available as part of the GCG. Wisconsin Package. (Accelrys Inc. Burlington, Mass.)

### Polynucleotides and Oligonucleotides

By an “isolated polynucleotide”, including DNA, RNA, or a combination of these, single or double stranded, in the sense or antisense orientation or a combination of both, dsRNA or otherwise, we mean a polynucleotide which is at least partially separated from the polynucleotide sequences with which it is associated or linked in its native state. Preferably, the isolated polynucleotide is at least 60% free, preferably at least 75% free, and most preferably at least 90% free from other components with which they are naturally associated. As the skilled addressee would be aware, an isolated polynucleotide can be an exogenous polynucleotide present in, for example, a transgenic organism which does not naturally comprise the polynucleotide. Furthermore, the terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

The term “mutated PPO nucleic acid” refers to a PPO nucleic acid having a sequence that is mutated from a wild-type PPO nucleic acid and that confers increased herbicide tolerance to a plant in which it is expressed. Furthermore, the term “mutated protoporphyrinogen oxidase (mutated PPO)” refers to the replacement of an amino acid of the wild-type primary sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129, or a variant, a derivative, a homologue, an orthologue, or paralogue thereof, with another amino acid. The expression “mutated amino acid” will be used below to designate the amino acid which is replaced by another amino acid, thereby designating the site of the mutation in the primary sequence of the protein.

In a preferred embodiment, the PPO nucleotide sequence encoding a mutated PPO comprises the sequence of SEQ ID NO: 118, 119, 120, 121, 122, 123, 124, 125, 126, 127, 128, or 130, or a variant or derivative thereof

Furthermore, it will be understood by the person skilled in the art that the PPO nucleotide sequences encompasse homologues, paralogues and and orthologues of SEQ ID NO: 118, 119, 120, 121, 122, 123, 124, 125, 126, 127, 128, or 130, as defined hereinafter.

The term “variant” with respect to a sequence (e.g., a polypeptide or nucleic acid sequence such as—for example—a transcription regulating nucleotide sequence of the invention) is intended to mean substantially similar sequences. For nucleotide sequences comprising an open reading frame, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis and for open reading frames, encode the native protein comprising the sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129, as well as those that encode a polypeptide having amino acid substitutions relative to the native protein, e.g. the mutated PPO according to the present invention as disclosed herein. Generally, nucleotide sequence variants of the invention will have at least 30, 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98% and 99% nucleotide “sequence identity” to the nucleotide sequence of SEQ ID NO: 118, 119, 120, 121, 122, 123, 124, 125, 126, 127, 128, or 130. The % identity of a polynucleotide is determined by GAP (Needleman and Wunsch, 1970) analysis (GCG program) with a gap creation penalty=5, and a gap extension penalty=0.3. Unless stated otherwise, the query sequence is at least 45 nucleotides in length, and the GAP analysis aligns the two sequences over a region of at least 45 nucleotides. Preferably, the query sequence is at least 150 nucleotides in length, and the GAP analysis aligns the two sequences over a region of at least 150 nucleotides. More preferably, the query sequence is at least 300 nucleotides in length and the GAP analysis aligns the two sequences over a region of at least 300 nucleotides. Even more preferably, the GAP analysis aligns the two sequences over their entire length.

### Polypeptides

By “substantially purified polypeptide” or “purified” a polypeptide is meant that has been separated from one or more lipids, nucleic acids, other polypeptides, or other contaminating molecules with which it is associated in its native state. It is preferred that the substantially purified polypeptide is at least 60% free, more preferably at least 75% free, and more preferably at least 90% free from other components with which it is naturally associated. As the skilled addressee will appreciate, the purified polypeptide can be a recombinantly produced polypeptide. The terms “polypeptide” and “protein” are generally used interchangeably and refer to a single polypeptide chain which may or may not be modified by addition of non-amino acid groups. It would be understood that such polypeptide chains may associate with other polypeptides or proteins or other molecules such as co-factors. The terms “proteins” and “polypeptides” as used herein also include variants, mutants, modifications, analogous and/or derivatives of the polypeptides of the invention as described herein.

The % identity of a polypeptide is determined by GAP (Needleman and Wunsch, 1970) analysis (GCG program) with a gap creation penalty=5, and a gap extension penalty=0.3. The query sequence is at least 25 amino acids in length, and the GAP analysis aligns the two sequences over a region of at least 25 amino acids. More preferably, the query sequence is at least 50 amino acids in length, and the GAP analysis aligns the two sequences over a region of at least 50 amino acids. More preferably, the query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids. Even more preferably, the GAP analysis aligns the two sequences over their entire length.

With regard to a defined polypeptide, it will be appreciated that % identity figures higher than those provided above will encompass preferred embodiments. Thus, where applicable, in light of the minimum % identity figures, it is preferred that the PPO polypeptide of the invention comprises an amino acid sequence which is at least 40%, more preferably at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129.

By “variant” polypeptide is intended a polypeptide derived from the protein of SEQ ID NO: 2, by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

“Derivatives” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. Thus, functional variants and fragments of the PPO polypeptides, and nucleic acid molecules encoding them, also are within the scope of the present invention, and unless specifically described otherwise, irrespective of the origin of said polypeptide and irrespective of whether it occurs naturally. Various assays for functionality of a PPO polypeptide can be employed. For example, a functional variant or fragment of the PPO polypeptide can be assayed to determine its ability to confer herbicides detoxification. By way of illustration, a herbicides detoxification rate can be defined as a catalytic rate sufficient to provide a determinable increase in tolerance to herbicides in a plant or plant part comprising a recombinant polynucleotide encoding the variant or fragment of the PPO polypeptide, wherein the plant or plant part expresses the variant or fragment at up to about 0.5%, illustratively, about 0.05 to about 0.5%, about 0.1 to about 0.4%, and about 0.2 to about 0.3%, of the total cellular protein relative to a similarly treated control plant that does not express the variant or fragment.

In a preferred embodiment, the mutated PPO polypeptide is a functional variant or fragment of a protoporphyrinogen oxidase having the amino acid sequence set forth in SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129, wherein the functional variant or fragment has at least about 80% amino acid sequence identity to SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129.

In other embodiments, the functional variant or fragment further has a herbicides detoxification rate defined as a catalytic rate sufficient to provide a determinable increase in tolerance to herbicides in a plant or plant part comprising a recombinant polynucleotide encoding the variant or fragment, wherein the plant or plant part expresses the variant or fragment at up to about 0.5% of the total cellular protein to a similarly treated control plant that does not express the variant or fragment.

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

In addition, one of ordinary skill in the art will further appreciate that changes can be introduced by mutation into the nucleotide sequences of the invention thereby leading to changes in the amino acid sequence of the encoded proteins without altering the biological activity of the proteins.

Thus, for example, an isolated polynucleotide molecule encoding a mutated PPO polypeptide having an amino acid sequence that differs from that of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129 can be created by introducing one or more nucleotide substitutions, additions, or deletions into the corresponding nucleotide sequence, such that one or more amino acid substitutions, additions or deletions are introduced into the encoded protein. Mutations can be introduced by standard techniques, such as site-directed mutagenesis and PCR-mediated mutagenesis. Such variant nucleotide sequences are also encompassed by the present invention. For example, preferably, conservative amino acid substitutions may be made at one or more predicted preferably nonessential amino acid residues. A “nonessential” amino acid residue is a residue that can be altered from the wild-type sequence of a protein without altering the biological activity, whereas an “essential” amino acid residue is required for biological activity.

A deletion refers to removal of one or more amino acids from a protein.

An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag•100 epitope, c-myc epitope, FLAG®-epitope, lacZ, CMP (calmodulin-binding peptide), HA epitope, protein C epitope and VSV epitope.

A substitution refers to replacement of amino acids of the protein with other amino acids having similar properties (such as similar hydrophobicity, hydrophilicity, antigenicity, propensity to form or break α-helical structures or β-sheet structures). Amino acid substitutions are typically of single residues, but may be clustered depending upon functional constraints placed upon the polypeptide and may range from 1 to 10 amino acids; insertions will usually be of the order of about 1 to 10 amino acid residues. A conservative amino acid substitution is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Such substitutions would not be made for conserved amino acid residues, or for amino acid residues residing within a conserved motif. Conservative substitution tables are well known in the art (see for example Creighton (1984) Proteins. W.H. Freeman and Company (Eds).

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, OH), QuickChange Site Directed mutagenesis (Stratagene, San Diego, CA), PCR-mediated site-directed mutagenesis or other site-directed mutagenesis protocols.

“Derivatives” further include peptides, oligopeptides, polypeptides which may, compared to the amino acid sequence of the naturally-occurring form of the protein, such as the protein of interest, comprise substitutions of amino acids with non-naturally occurring amino acid residues, or additions of non-naturally occurring amino acid residues. “Derivatives” of a protein also encompass peptides, oligopeptides, polypeptides which comprise naturally occurring altered (glycosylated, acylated, prenylated, phosphorylated, myristoylated, sulphated etc.) or non-naturally altered amino acid residues compared to the amino acid sequence of a naturally-occurring form of the polypeptide. A derivative may also comprise one or more non-amino acid substituents or additions compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein. Furthermore, “derivatives” also include fusions of the naturally-occurring form of the protein with tagging peptides such as FLAG, HIS6 or thioredoxin (for a review of tagging peptides, see Terpe, Appl. Microbiol. Biotechnol. 60, 523-533, 2003).

“Orthologues” and “paralogues” encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene.

It is well-known in the art that paralogues and orthologues may share distinct domains harboring suitable amino acid residues at given sites, such as binding pockets for particular substrates or binding motifs for interaction with other proteins.

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide family.

The term “motif” or “consensus sequence” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain).

Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004)), or Pfam (Bateman et al., Nucleic Acids Research 30(1): 276-280 (2002)). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788(2003)). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol 147(1); 195-7).

The proteins of the invention may be altered in various ways including amino acid substitutions, deletions, truncations, and insertions. Methods for such manipulations are generally known in the art. For example, amino acid sequence variants can be prepared by mutations in the DNA. Methods for mutagenesis and nucleotide sequence alterations are well known in the art. See, for example, Kunkel (1985) PNAS, 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D. C), herein incorporated by reference. Conservative substitutions, such as exchanging one amino acid with another having similar properties, may be preferable.

Alternatively, variant nucleotide sequences can be made by introducing mutations randomly along all or part of a coding sequence, such as by saturation mutagenesis, and the resultant mutants can be screened to identify mutants that encode proteins that retain activity. For example, following mutagenesis, the encoded protein can be expressed recombinantly, and the activity of the protein can be determined using standard assay techniques.

The inventors of the present invention have found that by substituting one or more of the amino acid residues in the non-actives sites of the PPO enzyme of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129, e.g. by employing one of the above described methods to mutate the PPO encoding nucleic acids, the tolerance or resistance to particular herbicides could be remarkably increased.

Preferred substitutions of mutated PPO are those that increase the herbicide tolerance of the plant, but leave the biological activity of the PPO enzymatic activity substantially unaffected.

Accordingly, in another object of the present invention refers to a mutated PPO polypeptide, comprising the sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129, a variant, derivative, orthologue, paralogue or homologue thereof, the key amino acid residues of which is substituted by any other amino acid.

It will be understood by the person skilled in the art that amino acids located in a close proximity to the positions of amino acids mentioned below may also be substituted.

Thus, in another embodiment the variant of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129, a variant, derivative, orthologue, paralogue or homologue thereof comprises a mutated PPO, wherein an amino acid ±3, ±2 or ±1 amino acid positions from a key amino acid is substituted by any other amino acid.

Based on techniques well-known in the art, a highly characteristic sequence pattern can be developed, by means of which further of mutated PPO candidates with the desired activity may be searched.

Searching for further mutated PPO candidates by applying a suitable sequence pattern would also be encompassed by the present invention. It will be understood by a skilled reader that the present sequence pattern is not limited by the exact distances between two adjacent amino acid residues of said pattern. Each of the distances between two neighbours in the above patterns may, for example, vary independently of each other by up to ±10, ±5, ±3, ±2 or ±1 amino acid positions without substantially affecting the desired activity.

Furthermore, by applying the method of site directed mutagenesis, e.g. saturation mutagenes (see e.g. Schenk et al., Biospektrum March 2006, pages 277-279), the inventors of the present invention have identified and generated specific amino acid subsitutions and combinations thereof, which—when introduced into a plant by transforming and expressing the respective mutated PPO encoding nucleic acid—confer increased herbicide resistance or tolerance to a herbicide to said plant.

Thus, in a particularly preferred embodiment, the variant or derivative of the mutated PPO refers to a PPO polypeptide comprising SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129, a orthologue, paralogue, or homologue thereof, wherein the amino acid sequence differs from the wildtype amino acid sequence of a PPO polypeptide at one or more positions corresponding to the following positions of SEQ ID NO: 1 or 2: 32, 53, 57, 61, 63, 64, 65, 67, 71, 76, 82, 83, 85, 86, 87, 88, 91, 103, 104, 106, 108, 116, 119, 126, 127, 129, 139, 159, 210, 211, 224, 245, 246, 248, 249, 252, 253, 254, 255, 257, 259, 260, 262, 264, 286, 291, 305, 308, 309, 323, 335, 343, 345, 358, 372, 373, 387, 391, 392, 400, 412, 414, 415, 425, 428, 431, 433, 434, 435, 436, 447, 451, 453, 464, 466, 482 of SEQ ID NO: 1 or 2, wherein said difference refers to a substitution of the amino acid at that positions by any other amino acid.

In a particularly preferred embodiment, the variant or derivative of the mutated PPO refers to a PPO polypeptide comprising SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129, a orthologue, paralogue, or homologue thereof, wherein the amino acid sequence differs from the wildtype amino acid sequence of a PPO polypeptide at one or more positions corresponding to the following positions of SEQ ID NO: 1 or 2: 40, 250, 391, 520, 521, 522, 523, 524, 525, 526, 527, 528, 529, 530, 531, 532, 533, 534 of SEQ ID NO: 1 or 2, wherein said difference refers to a deletion or an insertion of the amino acid at that positions.

Preferably, the difference corresponding to position 391 of SEQ ID NO:1 referes to an insertion, and the difference corresponding to positions 40, 250, 520, 521, 522, 523, 524, 525, 526, 527, 528, 529, 530, 531, 532, 533, 534 of SEQ ID NO: 1 or 2 refers to a deletion

Preferably, said differences at these amino acid positions (corresponding to the respective positions in SEQ ID NO: 2 or 4) include, but are not limited to, one or more of the following:

the amino acid corresponding to position G32 is substituted by any other amino acid

the amino acid corresponding to position V53 is substituted by any other amino acid

the amino acid corresponding to position A57 is substituted by any other amino acid

the amino acid corresponding to position K61 is substituted by any other amino acid

the amino acid corresponding to position K63 is substituted by any other amino acid

the amino acid corresponding to position S64 is substituted by any other amino acid

the amino acid corresponding to position H65 is substituted by any other amino acid

the amino acid corresponding to position L67 is substituted by any other amino acid

the amino acid corresponding to position L71 is substituted by any other amino acid

the amino acid corresponding to position S76 is substituted by any other amino acid

the amino acid corresponding to position L82 is substituted by any other amino acid

the amino acid corresponding to position K83 is substituted by any other amino acid

the amino acid corresponding to position V85 is substituted by any other amino acid

the amino acid corresponding to position K86 is substituted by any other amino acid

the amino acid corresponding to position K87 is substituted by any other amino acid

the amino acid corresponding to position D88 is substituted by any other amino acid

the amino acid corresponding to position I91 is substituted by any other amino acid

the amino acid corresponding to position E103 is substituted by any other amino acid

the amino acid corresponding to position A104 is substituted by any other amino acid

the amino acid corresponding to position V106 is substituted by any other amino acid

the amino acid corresponding to position S108 is substituted by any other amino acid

the amino acid corresponding to position R116 is substituted by any other amino acid

the amino acid corresponding to position Q119 is substituted by any other amino acid, preferably Leu

the amino acid corresponding to position K127 is substituted by any other amino acid

the amino acid corresponding to position N126 is substituted by any other amino acid

the amino acid corresponding to position Y129 is substituted by any other amino acid

the amino acid corresponding to position L139 is substituted by any other amino acid

the amino acid corresponding to position Q159 is substituted by any other amino acid

the amino acid corresponding to position G210 is substituted by any other amino acid, preferably by Ser;

the amino acid corresponding to position G211 is substituted by any other amino acid, preferably by Asp, Asn, Thr or Ala;

the amino acid corresponding to position E224 is substituted by any other amino acid

the amino acid corresponding to position L245 is substituted by any other amino acid

the amino acid corresponding to position K248 is substituted by any other amino acid

the amino acid corresponding to position S246 is substituted by any other amino acid

the amino acid corresponding to position E249 is substituted by any other amino acid

the amino acid corresponding to position G252 is substituted by any other amino acid

the amino acid corresponding to position E253 is substituted by any other amino acid

the amino acid corresponding to position N254 is substituted by any other amino acid

the amino acid corresponding to position A255 is substituted by any other amino acid

the amino acid corresponding to position I257 is substituted by any other amino acid

the amino acid corresponding to position K259 is substituted by any other amino acid

the amino acid corresponding to position P260 is substituted by any other amino acid

the amino acid corresponding to position V262 is substituted by any other amino acid

the amino acid corresponding to position G264 is substituted by any other amino acid

the amino acid corresponding to position D286 is substituted by any other amino acid

the amino acid corresponding to position Q291 is substituted by any other amino acid

the amino acid corresponding to position P305 is substituted by any other amino acid

the amino acid corresponding to position G308 is substituted by any other amino acid

the amino acid corresponding to position N309 is substituted by any other amino acid

the amino acid corresponding to position Q323 is substituted by any other amino acid

the amino acid corresponding to position R335 is substituted by any other amino acid

the amino acid corresponding to position M343 is substituted by any other amino acid

the amino acid corresponding to position F345 is substituted by any other amino acid

the amino acid corresponding to position T358 is substituted by any other amino acid

the amino acid corresponding to position D372 is substituted by any other amino acid

the amino acid corresponding to position K373 is substituted by any other amino acid

the amino acid corresponding to position S387 is substituted by any other amino acid, preferably Leu

the amino acid corresponding to position H391 is substituted by any other amino acid

the amino acid corresponding to position N392 is substituted by any other amino acid

the amino acid corresponding to position L400 is substituted by any other amino acid

the amino acid corresponding to position S412 is substituted by any other amino acid, preferably by Asn;

the amino acid corresponding to position M414 is substituted by any other amino acid

the amino acid corresponding to position C415 is substituted by any other amino acid

the amino acid corresponding to position R425 is substituted by any other amino acid

the amino acid corresponding to position K428 is substituted by any other amino acid

the amino acid corresponding to position N431 is substituted by any other amino acid

the amino acid corresponding to position S433 is substituted by any other amino acid

the amino acid corresponding to position M434 is substituted by any other amino acid

the amino acid corresponding to position D435 is substituted by any other amino acid

the amino acid corresponding to position E436 is substituted by any other amino acid

the amino acid corresponding to position Q447 is substituted by any other amino acid

the amino acid corresponding to position T451 is substituted by any other amino acid

the amino acid corresponding to position D453 is substituted by any other amino acid

the amino acid corresponding to position S464 is substituted by any other amino acid

the amino acid corresponding to position Q466 is substituted by any other amino acid

the amino acid corresponding to position D482 is substituted by any other amino acid

The corresponding residues in other species are shown in the following table:

| SEQ ID | Pos 1 | Pos 2 | Pos 3 | Pos 4 | Pos 5 | Pos 6 | Pos 7 | Pos 8 | Pos 9 | Pos 10 |
|---|---|---|---|---|---|---|---|---|---|---|
| 1 | G32 | V53 | A57 | K61 | K63 | S64 | H65 | L67 | L71 | S76 |
| 2 | G32 | V53 | A57 | K61 | K63 | S64 | H65 | L67 | L71 | S76 |
| 3 | G32 | V53 | A57 | K61 | K63 | S64 | H65 | L67 | L71 | S76 |
| 4 | G32 | V53 | A57 | K61 | K63 | S64 | H65 | L67 | L71 | S76 |
| 5 | G32 | V53 | A57 | K61 | K63 | S64 | H65 | L67 | L71 | S76 |
| 6 | G2 | V23 | A27 | K31 | K33 | S34 | H35 | L37 | L41 | S46 |
| 7 | V30 | V52 | A56 | K60 | K62 | S63 | N64 | L66 | L70 | S75 |
| 8 | K4 | V25 | A29 | K33 | K35 | L36 | H37 | V39 | L43 | E48 |
| 9 | S2 | V24 | A28 | K32 | K34 | S35 | H36 | L38 | V42 | E47 |
| 10 | N6 | V23 | A27 | K31 | K33 | S34 | H35 | L37 | M41 | G46 |
| 11 | T6 | V23 | A27 | K31 | K33 | S34 | H35 | L37 | V41 | G46 |
| 12 | L4 | V21 | A25 | K29 | K31 | S32 | H33 | F35 | V39 | G44 |
| 13 | K6 | V23 | A27 | K31 | K33 | S34 | H35 | L37 | V41 | G46 |
| 14 | Q6 | V23 | A27 | K31 | K33 | S34 | H35 | F37 | V41 | G46 |
| 15 | G6 | V23 | A27 | K31 | K33 | S34 | N35 | V37 | V41 | E46 |
| 16 | G11 | V23 | A27 | K31 | K33 | S34 | N35 | V37 | V41 | E46 |
| 17 | K6 | V23 | A27 | K31 | K33 | S34 | H35 | L37 | V41 | G46 |
| 18 | A5 | V23 | A27 | K31 | K33 | S34 | H35 | F37 | V41 | E46 |
| 19 | — | V18 | A22 | K26 | K28 | S29 | H30 | F32 | V36 | E41 |
| 20 | G6 | V23 | A27 | K31 | K33 | I34 | H35 | L37 | V41 | G46 |
| 21 | G6 | V20 | A24 | K28 | K30 | V31 | H32 | L34 | V38 | G43 |
| 22 | G4 | V26 | A30 | K34 | K36 | S37 | R38 | L40 | V44 | G49 |
| 23 | G7 | V28 | A32 | K36 | K38 | S39 | R40 | L42 | V46 | E51 |
| 24 | G4 | V26 | A30 | K34 | K36 | S37 | R38 | L40 | V44 | G49 |
| 25 | G50 | V72 | A76 | K80 | K82 | S83 | R84 | L86 | V90 | G95 |
| 26 | — | — | A3 | K7 | K9 | L10 | H11 | L13 | V17 | E22 |
| 27 | G34 | V53 | A57 | R61 | R63 | K64 | S65 | V67 | V71 | D76 |
| 28 | G36 | V55 | V59 | R63 | R65 | K66 | S67 | V69 | V73 | D78 |
| 29 | G4 | V26 | A30 | K34 | K36 | S37 | R38 | L40 | V44 | G49 |
| 30 | G36 | V55 | A59 | R63 | R65 | Q66 | S67 | V69 | V73 | D78 |
| 31 | G36 | V55 | A59 | R63 | R65 | Q66 | S67 | V69 | V73 | D78 |
| 32 | G8 | I31 | A35 | K39 | K41 | S42 | N43 | F45 | V49 | G54 |
| 33 | A41 | I69 | Q73 | A77 | R86 | S87 | S88 | P90 | V94 | D99 |
| 34 | A4 | V25 | A29 | K33 | K35 | S36 | N37 | V39 | V43 | G48 |
| 35 | A69 | V90 | A94 | K98 | K100 | S101 | K102 | L104 | V108 | G113 |
| 36 | L36 | V59 | V63 | R67 | R69 | K70 | S71 | V73 | V77 | D82 |
| 37 | — | V20 | A24 | R28 | R30 | K31 | R32 | V34 | V38 | D43 |
| 38 | H4 | I25 | A29 | R33 | K35 | S36 | Q37 | L39 | I43 | G48 |
| 39 | A2 | V23 | A27 | K31 | K33 | I34 | H35 | L37 | V41 | G46 |
| 40 | — | V20 | A24 | R28 | R30 | K31 | R32 | V34 | V38 | D43 |
| 41 | — | V20 | A24 | R28 | R30 | K31 | R32 | V34 | V38 | D43 |
| 42 | A7 | V28 | — | R29 | K31 | S32 | R33 | L35 | V39 | G44 |
| 43 | M1 | A22 | A26 | R30 | R32 | A33 | A34 | V36 | V40 | N45 |
| 44 | M1 | A22 | A26 | R30 | R32 | A33 | A34 | V36 | V40 | N45 |
| 45 | G32 | V53 | A57 | K61 | K63 | S64 | H65 | L67 | L71 | S76 |
| 46 | G32 | V53 | A57 | K61 | K63 | S64 | H65 | L67 | L71 | S76 |
| 47 | — | — | — | — | — | — | — | — | — | — |
| 48 | — | — | — | — | — | — | — | — | — | — |
| 49 | — | V14 | S18 | K22 | K24 | L25 | N26 | L28 | V32 | G37 |
| 50 | — | — | — | — | — | — | — | — | — | — |
| 51 | — | I11 | T15 | E19 | H21 | R22 | R23 | L25 | V29 | D34 |
| 52 | — | I11 | A15 | R19 | Q21 | E22 | H23 | H25 | L29 | G34 |
| 53 | — | I11 | A15 | R19 | Q21 | E22 | H23 | H25 | V29 | G34 |
| 54 | — | — | — | — | — | — | — | — | — | — |
| 55 | — | I11 | A15 | E19 | H21 | R22 | R23 | L25 | V29 | D34 |
| 56 | — | I10 | T14 | Y18 | K20 | Q21 | Q22 | V24 | V28 | N33 |
| 57 | S4 | I27 | T31 | R35 | T37 | Q38 | L39 | H41 | V45 | D50 |
| 58 | — | I15 | T19 | E23 | L25 | K26 | L27 | H29 | V33 | S38 |
| 59 | — | I12 | T16 | L20 | S22 | K23 | R24 | F26 | 130 | S35 |
| 60 | — | I12 | C16 | E20 | K22 | K23 | S24 | F26 | V30 | E35 |
| 61 | — | I11 | T15 | Q19 | Q21 | Q22 | Q23 | H25 | V29 | G34 |
| 62 | A2 | V23 | A27 | K31 | K33 | L34 | H35 | L37 | V41 | E46 |
| 63 | — | I12 | S16 | W20 | V22 | K23 | K24 | Y26 | I30 | N35 |
| 64 | M1 | I22 | A26 | H30 | S32 | R33 | M34 | K36 | V40 | S45 |
| 65 | — | P16 | S20 | R24 | L26 | H27 | P31 | L33 | L39 | G44 |
| 66 | — | V12 | S16 | H20 | Q22 | Q23 | A24 | H26 | V30 | K35 |
| 67 | — | I12 | S16 | F20 | K22 | R23 | A24 | A26 | V30 | E35 |
| 68 | — | I11 | A15 | Q19 | Q21 | R22 | K23 | K25 | V29 | P34 |
| 69 | — | I13 | S17 | T21 | F23 | K24 | R25 | L27 | V31 | A36 |
| 70 | — | I10 | T14 | R18 | A20 | R21 | E22 | H24 | L28 | P33 |
| 71 | — | V12 | T16 | R20 | K22 | S23 | H24 | L26 | L30 | G35 |
| 72 | P8 | I29 | T33 | R37 | A39 | R40 | E41 | H43 | L47 | P52 |
| 73 | — | I14 | A18 | T22 | H24 | K25 | R26 | Y28 | V32 | N37 |
| 74 | — | V15 | A19 | T23 | K25 | N26 | R27 | V29 | L33 | G38 |
| 75 | — | V15 | A19 | T23 | K25 | N26 | R27 | V29 | L33 | G38 |
| 76 | — | L13 | S17 | S21 | E23 | N24 | K25 | N27 | I31 | L36 |
| 77 | — | I15 | A19 | T23 | Y25 | K26 | R27 | Y29 | V33 | N38 |
| 78 | — | I10 | S14 | Y18 | K20 | K21 | G22 | A24 | V28 | K33 |
| 79 | — | V11 | S15 | Y19 | E21 | K22 | F23 | G25 | I32 | N37 |
| 80 | — | I14 | S18 | S22 | N24 | R25 | S28 | R30 | V37 | N42 |
| 81 | — | I15 | A19 | T23 | K25 | N26 | R27 | V29 | L33 | G38 |
| 82 | — | I11 | T15 | L19 | Q21 | K22 | Q23 | Q25 | V29 | K34 |
| 83 | — | L13 | S17 | S21 | E23 | N24 | K25 | N27 | I31 | L36 |
| 84 | — | I20 | T24 | R28 | Q30 | Q31 | R36 | G38 | L45 | S50 |
| 85 | — | I10 | V14 | Q18 | Q20 | K21 | A22 | V24 | L28 | T33 |
| 86 | — | I10 | S14 | Y18 | Q20 | R21 | Q22 | V24 | L28 | S33 |
| 87 | — | I15 | A19 | H23 | K25 | L26 | A27 | R29 | L33 | D38 |
| 88 | — | L14 | T18 | H22 | R24 | H25 | K26 | Q28 | V32 | E37 |
| 89 | — | I9 | S13 | H17 | Q19 | K20 | L21 | K23 | L27 | D32 |
| 90 | — | I11 | A15 | E19 | R21 | N22 | I29 | L31 | L35 | E40 |
| 91 | — | I12 | T16 | R20 | Q22 | Q23 | S31 | Q33 | L37 | S42 |
| 92 | — | I11 | S15 | A19 | H21 | Q22 | D23 | R25 | L29 | E34 |
| 93 | — | I17 | S21 | H25 | Q27 | E28 | Q35 | V37 | L41 | P46 |
| 94 | — | I11 | A15 | L19 | R21 | E22 | M29 | L31 | I35 | D40 |
| 95 | — | I11 | A15 | F19 | R21 | K22 | Q23 | W25 | L29 | A34 |
| 96 | — | L13 | T17 | Y21 | R23 | R24 | K25 | Q27 | V31 | D36 |
| 97 | — | V10 | S14 | Y18 | K20 | R21 | G22 | A24 | L28 | N33 |
| 98 | G41 | I65 | C69 | A73 | V75 | T76 | A81 | K83 | V87 | D92 |
| 99 | N4 | V25 | A29 | K33 | K35 | S36 | K37 | V39 | V43 | G48 |
| 100 | S47 | I68 | C72 | A76 | A78 | T79 | V84 | S86 | V90 | D95 |
| 101 | A2 | V23 | A27 | K31 | K33 | S34 | Q35 | L37 | V41 | G46 |
| 102 | A45 | I67 | C71 | A75 | S77 | T78 | H80 | G82 | V86 | E91 |
| 103 | T6 | V27 | A31 | K35 | K37 | L38 | H39 | I41 | V45 | E50 |
| 104 | — | I94 | C98 | A102 | S104 | T105 | L110 | T112 | V116 | D121 |
| 105 | S36 | I60 | C64 | A68 | A70 | T71 | G74 | T76 | V80 | A85 |
| 106 | M1 | V22 | V26 | R30 | R32 | K33 | S34 | V36 | V40 | D45 |
| 107 | S47 | I68 | C72 | A76 | A78 | T79 | V84 | S86 | V90 | D95 |
| 108 | A64 | I89 | C93 | A97 | C99 | T100 | L105 | P107 | V111 | D116 |
| 109 | T38 | I59 | C63 | A67 | A69 | T70 | G73 | G75 | V79 | D84 |
| 110 | L47 | V67 | V71 | M75 | R77 | K78 | S79 | V81 | V85 | D90 |
| 111 | T39 | I60 | C64 | A68 | A70 | T71 | G74 | S76 | V80 | D85 |
| 112 | — | V20 | A24 | K28 | K30 | V31 | H32 | L34 | V38 | G43 |
| 113 | T54 | I75 | C79 | A83 | A85 | T86 | G89 | S91 | V95 | D100 |
| 114 | S47 | I68 | C72 | A76 | A78 | T79 | V84 | S86 | V90 | D95 |
| 115 | A2 | V23 | A27 | K31 | K33 | S34 | Q35 | L37 | V41 | G46 |
| 116 | A64 | I89 | C93 | A97 | C99 | T100 | L105 | P107 | V111 | D116 |
| 117 | S3 | V24 | A28 | K32 | K34 | S35 | K36 | V38 | V42 | G47 |
| 129 | Y32 | I62 | V66 | R70 | S72 | K73 | S74 | V76 | V80 | D85 |
| SEQ ID | Pos 11 | Pos 12 | Pos 13 | Pos 14 | Pos 15 | Pos 16 | Pos 17 | Pos 18 | Pos 19 | Pos 20 |
| 1 | L82 | K83 | V85 | K86 | K87 | D88 | I91 | E103 | A104 | V106 |
| 2 | L82 | K83 | V85 | K86 | K87 | D88 | I91 | E103 | A104 | V106 |
| 3 | L82 | K83 | V85 | K86 | K87 | D88 | I91 | E103 | A104 | V106 |
| 4 | L82 | K83 | V85 | K86 | K87 | D88 | I91 | E103 | A104 | V106 |
| 5 | L82 | K83 | V85 | K86 | K87 | D88 | I91 | E103 | A104 | V106 |
| 6 | L52 | K53 | V55 | K56 | K57 | D58 | I61 | E73 | A74 | V76 |
| 7 | L81 | K82 | V84 | V85 | K86 | D87 | I90 | D102 | E103 | V105 |
| 8 | L54 | R55 | V57 | S58 | Q59 | H60 | V63 | E75 | I76 | V78 |
| 9 | L53 | R54 | V56 | N57 | H58 | D59 | I62 | E74 | M75 | V77 |
| 10 | L52 | R53 | V55 | S56 | Q57 | E58 | I61 | E73 | I74 | V76 |
| 11 | L52 | R53 | V55 | S56 | Q57 | D58 | I61 | E73 | I74 | V76 |
| 12 | L50 | R51 | V53 | S54 | H55 | D56 | I59 | E71 | K72 | V74 |
| 13 | L52 | R53 | V55 | S56 | R57 | D58 | V61 | E73 | I74 | V76 |
| 14 | L52 | R53 | V55 | S56 | Y57 | N58 | I61 | E73 | T74 | V76 |
| 15 | L52 | R53 | I55 | S56 | K57 | D58 | I61 | E73 | M74 | V76 |
| 16 | L52 | R53 | I55 | S56 | K57 | D58 | I61 | E73 | M74 | V76 |
| 17 | L52 | R53 | V55 | S56 | R57 | D58 | V61 | E73 | A74 | V76 |
| 18 | L52 | R53 | V55 | S56 | Y57 | K58 | I61 | E73 | P74 | V76 |
| 19 | L47 | R48 | V50 | S51 | Y52 | K53 | I56 | E68 | P69 | V71 |
| 20 | L52 | R53 | V55 | S56 | Q57 | D58 | I61 | E73 | G74 | V76 |
| 21 | L49 | R50 | L52 | S53 | Q54 | D55 | I58 | E70 | G71 | V73 |
| 22 | L55 | R56 | V58 | M59 | Q60 | N61 | I64 | E76 | P77 | V79 |
| 23 | L57 | T58 | V60 | M61 | Q62 | N63 | I66 | E78 | P79 | V81 |
| 24 | L55 | R56 | V58 | M59 | Q60 | N61 | I64 | E76 | P77 | V79 |
| 25 | L101 | R102 | V104 | M105 | Q106 | N107 | I110 | E122 | P123 | V125 |
| 26 | L28 | R29 | V31 | S32 | Q33 | D34 | I37 | E49 | A50 | V52 |
| 27 | I82 | R83 | N85 | S86 | E87 | A88 | L91 | E103 | L104 | V106 |
| 28 | I84 | R85 | N87 | S88 | E89 | G90 | L93 | E105 | L106 | A108 |
| 29 | L55 | R56 | V58 | M59 | Q60 | N61 | I64 | E76 | P77 | V79 |
| 30 | I84 | R85 | N87 | S88 | E89 | G90 | V93 | E105 | W106 | A108 |
| 31 | I84 | R85 | N87 | S88 | E89 | G90 | V93 | E105 | W106 | A108 |
| 32 | I60 | R61 | G63 | S64 | Q65 | E66 | I69 | — | V81 | A83 |
| 33 | I105 | T106 | V108 | E109 | R110 | D111 | L114 | D126 | A127 | L129 |
| 34 | L54 | R55 | V57 | S58 | H59 | H60 | V63 | E75 | V76 | V78 |
| 35 | L119 | R120 | V122 | M123 | H124 | N125 | I128 | E140 | P141 | V143 |
| 36 | I88 | R89 | N91 | S92 | D93 | S94 | L97 | A109 | L110 | A112 |
| 37 | I49 | R50 | N52 | S53 | E54 | G55 | I58 | E70 | L71 | A73 |
| 38 | I54 | K55 | F57 | A58 | Q59 | N60 | I63 | E75 | P76 | V78 |
| 39 | L52 | R53 | L55 | S56 | Q57 | D58 | I61 | E73 | G74 | V76 |
| 40 | I49 | R50 | N52 | S53 | E54 | G55 | I58 | E70 | L71 | A73 |
| 41 | I49 | R50 | N52 | S53 | E54 | G55 | I58 | E70 | L71 | A73 |
| 42 | L50 | R51 | V53 | M54 | H55 | K56 | I59 | E71 | P72 | V74 |
| 43 | L51 | K52 | V54 | S55 | E56 | N57 | I60 | D72 | P73 | I75 |
| 44 | L51 | K52 | V54 | S55 | E56 | N57 | I60 | D72 | P73 | I75 |
| 45 | L82 | K83 | V85 | K86 | K87 | D88 | I91 | E103 | A104 | V106 |
| 46 | L82 | K83 | V85 | K86 | K87 | D88 | I91 | E103 | A104 | V106 |
| 47 | — | — | — | — | — | — | — | — | — | — |
| 48 | — | — | — | — | — | — | — | A5 | L6 | A8 |
| 49 | I43 | R44 | S46 | S47 | Q48 | D49 | I52 | E64 | E65 | V67 |
| 50 | — | — | — | — | — | — | — | — | — | — |
| 51 | I40 | Q41 | E43 | R44 | I45 | D46 | L49 | S61 | G62 | F64 |
| 52 | I40 | R41 | E43 | S44 | S45 | E46 | L49 | A61 | A62 | L64 |
| 53 | I40 | R41 | E43 | S44 | S45 | E46 | L49 | A61 | A62 | L64 |
| 54 | — | — | — | — | — | — | — | — | — | — |
| 55 | I40 | Q41 | E43 | R44 | I45 | D46 | L49 | S61 | G62 | F64 |
| 56 | V39 | I40 | E42 | K43 | K44 | D45 | L48 | S60 | Q61 | L63 |
| 57 | I56 | K57 | E59 | E60 | V61 | D62 | L65 | E77 | L78 | V80 |
| 58 | I44 | E45 | I47 | R48 | Q49 | N50 | L53 | S65 | P66 | I68 |
| 59 | I41 | E42 | I44 | K45 | E46 | N47 | L50 | T62 | P63 | I65 |
| 60 | I41 | G42 | T44 | A45 | R46 | D47 | R50 | S62 | Q63 | T65 |
| 61 | I40 | R41 | E43 | R44 | T45 | D47 | L50 | T62 | P63 | V65 |
| 62 | P52 | R53 | V55 | S56 | Q57 | D58 | I61 | E73 | A74 | A76 |
| 63 | M41 | I42 | R44 | R45 | L46 | D47 | L50 | T62 | P63 | I65 |
| 64 | V51 | G52 | I54 | E55 | E56 | D57 | L60 | T71 | P72 | M74 |
| 65 | V50 | T51 | A53 | C54 | D55 | A56 | C59 | H71 | P72 | V74 |
| 66 | I41 | Q42 | I44 | R45 | E46 | D47 | L50 | D62 | K63 | I65 |
| 67 | M41 | R42 | R44 | R45 | F46 | K47 | L50 | T62 | P63 | F65 |
| 68 | I40 | Q41 | I43 | L44 | Q45 | D46 | L49 | S61 | L62 | I64 |
| 69 | M42 | R43 | I45 | V46 | T47 | E49 | V52 | D64 | P65 | M67 |
| 70 | I39 | R40 | E42 | R43 | D44 | G45 | L48 | K60 | P61 | L63 |
| 71 | I41 | R42 | I44 | A45 | Y46 | G47 | I50 | E62 | M63 | V65 |
| 72 | I58 | R59 | E61 | R62 | D63 | G64 | L67 | K79 | P80 | L82 |
| 73 | I43 | Q44 | I46 | T47 | T48 | E50 | I53 | D65 | A66 | L68 |
| 74 | I44 | R45 | V47 | R48 | E49 | D50 | L53 | E66 | D67 | I69 |
| 75 | I44 | R45 | V47 | R48 | E49 | D50 | L53 | E66 | D67 | I69 |
| 76 | L42 | Q43 | I45 | K46 | S47 | E48 | L51 | T63 | K64 | T66 |
| 77 | I44 | H45 | I47 | T48 | T49 | E51 | T54 | D66 | V67 | L69 |
| 78 | M39 | K40 | I42 | H43 | E44 | D45 | I48 | K60 | P61 | T63 |
| 79 | M43 | L44 | V46 | Q47 | K48 | D49 | I52 | K64 | P65 | T67 |
| 80 | I48 | T49 | G51 | R52 | A53 | D54 | L57 | T68 | P69 | L71 |
| 81 | V44 | R45 | V47 | R48 | E49 | E50 | L53 | D66 | D67 | I69 |
| 82 | I40 | S41 | F43 | R44 | E45 | E46 | L49 | D61 | P62 | V64 |
| 83 | L42 | Q43 | I45 | K46 | S47 | E48 | L51 | T63 | K64 | T66 |
| 84 | I56 | S57 | Q59 | S60 | K61 | D62 | R65 | T76 | P77 | L79 |
| 85 | L39 | R40 | V42 | S43 | H44 | D46 | L49 | S60 | D61 | L63 |
| 86 | M39 | R40 | L42 | R43 | K44 | N45 | T48 | N59 | E60 | L62 |
| 87 | I44 | R45 | L47 | S48 | D49 | S50 | R53 | E66 | E67 | L69 |
| 88 | M43 | Q44 | E46 | D47 | F48 | D49 | I52 | Y64 | P65 | V67 |
| 89 | I38 | Q39 | V41 | K42 | K43 | E44 | L47 | D58 | S59 | I61 |
| 90 | I46 | W47 | I49 | K50 | E51 | E52 | L55 | K67 | P68 | T70 |
| 91 | I48 | S49 | Q51 | S52 | K53 | D54 | R57 | V68 | P69 | L71 |
| 92 | I40 | T41 | G43 | K44 | A45 | G46 | L49 | T60 | P61 | L63 |
| 93 | I52 | G53 | I55 | S56 | Q57 | D58 | R61 | K73 | P74 | A76 |
| 94 | I46 | R47 | I49 | K50 | E51 | D52 | L55 | K67 | P68 | T70 |
| 95 | L40 | Q41 | R43 | Q44 | E45 | E46 | L49 | G61 | H62 | V64 |
| 96 | M42 | Q43 | E45 | E46 | V47 | D48 | V51 | Y63 | P64 | V66 |
| 97 | A39 | R40 | Y42 | Y43 | E44 | K45 | I48 | K60 | P61 | T63 |
| 98 | I98 | I99 | R101 | E102 | E103 | Q104 | L107 | S118 | D119 | M121 |
| 99 | L54 | R55 | V57 | M58 | H59 | N60 | I63 | E75 | P76 | V78 |
| 100 | I101 | T102 | V104 | E105 | R106 | D107 | L110 | S121 | D122 | I124 |
| 101 | L52 | R53 | V55 | S56 | R57 | E58 | I61 | E73 | I74 | V76 |
| 102 | I97 | S98 | V100 | E101 | R102 | D103 | L106 | S117 | D118 | M120 |
| 103 | L56 | R57 | I59 | S60 | Q61 | N62 | I65 | E77 | P78 | V80 |
| 104 | I127 | T128 | K130 | E131 | D132 | D133 | I136 | S147 | D148 | V150 |
| 105 | I91 | T92 | V94 | E95 | R96 | E99 | L102 | S113 | D114 | V116 |
| 106 | I51 | R52 | N54 | S55 | D56 | G57 | L60 | A72 | L73 | A75 |
| 107 | I101 | T102 | V104 | E105 | R106 | D107 | L110 | S121 | D122 | I124 |
| 108 | I122 | V123 | V125 | E126 | A127 | D128 | I131 | S142 | D143 | V145 |
| 109 | I90 | T91 | V93 | E94 | R95 | E98 | L101 | S112 | D113 | V115 |
| 110 | I96 | R97 | N99 | S100 | D101 | G102 | L105 | A117 | L118 | A120 |
| 111 | I91 | T92 | V94 | E95 | R96 | E99 | L102 | S113 | D114 | V116 |
| 112 | L49 | R50 | L52 | S53 | Q54 | D55 | I58 | E70 | G71 | V73 |
| 113 | I106 | T107 | V109 | E110 | R111 | E114 | L117 | S128 | D129 | V131 |
| 114 | I101 | T102 | V104 | E105 | R106 | D107 | L110 | S121 | D122 | I124 |
| 115 | L52 | R53 | V55 | S56 | R57 | D58 | I61 | E73 | I74 | V76 |
| 116 | I122 | V123 | V125 | E126 | A127 | D128 | I131 | S142 | D143 | V145 |
| 117 | L53 | R54 | V56 | M57 | H58 | N59 | I62 | E74 | P75 | V77 |
| 129 | I91 | R92 | N94 | S95 | D96 | S97 | L100 | A112 | L113 | A115 |
| SEQ ID | Pos 21 | Pos 22 | Pos 23 | Pos 24 | Pos 25 | Pos 26 | Pos 27 | Pos 28 | Pos 29 | Pos 30 |
| 1 | S108 | R116 | Q119 | N126 | K127 | Y129 | L139 | Q159 | G210 | G211 |
| 2 | S108 | R116 | Q119 | N126 | K127 | Y129 | L139 | Q159 | G210 | G211 |
| 3 | S108 | R116 | Q119 | N126 | K127 | Y129 | L139 | Q159 | — | G210 |
| 4 | S108 | R116 | Q119 | N126 | K127 | Y129 | L139 | Q159 | — | G210 |
| 5 | S108 | R116 | Q119 | N126 | K127 | Y129 | L139 | Q159 | G210 | G211 |
| 6 | S78 | R86 | Q89 | N96 | K97 | Y99 | L109 | Q129 | G180 | G181 |
| 7 | S107 | R115 | L118 | N125 | K126 | Y128 | L138 | Q158 | G209 | G210 |
| 8 | S80 | R88 | Q91 | N98 | K99 | Y101 | L111 | Q131 | G182 | G183 |
| 9 | S79 | R87 | Q90 | N97 | K98 | Y100 | I110 | Q130 | A181 | G182 |
| 10 | S78 | R86 | Q89 | K96 | K97 | Y99 | I109 | Q129 | A180 | G181 |
| 11 | G78 | 086 | Q89 | H96 | K97 | Y99 | V109 | H129 | A180 | A181 |
| 12 | T76 | R84 | Q87 | N94 | K95 | Y97 | I107 | Q127 | A175 | G176 |
| 13 | G78 | H86 | Q89 | H96 | K97 | Y99 | V109 | Q129 | A180 | A181 |
| 14 | T78 | R86 | Q89 | N96 | K97 | Y99 | L109 | Q129 | A177 | G178 |
| 15 | G78 | R86 | Q89 | Y96 | K97 | Y99 | I109 | Q129 | A180 | A181 |
| 16 | G78 | R86 | Q89 | Y96 | K97 | Y99 | I109 | Q129 | A180 | A181 |
| 17 | S78 | Q86 | Q89 | H96 | K97 | Y99 | V109 | R129 | A180 | A181 |
| 18 | C78 | R86 | Q89 | N96 | K97 | Y99 | V109 | Q129 | A180 | G181 |
| 19 | C73 | R81 | Q84 | N91 | K92 | Y94 | V104 | Q124 | A175 | G176 |
| 20 | F78 | R86 | Q89 | N96 | K97 | Y99 | L109 | Q129 | G178 | G179 |
| 21 | F75 | R83 | Q86 | N93 | K94 | Y96 | I106 | Q126 | G175 | G176 |
| 22 | S81 | R89 | Q92 | K99 | K100 | Y102 | L112 | Q132 | A182 | A183 |
| 23 | S83 | R91 | Q94 | K101 | K102 | Y104 | L114 | Q134 | A186 | A187 |
| 24 | S81 | R89 | Q92 | K99 | K100 | Y102 | L112 | Q132 | A182 | A183 |
| 25 | S127 | R135 | Q138 | K145 | K146 | Y148 | L158 | Q178 | A228 | A229 |
| 26 | S54 | R62 | Q65 | H72 | K73 | Y75 | I85 | Q105 | G151 | G152 |
| 27 | R108 | Q116 | Q119 | H126 | K127 | Y129 | I139 | A159 | A213 | G214 |
| 28 | R110 | Q118 | Q121 | H128 | K129 | Y131 | I141 | A161 | A215 | G216 |
| 29 | S81 | R89 | Q92 | K99 | K100 | Y102 | — | — | A152 | A153 |
| 30 | R110 | Q118 | Q121 | H128 | K129 | Y131 | I141 | A161 | A215 | G216 |
| 31 | R110 | Q118 | Q121 | H128 | K129 | Y131 | I141 | A161 | A215 | G216 |
| 32 | E85 | R93 | Q96 | S103 | K104 | Y106 | I116 | R136 | G188 | G189 |
| 33 | M131 | K138 | L141 | A148 | P149 | F151 | V161 | R181 | A226 | G227 |
| 34 | S80 | R88 | Q91 | N98 | K99 | Y101 | I111 | Q131 | A182 | G183 |
| 35 | T145 | R153 | Q156 | K163 | K164 | Y166 | I176 | E196 | A248 | A249 |
| 36 | R114 | Q122 | Q125 | H132 | K133 | Y135 | I145 | K165 | A219 | G220 |
| 37 | R75 | Q83 | Q86 | H93 | K94 | Y96 | I106 | K126 | G179 | G180 |
| 38 | K80 | R88 | Q91 | S98 | K99 | Y101 | L111 | N131 | G186 | A187 |
| 39 | F78 | R86 | Q89 | N96 | K97 | Y99 | I109 | Q129 | G178 | G179 |
| 40 | R75 | Q83 | Q86 | H93 | K94 | Y96 | I106 | K126 | G190 | G191 |
| 41 | R75 | Q83 | Q86 | H93 | K94 | Y96 | I106 | K126 | G190 | G191 |
| 42 | S76 | R84 | Q87 | K94 | K95 | Y97 | I107 | Q127 | A179 | A180 |
| 43 | R77 | R85 | Q88 | K95 | K96 | Y98 | L108 | K128 | G175 | S176 |
| 44 | R77 | R85 | Q88 | K95 | K96 | Y98 | L108 | K128 | G175 | S176 |
| 45 | S108 | R116 | Q119 | N126 | K127 | Y129 | L139 | Q159 | G210 | G211 |
| 46 | S108 | R116 | Q119 | N126 | K127 | Y129 | L139 | Q159 | — | G210 |
| 47 | — | — | — | — | — | — | — | A13 | A67 | G68 |
| 48 | R10 | E18 | L21 | H28 | K29 | Y31 | — | K42 | A96 | G97 |
| 49 | F69 | R77 | Q80 | Q87 | K88 | Y90 | L100 | Q120 | G168 | G169 |
| 50 | — | — | — | — | — | — | — | — | — | — |
| 51 | E66 | E74 | C77 | A85 | N86 | F88 | L98 | R118 | A162 | G163 |
| 52 | T66 | H74 | R77 | D85 | T86 | Y88 | L98 | R118 | A161 | G162 |
| 53 | T66 | H74 | R77 | D85 | T86 | Y88 | L98 | R118 | A161 | G162 |
| 54 | — | — | — | — | — | — | — | — | — | — |
| 55 | E66 | E74 | C77 | A85 | N86 | F88 | L98 | R118 | A162 | G163 |
| 56 | Q65 | Q73 | K76 | N84 | K85 | Y87 | L97 | Q117 | A160 | G161 |
| 57 | K82 | N90 | R93 | K101 | N102 | Y104 | A114 | K134 | A178 | G179 |
| 58 | A70 | D78 | K81 | K89 | N90 | Y92 | M102 | N122 | A166 | G167 |
| 59 | Q67 | K75 | L78 | N86 | K87 | Y89 | L99 | R119 | A163 | G164 |
| 60 | D67 | K75 | I78 | K86 | K87 | F89 | V99 | R119 | G163 | A164 |
| 61 | R67 | E75 | R78 | A85 | T86 | F89 | L99 | R119 | A163 | G164 |
| 62 | S78 | R86 | Q89 | H96 | K97 | Y99 | I109 | Q129 | G175 | G176 |
| 63 | K67 | S75 | M78 | A85 | S86 | Y89 | L99 | R119 | A163 | G164 |
| 64 | A76 | V84 | I87 | A94 | K95 | F98 | L108 | H128 | A170 | G171 |
| 65 | D76 | K85 | M88 | A95 | K96 | Y99 | L109 | H129 | S176 | G177 |
| 66 | A67 | D75 | I78 | S85 | K86 | F89 | L99 | R119 | A163 | G164 |
| 67 | E67 | A75 | R78 | A85 | R86 | Y89 | L99 | R119 | A161 | G162 |
| 68 | D66 | E75 | I78 | A85 | Q86 | Y89 | V99 | R119 | A163 | G164 |
| 69 | A69 | R77 | L80 | G87 | K88 | Y91 | I101 | R121 | S164 | G165 |
| 70 | A65 | E73 | R76 | A83 | K84 | F87 | A97 | R117 | A161 | G162 |
| 71 | R67 | R75 | Q78 | N85 | K86 | F88 | I98 | R118 | A173 | A174 |
| 72 | A84 | E92 | R95 | A102 | K103 | F106 | A116 | R136 | A180 | G181 |
| 73 | Q70 | R78 | I81 | S88 | K89 | F92 | I102 | R122 | A166 | G167 |
| 74 | T71 | R79 | I82 | S89 | K90 | Y93 | V103 | D123 | A167 | S168 |
| 75 | T71 | R79 | I82 | S89 | K90 | Y93 | V103 | D123 | A167 | S168 |
| 76 | D68 | L76 | A79 | S86 | S87 | F90 | L100 | R120 | A165 | A166 |
| 77 | Q71 | R79 | I82 | G89 | R90 | Y93 | I103 | R123 | A167 | G168 |
| 78 | N65 | E73 | L76 | A83 | R84 | F87 | L97 | R117 | A161 | G162 |
| 79 | D69 | E77 | L80 | A87 | R88 | F91 | L101 | R121 | A165 | G166 |
| 80 | G73 | K81 | L84 | L91 | P92 | Y94 | V104 | R124 | A176 | G177 |
| 81 | A71 | R79 | I82 | S89 | K90 | Y93 | V103 | D123 | A167 | S168 |
| 82 | T66 | R74 | L77 | A84 | S85 | Y88 | V98 | G118 | A164 | G165 |
| 83 | D68 | L76 | A79 | S86 | S87 | F90 | L100 | R120 | A165 | A166 |
| 84 | N81 | T89 | L92 | L99 | P100 | Y102 | V112 | R132 | A177 | G178 |
| 85 | E65 | V73 | I76 | S83 | K84 | Y87 | L97 | N117 | A161 | G162 |
| 86 | Q64 | E72 | L75 | N82 | N83 | Y86 | V96 | R116 | G159 | G160 |
| 87 | K71 | A79 | I82 | G89 | K90 | F93 | L103 | R123 | A172 | S173 |
| 88 | E69 | K77 | L80 | A87 | K88 | L91 | L101 | R121 | A164 | G165 |
| 89 | D63 | E71 | M74 | S81 | K82 | Y85 | L95 | S115 | A158 | G159 |
| 90 | D72 | S80 | L83 | A90 | R91 | F94 | L104 | R124 | A168 | G169 |
| 91 | N73 | A81 | L84 | L91 | P92 | Y94 | V104 | R124 | A169 | G170 |
| 92 | K65 | R73 | L76 | L83 | P84 | F86 | V96 | R116 | A168 | G169 |
| 93 | E78 | E86 | L89 | Y96 | R97 | L100 | V110 | R138 | T187 | S188 |
| 94 | D72 | D80 | L83 | A90 | R91 | F94 | L104 | R124 | A170 | G171 |
| 95 | E66 | E74 | I77 | A84 | K85 | Y88 | V97 | R117 | A162 | G163 |
| 96 | E68 | D76 | L79 | A86 | K87 | L90 | L100 | R120 | A163 | G164 |
| 97 | E65 | E73 | L76 | A83 | R84 | F87 | L97 | R117 | A160 | G161 |
| 98 | T123 | K131 | L134 | A141 | P142 | F144 | V154 | R174 | A219 | G220 |
| 99 | S80 | R88 | Q91 | K110 | K111 | Y113 | I123 | Q143 | A195 | A196 |
| 100 | T126 | K134 | L137 | A144 | P145 | F147 | V157 | R177 | A222 | G223 |
| 101 | S78 | Q86 | E89 | N96 | K97 | Y99 | I109 | Q129 | A180 | G181 |
| 102 | T122 | K130 | L133 | A140 | P141 | F143 | V153 | R173 | A218 | G219 |
| 103 | R82 | R90 | Q93 | H100 | K101 | Y103 | V113 | 0133 | G179 | G180 |
| 104 | T152 | K160 | L163 | A170 | P171 | F173 | V183 | R203 | A248 | G249 |
| 105 | T118 | K126 | L129 | A136 | P137 | F139 | V149 | R169 | A214 | G215 |
| 106 | R77 | E85 | L88 | H95 | K96 | Y98 | I108 | K128 | A182 | G183 |
| 107 | T126 | K134 | L137 | A144 | P145 | F147 | V157 | R177 | A222 | G223 |
| 108 | T147 | K155 | L158 | A165 | P166 | F168 | V178 | R198 | A243 | G244 |
| 109 | T117 | K125 | L128 | A135 | P136 | F138 | V148 | R168 | A213 | G214 |
| 110 | R122 | Q130 | L133 | H140 | K141 | Y143 | I153 | K173 | A227 | G228 |
| 111 | T118 | K126 | L129 | A136 | P137 | F139 | V149 | R169 | A214 | G215 |
| 112 | F75 | R83 | Q86 | N93 | K94 | Y96 | I106 | Q126 | G175 | G176 |
| 113 | T133 | K141 | L144 | A151 | P152 | F154 | V164 | R184 | A229 | G230 |
| 114 | T126 | K134 | X137 | A144 | P145 | F147 | V157 | R177 | A222 | G223 |
| 115 | S78 | Q86 | Q89 | N96 | K97 | Y99 | I109 | Q129 | A180 | G181 |
| 116 | T147 | K155 | L158 | A165 | P166 | F168 | V178 | R198 | A243 | G244 |
| 117 | S79 | R87 | Q90 | K97 | K98 | Y100 | I110 | Q130 | A182 | A183 |
| 129 | R117 | E125 | L128 | H135 | K136 | Y138 | I148 | K168 | A222 | G223 |
| SEQ ID | Pos 31 | Pos 32 | Pos 33 | Pos 34 | Pos 35 | Pos 36 | Pos 37 | Pos 38 | Pos 39 | Pos 40 |
| 1 | E224 | L245 | S246 | K248 | E249 | G252 | E253 | N254 | A255 | I257 |
| 2 | E224 | L245 | S246 | K248 | E249 | G252 | E253 | N254 | A255 | I257 |
| 3 | E223 | L244 | S245 | K247 | E248 | G251 | E252 | N253 | A254 | I256 |
| 4 | E223 | L244 | S245 | K247 | E248 | G251 | E252 | N253 | A254 | I256 |
| 5 | D224 | L245 | S246 | K248 | E249 | G252 | G253 | E254 | A256 | I258 |
| 6 | E194 | L215 | S216 | K218 | E219 | G222 | G223 | G224 | A226 | I228 |
| 7 | E223 | S244 | S245 | K247 | E248 | G251 | E252 | K253 | S255 | N257 |
| 8 | E196 | S217 | A218 | R220 | E221 | G224 | E225 | T226 | S229 | E231 |
| 9 | E195 | S216 | T217 | R219 | G220 | G223 | E224 | T225 | S228 | V230 |
| 10 | E194 | S215 | A216 | R218 | E219 | G222 | E223 | R224 | T226 | E228 |
| 11 | E194 | F215 | A216 | R218 | E219 | G222 | E223 | N224 | A227 | R229 |
| 12 | D189 | S210 | A211 | K213 | G214 | G217 | E218 | T219 | S222 | E224 |
| 13 | E194 | F215 | G216 | K218 | D219 | G222 | E223 | T224 | V227 | R229 |
| 14 | E191 | S212 | S213 | K215 | E216 | G219 | D220 | T221 | S224 | E226 |
| 15 | E194 | S215 | A216 | K218 | E219 | A222 | E223 | A224 | S227 | E229 |
| 16 | E194 | S215 | A216 | K218 | E219 | A222 | E223 | A224 | S227 | E229 |
| 17 | E194 | F215 | G216 | R218 | D219 | G222 | E223 | T224 | A227 | R229 |
| 18 | E194 | S215 | T216 | K218 | E219 | G222 | V223 | R224 | T227 | G229 |
| 19 | E189 | S210 | T211 | K213 | E214 | G217 | V218 | R219 | T222 | G224 |
| 20 | E192 | S213 | P214 | N216 | E217 | G220 | G221 | P222 | T225 | A227 |
| 21 | D189 | S210 | P211 | K213 | E214 | G217 | G218 | P219 | T222 | I224 |
| 22 | D196 | A217 | A218 | G220 | G221 | R224 | D225 | T226 | S229 | G231 |
| 23 | D200 | A221 | A222 | G224 | G225 | G228 | E229 | A230 | S233 | G235 |
| 24 | D196 | A214 | A215 | G217 | G218 | R221 | D222 | T223 | S226 | G228 |
| 25 | D242 | A260 | A261 | G263 | G264 | R267 | D268 | T269 | S272 | G274 |
| 26 | E165 | S186 | S187 | G189 | G190 | K193 | — | — | P194 | G196 |
| 27 | A227 | T248 | A249 | G251 | D252 | K255 | T256 | G257 | L260 | G262 |
| 28 | A229 | T250 | A251 | G253 | D254 | K257 | T258 | R259 | S262 | A264 |
| 29 | D166 | A187 | A188 | G190 | G191 | R194 | D195 | T196 | S199 | G201 |
| 30 | A229 | A250 | A251 | G253 | D254 | K257 | T258 | R259 | S262 | G264 |
| 31 | A229 | A250 | A251 | G253 | D254 | K257 | T258 | R259 | S262 | G264 |
| 32 | E202 | L223 | D224 | R226 | K227 | T231 | E232 | R233 | A234 | V236 |
| 33 | K240 | E261 | R262 | K264 | N265 | P269 | R270 | D271 | P272 | L274 |
| 34 | D196 | S217 | A218 | K220 | E221 | K226 | K227 | G228 | S229 | E231 |
| 35 | D283 | A304 | A305 | — | — | — | — | — | — | — |
| 36 | G233 | T254 | A255 | G257 | D258 | A263 | D264 | T265 | S266 | G268 |
| 37 | A193 | S214 | T215 | G217 | D218 | G223 | G224 | A225 | S226 | G228 |
| 38 | E200 | S221 | R222 | K224 | A225 | A229 | K230 | H231 | V232 | H234 |
| 39 | E192 | S213 | P214 | K216 | E217 | P222 | P223 | K224 | T225 | V227 |
| 40 | A204 | S225 | T226 | G228 | D229 | G234 | G235 | A236 | S237 | G239 |
| 41 | A204 | S225 | T226 | G228 | D229 | G234 | G235 | A236 | S237 | G239 |
| 42 | D193 | A214 | A215 | G217 | G218 | T223 | R224 | T225 | S226 | G228 |
| 43 | E189 | S210 | K211 | K213 | K214 | S227 | D228 | F229 | P230 | R232 |
| 44 | E189 | S210 | K211 | K213 | K214 | S227 | D228 | F229 | P230 | R232 |
| 45 | E224 | — | — | — | — | — | — | — | — | — |
| 46 | E223 | — | — | — | — | — | — | — | — | — |
| 47 | A81 | A102 | A103 | G105 | D106 | R111 | H112 | D113 | S114 | G116 |
| 48 | G110 | T131 | A132 | G134 | D135 | G140 | G141 | A142 | S143 | G145 |
| 49 | D182 | S203 | A204 | R206 | G207 | N212 | K213 | G214 | S215 | T217 |
| 50 | — | S2 | S3 | G5 | E6 | H11 | E12 | A13 | T14 | R16 |
| 51 | K176 | K197 | Q198 | Y200 | H201 | — | — | — | — | — |
| 52 | R175 | T196 | S197 | D199 | G200 | — | — | — | — | — |
| 53 | R175 | T196 | S197 | D199 | G200 | — | — | — | — | — |
| 54 | — | — | — | — | | — | — | — | — | — |
| 55 | K176 | K197 | 0198 | Y200 | H201 | — | — | — | — | — |
| 56 | K174 | E195 | R196 | K198 | R199 | — | — | — | — | — |
| 57 | K192 | A213 | R214 | A216 | R217 | — | — | — | — | — |
| 58 | K180 | E201 | R202 | K204 | R205 | — | — | — | — | — |
| 59 | K177 | E198 | R199 | K201 | R202 | — | — | — | — | — |
| 60 | P177 | E198 | K199 | A201 | A202 | — | — | — | — | — |
| 61 | R177 | T198 | G199 | T201 | D202 | S205 | T206 | A207 | T208 | R210 |
| 62 | — | — | — | — | — | — | — | — | — | — |
| 63 | K177 | E198 | R199 | Q201 | R202 | E205 | S206 | K207 | Q208 | A210 |
| 64 | K184 | A205 | A206 | A208 | N209 | P212 | V213 | T214 | L215 | A217 |
| 65 | K190 | K211 | T212 | P214 | D215 | Y218 | K219 | P220 | Y221 | G223 |
| 66 | K177 | D198 | Q199 | N201 | S202 | S205 | K206 | H207 | K208 | Q210 |
| 67 | R175 | E196 | R197 | R199 | R200 | A203 | P204 | K205 | I206 | A208 |
| 68 | K177 | A198 | A199 | A201 | N202 | P205 | K206 | A207 | K208 | Y210 |
| 69 | S178 | A199 | K200 | A202 | A203 | K206 | K207 | A208 | K209 | I211 |
| 70 | L175 | A196 | R197 | A199 | T200 | A203 | G204 | P205 | A206 | K208 |
| 71 | — | — | — | — | — | — | — | — | — | — |
| 72 | L194 | A215 | R216 | A218 | T219 | A222 | G223 | P224 | A225 | K227 |
| 73 | S180 | P201 | K202 | Q204 | V205 | P208 | K209 | M210 | R211 | R213 |
| 74 | L181 | K202 | R203 | K205 | R206 | G209 | S210 | S211 | P212 | G214 |
| 75 | L181 | K202 | R203 | K205 | R206 | G209 | S210 | S211 | P212 | G214 |
| 76 | S179 | Q200 | P201 | E203 | K204 | K207 | S208 | — | — | — |
| 77 | S181 | Q202 | K203 | L205 | I206 | P209 | K210 | M211 | R212 | R214 |
| 78 | A175 | E196 | A197 | K199 | S200 | K203 | S204 | G205 | P206 | G208 |
| 79 | A179 | E200 | K201 | D203 | A204 | G207 | P208 | G209 | — | — |
| 80 | R190 | N211 | K212 | A214 | P215 | T218 | P219 | D220 | P221 | 1223 |
| 81 | L181 | K202 | R203 | K205 | R206 | G209 | S210 | A211 | P212 | G214 |
| 82 | L178 | K199 | Q200 | — | — | G204 | T205 | A206 | Y207 | K209 |
| 83 | S179 | Q200 | P201 | E203 | K204 | K207 | S208 | — | — | — |
| 84 | R191 | Q212 | R213 | Q215 | P216 | A219 | A220 | I221 | Q222 | P224 |
| 85 | K175 | G196 | A197 | R199 | R200 | — | — | — | — | — |
| 86 | E173 | R194 | G195 | — | — | — | — | — | — | — |
| 87 | R186 | Q207 | K208 | — | — | — | — | — | — | A210 |
| 88 | K178 | A199 | P200 | — | — | — | — | K201 | S202 | R204 |
| 89 | A172 | N193 | T194 | — | — | — | — | — | — | — |
| 90 | R182 | K203 | K204 | E207 | A208 | G211 | K212 | A213 | V214 | S216 |
| 91 | R183 | S204 | R205 | H216 | Q217 | A220 | N221 | S222 | P223 | Q225 |
| 92 | R182 | Q203 | R204 | Q206 | Q207 | P210 | T211 | D212 | P213 | L215 |
| 93 | R201 | Q222 | T223 | — | — | — | K224 | S225 | A226 | A228 |
| 94 | R184 | K205 | K206 | E209 | I210 | G213 | K214 | Q215 | V216 | S218 |
| 95 | R176 | K197 | K198 | — | — | — | — | S199 | P200 | T202 |
| 96 | K177 | Q198 | P199 | — | — | — | — | K200 | T201 | R203 |
| 97 | T174 | E195 | A196 | — | K197 | G200 | A201 | K202 | S203 | G205 |
| 98 | K233 | A254 | K255 | A258 | P259 | T262 | R263 | D264 | P265 | L267 |
| 99 | D209 | A230 | A231 | K235 | N236 | T239 | K240 | S241 | S242 | G244 |
| 100 | R236 | E257 | R258 | T261 | P262 | P265 | R266 | D267 | P268 | L270 |
| 101 | E194 | S215 | A216 | T219 | N220 | E223 | T224 | K225 | N226 | V228 |
| 102 | K232 | A253 | K254 | S257 | T258 | P261 | R262 | D263 | P264 | L266 |
| 103 | E193 | S214 | S215 | — | R216 | K219 | K220 | S221 | P222 | G224 |
| 104 | R262 | E283 | K284 | K287 | P288 | P291 | R292 | D293 | P294 | L296 |
| 105 | K228 | D249 | R250 | N253 | P254 | P257 | R258 | D259 | P260 | L262 |
| 106 | A196 | T217 | A218 | S222 | T223 | G226 | S227 | A228 | V229 | G231 |
| 107 | R236 | E257 | R258 | T261 | P262 | P265 | R266 | D267 | P268 | L270 |
| 108 | K257 | E278 | R279 | N282 | P283 | P286 | R287 | D288 | Q289 | L291 |
| 109 | K227 | D248 | K249 | N252 | P253 | P256 | R257 | D258 | P259 | L261 |
| 110 | G241 | T262 | A263 | S267 | A268 | G271 | G272 | A273 | S274 | G276 |
| 111 | K228 | D249 | K250 | N253 | P254 | P257 | R258 | D259 | P260 | L262 |
| 112 | D189 | S210 | P211 | K215 | K216 | P219 | P220 | R221 | T222 | 1224 |
| 113 | K243 | D264 | K265 | N268 | P269 | P272 | R273 | D274 | P275 | L277 |
| 114 | R236 | E257 | R258 | T261 | P262 | P265 | R266 | D267 | P268 | L270 |
| 115 | G194 | S215 | A216 | T219 | N220 | E223 | T224 | K225 | N226 | V228 |
| 116 | K257 | E278 | R279 | N282 | P283 | P286 | R287 | D288 | Q289 | L291 |
| 117 | D196 | A217 | A218 | K222 | N223 | T226 | K227 | S228 | S229 | G231 |
| 129 | G236 | T257 | A258 | G260 | D261 | G264 | S265 | A266 | V267 | G269 |
| SEQ ID | Pos 41 | Pos 42 | Pos 43 | Pos 44 | Pos 45 | Pos 46 | Pos 47 | Pos 48 | Pos 49 | Pos 50 |
| 1 | K259 | P260 | V262 | G264 | D286 | Q291 | P305 | G308 | N309 | Q323 |
| 2 | K259 | P260 | V262 | G264 | D286 | Q291 | P305 | G308 | N309 | Q323 |
| 3 | K258 | P259 | V261 | G263 | D285 | Q290 | P304 | G307 | N308 | Q322 |
| 4 | K258 | P259 | V261 | G263 | D285 | Q290 | P304 | G307 | N308 | Q322 |
| 5 | K260 | P261 | V263 | G265 | D287 | Q292 | P306 | G309 | N310 | Q324 |
| 6 | K230 | P231 | V233 | G235 | D257 | Q262 | P276 | G279 | N280 | Q294 |
| 7 | K259 | P260 | V262 | G264 | D286 | Q291 | L305 | E308 | N309 | Q323 |
| 8 | K233 | K234 | Q236 | G238 | D260 | K265 | S279 | E282 | N283 | K297 |
| 9 | K232 | K233 | Q235 | G237 | D259 | E264 | S278 | E281 | N282 | Q296 |
| 10 | R230 | K231 | L233 | G235 | D257 | K262 | S276 | E279 | N280 | Q294 |
| 11 | N231 | K232 | K234 | G236 | D258 | N263 | S277 | Q280 | N281 | — |
| 12 | G226 | K227 | Q229 | G231 | D253 | N258 | S272 | E275 | N276 | Q290 |
| 13 | N231 | K232 | Q234 | G236 | D258 | N263 | S277 | Q280 | N281 | — |
| 14 | G228 | K229 | Q231 | G233 | H255 | N260 | S274 | E277 | N278 | Q292 |
| 15 | K231 | H232 | Q234 | G236 | D258 | K263 | S277 | E280 | N281 | Q294 |
| 16 | K231 | H232 | Q234 | G236 | D258 | K263 | S277 | E280 | N281 | Q294 |
| 17 | S231 | K232 | Q234 | G236 | D258 | N263 | S277 | E280 | N281 | — |
| 18 | S231 | K232 | Q234 | G236 | G258 | R263 | Y276 | Q279 | N280 | L294 |
| 19 | S226 | K227 | R229 | G231 | G253 | R258 | Y271 | Q274 | N275 | L289 |
| 20 | K229 | K230 | Q232 | G234 | D256 | N261 | S275 | D278 | S279 | E293 |
| 21 | K226 | K227 | Q229 | G231 | D253 | N258 | S272 | D275 | S276 | E290 |
| 22 | K233 | K234 | S236 | G238 | D260 | D265 | Q279 | E280 | N281 | Q295 |
| 23 | K237 | R238 | S240 | R242 | D264 | D269 | Q283 | E284 | N285 | Q299 |
| 24 | K230 | K231 | S233 | G235 | D257 | D262 | Q276 | E277 | N278 | Q292 |
| 25 | K276 | K277 | S279 | G281 | D303 | D308 | Q322 | E323 | N324 | Q338 |
| 26 | S198 | K199 | R201 | G203 | H225 | Q230 | A244 | G246 | N247 | Q261 |
| 27 | R264 | R265 | R267 | A269 | D291 | G296 | P310 | G313 | G314 | D328 |
| 28 | R266 | R267 | R269 | V271 | D293 | G298 | P312 | G315 | G316 | D330 |
| 29 | K203 | K204 | S206 | G208 | D230 | D235 | Q249 | E250 | N251 | Q265 |
| 30 | R266 | R267 | R269 | V271 | D293 | G298 | P312 | G315 | R316 | D330 |
| 31 | R266 | R267 | R269 | V271 | D293 | G298 | P312 | G315 | R316 | D330 |
| 32 | K238 | R239 | P241 | G243 | E265 | N270 | P284 | D287 | S288 | S307 |
| 33 | T276 | P277 | G279 | T281 | D302 | S307 | — | G319 | G320 | V334 |
| 34 | K233 | K234 | Q236 | G238 | D260 | E265 | S279 | E282 | N283 | S299 |
| 35 | — | — | — | — | D322 | D327 | Q341 | E342 | N343 | L359 |
| 36 | G270 | R271 | K273 | A275 | G297 | G302 | S316 | D319 | G320 | Q339 |
| 37 | G230 | R231 | K233 | V235 | G257 | G262 | S276 | G279 | G280 | Q299 |
| 38 | Q236 | K237 | Q239 | G241 | E263 | H268 | P282 | N285 | N286 | Q305 |
| 39 | K229 | K230 | Q232 | G234 | D256 | N261 | S275 | D278 | S279 | E295 |
| 40 | G241 | R242 | K244 | V246 | G268 | G273 | S287 | G290 | G291 | Q310 |
| 41 | G241 | R242 | K244 | V246 | G268 | G273 | S287 | G290 | G291 | Q310 |
| 42 | K230 | K231 | S233 | G235 | D257 | D262 | Q276 | E277 | N278 | L294 |
| 43 | R234 | P235 | R237 | G239 | E261 | N266 | P280 | Q283 | S284 | K302 |
| 44 | R234 | P235 | R237 | G239 | E261 | N266 | P280 | Q283 | S284 | K302 |
| 45 | — | — | — | — | — | — | — | — | — | — |
| 46 | — | — | — | — | — | — | — | — | — | — |
| 47 | R118 | R119 | R121 | V123 | D145 | G150 | P164 | G167 | R168 | Q187 |
| 48 | G147 | R148 | K150 | A152 | S174 | G179 | S193 | D196 | G197 | A216 |
| 49 | E219 | R220 | K222 | K224 | N246 | R251 | S265 | D268 | S269 | T286 |
| 50 | P18 | K19 | Q21 | G23 | E45 | H50 | D64 | S67 | N68 | Q87 |
| 51 | — | — | R205 | S207 | H229 | N234 | — | N246 | P247 | R261 |
| 52 | P203 | D204 | P206 | G208 | D229 | N234 | — | T245 | A246 | R261 |
| 53 | P203 | D204 | P206 | G208 | D229 | N234 | — | T245 | A246 | R261 |
| 54 | — | — | — | — | — | — | — | — | — | — |
| 55 | — | — | R205 | S207 | H229 | N234 | — | N246 | P247 | R261 |
| 56 | A203 | K204 | R206 | K208 | E229 | N234 | P242 | K245 | G246 | C260 |
| 57 | — | — | R221 | G223 | G245 | G250 | V257 | D260 | K261 | Q275 |
| 58 | S209 | K210 | K212 | K214 | D235 | S240 | — | E251 | K252 | E262 |
| 59 | S206 | K207 | S209 | R211 | D232 | S237 | — | E248 | G249 | 1263 |
| 60 | — | F205 | K207 | V209 | D230 | G235 | — | D246 | G247 | E261 |
| 61 | — | — | — | R211 | A233 | E237 | — | G249 | T250 | — |
| 62 | — | — | — | — | — | — | — | — | — | — |
| 63 | — | — | — | K211 | N233 | S237 | — | K249 | Q250 | I264 |
| 64 | — | — | K218 | K220 | S242 | D250 | — | G262 | G263 | T276 |
| 65 | — | K224 | L226 | A228 | P252 | H260 | — | G272 | A273 | G280 |
| 66 | — | — | K211 | R213 | G235 | E240 | — | K253 | R254 | G269 |
| 67 | — | — | — | R209 | T231 | A235 | — | A246 | A247 | L261 |
| 68 | — | — | — | — | P232 | G236 | — | D247 | M248 | K258 |
| 69 | P213 | K214 | R216 | V218 | Q240 | N245 | — | T256 | G257 | I271 |
| 70 | R210 | P211 | R213 | R215 | G237 | G242 | A253 | G256 | A257 | G272 |
| 71 | — | — | — | — | — | — | — | — | — | — |
| 72 | R229 | P230 | R232 | R234 | G256 | G261 | A272 | G275 | A276 | G291 |
| 73 | — | — | — | — | G235 | E240 | — | S251 | W252 | I267 |
| 74 | — | — | — | — | D236 | N240 | — | E251 | G252 | M266 |
| 75 | — | — | — | — | D236 | N240 | — | E251 | G252 | M266 |
| 76 | — | — | — | R209 | P231 | G235 | — | V247 | Q248 | E263 |
| 77 | — | — | — | — | G236 | G241 | — | G251 | T252 | I267 |
| 78 | — | — | G210 | V212 | S234 | E238 | — | K249 | G250 | E264 |
| 79 | — | — | — | V211 | E233 | G237 | — | G248 | K249 | E263 |
| 80 | K225 | T226 | P228 | E230 | R252 | N256 | — | Q268 | T269 | E284 |
| 81 | — | — | — | — | D236 | N240 | — | E251 | G252 | M266 |
| 82 | — | — | — | R210 | R232 | G236 | — | D247 | H248 | G262 |
| 83 | — | — | — | R209 | P231 | G235 | — | V247 | Q248 | E263 |
| 84 | — | P225 | R227 | Q229 | S251 | G255 | — | E266 | L267 | A282 |
| 85 | — | — | — | R201 | — | R226 | — | G238 | G239 | F252 |
| 86 | — | — | F197 | R199 | I219 | D224 | — | — | K236 | E250 |
| 87 | A212 | P213 | A216 | E218 | D239 | N244 | — | — | F257 | I270 |
| 88 | K206 | R207 | T209 | E211 | D233 | G238 | — | — | K250 | I260 |
| 89 | — | — | R196 | L198 | — | N222 | — | — | — | T244 |
| 90 | A218 | G219 | G221 | V223 | A245 | G250 | — | — | P263 | I273 |
| 91 | P227 | P228 | R230 | Q232 | S253 | G258 | R272 | — | G274 | V293 |
| 92 | T217 | V218 | R220 | E222 | E243 | N248 | — | — | T261 | V275 |
| 93 | G230 | A231 | Y233 | L235 | T257 | E263 | — | — | — | Q289 |
| 94 | A220 | G221 | G223 | V225 | D247 | G252 | — | — | — | L275 |
| 95 | K204 | T205 | — | — | D227 | N232 | — | — | W245 | W254 |
| 96 | K205 | R206 | T208 | A210 | E232 | N237 | — | — | W250 | I259 |
| 97 | A207 | G208 | G210 | V212 | E233 | E238 | — | — | K250 | E264 |
| 98 | K269 | P270 | G272 | T274 | D295 | S300 | — | — | E313 | V327 |
| 99 | K246 | K247 | S249 | G251 | E273 | D278 | E292 | — | N294 | L310 |
| 100 | K272 | P273 | G275 | T277 | S298 | S303 | — | — | G316 | L330 |
| 101 | R230 | K231 | L233 | G235 | D257 | K262 | S276 | E279 | N280 | S296 |
| 102 | T268 | P269 | G271 | T273 | S294 | S299 | — | — | G312 | L326 |
| 103 | S226 | K227 | R229 | G231 | H253 | Q258 | T272 | G274 | N275 | Q291 |
| 104 | K298 | P299 | G301 | T303 | S324 | S329 | — | — | E342 | V356 |
| 105 | T264 | P265 | G267 | T269 | S290 | S295 | — | — | G308 | V322 |
| 106 | G233 | R234 | K236 | V238 | G260 | A265 | S279 | N282 | G283 | Q302 |
| 107 | K272 | P273 | G275 | T277 | S298 | S303 | — | — | G316 | L330 |
| 108 | K293 | P294 | G296 | T298 | S319 | S324 | — | — | E337 | V351 |
| 109 | A263 | P264 | G266 | T268 | S289 | S294 | — | — | G307 | V321 |
| 110 | G278 | R279 | K281 | A283 | T305 | G310 | S324 | D327 | G328 | Q347 |
| 111 | A264 | P265 | G267 | T269 | S290 | S295 | — | — | G308 | V322 |
| 112 | K226 | K227 | Q229 | G231 | D253 | N258 | S272 | D275 | S276 | E292 |
| 113 | A279 | P280 | G282 | T284 | S305 | S310 | — | — | G323 | V337 |
| 114 | K272 | P273 | G275 | T277 | S298 | S303 | — | — | G316 | L330 |
| 115 | R230 | K231 | L233 | G235 | D257 | K262 | S276 | E279 | N280 | S296 |
| 116 | K293 | P294 | G296 | T298 | S319 | S324 | — | — | E337 | V351 |
| 117 | K233 | K234 | S236 | G238 | D260 | D265 | Q279 | — | N281 | L297 |
| 129 | G271 | R272 | K274 | A276 | G298 | G303 | S317 | D320 | G321 | H340 |

| SEQ ID | Pos 51 | Pos 52 | Pos 53 | Pos 54 | Pos 55 | Pos 56 | Pos 57 | Pos 58 | Pos 59 | Pos 60 | Pos 61 | Pos 62 | Pos 63 |
|---|---|---|---|---|---|---|---|---|---|---|---|---|---|
| 1 | R335 | M343 | F345 | T358 | D372 | K373 | S387 | H391 | N392 | L400 | S412 | M414 | C415 |
| 2 | R335 | M343 | F345 | T358 | D372 | K373 | S387 | H391 | N392 | L400 | S412 | M414 | C415 |
| 3 | R334 | M342 | F344 | T357 | D371 | K372 | S386 | H390 | N391 | L399 | S411 | M413 | C414 |
| 4 | R334 | M342 | F344 | T357 | D371 | K372 | S386 | H390 | N391 | L399 | S411 | M413 | C414 |
| 5 | R336 | M344 | F346 | T359 | D373 | K374 | S388 | H392 | N393 | L401 | S413 | M415 | C416 |
| 6 | R306 | M314 | F316 | S329 | D343 | K344 | S358 | H362 | N363 | L371 | S383 | M385 | C386 |
| 7 | N335 | M343 | V345 | S358 | T372 | N373 | S387 | H391 | N392 | L400 | S412 | V414 | Y415 |
| 8 | C311 | M319 | K321 | S334 | E348 | N349 | S363 | Q367 | N368 | L376 | N388 | L390 | Y391 |
| 9 | C310 | T318 | N320 | S333 | D347 | N348 | S362 | Q366 | N367 | L375 | N387 | L389 | Y390 |
| 10 | C308 | M316 | G318 | S331 | E345 | N346 | S360 | Q364 | N365 | L373 | N385 | L387 | Y388 |
| 11 | Y305 | T313 | R315 | S328 | E342 | N343 | S357 | K361 | N362 | L370 | S382 | L384 | Y385 |
| 12 | C304 | T312 | R314 | V327 | E341 | N342 | S356 | K360 | N361 | L369 | S381 | L383 | Y384 |
| 13 | G305 | K313 | K315 | T328 | E342 | N343 | S357 | Q361 | N362 | L370 | S382 | M384 | H385 |
| 14 | C306 | T314 | R316 | N329 | E343 | N344 | S358 | E362 | N363 | L371 | S383 | Q385 | Y386 |
| 15 | C308 | T316 | G318 | 1331 | E345 | N346 | S360 | Q364 | N365 | L373 | K385 | L387 | F388 |
| 16 | C308 | T316 | G318 | 1331 | E345 | N346 | S360 | Q364 | N365 | L373 | K385 | L387 | F388 |
| 17 | G305 | T313 | R315 | T328 | E342 | N343 | S357 | Q361 | N362 | L370 | S382 | L384 | H385 |
| 18 | C306 | M314 | G316 | A329 | E343 | S344 | S358 | Q362 | N363 | L371 | N383 | E385 | Y386 |
| 19 | C301 | M309 | G311 | A324 | E338 | S339 | S353 | Q357 | N358 | L366 | N378 | E380 | Y381 |
| 20 | C307 | A315 | R317 | D330 | E344 | N345 | S359 | Q363 | H364 | L372 | N384 | V386 | Y387 |
| 21 | C304 | A312 | R314 | D327 | E341 | S342 | S356 | K360 | H361 | L369 | N381 | V383 | Y384 |
| 22 | C309 | M317 | G319 | N332 | E346 | K347 | S361 | K365 | H366 | L374 | S386 | V388 | H389 |
| 23 | C313 | T321 | G323 | N336 | E350 | K351 | S365 | K369 | H370 | L378 | S390 | L392 | H393 |
| 24 | C302 | M310 | G312 | N325 | E339 | K340 | S354 | K358 | H359 | L367 | S367 | V381 | H382 |
| 25 | C348 | M356 | G358 | N371 | E385 | K386 | S400 | K404 | H405 | L413 | S425 | V427 | H428 |
| 26 | N274 | T282 | T284 | S297 | E311 | N312 | A326 | E330 | N331 | L339 | E351 | V353 | Y354 |
| 27 | S345 | R353 | G355 | N368 | E382 | D383 | Y397 | Q401 | H403 | L411 | D423 | Q425 | Y426 |
| 28 | S347 | T355 | G357 | D370 | E384 | D385 | Y399 | Q403 | H405 | L413 | D425 | Q427 | Y428 |
| 29 | C279 | M287 | G289 | N302 | E316 | K317 | S331 | K335 | H336 | L344 | S356 | V358 | H359 |
| 30 | S347 | T355 | G357 | D370 | D384 | D385 | Y399 | Q403 | H405 | L413 | D425 | Q427 | Y428 |
| 31 | S347 | T355 | G357 | D370 | D384 | D385 | Y399 | Q403 | H405 | L413 | D425 | Q427 | Y428 |
| 32 | G319 | T327 | K329 | T342 | E356 | N357 | S371 | E375 | G376 | L384 | S396 | Q398 | Y399 |
| 33 | Y346 | P354 | S356 | Y367 | D381 | S382 | R402 | — | — | I412 | P424 | R426 | V427 |
| 34 | C311 | N319 | G321 | S334 | E348 | D349 | S363 | Q367 | N368 | L376 | K388 | S390 | Y391 |
| 35 | C371 | M379 | G381 | K394 | E408 | K409 | S423 | K427 | H428 | L436 | S448 | L450 | H451 |
| 36 | S351 | T359 | G361 | D374 | E388 | D389 | Y403 | K408 | Y409 | L417 | N429 | Q431 | H432 |
| 37 | S311 | T319 | G321 | D334 | E348 | D349 | Y363 | K368 | H369 | L377 | N389 | Q391 | Y392 |
| 38 | — | — | — | T315 | Q329 | D330 | S344 | K348 | N349 | L357 | T369 | Q371 | Y372 |
| 39 | C307 | A315 | R317 | D330 | E344 | S345 | S359 | K363 | H364 | L372 | N384 | V386 | Y387 |
| 40 | S322 | T330 | G332 | D345 | E359 | D360 | Y374 | K379 | H380 | L388 | N400 | Q402 | Y403 |
| 41 | S322 | T330 | G332 | D345 | E359 | D360 | Y374 | K379 | H380 | L388 | N400 | Q402 | Y403 |
| 42 | C306 | V314 | G316 | K329 | E343 | K344 | S358 | K362 | H363 | L371 | S383 | L385 | H386 |
| 43 | D314 | V322 | D324 | I337 | D351 | S352 | S366 | A371 | N372 | L380 | E392 | Q394 | L395 |
| 44 | D314 | V322 | D324 | I337 | D351 | S352 | S366 | A371 | N372 | L380 | E392 | Q394 | L395 |
| 45 | — | — | — | — | — | — | — | — | — | — | — | — | — |
| 46 | — | — | — | — | — | — | — | — | — | — | — | — | — |
| 47 | S199 | T207 | G209 | D222 | D236 | D237 | Y251 | K256 | H257 | L265 | D277 | Q279 | Y280 |
| 48 | S228 | T236 | G238 | D251 | E265 | D266 | F280 | K285 | H286 | L294 | N306 | Q308 | Y309 |
| 49 | S298 | — | — | — | — | — | — | — | — | — | — | — | — |
| 50 | R99 | S107 | C109 | M122 | E136 | N137 | S151 | G155 | S156 | L164 | K176 | Q178 | Y179 |
| 51 | H273 | — | — | E293 | E307 | Q308 | A322 | — | — | L333 | E345 | H347 | V348 |
| 52 | H273 | — | — | T293 | D307 | A308 | P322 | — | — | I333 | E345 | H347 | V348 |
| 53 | H273 | — | — | T293 | D307 | A308 | P322 | — | — | I333 | E345 | H347 | V348 |
| 54 | S6 | T14 | G16 | D29 | D43 | D44 | Y58 | K63 | H64 | L72 | D84 | Q86 | Y87 |
| 55 | H273 | — | — | E293 | E307 | Q308 | A322 | — | — | L333 | E345 | H347 | V348 |
| 56 | Y272 | S280 | L282 | L293 | D307 | D308 | A322 | — | — | I332 | Q344 | K346 | V347 |
| 57 | P287 | G295 | L297 | E309 | E323 | Q324 | A338 | — | — | L348 | L360 | H362 | V363 |
| 58 | H274 | — | — | I296 | D310 | Q311 | K325 | — | — | I335 | E347 | Y349 | V350 |
| 59 | Y275 | S282 | F284 | Y296 | K310 | N311 | E325 | — | — | L335 | N347 | F349 | A350 |
| 60 | P273 | — | — | Q295 | Q309 | D310 | K324 | — | — | L334 | D346 | Y348 | I349 |
| 61 | H272 | — | — | P292 | A306 | D307 | S321 | Q325 | — | — | A344 | H346 | V347 |
| 62 | — | — | — | — | — | — | — | — | — | — | — | — | — |
| 63 | Y276 | F282 | E284 | Y297 | K311 | D312 | S326 | K330 | — | I336 | E348 | M350 | A351 |
| 64 | H286 | L292 | E294 | E307 | S321 | E322 | S336 | K340 | — | L346 | A358 | K360 | V361 |
| 65 | H292 | M301 | A303 | K316 | S330 | Q331 | T345 | R349 | — | N355 | P368 | K370 | V371 |
| 66 | H281 | L288 | E290 | D303 | E317 | Q318 | F332 | S336 | — | L342 | E354 | H356 | V357 |
| 67 | Y273 | L279 | R281 | V294 | T308 | A309 | E323 | R327 | — | I333 | Q345 | F347 | V348 |
| 68 | H270 | — | — | D287 | S301 | A302 | E316 | R320 | — | L326 | E338 | H340 | G341 |
| 69 | Y283 | I289 | E291 | R304 | N318 | Q319 | S333 | R337 | — | L343 | A355 | R357 | V358 |
| 70 | E294 | I301 | A303 | E316 | E330 | Q331 | S345 | R349 | — | L355 | A367 | H369 | I370 |
| 71 | — | — | — | T211 | R225 | M226 | — | — | — | M233 | — | — | — |
| 72 | E310 | I317 | A319 | E332 | E346 | Q347 | S361 | R365 | — | L371 | A383 | H385 | I386 |
| 73 | F278 | I284 | S286 | P299 | D313 | Q314 | S328 | R332 | — | L338 | H350 | H352 | V353 |
| 74 | P278 | L284 | G286 | P299 | E313 | Q314 | T328 | R332 | — | I338 | E350 | K352 | V353 |
| 75 | P278 | L284 | G286 | P299 | E313 | Q314 | T328 | R332 | — | I338 | E350 | K352 | V353 |
| 76 | H375 | V283 | I285 | Y298 | R312 | Q313 | E327 | R331 | — | L337 | E349 | S351 | V352 |
| 77 | Y279 | V285 | A287 | P300 | D314 | Q315 | S329 | R333 | — | L339 | P351 | R353 | V354 |
| 78 | Y276 | L282 | D284 | E297 | K311 | G312 | R326 | R330 | — | L336 | S348 | K350 | A351 |
| 79 | Y275 | V281 | N283 | E296 | K310 | G311 | R325 | R329 | — | L335 | E347 | K349 | A350 |
| 80 | G295 | L301 | P303 | P316 | S330 | A331 | R345 | I349 | — | I355 | E367 | W369 | Q370 |
| 81 | P278 | L284 | G286 | P299 | E313 | Q314 | T328 | R332 | — | I338 | D350 | K352 | V353 |
| 82 | H274 | W281 | E283 | E296 | E310 | Q311 | S325 | P329 | — | L335 | D347 | H349 | Q350 |
| 83 | H275 | V283 | I285 | Y298 | R312 | Q313 | E327 | R331 | — | L337 | E349 | S351 | V352 |
| 84 | Y293 | L299 | E301 | L314 | E328 | A329 | R343 | L347 | — | I353 | Q365 | Y367 | H368 |
| 85 | F264 | L270 | P272 | H285 | E299 | D300 | K314 | P318 | — | I324 | D336 | Q338 | V339 |
| 86 | H262 | I268 | F270 | N283 | A297 | E298 | W312 | — | — | I322 | S334 | E336 | V337 |
| 87 | A282 | I288 | P290 | P303 | D317 | R318 | S332 | — | — | L342 | D354 | H356 | V357 |
| 88 | Y272 | L278 | F280 | Y293 | T307 | G308 | S321 | — | — | L331 | E343 | G345 | A346 |
| 89 | Y256 | I262 | D264 | N277 | K291 | D292 | K306 | — | — | I316 | K328 | Q330 | F331 |
| 90 | H285 | V291 | G293 | P306 | E320 | R321 | K335 | — | — | L345 | E357 | K359 | V360 |
| 91 | Y305 | L311 | D313 | P326 | E340 | A341 | R355 | — | — | I365 | Q377 | Y379 | H380 |
| 92 | Y287 | L293 | N295 | P308 | D322 | A323 | R337 | — | — | I347 | Q359 | W361 | Q362 |
| 93 | P301 | L307 | A309 | E322 | E336 | N337 | A351 | — | — | A361 | E373 | Q375 | V376 |
| 94 | Y287 | L293 | G295 | P308 | E322 | K323 | K337 | — | — | L347 | E359 | K361 | V362 |
| 95 | G266 | L272 | P274 | P287 | M301 | Q302 | R316 | — | — | L326 | A338 | Q340 | V341 |
| 96 | Y271 | L277 | F279 | Y292 | T306 | G307 | S320 | — | — | L330 | K342 | G344 | A345 |
| 97 | Y276 | L282 | E284 | E297 | R311 | G312 | K326 | — | — | L336 | E348 | K350 | A351 |
| 98 | H339 | L345 | P347 | Y360 | E374 | A375 | R395 | — | — | I405 | P417 | R419 | V420 |
| 99 | C322 | M330 | G332 | K345 | E359 | K360 | S374 | K378 | H379 | L387 | S399 | L401 | H402 |
| 100 | H342 | L348 | P350 | Y363 | E377 | A378 | R398 | — | — | I408 | S420 | R422 | V423 |
| 101 | C308 | T316 | G318 | S331 | E345 | N346 | S360 | Q364 | N365 | L373 | N385 | L387 | Y388 |
| 102 | Y338 | L344 | P346 | Y359 | E373 | A374 | R394 | — | — | I404 | P416 | R418 | V419 |
| 103 | G303 | T311 | T313 | S326 | E340 | N341 | A355 | E359 | N360 | L368 | E380 | L382 | Y383 |
| 104 | Y368 | L374 | P376 | H389 | E403 | A404 | R424 | — | — | I434 | P446 | R448 | T449 |
| 105 | Y334 | L340 | P342 | Y355 | E369 | A370 | R390 | — | — | I400 | A412 | R414 | V415 |
| 106 | S314 | T322 | G324 | D337 | E351 | D352 | Y366 | K371 | H372 | L380 | N392 | Q394 | H395 |
| 107 | H342 | L348 | P350 | Y363 | E377 | A378 | R398 | — | — | I408 | S420 | R422 | V423 |
| 108 | Y363 | L369 | P371 | Y384 | E398 | A399 | R419 | — | — | I429 | P441 | R443 | I444 |
| 109 | Y333 | L339 | P341 | Y354 | E368 | A369 | R389 | — | — | I399 | A411 | R413 | V414 |
| 110 | S359 | T367 | G369 | D382 | E396 | D397 | F411 | K416 | H417 | L425 | N437 | Q439 | H440 |
| 111 | Y334 | L340 | P342 | Y355 | E369 | A370 | R390 | — | — | I400 | A412 | R414 | V415 |
| 112 | C304 | A312 | R314 | D327 | E341 | S342 | S356 | K360 | H361 | L369 | N381 | V383 | Y384 |
| 113 | Y349 | L355 | P357 | Y370 | E384 | A385 | R405 | — | — | I415 | A427 | R429 | V430 |
| 114 | H342 | L348 | P350 | Y363 | E377 | A378 | R398 | — | — | I408 | S420 | R422 | V423 |
| 115 | C308 | T316 | G318 | S331 | E345 | N346 | S360 | Q364 | N365 | L373 | N385 | L387 | Y388 |
| 116 | Y363 | L369 | P371 | Y384 | E398 | A399 | R419 | — | — | I429 | P441 | R443 | I444 |
| 117 | C309 | M317 | G319 | K332 | E346 | K347 | T361 | K365 | H366 | L374 | S386 | L388 | H389 |
| 129 | S352 | T360 | G362 | D375 | E389 | D390 | Y404 | Q408 | H410 | L418 | N430 | Q432 | H433 |
| SEQ ID | Pos 64 | Pos 65 | Pos 66 | Pos 67 | Pos 68 | Pos 69 | Pos 70 | Pos 71 | Pos 72 | Pos 73 | Pos 74 | Pos 75 | Pos 76 |
| 1 | R425 | K428 | N431 | S433 | T434 | D435 | E436 | Q447 | T451 | D453 | S464 | A466 | D482 |
| 2 | R425 | K428 | N431 | S433 | T434 | D435 | E436 | Q447 | T451 | D453 | S464 | A466 | D482 |
| 3 | R424 | K427 | N430 | S432 | T433 | D434 | E435 | Q446 | T450 | D452 | S463 | A465 | D481 |
| 4 | R424 | K427 | N430 | S432 | T433 | D434 | E435 | Q446 | T450 | D452 | S463 | A465 | D481 |
| 5 | R426 | K429 | K432 | S434 | T425 | D436 | E437 | Q448 | R452 | D454 | S465 | A467 | D483 |
| 6 | R396 | K399 | N402 | S404 | T405 | D406 | E407 | Q418 | T422 | D424 | S435 | A437 | D453 |
| 7 | R425 | E428 | K431 | S433 | T434 | D435 | E426 | Q447 | T451 | G453 | S363 | A466 | E482 |
| 8 | R401 | E404 | K407 | S409 | T410 | D411 | E412 | Q423 | A427 | G429 | S440 | A442 | D458 |
| 9 | R400 | E403 | K406 | S408 | T409 | D410 | D411 | Q422 | A426 | G428 | S439 | A441 | D457 |
| 10 | R398 | E401 | K404 | S406 | T407 | D408 | E409 | Q420 | V424 | G426 | S437 | A439 | E455 |
| 11 | Q395 | E398 | Q401 | S403 | T404 | D405 | E406 | K417 | A421 | G423 | S434 | G436 | D452 |
| 12 | R394 | E397 | K400 | S402 | T403 | D404 | E405 | H416 | A420 | G422 | S433 | A435 | E451 |
| 13 | R395 | E398 | Q401 | S403 | T404 | D405 | E406 | K417 | A421 | G423 | S434 | G436 | D452 |
| 14 | R396 | E399 | K402 | S404 | K405 | D406 | E407 | Q418 | A422 | G424 | S435 | A437 | E453 |
| 15 | R398 | E401 | K404 | S406 | T407 | D408 | E409 | Q420 | V424 | G426 | S437 | A439 | E455 |
| 16 | R398 | E401 | K404 | S406 | T407 | D408 | E409 | Q420 | V424 | G426 | S437 | A439 | E455 |
| 17 | R395 | E398 | Q401 | S403 | T404 | D405 | E406 | K417 | A421 | G423 | S434 | G436 | D452 |
| 18 | R396 | E399 | K402 | S404 | T405 | D406 | E407 | Q418 | V422 | G424 | S435 | A437 | E453 |
| 19 | R391 | E394 | K397 | S399 | T400 | D401 | E402 | Q413 | V417 | G419 | S430 | A432 | E448 |
| 20 | R397 | E400 | K403 | S405 | R406 | T407 | E408 | Q419 | A423 | G425 | S436 | A438 | D454 |
| 21 | R394 | E397 | K400 | S402 | R403 | T404 | E405 | Q416 | A420 | G422 | S433 | A435 | D451 |
| 22 | R399 | E402 | K405 | S407 | T408 | D409 | E410 | R421 | V425 | G427 | R438 | A440 | D456 |
| 23 | R403 | E406 | K409 | S411 | T412 | D413 | E414 | R425 | V429 | G431 | R442 | A444 | D460 |
| 24 | R392 | E395 | K398 | S400 | T401 | D402 | E403 | R414 | V418 | G420 | R431 | A433 | D449 |
| 25 | R438 | E441 | K444 | S446 | T447 | D448 | E449 | R460 | V464 | G466 | R477 | A479 | D495 |
| 26 | R364 | E367 | K370 | S372 | R373 | D374 | E375 | Q386 | T390 | G392 | S403 | A405 | E421 |
| 27 | H436 | D439 | G442 | P444 | T445 | S446 | I447 | K458 | V462 | G464 | R475 | A477 | E493 |
| 28 | H438 | D441 | G444 | P446 | T447 | S448 | I449 | K460 | V464 | G466 | G477 | A479 | E495 |
| 29 | R369 | E372 | K375 | S377 | T378 | D379 | E380 | R391 | V395 | G397 | R408 | A410 | D426 |
| 30 | H438 | D441 | G444 | P446 | T447 | S448 | I449 | K460 | V464 | G466 | G477 | A479 | E495 |
| 31 | H438 | D441 | G444 | P446 | T447 | S448 | I449 | K460 | V464 | G466 | G477 | A479 | E495 |
| 32 | R409 | D412 | G415 | S417 | L418 | E419 | E420 | K431 | V435 | G437 | S448 | A450 | D466 |
| 33 | T437 | A440 | S443 | T445 | E446 | S447 | E448 | K459 | K463 | A467 | P478 | A480 | T496 |
| 34 | — | — | — | — | — | — | — | — | C404 | — | S410 | A412 | E428 |
| 35 | R461 | E464 | K467 | S469 | T470 | D471 | E472 | R483 | V487 | G489 | R500 | A502 | D518 |
| 36 | H442 | D445 | A448 | P450 | T451 | A452 | I453 | K464 | V468 | G470 | K481 | A483 | G499 |
| 37 | H402 | D405 | G408 | P410 | T411 | A412 | I413 | K424 | V428 | G430 | R441 | A443 | A459 |
| 38 | R382 | K385 | K388 | Q390 | L391 | K392 | D393 | K404 | V408 | S410 | N421 | A423 | D439 |
| 39 | R397 | E400 | K403 | — | — | — | — | — | — | — | — | — | — |
| 40 | H413 | D416 | G419 | P421 | T422 | A423 | I424 | K435 | V439 | G441 | R452 | A454 | A470 |
| 41 | H413 | D416 | G419 | P421 | T422 | A423 | I424 | K435 | V439 | G441 | R452 | A454 | A470 |
| 42 | R396 | E399 | K402 | S404 | T405 | — | — | — | — | — | — | — | — |
| 43 | R405 | L408 | S411 | S413 | K414 | E415 | E416 | R427 | V431 | G433 | E444 | A446 | Q462 |
| 44 | R405 | L408 | S411 | S413 | K414 | E415 | E416 | R427 | V431 | G433 | E444 | A446 | Q462 |
| 45 | — | — | — | — | — | — | — | — | — | — | — | — | — |
| 46 | — | — | — | — | — | — | — | — | — | — | — | — | — |
| 47 | H290 | D293 | G296 | P298 | T299 | S300 | I301 | K312 | V316 | G318 | G329 | A331 | E347 |
| 48 | H319 | D322 | G325 | P327 | T328 | A329 | I330 | K341 | V345 | G347 | R358 | A360 | G376 |
| 49 | — | — | — | — | — | — | — | — | — | — | — | — | — |
| 50 | R189 | N192 | S195 | S197 | L198 | D199 | E200 | K211 | V215 | G217 | G228 | A230 | E246 |
| 51 | R358 | E361 | L364 | P366 | E367 | D368 | R369 | R380 | I384 | G386 | E397 | S399 | H415 |
| 52 | R358 | H361 | T364 | D366 | A367 | A368 | A369 | S380 | V384 | A386 | P397 | A399 | D415 |
| 53 | R358 | H361 | T364 | D366 | A367 | A368 | A369 | S380 | V384 | A386 | P397 | A399 | D415 |
| 54 | H97 | D100 | G103 | P105 | T106 | S107 | I108 | K119 | V123 | G125 | G136 | A138 | E154 |
| 55 | R358 | E361 | L364 | P366 | E367 | D368 | R369 | R380 | I384 | G386 | E397 | S399 | H415 |
| 56 | R357 | D360 | L363 | D365 | D366 | E367 | E368 | D379 | L383 | G385 | P396 | A398 | D414 |
| 57 | R373 | Q276 | S379 | P381 | A382 | D383 | Q384 | Q395 | V399 | G401 | P412 | A414 | A430 |
| 58 | R360 | D363 | L366 | D368 | D369 | Q370 | E371 | A382 | V386 | G388 | K399 | A401 | N417 |
| 59 | R360 | D363 | K366 | D368 | R369 | N370 | K371 | Q382 | I386 | S388 | E399 | A401 | D417 |
| 60 | R359 | E362 | A365 | E367 | R368 | G369 | E370 | K381 | V385 | G387 | K398 | A400 | D416 |
| 61 | R357 | T360 | A363 | S365 | D366 | A367 | A368 | A379 | V385 | G385 | P396 | A398 | E414 |
| 62 | — | — | — | — | — | — | — | — | — | — | — | — | — |
| 63 | R361 | Q364 | E367 | E369 | K370 | S371 | D372 | Q383 | I387 | G389 | Q400 | A402 | E418 |
| 64 | K371 | E374 | Q377 | D379 | D380 | Q381 | E382 | P393 | I397 | G399 | Q410 | A412 | S428 |
| 65 | R381 | D384 | F387 | R389 | A390 | K391 | E392 | H403 | V407 | G409 | K420 | G422 | A438 |
| 66 | R367 | E370 | E373 | P375 | E376 | P377 | T378 | R389 | V393 | G395 | S406 | A408 | I424 |
| 67 | R358 | E361 | R364 | P366 | N367 | E368 | E369 | D380 | L384 | G386 | E397 | A399 | D415 |
| 68 | R351 | E354 | S357 | D359 | T360 | D361 | Q362 | T373 | I377 | D379 | P390 | A392 | T408 |
| 69 | I368 | I371 | E374 | S376 | D377 | D378 | E379 | A390 | A394 | G396 | P407 | A409 | E425 |
| 70 | R380 | E383 | R386 | E388 | L389 | P390 | S391 | E402 | V406 | G408 | P419 | A421 | E437 |
| 71 | — | — | — | — | — | — | — | — | — | — | — | — | — |
| 72 | Q396 | E399 | R402 | E404 | L405 | P406 | S407 | E418 | V422 | G424 | P435 | A437 | E453 |
| 73 | I363 | E366 | E369 | S371 | D372 | E373 | T374 | Q385 | I389 | G391 | R402 | A404 | V420 |
| 74 | T363 | K366 | Q369 | F371 | D372 | E373 | D374 | E385 | V389 | G391 | E402 | A404 | R420 |
| 75 | T363 | K366 | Q369 | F371 | D372 | E373 | D374 | E385 | V389 | G391 | E402 | A404 | R420 |
| 76 | R362 | E365 | D368 | P370 | Q371 | N372 | E373 | D384 | I388 | G390 | P401 | S403 | S419 |
| 77 | I364 | E367 | E370 | S372 | E373 | E374 | A375 | E386 | I390 | G392 | H403 | A405 | E421 |
| 78 | R361 | E364 | L367 | P369 | D370 | E371 | E372 | R383 | I387 | H389 | E400 | G402 | F418 |
| 79 | R360 | H363 | L366 | G368 | E369 | E370 | E371 | R382 | I386 | H388 | P399 | G401 | F417 |
| 80 | T380 | E383 | E386 | D388 | D389 | D390 | Q391 | Q402 | A406 | V409 | R420 | A422 | D438 |
| 81 | T363 | K366 | Q369 | F371 | D372 | E373 | D374 | E385 | V389 | G391 | E402 | A404 | R420 |
| 82 | R360 | E363 | A366 | P368 | Q369 | D370 | R371 | A382 | L386 | G388 | P399 | A401 | K417 |
| 83 | R362 | E365 | D368 | P370 | Q371 | N372 | E373 | D384 | I388 | G390 | P401 | S403 | S419 |
| 84 | T378 | P390 | A393 | S395 | P396 | E397 | E398 | Q409 | T413 | A416 | P427 | A429 | Q445 |
| 85 | Q349 | A352 | Q355 | S357 | E358 | T359 | A360 | R371 | I375 | A378 | D389 | A391 | D407 |
| 86 | R347 | E350 | Q353 | E355 | E356 | R357 | S358 | Q369 | I373 | A375 | Q387 | A389 | E405 |
| 87 | T367 | K370 | Q373 | F375 | D376 | E377 | D378 | S389 | V393 | G395 | A406 | A408 | E424 |
| 88 | R356 | E359 | D362 | T364 | D365 | E366 | E367 | T378 | K384 | T386 | S397 | A399 | D415 |
| 89 | Q341 | D344 | Q347 | T349 | D350 | E351 | E352 | Q363 | I367 | G369 | E380 | S382 | E398 |
| 90 | Q370 | E373 | K376 | S378 | D379 | A380 | E381 | A392 | I396 | A398 | P409 | A411 | Q427 |
| 91 | T390 | P402 | A405 | S407 | P408 | D409 | E410 | Q421 | T425 | P427 | P439 | A441 | Q457 |
| 92 | T372 | E375 | N378 | D380 | N381 | E382 | Q383 | R394 | K398 | D400 | K412 | A414 | N430 |
| 93 | M386 | E389 | E392 | S394 | D395 | D396 | E397 | D408 | V412 | G414 | N425 | S427 | E443 |
| 94 | C372 | E375 | R378 | S380 | D381 | A382 | E383 | T394 | I398 | E400 | E411 | A413 | E429 |
| 95 | Q351 | D353 | G356 | D358 | D359 | D360 | D361 | P372 | I376 | G378 | P389 | A391 | D407 |
| 96 | C355 | E358 | N361 | T363 | D364 | E365 | E366 | T377 | K383 | T385 | S396 | A398 | D414 |
| 97 | R361 | E364 | L367 | S369 | E370 | E371 | E372 | R383 | I387 | H389 | E400 | G402 | F418 |
| 98 | T430 | G433 | S436 | S438 | E439 | G440 | E441 | K452 | S458 | T460 | P471 | A473 | K489 |
| 99 | R412 | E415 | K418 | S420 | T421 | D422 | E423 | R434 | I438 | G440 | N451 | A453 | D469 |
| 100 | T433 | G436 | S439 | T441 | E442 | G443 | E444 | K455 | N461 | K463 | P474 | A476 | K492 |
| 101 | R398 | E401 | K404 | S406 | T407 | D408 | E409 | Q420 | V434 | G426 | S437 | A439 | E455 |
| 102 | T429 | G432 | S435 | T437 | E438 | S439 | Q440 | K451 | K457 | G459 | P470 | A472 | K488 |
| 103 | R393 | E396 | N399 | S401 | R402 | D403 | E404 | Q415 | A419 | G421 | S432 | A434 | E450 |
| 104 | T459 | G462 | D465 | T467 | Q468 | D469 | E470 | R481 | S487 | K489 | P500 | A502 | K518 |
| 105 | T425 | G428 | S431 | T433 | E434 | S435 | D436 | K447 | T453 | P455 | P466 | A468 | K484 |
| 106 | G405 | D408 | G411 | P413 | T414 | A415 | I416 | K427 | V431 | G433 | K444 | A446 | G462 |
| 107 | T433 | G436 | S439 | T441 | E442 | G443 | E444 | K455 | N461 | K463 | P474 | A476 | K492 |
| 108 | K454 | G457 | N460 | S462 | K463 | D464 | E465 | R476 | D482 | K484 | P495 | A497 | K513 |
| 109 | T424 | G427 | S430 | T432 | E433 | S434 | D435 | K446 | R452 | A454 | P465 | A467 | K483 |
| 110 | H450 | D453 | G456 | P458 | T459 | A460 | I461 | K472 | V476 | G478 | R489 | A491 | G507 |
| 111 | T425 | G428 | S431 | T433 | E434 | S435 | D436 | K447 | R453 | A455 | P466 | A468 | K484 |
| 112 | R394 | E397 | K400 | S402 | R403 | T404 | E405 | Q416 | A420 | G422 | S433 | A435 | D451 |
| 113 | T440 | G443 | S446 | T448 | E449 | S450 | D451 | K462 | R468 | A470 | P481 | A483 | K499 |
| 114 | T433 | G436 | S439 | T441 | E442 | G443 | E444 | K455 | N461 | K463 | P474 | A476 | K492 |
| 115 | R398 | K401 | K404 | S406 | T407 | D408 | E409 | Q420 | V424 | G426 | S437 | A439 | E455 |
| 116 | K454 | G457 | N460 | S462 | K463 | D464 | E465 | R476 | D482 | K484 | P495 | A497 | K513 |
| 117 | R399 | E402 | K405 | S407 | T408 | D409 | E410 | R421 | V425 | G427 | N438 | A440 | D456 |
| 129 | H443 | D446 | G449 | P451 | T452 | S453 | I454 | K465 | V469 | G471 | R482 | A484 | G500 |

In another embodiment, said differences at these amino acid positions (corresponding to the respective positions in SEQ ID NO: 2 or 4) include, but are not limited to, one or more of the following:

the amino acid corresponding to position P40 is deleted;

the amino acid corresponding to position K250 is deleted;

the amino acid corresponding to position Q391 is inserted

the amino acid corresponding to position Y520 is deleted;

the amino acid corresponding to position L521 is deleted;

the amino acid corresponding to position D522 is deleted;

the amino acid corresponding to position S523 is deleted;

the amino acid corresponding to position H524 is deleted;

the amino acid corresponding to position 1525 is deleted;

the amino acid corresponding to position Y526 is deleted;

the amino acid corresponding to position V527 is deleted;

the amino acid corresponding to position K528 is deleted;

the amino acid corresponding to position M529 is deleted;

the amino acid corresponding to position D530 is deleted;

the amino acid corresponding to position E531 is deleted;

the amino acid corresponding to position K532 is deleted;

the amino acid corresponding to position T533 is deleted;

the amino acid corresponding to position A534 is deleted;

Corresponding insertions and deletion positions in other species can be found in the following table

| SEQ ID | Pos 1 | Pos 2 | Pos 3 | Pos 4 | Pos 5 | Pos 6 | Pos 7 | Pos 8 | Pos 9 |
|---|---|---|---|---|---|---|---|---|---|
| 1 | P40 | K250 | H391 | Y520 | L521 | D522 | S523 | H524 | I525 |
| 2 | P40 | K250 | H391 | Y520 | L521 | D522 | S523 | H524 | I525 |
| 3 | P40 | K249 | H390 | Y519 | L520 | D521 | S522 | H523 | I524 |
| 4 | P40 | K249 | H390 | Y519 | L520 | D521 | S522 | H523 | I524 |
| 5 | P40 | K250 | H392 | Y521 | L522 | D523 | S524 | H525 | L526 |
| 7 | V36 | K249 | H391 | Y520 | L521 | E522 | S523 | — | — |
| 8 | S9 | K222 | Q367 | Y496 | L497 | N498 | S499 | S500 | S501 |
| 9 | P11 | K221 | Q366 | Y495 | L496 | E497 | S498 | S499 | S500 |
| 10 | Q10 | N220 | Q364 | Y493 | L494 | E495 | S496 | S497 | H498 |
| 11 | R11 | K220 | K361 | Y490 | L491 | N492 | S493 | A494 | S495 |
| 12 | — | K215 | K360 | Y489 | L490 | E491 | S492 | P493 | S494 |
| 13 | R11 | K220 | Q361 | Y490 | L491 | N492 | N493 | A494 | S495 |
| 14 | R11 | K217 | E362 | Y491 | L492 | E493 | S494 | S495 | S496 |
| 15 | R11 | K220 | Q364 | Y493 | L494 | E495 | N496 | S497 | S498 |
| 16 | G11 | K220 | Q364 | Y493 | L494 | E495 | N496 | S497 | S498 |
| 17 | R11 | K220 | Q361 | Y490 | L491 | N492 | N493 | A494 | S495 |
| 18 | R10 | S220 | Q362 | Y491 | L492 | E493 | S494 | S495 | T496 |
| 19 | Q5 | S215 | Q357 | Y486 | L487 | E488 | S489 | S490 | T491 |
| 20 | S11 | K218 | Q363 | Y492 | L493 | E494 | S495 | V496 | S497 |
| 21 | — | K215 | K360 | Y489 | L490 | E491 | A492 | V493 | S494 |
| 22 | E12 | K222 | K365 | Y494 | L495 | E496 | S497 | C498 | S499 |
| 23 | K12 | K226 | K369 | Y498 | L499 | E500 | S501 | C502 | S503 |
| 24 | E12 | K219 | K358 | Y487 | L488 | E489 | S490 | C491 | S492 |
| 25 | E58 | K265 | K404 | Y533 | L534 | E535 | S536 | C537 | S538 |
| 26 | — | K191 | E330 | Y459 | L460 | D461 | S462 | Y463 | S464 |
| 27 | R39 | P253 | Q401 | Y531 | L532 | E533 | S534 | Q535 | T536 |
| 28 | R41 | P255 | Q403 | Y533 | L534 | E535 | S536 | H537 | T538 |
| 29 | E12 | K192 | K335 | Y464 | L465 | E466 | S467 | C468 | S469 |
| 30 | R41 | P255 | Q403 | Y533 | L534 | E535 | S536 | H537 | T538 |
| 31 | R41 | P255 | Q403 | Y533 | L534 | E535 | S536 | H537 | T538 |
| 32 | L16 | K227 | E375 | Y504 | I505 | N506 | S507 | S508 | V509 |
| 33 | V49 | N265 | — | F536 | L537 | S538 | K539 | Y540 | A541 |
| 34 | G11 | K222 | Q367 | H466 | L467 | N468 | S469 | S470 | A471 |
| 35 | K74 | — | K427 | Y556 | L557 | E558 | S559 | C560 | S561 |
| 36 | R45 | S259 | Q407 | Y537 | L538 | E539 | S540 | H541 | Q542 |
| 37 | R8 | S219 | Q367 | Y497 | L498 | E499 | S500 | C501 | T502 |
| 39 | Q10 | Q220 | K363 | — | — | — | — | — | — |
| 40 | P7 | K232 | K379 | D505 | — | — | — | — | — |
| 41 | P7 | K232 | K379 | D505 | — | — | — | — | — |
| 42 | A15 | G221 | K362 | — | — | — | — | — | — |
| 43 | A9 | E225 | A371 | Y500 | L501 | Q502 | G503 | S504 | G505 |
| 44 | V9 | E225 | A371 | Y500 | L501 | Q502 | G503 | S504 | G505 |
| 45 | P40 | — | — | — | — | — | — | — | — |
| 46 | P40 | — | — | — | — | — | — | — | — |
| 47 | — | K109 | K256 | Y385 | L386 | E387 | S388 | H389 | T390 |
| 48 | — | K138 | K285 | Y405 | L406 | E407 | S408 | G409 | I410 |
| 49 | G1 | E210 | — | — | — | — | — | — | — |
| 50 | — | — | G155 | — | — | — | — | — | — |
| 51 | — | — | — | H453 | L454 | R455 | E456 | E457 | A458 |
| 52 | — | — | — | T453 | D454 | E455 | R456 | A457 | A458 |
| 53 | — | — | — | T453 | D454 | E455 | R456 | A457 | T458 |
| 54 | — | — | K63 | Y192 | L193 | E194 | S195 | H196 | T197 |
| 55 | — | — | — | H453 | L454 | R455 | E456 | E457 | A458 |
| 56 | — | — | — | E452 | K453 | — | — | — | — |
| 57 | G14 | — | — | — | — | — | — | — | — |
| 58 | G2 | — | — | Q455 | — | — | — | — | — |
| 59 | — | — | — | Q455 | F456 | T457 | M458 | H459 | N460 |
| 60 | — | — | — | R454 | I455 | S456 | D457 | — | — |
| 61 | — | — | — | H452 | L453 | A454 | G455 | A456 | D457 |
| 62 | Q10 | — | — | — | — | — | — | — | — |
| 63 | — | — | — | — | — | — | — | — | — |
| 64 | E9 | — | — | N466 | T467 | R468 | T469 | E470 | E471 |
| 65 | E3 | — | — | Y476 | L477 | K478 | T479 | L480 | — |
| 66 | — | — | — | S462 | L463 | — | — | — | — |
| 67 | — | — | — | D453 | R454 | A455 | R456 | S457 | — |
| 68 | — | — | — | — | — | — | — | — | — |
| 69 | — | G204 | — | Y463 | L464 | K465 | T466 | R467 | S468 |
| 70 | — | R201 | — | — | — | — | — | — | — |
| 71 | — | — | — | — | — | — | — | — | — |
| 72 | T16 | R220 | — | — | — | — | — | — | — |
| 73 | M1 | — | — | A458 | L459 | A460 | A461 | H462 | G463 |
| 74 | A2 | — | — | E456 | D457 | G458 | G459 | T460 | P461 |
| 75 | A2 | — | — | E456 | D457 | G458 | G459 | T460 | P461 |
| 76 | — | — | — | — | — | — | — | — | — |
| 77 | M2 | — | — | A459 | L460 | D461 | A462 | Q463 | G464 |
| 78 | — | — | — | G456 | — | — | — | — | — |
| 79 | — | — | — | E455 | — | — | — | — | — |
| 80 | M1 | V216 | — | H476 | L477 | H478 | D479 | C480 | Q481 |
| 81 | A2 | — | — | E456 | D457 | G458 | G459 | T460 | P461 |
| 82 | — | — | — | D455 | I456 | D457 | V458 | — | — |
| 83 | — | — | — | — | — | — | — | — | — |
| 84 | E7 | — | — | Q484 | V485 | — | — | — | — |
| 85 | — | — | — | K443 | — | — | — | — | — |
| 86 | — | — | — | R441 | A442 | A443 | S444 | R445 | S446 |
| 87 | A2 | — | — | Q460 | S461 | P462 | G463 | — | — |
| 88 | M1 | K201 | — | — | — | — | — | — | — |
| 89 | — | — | — | N434 | K435 | F436 | — | — | — |
| 90 | — | A209 | — | Y465 | L466 | K467 | G468 | R469 | — |
| 91 | — | D218 | — | V496 | R497 | R498 | — | — | — |
| 92 | — | K208 | — | Q468 | L469 | S470 | I471 | I472 | N473 |
| 93 | S4 | K221 | — | D481 | L482 | T483 | A484 | R485 | V486 |
| 94 | — | A211 | — | F467 | L468 | Q469 | S470 | R471 | — |
| 95 | — | — | — | Q445 | R446 | — | — | — | — |
| 96 | — | K200 | — | S452 | V453 | S454 | — | — | — |
| 97 | — | K198 | — | S456 | L457 | C458 | — | — | — |
| 98 | G49 | K260 | — | F529 | M530 | S531 | R532 | Y533 | A534 |
| 99 | P12 | G237 | K378 | Y507 | L508 | E509 | S510 | C511 | S512 |
| 100 | E55 | K263 | — | F532 | L533 | S534 | Q535 | Y536 | A537 |
| 101 | H10 | E222 | Q364 | Y493 | L494 | E495 | S496 | S497 | H498 |
| 102 | D51 | K259 | — | F528 | L529 | S530 | R531 | G532 | V533 |
| 103 | K14 | — | E359 | Y488 | L489 | D490 | S491 | Y492 | S493 |
| 104 | E81 | K289 | — | F558 | L559 | S560 | Q561 | Y562 | S563 |
| 105 | V44 | K255 | — | F524 | L525 | T526 | K527 | Y528 | A529 |
| 106 | A9 | K224 | K371 | Y500 | L501 | E502 | L503 | G504 | I505 |
| 107 | E55 | K263 | — | F532 | L533 | S534 | Q535 | Y536 | A537 |
| 108 | A76 | K284 | — | F553 | L554 | S555 | Q556 | Y557 | S558 |
| 109 | V46 | K254 | — | F523 | L524 | T525 | K526 | Y527 | A528 |
| 110 | P55 | K269 | K416 | Y545 | L546 | E547 | S548 | G549 | I550 |
| 111 | V47 | K255 | — | F524 | L525 | T526 | K527 | Y528 | A529 |
| 112 | E7 | Q217 | K360 | Y489 | L490 | E491 | A492 | V493 | S494 |
| 113 | V62 | K270 | — | F539 | L540 | T541 | K542 | Y543 | A544 |
| 114 | E55 | K263 | — | F532 | L533 | S534 | Q535 | Y536 | A537 |
| 115 | H10 | E222 | Q364 | Y493 | L494 | E495 | S496 | S497 | H498 |
| 116 | A76 | K284 | — | F553 | L554 | S555 | Q556 | Y557 | S558 |
| 117 | K11 | G224 | K365 | Y494 | L495 | E496 | S497 | C498 | S499 |
| 129 | P40 | K262 | Q408 | Y538 | L539 | E540 | S541 | G542 | I543 |
| SEQ ID | Pos 10 | Pos. 11 | Pos 12 | Pos 13 | Pos 14 | Pos 15 | Pos 16 | Pos 17 | Pos 18 |
| 1 | Y526 | V527 | K528 | M529 | D530 | E531 | K532 | T533 | A534 |
| 2 | Y526 | V527 | K528 | M529 | D530 | E531 | K532 | T533 | A534 |
| 3 | Y525 | V526 | K527 | M528 | D529 | E530 | K531 | T532 | A533 |
| 4 | Y525 | V526 | K527 | M528 | D529 | E530 | K531 | T532 | A533 |
| 5 | Y527 | V528 | K529 | M530 | N531 | E532 | K533 | T534 | A535 |
| 7 | — | N524 | K525 | M526 | T527 | E528 | E529 | T530 | I531 |
| 8 | D502 | G503 | K504 | M505 | F506 | K507 | E508 | — | — |
| 9 | D501 | D502 | K503 | M504 | L505 | K506 | E507 | G508 | P509 |
| 10 | Q499 | K500 | L501 | L502 | K503 | D504 | — | — | — |
| 11 | D496 | N497 | T498 | V499 | P500 | D501 | K502 | — | — |
| 12 | D495 | E496 | K497 | T498 | R499 | H500 | K501 | G502 | — |
| 13 | D496 | N497 | S498 | V499 | — | — | — | — | — |
| 14 | D497 | G498 | K499 | I500 | L501 | Q502 | Q503 | G504 | S505 |
| 15 | D499 | D500 | K501 | M502 | L503 | K504 | E505 | G506 | S507 |
| 16 | D499 | D500 | K501 | M502 | L503 | K504 | E505 | G506 | S507 |
| 17 | D496 | N497 | T498 | V499 | A500 | D501 | K502 | — | — |
| 18 | D497 | Q498 | S499 | C500 | A501 | E502 | — | — | — |
| 19 | D492 | Q493 | S494 | C495 | A496 | E497 | — | — | — |
| 20 | T498 | D499 | S500 | K501 | R502 | H503 | C504 | — | — |
| 21 | A495 | D496 | T497 | K498 | N499 | H500 | S501 | — | — |
| 22 | N500 | D501 | K502 | K503 | P504 | N505 | D506 | S507 | L508 |
| 23 | N504 | D505 | K506 | K507 | P508 | D509 | E510 | S511 | L512 |
| 24 | N493 | D494 | K495 | K496 | P497 | N498 | D499 | S500 | L501 |
| 25 | N539 | D540 | K541 | K542 | P543 | N544 | D545 | S546 | L547 |
| 26 | N465 | Q466 | K467 | — | — | — | — | — | — |
| 27 | K537 | H538 | N539 | N540 | S541 | H542 | — | — | — |
| 28 | K539 | H540 | N541 | N542 | L543 | H544 | — | — | — |
| 29 | N470 | D471 | K472 | K473 | P474 | N475 | D476 | S477 | — |
| 30 | K539 | H540 | N541 | N542 | S543 | H544 | — | — | — |
| 31 | K539 | H540 | N541 | N542 | S543 | H544 | — | — | — |
| 32 | — | — | — | — | — | — | — | — | — |
| 33 | R542 | S543 | F544 | — | — | — | — | — | — |
| 34 | D472 | D473 | K474 | M475 | L476 | K477 | K478 | G479 | S480 |
| 35 | N562 | D563 | K564 | K565 | P566 | E567 | D568 | S569 | — |
| 36 | A543 | R544 | — | — | — | — | — | — | — |
| 37 | D503 | Q504 | D505 | N506 | — | — | — | — | — |
| 39 | — | — | — | — | — | — | — | — | — |
| 40 | — | — | — | — | — | — | — | — | — |
| 41 | — | — | — | — | — | — | — | — | — |
| 42 | — | — | — | — | — | — | — | — | — |
| 43 | G506 | K507 | K508 | V509 | F510 | T511 | M512 | A513 | S514 |
| 44 | G506 | K507 | K508 | V509 | F510 | T511 | M512 | A513 | S514 |
| 45 | — | — | — | — | — | — | — | — | — |
| 46 | — | — | — | — | — | — | — | — | — |
| 47 | K391 | H392 | N393 | N394 | S395 | H396 | — | — | — |
| 48 | K411 | Q412 | V413 | S414 | — | — | — | — | — |
| 49 | — | — | — | — | — | — | — | — | — |
| 50 | — | — | — | — | — | — | — | — | — |
| 51 | A459 | G460 | G461 | L462 | A463 | K464 | L465 | V466 | L467 |
| 52 | Q459 | P460 | H461 | — | — | — | — | — | — |
| 53 | Q459 | P460 | H461 | — | — | — | — | — | — |
| 54 | K198 | H199 | N200 | N201 | S202 | H203 | — | — | — |
| 55 | A459 | G460 | G461 | L462 | A463 | K464 | L465 | V466 | L467 |
| 56 | — | — | — | — | — | — | — | — | — |
| 57 | — | — | — | — | — | — | — | — | — |
| 58 | — | — | — | — | — | — | — | — | — |
| 59 | V461 | Q462 | — | — | — | — | — | — | — |
| 60 | — | — | — | — | — | — | — | — | — |
| 61 | R458 | A459 | V460 | A461 | A462 | T463 | G464 | P465 | — |
| 62 | — | — | — | — | — | — | — | — | — |
| 63 | — | — | — | — | — | — | — | — | — |
| 64 | S472 | — | — | — | — | — | — | — | — |
| 65 | — | — | — | — | — | — | — | — | — |
| 66 | — | — | — | — | — | — | — | — | — |
| 67 | — | — | — | — | — | — | — | — | — |
| 68 | — | — | — | — | — | — | — | — | — |
| 69 | A469 | V470 | A471 | S472 | L473 | R474 | — | — | — |
| 70 | — | — | — | — | — | — | — | — | — |
| 71 | — | — | — | — | — | — | — | — | — |
| 72 | — | — | — | — | — | — | — | — | — |
| 73 | T464 | T465 | A466 | V467 | S468 | T469 | E470 | T471 | A472 |
| 74 | G462 | — | — | — | — | — | — | — | — |
| 75 | G462 | — | — | — | — | — | — | — | — |
| 76 | — | — | — | — | — | — | — | — | — |
| 77 | T465 | T466 | A467 | D468 | T469 | L470 | E471 | Q472 | A473 |
| 78 | — | — | — | — | — | — | — | — | — |
| 79 | — | — | — | — | — | — | — | — | — |
| 80 | T482 | A483 | N484 | — | — | — | — | — | — |
| 81 | G462 | — | — | — | — | — | — | — | — |
| 82 | — | — | — | — | — | — | — | — | — |
| 83 | — | — | — | — | — | — | — | — | — |
| 84 | — | — | — | — | — | — | — | — | — |
| 85 | — | — | — | — | — | — | — | — | — |
| 86 | I447 | A448 | — | — | — | — | — | — | — |
| 87 | — | — | — | — | — | — | — | — | — |
| 88 | — | — | — | — | — | — | — | — | — |
| 89 | — | — | — | — | — | — | — | — | — |
| 90 | — | — | — | — | — | — | — | — | — |
| 91 | — | — | — | — | — | — | — | — | — |
| 92 | D474 | N475 | — | — | — | — | — | — | — |
| 93 | — | — | — | — | — | — | — | — | — |
| 94 | — | — | — | — | — | — | — | — | — |
| 95 | — | — | — | — | — | — | — | — | — |
| 96 | — | — | — | — | — | — | — | — | — |
| 97 | — | — | — | — | — | — | — | — | — |
| 98 | Y535 | K536 | — | — | — | — | — | — | — |
| 99 | N513 | D514 | N515 | K516 | P517 | E518 | D519 | S520 | L521 |
| 100 | Y538 | K539 | — | — | — | — | — | — | — |
| 101 | D499 | K500 | L501 | L502 | K503 | — | — | — | — |
| 102 | Y534 | K535 | — | — | — | — | — | — | — |
| 103 | D494 | E495 | K496 | R497 | C498 | — | — | — | — |
| 104 | D564 | K565 | — | — | — | — | — | — | — |
| 105 | Y530 | K531 | — | — | — | — | — | — | — |
| 106 | K506 | R507 | D508 | N509 | — | — | — | — | — |
| 107 | Y538 | K539 | — | — | — | — | — | — | — |
| 108 | D559 | K560 | — | — | — | — | — | — | — |
| 109 | Y529 | K530 | — | — | — | — | — | — | — |
| 110 | K551 | H552 | V553 | N554 | — | — | — | — | — |
| 111 | Y530 | K531 | — | — | — | — | — | — | — |
| 112 | A495 | D496 | T497 | K498 | N499 | H500 | S501 | — | — |
| 113 | Y545 | K546 | — | — | — | — | — | — | — |
| 114 | Y538 | K539 | — | — | — | — | — | — | — |
| 115 | D499 | K500 | L501 | L502 | K503 | — | — | — | — |
| 116 | D559 | K560 | — | — | — | — | — | — | — |
| 117 | N500 | D501 | K502 | K503 | S504 | E505 | D506 | S507 | L508 |
| 129 | — | — | K544 | Q545 | D546 | N547 | — | — | — |

The inventors of the present invention have surprisingly found out that a substitution of amino acids at Gly210/211, Q119, S387, or S412, or at other non-active sites, in particular when combined with a substitution at positions at other non-active sites or when combined with mutations at active sites, i.e. corresponding to positions 128, 397, and/or 420 of the Amaranthus type II PPO increase herbicide tolerance.

Consequently, in preferred embodiments, combinations of mutations corresponding to the respective positions in SEQ ID NO: 2 or 4 are encompassed by the present invention and are shown in the following Table AA.

TABLE AA

| double | Triple | quadruple | quintuple |
|---|---|---|---|
| Q119L, R128A | Q119T, G210T, S387L | Q119L, R128A, Q323H, | Q119T, S387L, S412N, |
| | | S412N | M414Q, E436I |
| Q119L, Q323H | Q119T, G211A, S387L | Q119L, Q323H, L397D, | |
| | | S412N | |
| Q119T, S387L | Q119T, G211A, F420M | Q119L, Q323H, S412N, | |
| | | F420M | |
| Q119L, L397D | Q119L, R128A, F420M | Q119T, S387L, S412N, | |
| | | M414Q | |
| Q119L, S412N | Q119T, G210T, F420M | Q119T, S387L, M414Q, | |
| | | E436I | |
| Q119T, M414Q | Q119L, Q323H, S412N | R128A, G211A, S387L, | |
| | | F420M | |
| Q119L, F420M | Q119T, S387L, S412N | | |
| Q119T, E436I | Q119T, S387L, M414Q | | |
| K127N, R128C | Q119T, S387L, F420M | | |
| R128A, G210T | Q119T, S387L, E436I | | |
| R128A, G211A | Q119L, L397D, F420M | | |
| R128A, S387L | Q119T, S412N, E436I | | |
| R128A, S412N | Q119T, M414Q, E436I | | |
| D202A, S387V | R128A, G210T, F420V | | |
| G210C, G211A | R128A, G211A, F420M | | |
| G210T, S387L | R128A, S412N, F420M | | |
| G210C, L397Q | R128A, F420V, d_H524 | | |
| G210T, S412N | R128A, F420V, d_V527 | | |
| G210T, M414Q | G210T, G211A, M414Q | | |
| G210S, F420M | G211A, S387L, F420M | | |
| G211A, S387L | G211C, L397Q, F420V | | |
| G211C, L397Q | S387V, S412N, M414Q | | |
| G211A, M414Q | S387L, S412N, E436I | | |
| G211A, L397Q | S387L, M414Q, E436I | | |
| G211A, F420M | L397D, S412N, F420M | | |
| Q323H, S412N | L397E, F420M, d, H524 | | |
| M343T, F420M | L397E, F420M, d, V527 | | |
| D372E, K373D | i_Q391, H392K, N393H | | |
| D372E, F420M | R128A, G211N, F420M | | |
| S387L, L397Q | R128A, G210S, F420M | | |
| S387V, S412N | R128A, G211C, F420M | | |
| S387L, M414Q | | | |
| S387L, F420M | | | |
| S387V, R425Y | | | |
| S387L, E436I | | | |
| L397D, S412N | | | |
| S412N, M414Q | | | |
| S412N, F420M | | | |
| S412N, E436I | | | |
| M414Q, E436I | | | |
| M414Q, F420M | | | |
| F420M, d_H524 | | | |
| F420M, d_V527 | | | |
| E436I, F420M | | | |
| G211C, F420V | | | |
| G211D, F420M | | | |
| G211N, F420M | | | |
| G210T, G211C | | | |

It will be within the knowledge of the skilled artisan to identify conserved regions and motifs shared between the homologues, orthologues and paralogues. Having identified such conserved regions that may represent suitable binding motifs, amino acids can be chosen to be substituted by any other amino acid, for example by conserved amino acids, preferably by the amino acid substitutions described SUPRA using SEQ ID NO:2 or 4 as reference.

Accordingly, the plant or plant of the present invention comprises a polynucleotide encoding a mutated PPO polypeptide which comprises one or more of the following motifs.

i) Motif 1:
(SEQ ID NO: 131)
GT[C/S]GGDP

Wherein the glycine at position 4, and/or 5 within said motif of the corresponding wildtype sequence is substituted by any other amino acid

ii)
Motif 2:
(SEQ ID NO: 132)
[A/S/C]PS[D/N][X][X]L

Wherein the serine at position 3 within said motif of the corresponding wildtype sequence is substituted by any other amino acid

iii) Motif 3:
(SEQ ID NO: 133)
[R/Q][E/D]KQQ[L/Y]P

Wherein the glutamine at position 4 within said motif of the corresponding wildtype sequence is substituted by any other amino acid

iv) Motif 4:
(SEQ ID NO: 134)
L[I/V]PSKE

wherein the serine at position 4 within said motif of the corresponding wildtype sequence is substituted by any other amino acid.

Preferably, the mutated PPO polypeptide of the present invention in addition comprises one or more of the following motifs
a. Motif 5: SQ[N/K/H]KRYI (SEQ ID NO: 135), wherein the Arg at position 5 within said motif is substituted by any other amino acid;
b. Motif 6: TLGTLFSS (SEQ ID NO: 136), wherein the Leu at position 2 within said motif is substituted by by any other amino acid;
c. Motif 7: [F/Y]TTF[V/I]GG (SEQ ID NO: 137), wherein the Phe at position 4 within said motif is substituted by by any other amino acid.

Another object refers to a method of identifying a nucleotide sequence encoding a mutated PPO which is resistant or tolerant to a herbicide, the method comprising:
a) generating a library of mutated PPO-encoding nucleic acids,
b) screening a population of the resulting mutated PPO-encoding nucleic acids by expressing each of said nucleic acids in a cell or plant and treating said cell or plant with a herbicide,
c) comparing the herbicide-tolerance levels provided by said population of mutated PPO encoding nucleic acids with the herbicide-tolerance level provided by a control PPO-encoding nucleic acid,
d) selecting at least one mutated PPO-encoding nucleic acid that provides a significantly increased level of tolerance to a herbicide as compared to that provided by the control PPO-encoding nucleic acid.

Herbicide-tolerance levels may also be determined by measuring the detoxification rate in a cell, tissue, or plant.

Detoxification rate is the rate of herbicide degradation within a certain timeframe in a respective tissue. The degradation and product formation can be determined analytically for instance by liquid chromatographie (LC) coupled to a high resolution (HR) mass spectromter (MS). Product can be determined by comparison to authentic standards and/or by structure elucidation.

In a preferred embodiment, the mutated PPO-encoding nucleic acid selected in step d) provides at least 2-fold as much resistance or tolerance of a cell or plant to a herbicide as compared to that provided by the control PPO-encoding nucleic acid.

In a further preferred embodiment, the mutated PPO-encoding nucleic acid selected in step d) provides at least 2-fold, at least 5-fold, at least 10-fold, at least 20-fold, at least 50-fold, at least 100-fold, at least 500-fold, as much resistance or tolerance of a cell or plant to a herbicide as compared to that provided by the control PPO-encoding nucleic acid.

The resistance or tolerance can be determined by generating a transgenic plant or host cell, preferably a plant cell, comprising a nucleic acid sequence of the library of step a) and comparing said transgenic plant with a control plant or host cell, preferably a plant cell.

Many methods well known to the skilled artisan are available for obtaining suitable candidate nucleic acids for identifying a nucleotide sequence encoding a mutated PPO from a variety of different potential source organisms including microbes, plants, fungi, algae, mixed cultures etc. as well as environmental sources of DNA such as soil. These methods include inter alia the preparation of cDNA or genomic DNA libraries, the use of suitably degenerate oligonucleotide primers, the use of probes based upon known sequences or complementation assays (for example, for growth upon tyrosine) as well as the use of mutagenesis and shuffling in order to provide recombined or shuffled mutated PPO-encoding sequences.

Nucleic acids comprising candidate and control PPO encoding sequences can be expressed in yeast, in a bacterial host strain, in an alga or in a higher plant such as tobacco or Arabidopsis and the relative levels of inherent tolerance of the PPO encoding sequences screened according to a visible indicator phenotype of the transformed strain or plant in the presence of different concentrations of the selected herbicide. Dose responses and relative shifts in dose responses associated with these indicator phenotypes (formation of brown color, growth inhibition, herbicidal effect etc) are conveniently expressed in terms, for example, of GR50 (concentration for 50% reduction of growth) or MIC (minimum inhibitory concentration) values where increases in values correspond to increases in inherent tolerance of the expressed PPO. For example, in a relatively rapid assay system based upon transformation of a bacterium such as E. coli, each mutated PPO encoding sequence may be expressed, for example, as a DNA sequence under expression control of a controllable promoter such as the lacZ promoter and taking suitable account, for example by the use of synthetic DNA, of such issues as codon usage in order to obtain as comparable a level of expression as possible of different PPO sequences. Such strains expressing nucleic acids comprising alternative candidate PPO sequences may be plated out on different concentrations of the selected herbicide in, optionally, a tyrosine supplemented medium and the relative levels of inherent tolerance of the expressed PPO enzymes estimated on the basis of the extent and MIC for inhibition of the formation of the brown, ochronotic pigment, or by measuring the herbicide degradation via LC-HRMS (liquid chromatography high resolution mass spectrometry).

In another embodiment, candidate nucleic acids are transformed into plant material to generate a transgenic plant, regenerated into morphologically normal fertile plants which are then measured for differential tolerance to selected herbicides as described in the Example section hereinafter. Many suitable methods for transformation using suitable selection markers such as kanamycin, binary vectors such as from Agrobacterium and plant regeneration as, for example, from tobacco leaf discs are well known in the art. Optionally, a control population of plants is likewise transformed with a nucleic acid expressing the control PPO. The average, and distribution, of herbicide tolerance levels of a range of primary plant transformation events or their progeny to herbicides described supra are evaluated in the normal manner based upon plant damage, meristematic bleaching symptoms etc. at a range of different concentrations of herbicides. These data can be expressed in terms of, for example, GR50 values derived from dose/response curves having “dose” plotted on the x-axis and “percentage kill”, “herbicidal effect”, “numbers of emerging green plants” etc. plotted on the y-axis where increased GR50 values correspond to increased levels of inherent tolerance of the expressed PPO. Herbicides can suitably be applied pre-emergence or post-emergence.

Another object of the present invention refers to an isolated and/or recombinantly produced and/or synthetic nucleic acid molecule comprising a nucleic acid molecule encoding a mutated PPO polypeptide selected from the group consisting of:
(a) a nucleic acid molecule encoding a mutated PPO polypeptide comprising the sequence of—SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129, or a variant, paralogue, orthologue or homolog thereof;
(b) a nucleic acid molecule, which, as a result of the degeneracy of the genetic code, can be derived from a nucleic acid molecule encoding a mutated PPO polypeptide comprising the sequence of—SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129, or a variant, paralogue, orthologue or homolog thereof- and confers increased herbicide tolerance or resistance, as compared to a corresponding, e.g. non-transformed, wild type plant cell, a plant or a part thereof;
(c) a nucleic acid molecule encoding a mutated PPO polypeptide having 30% or more identity, preferably at least 40%, 50%, 60%, 70%, 75%, 80%, 85%, 90%, 95%, 96%, 97%, 98%, 99%, 99.5% or more, with the amino acid sequence of the PPO polypeptide sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129, and confers increased herbicide tolerance or resistance, as compared to a corresponding, e.g. non-transformed, wild type plant cell, a plant or a part thereof;
(d) nucleic acid molecule which hybridizes with a nucleic acid molecule of (a), (b), or (c), under stringent hybridization conditions and confers increased herbicide tolerance or resistance, as compared to a corresponding, e.g. non-transformed, wild type plant cell, a plant or a part thereof;
wherein the amino acid sequence of the encoded mutated PPO polypeptide differs from the wildtype amino acid sequence of a PPO polypeptide at one or more positions corresponding to positions 32, 53, 57, 61, 63, 64, 65, 67, 71, 76, 82, 83, 85, 86, 87, 88, 91, 103, 104, 106, 108, 116, 119, 126, 127, 129, 139, 159, 210, 211, 224, 245, 246, 248, 249, 252, 253, 254, 255, 257, 259, 260, 262, 264, 286, 291, 305, 308, 309, 323, 335, 343, 345, 358, 372, 373, 387, 391, 392, 400, 412, 414, 415, 425, 428, 431, 433, 434, 435, 436, 447, 451, 453, 464, 466, 482 of SEQ ID NO: 1 or 2, wherein said difference refers to a substitution of the amino acid at that positions by any other amino acid.

In a preferred embodiment, the amino acid sequence of the encoded mutated PPO polypeptide additionally differs from the wildtype amino acid sequence of a PPO polypeptide at one or more positions corresponding to positions 128, 397, or 420 of SEQ ID NO: 1 or 2, wherein said difference refers to a substitution of the amino acid at that positions by any other amino acid.

In another preferred embodiment, the amino acids at positions corresponding to positions 128, 211, 387, and 420 of SEQ ID NO: 1 or 2 are substituted by any other amino acid. Preferably, the amino acid corresponding to position 128 is substituted by Ala, the amino acid corresponding to position 211 is substituted by Ala, the amino acid corresponding to position 387 is substituted by Leu, and the amino acid corresponding to position 420 is substituted by Met.

In another preferred embodiment, the amino acids at positions corresponding to positions 128, 211, and 420 of SEQ ID NO: 1 or 2 are substituted by any other amino acid. Preferably, the amino acid corresponding to position 128 is substituted by Ala, the amino acid corresponding to position 211 is substituted by Asn or Cys, and the amino acid corresponding to position 420 is substituted by Met.

In another preferred embodiment, the amino acids at positions corresponding to positions 128, 210, and 420 of SEQ ID NO: 1 or 2 are substituted by any other amino acid. Preferably, the amino acid corresponding to position 128 is substituted by Ala, the amino acid corresponding to position 210 is substituted by Ser, and the amino acid corresponding to position 420 is substituted by Met.

In another preferred embodiment, the amino acids at positions corresponding to positions 128, 412, and 420 of SEQ ID NO: 1 or 2 are substituted by any other amino acid. Preferably, the amino acid corresponding to position 128 is substituted by Ala, the amino acid corresponding to position 412 is substituted by Asn, and the amino acid corresponding to position 420 is substituted by Met.

In another preferred embodiment, the amino acids at positions corresponding to positions 119, 128, and 420 of SEQ ID NO: 1 or 2 are substituted by any other amino acid. Preferably, the amino acid corresponding to position 119 is substituted by Leu, the amino acid corresponding to position 128 is substituted by Ala, and the amino acid corresponding to position 420 is substituted by Met.

In another preferred embodiment, the amino acids at positions corresponding to positions 211, 397 and 420 of SEQ ID NO: 1 or 2 are substituted by any other amino acid. Preferably, the amino acid corresponding to position 211 is substituted by Ala, the amino acid corresponding to position 397 is substituted by Gln, and the amino acid corresponding to position 420 is substituted by Met.

In another preferred embodiment, amino acids at positions corresponding to positions 211, and 420 of SEQ ID NO: 1 or 2 are substituted by any other amino acid.

Preferably, the amino acid corresponding to position 211 is substituted by Cys, and the amino acid corresponding to position 420 is substituted by Val.

Preferably, the amino acid corresponding to position 211 is substituted by Ala, and the amino acid corresponding to position 420 is substituted by Val.

Preferably, the amino acid corresponding to position 211 is substituted by Asp, and the amino acid corresponding to position 420 is substituted by Met.

Preferably, the amino acid corresponding to position 211 is substituted by Asn, and the amino acid corresponding to position 420 is substituted by Met.

In another preferred embodiment, amino acids at positions corresponding to positions 210, and 211 of SEQ ID NO: 1 or 2 are substituted by any other amino acid. Preferably, the amino acid corresponding to position 210 is substituted by Thr, and the amino acid corresponding to position 211 is substituted by Cys.

In a preferred embodiment, the encoded mutated PPO is a variant of SEQ ID NO:2 or 4, which includes one or more of the following:

the amino acid corresponding to position G32 is substituted by any other amino acid

the amino acid corresponding to position V53 is substituted by any other amino acid

the amino acid corresponding to position A57 is substituted by any other amino acid

the amino acid corresponding to position K61 is substituted by any other amino acid

the amino acid corresponding to position K63 is substituted by any other amino acid

the amino acid corresponding to position S64 is substituted by any other amino acid

the amino acid corresponding to position H65 is substituted by any other amino acid

the amino acid corresponding to position L67 is substituted by any other amino acid

the amino acid corresponding to position L71 is substituted by any other amino acid

the amino acid corresponding to position S76 is substituted by any other amino acid

the amino acid corresponding to position L82 is substituted by any other amino acid

the amino acid corresponding to position K83 is substituted by any other amino acid

the amino acid corresponding to position V85 is substituted by any other amino acid

the amino acid corresponding to position K86 is substituted by any other amino acid

the amino acid corresponding to position K87 is substituted by any other amino acid

the amino acid corresponding to position D88 is substituted by any other amino acid

the amino acid corresponding to position I91 is substituted by any other amino acid

the amino acid corresponding to position E103 is substituted by any other amino acid

the amino acid corresponding to position A104 is substituted by any other amino acid

the amino acid corresponding to position V106 is substituted by any other amino acid

the amino acid corresponding to position S108 is substituted by any other amino acid

the amino acid corresponding to position R116 is substituted by any other amino acid

the amino acid corresponding to position Q119 is substituted by any other amino acid, preferably Leu

the amino acid corresponding to position K127 is substituted by any other amino acid

the amino acid corresponding to position N126 is substituted by any other amino acid

the amino acid corresponding to position Y129 is substituted by any other amino acid

the amino acid corresponding to position L139 is substituted by any other amino acid

the amino acid corresponding to position Q159 is substituted by any other amino acid

the amino acid corresponding to position G210 is substituted by any other amino acid, preferably Ser;

the amino acid corresponding to position G211 is substituted by any other amino acid, preferably Cys, Ala, Asp, Thr, or Asn;

the amino acid corresponding to position E224 is substituted by any other amino acid

the amino acid corresponding to position L245 is substituted by any other amino acid

the amino acid corresponding to position K248 is substituted by any other amino acid

the amino acid corresponding to position S246 is substituted by any other amino acid

the amino acid corresponding to position E249 is substituted by any other amino acid

the amino acid corresponding to position G252 is substituted by any other amino acid

the amino acid corresponding to position E253 is substituted by any other amino acid

the amino acid corresponding to position N254 is substituted by any other amino acid

the amino acid corresponding to position A255 is substituted by any other amino acid

the amino acid corresponding to position 1257 is substituted by any other amino acid

the amino acid corresponding to position K259 is substituted by any other amino acid

the amino acid corresponding to position P260 is substituted by any other amino acid

the amino acid corresponding to position V262 is substituted by any other amino acid

the amino acid corresponding to position G264 is substituted by any other amino acid

the amino acid corresponding to position D286 is substituted by any other amino acid

the amino acid corresponding to position Q291 is substituted by any other amino acid

the amino acid corresponding to position P305 is substituted by any other amino acid

the amino acid corresponding to position G308 is substituted by any other amino acid

the amino acid corresponding to position N309 is substituted by any other amino acid

the amino acid corresponding to position Q323 is substituted by any other amino acid

the amino acid corresponding to position R335 is substituted by any other amino acid

the amino acid corresponding to position M343 is substituted by any other amino acid

the amino acid corresponding to position F345 is substituted by any other amino acid

the amino acid corresponding to position T358 is substituted by any other amino acid

the amino acid corresponding to position D372 is substituted by any other amino acid

the amino acid corresponding to position K373 is substituted by any other amino acid

the amino acid corresponding to position S387 is substituted by any other amino acid, preferably Leu;

the amino acid corresponding to position H391 is substituted by any other amino acid

the amino acid corresponding to position N392 is substituted by any other amino acid

the amino acid corresponding to position L400 is substituted by any other amino acid

the amino acid corresponding to position S412 is substituted by any other amino acid, preferably Leu;

the amino acid corresponding to position M414 is substituted by any other amino acid

the amino acid corresponding to position C415 is substituted by any other amino acid

the amino acid corresponding to position R425 is substituted by any other amino acid

the amino acid corresponding to position K428 is substituted by any other amino acid

the amino acid corresponding to position N431 is substituted by any other amino acid

the amino acid corresponding to position S433 is substituted by any other amino acid

the amino acid corresponding to position M434 is substituted by any other amino acid

the amino acid corresponding to position D435 is substituted by any other amino acid

the amino acid corresponding to position E436 is substituted by any other amino acid

the amino acid corresponding to position Q447 is substituted by any other amino acid

the amino acid corresponding to position T451 is substituted by any other amino acid

the amino acid corresponding to position D453 is substituted by any other amino acid

the amino acid corresponding to position S464 is substituted by any other amino acid

the amino acid corresponding to position Q466 is substituted by any other amino acid

the amino acid corresponding to position D482 is substituted by any other amino acid

In another preferred embodiment, the encoded mutated PPO is a variant of SEQ ID NO:2 or 4, which includes one or more of the following:

the amino acid corresponding to position P40 is deleted;

the amino acid corresponding to position K250 is deleted;

the amino acid corresponding to position Q391 is inserted

the amino acid corresponding to position Y520 is deleted;

the amino acid corresponding to position L521 is deleted;

the amino acid corresponding to position D522 is deleted;

the amino acid corresponding to position S523 is deleted;

the amino acid corresponding to position H524 is deleted;

the amino acid corresponding to position I525 is deleted;

the amino acid corresponding to position Y526 is deleted;

the amino acid corresponding to position V527 is deleted;

the amino acid corresponding to position K528 is deleted;

the amino acid corresponding to position M529 is deleted;

the amino acid corresponding to position D530 is deleted;

the amino acid corresponding to position E531 is deleted;

the amino acid corresponding to position K532 is deleted;

the amino acid corresponding to position T533 is deleted;

the amino acid corresponding to position A534 is deleted;

In other aspects, the present invention encompasses a progeny or a descendant of a herbicide-tolerant plant of the present invention as well as seeds derived from the herbicide-tolerant plants of the invention and cells derived from the herbicide-tolerant plants of the invention.

In some embodiments, the present invention provides a progeny or descendant plant derived from a plant comprising in at least some of its cells a polynucleotide operably linked to a promoter operable in plant cells, the promoter capable of expressing a mutated PPO polypeptide encoded by the polynucleotide, wherein the progeny or descendant plant comprises in at least some of its cells the recombinant polynucleotide operably linked to the promoter, the expression of the mutated PPO polypeptide conferring to the progeny or descendant plant tolerance to the herbicides.

In one embodiment, seeds of the present invention preferably comprise the herbicide-tolerance characteristics of the herbicide-tolerant plant. In other embodiments, a seed is capable of germination into a plant comprising in at least some of its cells a polynucleotide operably linked to a promoter operable in plant cells, the promoter capable of expressing a mutated PPO polypeptide encoded by the polynucleotide, the expression of the mutated PPO polypeptide conferring to the progeny or descendant plant tolerance to the herbicides.

In some embodiments, plant cells of the present invention are capable of regenerating a plant or plant part. In other embodiments, plant cells are not capable of regenerating a plant or plant part. Examples of cells not capable of regenerating a plant include, but are not limited to, endosperm, seed coat (testa & pericarp), and root cap.

In another embodiment, the present invention provides a plant cell of or capable of regenerating a plant comprising in at least some of its cells a polynucleotide operably linked to a promoter operable in plant cells, the promoter capable of expressing a mutated PPO polypeptide encoded by the polynucleotide, the expression of the mutated PPO polypeptide conferring to the plant tolerance to the herbicides, wherein the plant cell comprises the recombinant polynucleotide operably linked to a promoter.

In other embodiments, the present invention provides a plant cell comprising a polynucleotide operably linked to a promoter operable in plant cells, the promoter capable of expressing a mutated PPO polypeptide encoded by the polynucleotide, the expression of the mutated PPO polypeptide conferring to the cell tolerance to the herbicides.

In another embodiment, the invention refers to a plant cell transformed by a nucleic acid encoding a mutated PPO polypeptide according to the present invention, wherein expression of the nucleic acid in the plant cell results in increased resistance or tolerance to a herbicide as compared to a wild type variety of the plant cell. Preferably, the mutated PPO polypeptide encoding nucleic acid comprises a polynucleotide sequence selected from the group consisting of: a) a polynucleotide as shown in SEQ ID NO: 118, 119, 120, 121, 122, 123, 124, 125, 126, 127, 128, or 130, or a variant or derivative thereof; b) a polynucleotide encoding a polypeptide as shown in SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129, or a variant or derivative thereof; c) a polynucleotide comprising at least 60 consecutive nucleotides of any of a) or b); and d) a polynucleotide complementary to the polynucleotide of any of a) through c).

In some aspects, the present invention provides a plant product prepared from the herbicide-tolerant plants hereof. In some embodiments, examples of plant products include, without limitation, grain, oil, and meal. In one embodiment, a plant product is plant grain (e.g., grain suitable for use as feed or for processing), plant oil (e.g., oil suitable for use as food or biodiesel), or plant meal (e.g., meal suitable for use as feed).

In one embodiment, a plant product prepared from a plant or plant part is provided, wherein the plant or plant part comprises in at least some of its cells a polynucleotide operably linked to a promoter operable in plant cells, the promoter capable of expressing a mutated PPO polypeptide encoded by the polynucleotide, the expression of the mutated PPO polypeptide conferring to the a plant or plant part tolerance to the herbicides.

In another embodiment, the invention refers to a method of producing a transgenic plant cell with an increased resistance to a herbicide as compared to a wild type variety of the plant cell comprising, transforming the plant cell with an expression cassette comprising a polynucleotide operably linked to a promoter operable in plant cells, the promoter capable of expressing a mutated PPO polypeptide encoded by the polynucleotide.

In another embodiment, the invention refers to a method of producing a transgenic plant comprising, (a) transforming a plant cell with an expression cassette comprising a polynucleotide operably linked to a promoter operable in plant cells, the promoter capable of expressing a mutated PPO polypeptide encoded by the polynucleotide, and (b) generating a plant with an increased resistance to herbicide from the plant cell. In some aspects, the present invention provides a method for producing a herbicide-tolerant plant. In one embodiment, the method comprises: regenerating a plant from a plant cell transformed with a polynucleotide operably linked to a promoter operable in plant cells, the promoter capable of expressing a mutated PPO polypeptide encoded by the polynucleotide, the expression of the mutated PPO polypeptide conferring to the plant tolerance to the herbicides.

The term “expression/expressing” or “gene expression” means the transcription of a specific gene or specific genes or specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product.

To obtain the desired effect, i.e. plants that are tolerant or resistant to the herbicide derivative herbicide of the present invention, it will be understood that the at least one nucleic acid is “over-expressed” by methods and means known to the person skilled in the art.

The term “increased expression” or “overexpression” as used herein means any form of expression that is additional to the wild-type expression level. Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the polypeptide of interest. For example, endogenous promoters may be altered in vivo by mutation, deletion, and/or substitution (see, Kmiec, U.S. Pat. No. 5,565,350; Zarling et al., WO9322443), or isolated promoters may be introduced into a plant cell in the proper orientation and distance from a gene of the present invention so as to control the expression of the gene. If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene. An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell biol. 8: 4395-4405; Callis et al. (1987) Genes Dev 1:1183-1200). Such intron enhancement of gene expression is typically greatest when placed near the 5′ end of the transcription unit. Use of the maize introns Adhl-S intron 1, 2, and 6, the Bronze-1 intron are known in the art. For general information see: The Maize Handbook, Chapter 116, Freeling and Walbot, Eds., Springer, N.Y. (1994).

Where appropriate, nucleic acid sequences may be optimized for increased expression in a transformed plant. For example, coding sequences that comprise plant-preferred codons for improved expression in a plant can be provided. See, for example, Campbell and Gowri (1990) Plant Physiol., 92: 1-11 for a discussion of host-preferred codon usage. Methods also are known in the art for preparing plant-preferred genes. See, for example, U.S. Pat. Nos. 5,380,831, and 5,436,391, and Murray et al. (1989) Nucleic Acids Res. 17:477-498, herein incorporated by reference.

Consequently, mutated PPO nucleic acids of the invention are provided in expression cassettes for expression in the plant of interest. The cassette will include regulatory sequences operably linked to a mutated PPO nucleic acid sequence of the invention. The term “regulatory element” as used herein refers to a polynucleotide that is capable of regulating the transcription of an operably linked polynucleotide. It includes, but not limited to, promoters, enhancers, introns, 5′ UTRs, and 3′ UTRs. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes.

Such an expression cassette is provided with a plurality of restriction sites for insertion of the mutated PPO nucleic acid sequence to be under the transcriptional regulation of the regulatory regions. The expression cassette may additionally contain selectable marker genes. The expression cassette of the present invention will include in the 5′-3′ direction of transcription, a transcriptional and translational initiation region (i.e., a promoter), a mutated PPO encoding nucleic acid sequence of the invention, and a transcriptional and translational termination region (i.e., termination region) functional in plants. The promoter may be native or analogous, or foreign or heterologous, to the plant host and/or to the mutated PPO nucleic acid sequence of the invention. Additionally, the promoter may be the natural sequence or alternatively a synthetic sequence. Where the promoter is “foreign” or “heterologous” to the plant host, it is intended that the promoter is not found in the native plant into which the promoter is introduced. Where the promoter is “foreign” or “heterologous” to the mutated PPO nucleic acid sequence of the invention, it is intended that the promoter is not the native or naturally occurring promoter for the operably linked mutatedTriA nucleic acid sequence of the invention. As used herein, a chimeric gene comprises a coding sequence operably linked to a transcription initiation region that is heterologous to the coding sequence. While it may be preferable to express the mutated PPO nucleic acids of the invention using heterologous promoters, the native promoter sequences may be used. Such constructs would change expression levels of the mutated PPO protein in the plant or plant cell. Thus, the phenotype of the plant or plant cell is altered.

The termination region may be native with the transcriptional initiation region, may be native with the operably linked mutated PPO sequence of interest, may be native with the plant host, or may be derived from another source (i.e., foreign or heterologous to the promoter, the mutated PPO nucleic acid sequence of interest, the plant host, or any combination thereof). Convenient termination regions are available from the Ti-plasmid of A. tumefaciens, such as the octopine synthase and nopaline synthase termination regions. See also Guerineau et al. (1991) Mol. Gen. Genet. 262: 141-144; Proudfoot (1991) Cell 64:671-674; Sanfacon et al. (1991) Genes Dev. 5: 141-149; Mogen et al. (1990) Plant Cell 2: 1261-1272; Munroe et al. (1990) Gene 91: 151-158; Ballas t al. (1989) Nucleic Acids Res. 17:7891-7903; and Joshi et al. (1987) Nucleic Acid Res. 15:9627-9639. Where appropriate, the gene(s) may be optimized for increased expression in the transformed plant. That is, the genes can be synthesized using plant-preferred codons for improved expression. See, for example, Campbell and Gowri (1990) Plant Physiol. 92: 1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Thus, the present invention provides an expression cassette comprising a mutated PPO nucleic acid nucleic acid molecule according to the present invention and a promoter operable in plant cells.

While the polynucleotides of the invention may find use as selectable marker genes for plant transformation, the expression cassettes of the invention can include another selectable marker gene for the selection of transformed cells. Selectable marker genes, including those of the present invention, are utilized for the selection of transformed cells or tissues. Marker genes include, but are not limited to, genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). See generally, Yarranton (1992) Curr. Opin. Biotech. 3:506-511; Christophers on et al (1992) Proc. Natl. Acad. ScL USA 89:6314-6318; Yao et al. (1992) Cell 71:63-72; Reznikoff (1992) Mol Microbiol 6:2419-2422; Barkley et al (1980) in The Operon, pp. 177-220; Hu et al (1987) Cell 48:555-566; Brown et al (1987) Cell 49:603-612; Figge et al (1988) Cell 52:713-722; Deuschle et al (1989) Proc. Natl Acad. AcL USA 86:5400-5404; Fuerst et al (1989) Proc. Natl Acad. ScL USA 86:2549-2553; Deuschle et al (1990) Science 248:480-483; Gossen (1993) Ph.D. Thesis, University of Heidelberg; Reines et al (1993) Proc. Natl Acad. ScL USA 90: 1917-1921; Labow et al (1990) Mol Cell Biol 10:3343-3356; Zambretti et al (1992) Proc. Natl Acad. ScL USA 89:3952-3956; Bairn et al (1991) Proc. Natl Acad. ScL USA 88:5072-5076; Wyborski et al (1991) Nucleic Acids Res. 19:4647-4653; Hillenand-Wissman (1989) Topics Mol Struc. Biol 10: 143-162; Degenkolb et al (1991) Antimicrob. Agents Chemother. 35: 1591-1595; Kleinschnidt et al (1988) Biochemistry 27: 1094-1104; Bonin (1993) Ph.D. Thesis, University of Heidelberg; Gossen et al (1992) Proc. Natl Acad. ScL USA 89:5547-5551; Oliva et al (1992) Antimicrob. Agents Chemother. 36:913-919; Hlavka et al (1985) Handbook of Experimental Pharmacology, Vol. 78 (Springer-Verlag, Berlin); Gill et al (1988) Nature 334:721-724. Such disclosures are herein incorporated by reference. The above list of selectable marker genes is not meant to be limiting. Any selectable marker gene can be used in the present invention.

Further, additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. Also, if desired, sequences can be readily modified to avoid predicted hairpin secondary mRNA structures. Nucleotide sequences for enhancing gene expression can also be used in the plant expression vectors. These include, for example, introns of the maize Adh gene Adhl-S intron 1, 2, and 6 (Callis et al. Genes and Development 1: 1183-1200, 1987), and leader sequences, (W-sequence) from the Tobacco Mosaic virus (TMV), Maize Chlorotic Mottle Virus and Alfalfa Mosaic Virus (Gallie et al. Nucleic Acid Res. 15:8693-8711, 1987 and Skuzeski et al. Plant Mol. Biol. 15:65-79, 1990). The first intron from the shrunken-1 locus of maize has been shown to increase expression of genes in chimeric gene constructs. U.S. Pat. Nos. 5,424,412 and 5,593,874 disclose the use of specific introns in gene expression constructs, and Gallie et al. (Plant Physiol. 106:929-939, 1994) also have shown that introns are useful for regulating gene expression on a tissue specific basis. To further enhance or to optimize gene expression, the plant expression vectors of the invention also may contain DNA sequences containing matrix attachment regions (MARs). Plant cells transformed with such modified expression systems, then, may exhibit overexpression or constitutive expression of a nucleotide sequence of the invention.

The invention further provides an isolated recombinant expression vector comprising the expression cassette containing a mutated PPO nucleic acid nucleic acid as described above, wherein expression of the vector in a host cell results in increased tolerance to a herbicide as compared to a wild type variety of the host cell. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses, and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operably linked to the nucleic acid sequence to be expressed. Regulatory sequences include those that direct constitutive expression of a nucleotide sequence in many types of host cells and those that direct expression of the nucleotide sequence only in certain host cells or under certain conditions. It will be appreciated by those skilled in the art that the design of the expression vector can depend on such factors as the choice of the host cell to be transformed, the level of expression of polypeptide desired, etc. The expression vectors of the invention can be introduced into host cells to thereby produce polypeptides or peptides, including fusion polypeptides or peptides, encoded by nucleic acids as described herein (e.g., mutated PPO polypeptides, fusion polypeptides, etc.)

Expression vectors may additionally contain 5′ leader sequences in the expression construct. Such leader sequences can act to enhance translation. Translation leaders are known in the art and include: picornavirus leaders, for example, EMCV leader (Encephalomyo carditis 5′ noncoding region) (Elroy-Stein et al. (1989) PNAS, 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968.

Other methods known to enhance translation also can be utilized, for example, introns, and the like. In preparing an expression vector, the various nucleic acid fragments may be manipulated, so as to provide for the nucleic acid sequences in the proper orientation and, as appropriate, in the proper reading frame. Toward this end, adapters or linkers may be employed to join the nucleic acid fragments or other manipulations may be involved to provide for convenient restriction sites, removal of superfluous nucleic acid, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, tissue-preferred, or other promoters for expression in plants.

Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2: 163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to target enhanced expression within a particular plant tissue. Such tissue-preferred promoters include, but are not limited to, leaf-preferred promoters, root-preferred promoters, seed-preferred promoters, and stem-preferred promoters. Some examples of tissue-preferred promoters are described by, e.g., Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2): 157-168; Rinehart et al. (1996) Plant Physiol. 1 12(3): 1331-1341; Van Camp et al. (1996) Plant Physiol. 112(2):525-535; Canevascini et al. (1996) Plant Physiol. 1 12(2):513-524; Yamamoto et al. (1994) Plant Cell Physiol. 35(5):773-778; Lam (1994) Results Probl. Cell Differ. 20:181-196; Orozco ef al. (1993) Plant Mol Biol. 23(6): 1 129-1138; Matsuoka et al. (1993) Voc Natl. Acad. ScL USA 90(20):9586-9590; and Guevara-Garcia et al. (1993) Plant J 4(3):495-505. Promoters can be modified, if necessary, for weak expression.

In some embodiments, the nucleic acids of interest can be targeted to the chloroplast for expression. In this manner, where the nucleic acid of interest is not directly inserted into the chloroplast, the expression vector will additionally contain a chloroplast-targeting sequence comprising a nucleotide sequence that encodes a chloroplast transit peptide to direct the gene product of interest to the chloroplasts. Such transit peptides are known in the art. With respect to chloroplast-targeting sequences, “operably linked” means that the nucleic acid sequence encoding a transit peptide (i.e., the chloroplast-targeting sequence) is linked to the desired coding sequence of the invention such that the two sequences are contiguous and in the same reading frame. See, for example, Von Heijne et al. (1991) Plant Mol. Biol. Rep. 9: 104-126; Clark et al. (1989) J Biol. Chem. 264:17544-17550; Della-Cioppa et al. (1987) Plant Physiol. 84:965-968; Romer et al. (1993) Biochem. Biophys. Res. Commun. 196: 1414-1421; and Shah et al. (1986) Science 233:478-481. For example, a chloroplast transit peptide known in the art can be fused to the amino acid sequence of a PPO polypeptide of the invention by operably linking a choloroplast-targeting sequence to the 5′-end of a nucleotide sequence encoding the PPO polypeptide.

Chloroplast targeting sequences are known in the art and include the chloroplast small subunit of ribulose-1,5-bisphosphate carboxylase (Rubisco) (de Castro Silva Filho et al. (1996) Plant Mol. Biol. 30:769-780; Schnell et al. (1991) J Biol. Chem. 266(5):3335-3342); EPSPS (Archer et al. (1990) J Bioenerg. Biomemb. 22(6):789-810); tryptophan synthase (Zhao et al. (1995) J Biol. Chem. 270(11):6081-6087); plastocyanin (Lawrence et al. (1997) J Biol. Chem. 272(33):20357-20363); chorismate synthase (Schmidt et al. (1993) J Biol. Chem. 268(36):27447-27457); and the light harvesting chlorophyll a/b binding protein (LHBP) (Lamppa et al. (1988) J Biol. Chem. 263: 14996-14999). See also Von Heijne et al. (1991) Plant Mol. Biol. Rep. 9: 104-126; Clark et al. (1989) J Biol. Chem. 264: 17544-17550; Della-Cioppa et al. (1987) Plant Physiol. 84:965-968; Romer et al. (1993) Biochem Biophys. Res. Commun. 196: 1414-1421; and Shah et al. (1986) Science 233:478-481.

Methods for transformation of chloroplasts are known in the art. See, for example, Svab et al. (1990) Proc. Natl. Acad. ScL USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305.

The nucleic acids of interest to be targeted to the chloroplast may be optimized for expression in the chloroplast to account for differences in codon usage between the plant nucleus and this organelle. In this manner, the nucleic acids of interest may be synthesized using chloroplast-preferred codons. See, for example, U.S. Pat. No. 5,380,831, herein incorporated by reference.

Numerous plant transformation vectors and methods for transforming plants are available. See, for example, An, G. et al. (1986) Plant PysioL, 81:301-305; Fry, J., et al. 1987) Plant Cell Rep. 6:321-325; Block, M. (1988) Theor. Appl. Genet 0.16: 161-1 1 A; Hinchee, et al. (1990) Stadler. Genet. Symp. 2032Y2.203-2Y2; Cousins, et al. (1991) Aust. J. Plant Physiol. 18:481-494; Chee, P. P. and Slightom, J. L. (1992) Gene. 1 18:255-260; Christou, et al. (1992) Trends. Biotechnol. 10:239-246; Halluin, et al. (1992) Bio/Technol. 10:309-314; Dhir, et al. (1992) Plant Physiol. 99:81-88; Casas et al. (1993) Proc. Nat. Acad Sd. USA 90: 1 1212-1 1216; Christou, P. (1993) In Vitro Cell. Dev. Biol.-Plant; 29P. 119-124; Davies, et al. (1993) Plant Cell Rep. 12: 180-183; Dong, J. A. and Mchughen, A. (1993) Plant ScL 91: 139-148; Franklin, C. I. and Trieu, T. N. (1993) Plant. Physiol. 102: 167; Golovkin, et al. (1993) Plant ScL 90:41-52; Guo Chin ScL Bull. 38:2072-2078; Asano, et al. (1994) Plant Cell Rep. 13; Ayeres N. M. and Park, W. D. (1994) Crit. Rev. Plant. Sci. 13:219-239; Barcelo, et al. (1994) Plant. J. 5:583-592; Becker, et al. (1994) Plant. J. 5:299-307; Borkowska et al. (1994) Acta. Physiol Plant. 16:225-230; Christou, P. (1994) Agro. Food. Ind. Hi Tech. 5: 17-27; Eapen et al. (1994) Plant Cell Rep. 13:582-586; Hartman, et al. (1994) Bio-Technology 12: 919923; Ritala, et al. (1994) Plant. Mol. Biol. 24:317-325; and Wan, Y. C. and Lemaux, P. G. (1994) Plant Physiol. 104:3748.

In some embodiments, the methods of the invention involve introducing a polynucleotide construct into a plant. By “introducing” is intended presenting to the plant the polynucleotide construct in such a manner that the construct gains access to the interior of a cell of the plant. The methods of the invention do not depend on a particular method for introducing a polynucleotide construct to a plant, only that the polynucleotide construct gains access to the interior of at least one cell of the plant. Methods for introducing polynucleotide constructs into plants are known in the art including, but not limited to, stable transformation methods, transient transformation methods, and virus-mediated methods. The term “introduction” or “transformation” as referred to herein further means the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

By “stable transformation” is intended that the polynucleotide construct introduced into a plant integrates into the genome of the plant and is capable of being inherited by descendent thereof. By “transient transformation” is intended that a polynucleotide construct introduced into a plant does not integrate into the genome of the plant.

For the transformation of plants and plant cells, the nucleotide sequences of the invention are inserted using standard techniques into any vector known in the art that is suitable for expression of the nucleotide sequences in a plant or plant cell. The selection of the vector depends on the preferred transformation technique and the target plant species to be transformed. In an embodiment of the invention, the encoding nucleotide sequence is operably linked to a plant promoter, e.g. a promoter known in the art for high-level expression in a plant cell, and this construct is then introduced into a plant cell that is susceptible to herbicides; and a transformed plant is regenerated. In some embodiments, the transformed plant is tolerant to exposure to a level of herbicides that would kill or significantly injure a plant regenerated from an untransformed cell. This method can be applied to any plant species or crops.

Methodologies for constructing plant expression vectors and introducing foreign nucleic acids into plants are generally known in the art. For example, foreign DNA can be introduced into plants, using tumor-inducing (Ti) plasmid vectors. Other methods utilized for foreign DNA delivery involve the use of PEG mediated protoplast transformation, electroporation, microinjection whiskers, and biolistics or microprojectile bombardment for direct DNA uptake. Such methods are known in the art. (U.S. Pat. No. 5,405,765 to Vasil et al.; Bilang et a (1991) Gene 100: 247-250; Scheid et al. al, (1991) MoL Gen. Genet., 228: 104-1 12; Guerche et al., (1987) Plant Science 52: 1 1 1-1 16; Neuhause et al., (1987) Theor. Appl Genet. 75: 30-36; Klein et al., (1987) Nature 327: 70-73; Howell et al., (1980) Science 208: 1265; Horsch et al., (1985) Science 227: 1229-1231; DeBlock et al., (1989) Plant Physiology 91: 694-701; Methods for Plant Molecular Biology (Weissbach and Weissbach, eds.) Academic Press, Inc. (1988) and Methods in Plant Molecular Biology (Schuler and Zielinski, eds.) Academic Press, Inc. (1989).

Other suitable methods of introducing nucleotide sequences into plant cells include microinjection as described by, e.g., Crossway et al. (1986) Biotechniques 4:320-334, electroporation as described by e.g., Riggs et al. (1986) Proc. Natl. Acad. ScL USA 83:5602-5606, Agrobacterium-mediated transformation as described by e.g., Townsend et al., U.S. Pat. No. 5,563,055, Zhao et al., U.S. Pat. No. 5,981,840, direct gene transfer as described by, e.g., Paszkowski et al. (1984) EMBO J. 3:2717-2722, and ballistic particle acceleration as described by, e.g., U.S. Pat. Nos. 4,945,050; 5,879,918; 5,886,244; and U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Led transformation (WO 00/28058). Also see, Weissinger et al., (1988) Ann. Rev. Genet. 22:421-477; Sanford et al, (1987) Particulate Science and Technology 5:27-37 (onion); Christou et al, (1988) Plant Physiol. 87:671-674 (soybean); McCabe et al., (1988) Bio/Technology 6:923-926 (soybean); Finer and McMullen (1991) In Vitro Cell Dev. Biol. 27P: 175-182 (soybean); Singh et al, (1998) Theor. Appl. Genet. 96:319-324 (soybean); Datta et al., (1990) Biotechnology 8:736-740 (rice); Klein et al., (1988) PNAS, 85:4305-4309 (maize); Klein et al., (1988) Biotechnology 6:559-563 (maize); U.S. Pat. Nos. 5,240,855; 5,322,783; and U.S. Pat. No. 5,324,646; Tomes et al., (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al., (1988) Plant Physiol. 91:440-444 (maize); Fromm et al., (1990) Biotechnology 8:833-839 (maize); Hooykaas-Van Slogteren et al., (1984) Nature (London) 311:763-764; Bowen et al, U.S. Pat. No. 5,736,369 (cereals); Bytebier et al, (1987) PNAS 84:5345-5349 (Liliaceae); De Wet et al., (1985) in The Experimental Manipulation of Ovule Tissues, ed. Chapman et al, (Longman, New York), pp. 197-209 (pollen); Kaeppler et al., (1990) Plant Cell Reports 9:415-418 and Kaeppler et al., (1992) Theor. Apph Genet. 84:560-566 (whisker-mediated transformation); D'Halluin et al., (1992) Plant Cell 4: 1495-1505 (electroporation); Li et al., (1993) Plant Cell Reports 12:250-255 and Christou and Ford (1995) Annals of Botany 75:407-413 (rice); Osjoda et al, (1996) Nature Biotechnology 14:745-750 (maize via Agrobacterium tumefaciens); each of which is herein incorporated by reference.

Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-743). Methods for Agrobacterium-mediated transformation of rice include well known methods for rice transformation, such as those described in any of the following: European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Höfgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

One transformation method known to those of skill in the art is the dipping of a flowering plant into an Agrobacteria solution, wherein the Agrobacteria contains the PPO nucleic acid, followed by breeding of the transformed gametes. Agrobacterium mediated plant transformation can be performed using for example the GV3101(pMP90) (Koncz and Schell, 1986, Mol. Gen. Genet. 204:383-396) or LBA4404 (Clontech) Agrobacterium tumefaciens strain. Transformation can be performed by standard transformation and regeneration techniques (Deblaere et al., 1994, Nucl. Acids. Res. 13:4777-4788; Gelvin, Stanton B. and Schilperoort, Robert A, Plant Molecular Biology Manual, 2nd Ed.—Dordrecht: Kluwer Academic Publ., 1995.—in Sect., Ringbuc Zentrale Signatur: BT11-P ISBN 0-7923-2731-4; Glick, Bernard R. and Thompson, John E., Methods in Plant Molecular Biology and Biotechnology, Boca Raton: CRC Press, 1993 360 S., ISBN 0-8493-5164-2). For example, rapeseed can be transformed via cotyledon or hypocotyl transformation (Moloney et al., 1989, Plant Cell Report 8:238-242; De Block et al., 1989, Plant Physiol. 91:694-701). Use of antibiotics for Agrobacterium and plant selection depends on the binary vector and the Agrobacterium strain used for transformation. Rapeseed selection is normally performed using kanamycin as selectable plant marker. Agrobacterium mediated gene transfer to flax can be performed using, for example, a technique described by Mlynarova et al., 1994, Plant Cell Report 13:282-285. Additionally, transformation of soybean can be performed using for example a technique described in European Patent No. 0424 047, U.S. Pat. No. 5,322,783, European Patent No. 0397 687, U.S. Pat. No. 5,376,543, or U.S. Pat. No. 5,169,770. Transformation of maize can be achieved by particle bombardment, polyethylene glycol mediated DNA uptake, or via the silicon carbide fiber technique. (See, for example, Freeling and Walbot “The maize handbook” Springer Verlag: New York (1993) ISBN 3-540-97826-7). A specific example of maize transformation is found in U.S. Pat. No. 5,990,387, and a specific example of wheat transformation can be found in PCT Application No. WO 93/07256.

In some embodiments, polynucleotides of the present invention may be introduced into plants by contacting plants with a virus or viral nucleic acids. Generally, such methods involve incorporating a polynucleotide construct of the invention within a viral DNA or RNA molecule. It is recognized that the polypeptides of the invention may be initially synthesized as part of a viral polyprotein, which later may be processed by proteolysis in vivo or in vitro to produce the desired recombinant polypeptide. Further, it is recognized that promoters of the invention also encompass promoters utilized for transcription by viral RNA polymerases. Methods for introducing polynucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference. The cells that have been transformed may be grown into plants in accordance with conventional ways. See, for example, McCormick et a (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved.

The present invention may be used for transformation of any plant species, including, but not limited to, monocots and dicots. Examples of plant species of interest include, but are not limited to, corn or maize (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet e.g., pearl millet (Pennisetum glaucum), proso millet (Panicum miliaceum), foxtail millet (Setaria italica), finger millet (Eleusine coracana)), sunflower (Helianthus annu), saffiower (Carthamus tinctorius), wheat (Triticum aestivum, T. Turgidum ssp. durum), soybean (Glycine max), tobacco (Nicotiana tabacum), potato (Solarium tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers. Preferably, plants of the present invention are crop plants (for example, sunflower, Brassica sp., cotton, sugar, beet, soybean, peanut, alfalfa, safflower, tobacco, corn, rice, wheat, rye, barley triticale, sorghum, millet, etc.).

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic [Feldman, KA and Marks MD (1987). Mol Gen Genet 208:274-289; Feldmann K (1992). In: C Koncz, N-H Chua and J Shell, eds, Methods in Arabidopsis Research. Word Scientific, Singapore, pp. 274-2891. Alternative methods are based on the repeated removal of the inflorescences and incubation of the excision site in the center of the rosette with transformed agrobacteria, whereby transformed seeds can likewise be obtained at a later point in time (Chang (1994). Plant J. 5: 551-558; Katavic (1994). Mol Gen Genet, 245: 363-370). However, an especially effective method is the vacuum infiltration method with its modifications such as the “floral dip” method. In the case of vacuum infiltration of Arabidopsis, intact plants under reduced pressure are treated with an agrobacterial suspension [Bechthold, N (1993). C R Acad Sci Paris Life Sci, 316: 1194-11991, while in the case of the “floral dip” method the developing floral tissue is incubated briefly with a surfactant-treated agrobacterial suspension [Clough, SJ and Bent AF (1998) The Plant J. 16, 735-743]. A certain proportion of transgenic seeds are harvested in both cases, and these seeds can be distinguished from non-transgenic seeds by growing under the above-described selective conditions. In addition the stable transformation of plastids is of advantages because plastids are inherited maternally is most crops reducing or eliminating the risk of transgene flow through pollen. The transformation of the chloroplast genome is generally achieved by a process which has been schematically displayed in Klaus et al., 2004 [Nature Biotechnology 22 (2), 225-229]. Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview is given in Bock (2001) Transgenic plastids in basic research and plant biotechnology. J Mol Biol. 2001 Sep. 21; 312 (3):425-38 or Maliga, P (2003) Progress towards commercialization of plastid transformation technology. Trends Biotechnol. 21, 20-28. Further biotechnological progress has recently been reported in form of marker free plastid transformants, which can be produced by a transient co-integrated maker gene (Klaus et al., 2004, Nature Biotechnology 22(2), 225-229). The genetically modified plant cells can be regenerated via all methods with which the skilled worker is familiar. Suitable methods can be found in the abovementioned publications by S. D. Kung and R. Wu, Potrykus or Höfgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

Preferably, the expression of the nucleic acid in the plant results in the plant's increased resistance to herbicide as compared to a wild type variety of the plant.

In another embodiment, the invention refers to a plant, comprising a plant cell according to the present invention, wherein expression of the nucleic acid in the plant results in the plant's increased resistance to herbicide as compared to a wild type variety of the plant.

The plants described herein can be either transgenic crop plants or non-transgenic plants.

In addition to the general definition, give SUPRA, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either
(a) the nucleic acid sequences encoding proteins useful in the methods of the invention, or
(b) genetic control sequence(s) which is operably linked with the nucleic acid sequence according to the invention, for example a promoter, or
(c) a) and b)

are not located in their natural genetic environment or have been modified by recombinant methods, it being possible for the modification to take the form of, for example, a substitution, addition, deletion, inversion or insertion of one or more nucleotide residues in order to allow for the expression of the mutated PPO of the present invention. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815.

A transgenic plant for the purposes of the invention is thus understood as meaning, as above, that the nucleic acids of the invention are not at their natural locus in the genome of said plant, it being possible for the nucleic acids to be expressed homologously or heterologously. However, as mentioned, transgenic also means that, while the nucleic acids according to the invention or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. Preferred transgenic plants are mentioned herein. Furthermore, the term “transgenic” refers to any plant, plant cell, callus, plant tissue, or plant part, that contains all or part of at least one recombinant polynucleotide. In many cases, all or part of the recombinant polynucleotide is stably integrated into a chromosome or stable extra-chromosomal element, so that it is passed on to successive generations. For the purposes of the invention, the term “recombinant polynucleotide” refers to a polynucleotide that has been altered, rearranged, or modified by genetic engineering. Examples include any cloned polynucleotide, or polynucleotides, that are linked or joined to heterologous sequences. The term “recombinant” does not refer to alterations of polynucleotides that result from naturally occurring events, such as spontaneous mutations, or from non-spontaneous mutagenesis followed by selective breeding.

Plants containing mutations arising due to non-spontaneous mutagenesis and selective breeding are referred to herein as non-transgenic plants and are included in the present invention. In embodiments wherein the plant is transgenic and comprises multiple mutated PPO nucleic acids, the nucleic acids can be derived from different genomes or from the same genome. Alternatively, in embodiments wherein the plant is non-transgenic and comprises multiple mutated PPO nucleic acids, the nucleic acids are located on different genomes or on the same genome.

In certain embodiments, the present invention involves herbidicide-resistant plants that are produced by mutation breeding. Such plants comprise a polynucleotide encoding a mutated PPO and are tolerant to one or more PPO-inhibiting herbicides. Such methods can involve, for example, exposing the plants or seeds to a mutagen, particularly a chemical mutagen such as, for example, ethyl methanesulfonate (EMS) and selecting for plants that have enhanced tolerance to at least one or more PPO-inhibiting herbicide [see Example 1]. However, the present invention is not limited to herbicide-tolerant plants that are produced by a mutagenesis method involving the chemical mutagen EMS. Any mutagenesis method known in the art may be used to produce the herbicide-resistant plants of the present invention. Such mutagenesis methods can involve, for example, the use of any one or more of the following mutagens: radiation, such as X-rays, Gamma rays (e.g., cobalt 60 or cesium 137), neutrons, (e.g., product of nuclear fission by uranium 235 in an atomic reactor), Beta radiation (e.g., emitted from radioisotopes such as phosphorus 32 or carbon 14), and ultraviolet radiation (preferably from 250 to 290 nm), and chemical mutagens such as base analogues (e.g., 5-bromo-uracil), related compounds (e.g., 8-ethoxy caffeine), antibiotics (e.g., streptonigrin), alkylating agents (e.g., sulfur mustards, nitrogen mustards, epoxides, ethylenamines, sulfates, sulfonates, sulfones, lactones), azide, hydroxylamine, nitrous acid, or acridines. Herbicide-resistant plants can also be produced by using tissue culture methods to select for plant cells comprising herbicide-resistance mutations and then regenerating herbicide-resistant plants therefrom. See, for example, U.S. Pat. Nos. 5,773,702 and 5,859,348, both of which are herein incorporated in their entirety by reference. Further details of mutation breeding can be found in “Principals of Cultivar Development” Fehr, 1993 Macmillan Publishing Company the disclosure of which is incorporated herein by reference

Alternatively, herbicide-resistant plants according to the present invention can also be produced by using genome editing methods to select for plant cells comprising herbicide-resistance mutations and then regenerating herbicide-resistant plants therefrom. “Genome Editing” refers to a type of genetic engineering in which DNA is inserted, deleted or replaced in the genome of an organism using engineered nucleases. These nucleases are known to the skilled artisan to create site-specific double-strand breaks at desired locations in the genome. The induced double-strand breaks are repaired through nonhomologous end-joining or homologous recombination, resulting in targeted mutations. Known in the art are currently four families of engineered nucleases which can be used for the purposes of the present invention: meganucleases, zinc finger nucleases (ZFNs), transcription activator-like effector-based nucleases (TALEN), and the CRISPR-Cas system.—For references, see, for example, Esvelt, K M. and Wang, HH. (2013) “Genome-scale engineering for systems and synthetic biology”, Mol Syst Biol. 9 (1): 641; Tan, W S. et al., (2012) “Precision editing of large animal genomes”, Adv Genet. 80: 37-97; Puchta, H. and Fauser, F. (2013) “Gene targeting in plants: 25 years later”, Int. J. Dev. Biol. 57: 629-637; Boglioli, Elsy and Richard, Magali “Rewriting the book of life: a new era in precision genome editing”, Boston Consulting Group, Retrieved Nov. 30, 2015; Method of the Year 2011. Nat Meth 9 (1), 1-1.

The plant of the present invention comprises at least one mutated PPO nucleic acid or over-expressed wild-type PPO nucleic acid, and has increased tolerance to a PPO-inhibiting herbicide as compared to a wild-type variety of the plant. It is possible for the plants of the present invention to have multiple mutated PPO nucleic acids from different genomes since these plants can contain more than one genome. For example, a plant contains two genomes, usually referred to as the A and B genomes. Because PPO is a required metabolic enzyme, it is assumed that each genome has at least one gene coding for the PPO enzyme (i.e. at least one PPO gene). As used herein, the term “PPO gene locus” refers to the position of a PPO gene on a genome, and the terms “PPO gene” and “PPO nucleic acid” refer to a nucleic acid encoding the PPO enzyme. The PPO nucleic acid on each genome differs in its nucleotide sequence from a PPO nucleic acid on another genome. One of skill in the art can determine the genome of origin of each PPO nucleic acid through genetic crossing and/or either sequencing methods or exonuclease digestion methods known to those of skill in the art.

The present invention includes plants comprising one, two, three, or more mutated PPO alleles, wherein the plant has increased tolerance to a PPO-inhibiting herbicide as compared to a wild-type variety of the plant. The mutated PPO alleles can comprise a nucleotide sequence selected from the group consisting of a polynucleotide as defined in SEQ ID NO: 118, 119, 120, 121, 122, 123, 124, 125, 126, 127, 128 or 130, or a variant or derivative thereof, a polynucleotide encoding a polypeptide as defined in SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117 or 129, or a variant or derivative, homologue, orthologue, paralogue thereof, a polynucleotide comprising at least 60 consecutive nucleotides of any of the aforementioned polynucleotides; and a polynucleotide complementary to any of the aforementioned polynucleotides.

“Alleles” or “allelic variants” are alternative forms of a given gene, located at the same chromosomal position. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms

The term “variety” refers to a group of plants within a species defined by the sharing of a common set of characteristics or traits accepted by those skilled in the art as sufficient to distinguish one cultivar or variety from another cultivar or variety. There is no implication in either term that all plants of any given cultivar or variety will be genetically identical at either the whole gene or molecular level or that any given plant will be homozygous at all loci. A cultivar or variety is considered “true breeding” for a particular trait if, when the true-breeding cultivar or variety is self-pollinated, all of the progeny contain the trait. The terms “breeding line” or “line” refer to a group of plants within a cultivar defined by the sharing of a common set of characteristics or traits accepted by those skilled in the art as sufficient to distinguish one breeding line or line from another breeding line or line. There is no implication in either term that all plants of any given breeding line or line will be genetically identical at either the whole gene or molecular level or that any given plant will be homozygous at all loci. A breeding line or line is considered “true breeding” for a particular trait if, when the true-breeding line or breeding line is self-pollinated, all of the progeny contain the trait. In the present invention, the trait arises from a mutation in a PPO gene of the plant or seed.

The herbicide-resistant plants of the invention that comprise polynucleotides encoding mutated PPO polypeptides also find use in methods for increasing the herbicide-resistance of a plant through conventional plant breeding involving sexual reproduction. The methods comprise crossing a first plant that is a herbicide-resistant plant of the invention to a second plant that may or may not be resistant to the same herbicide or herbicides as the first plant or may be resistant to different herbicide or herbicides than the first plant. The second plant can be any plant that is capable of producing viable progeny plants (i.e., seeds) when crossed with the first plant. Typically, but not necessarily, the first and second plants are of the same species. The methods can optionally involve selecting for progeny plants that comprise the mutated PPO polypeptides of the first plant and the herbicide resistance characteristics of the second plant. The progeny plants produced by this method of the present invention have increased resistance to a herbicide when compared to either the first or second plant or both. When the first and second plants are resistant to different herbicides, the progeny plants will have the combined herbicide tolerance characteristics of the first and second plants. The methods of the invention can further involve one or more generations of backcrossing the progeny plants of the first cross to a plant of the same line or genotype as either the first or second plant. Alternatively, the progeny of the first cross or any subsequent cross can be crossed to a third plant that is of a different line or genotype than either the first or second plant.

The present invention also provides plants, plant organs, plant tissues, plant cells, seeds, and non-human host cells that are transformed with the at least one polynucleotide molecule, expression cassette, or transformation vector of the invention. Such transformed plants, plant organs, plant tissues, plant cells, seeds, and non-human host cells have enhanced tolerance or resistance to at least one herbicide, at levels of the herbicide that kill or inhibit the growth of an untransformed plant, plant tissue, plant cell, or non-human host cell, respectively. Preferably, the transformed plants, plant tissues, plant cells, and seeds of the invention are Arabidopsis thaliana and crop plants.

In another embodiment, the invention refers to a seed produced by a transgenic plant comprising a plant cell of the present invention, wherein the seed is true breeding for an increased resistance to a herbicide as compared to a wild type variety of the seed.

In other aspects, herbicide-tolerant plants of the present invention can be employed as herbicide-tolerance trait donor lines for development, as by traditional plant breeding, to produce other varietal and/or hybrid crops containing such trait or traits. All such resulting variety or hybrids crops, containing the ancestral herbicide-tolerance trait or traits can be referred to herein as progeny or descendant of the ancestral, herbicide-tolerant line(s).

In other embodiments, the present invention provides a method for producing a herbicide-tolerant plant. The method comprises: crossing a first herbicide-tolerant plant with a second plant to produce a herbicide-tolerant progeny plant, wherein the first plant and the progeny plant comprise in at least some of their cells a polynucleotide operably linked to a promoter operable in plant cells, the recombinant polynucleotide being effective in the cells of the first plant to express a mutated PPO polypeptide encoded by the polynucleotide, the expression of the mutated PPO polypeptide conferring to the plant tolerance to herbicides.

Traditional plant breeding might be employed whereby the herbicide-tolerant trait is introduced in the progeny plant resulting therefrom. In one embodiment, the present invention provides a method for producing a herbicide-tolerant progeny plant, the method comprising: crossing a parent plant with a herbicide-tolerant plant to introduce the herbicide-tolerance characteristics of the herbicide-tolerant plant into the germplasm of the progeny plant, wherein the progeny plant has increased tolerance to the herbicides relative to the parent plant. In other embodiments, the method further comprises the step of introgressing the herbicide-tolerance characteristics through traditional plant breeding techniques to obtain a descendent plant having the herbicide-tolerance characteristics.

In other aspects, plants of the invention include those plants which, in addition to being tolerant to PPO-inhibiting herbicides, have been subjected to further genetic modifications by breeding, mutagenesis or genetic engineering, e.g. have been rendered tolerant to applications of specific other classes of herbicides, such as AHAS inhibitors; auxinic herbicides; bleaching herbicides such as hydroxyphenylpyruvate dioxygenase (HPPD) inhibitors or phytoene desaturase (PDS) inhibitors; EPSPS inhibitors such as glyphosate; glutamine synthetase (GS) inhibitors such as glufosinate; lipid biosynthesis inhibitors such as acetyl CoA carboxylase (ACCase) inhibitors; or oxynil (i.e. bromoxynil or ioxynil) herbicides as a result of conventional methods of breeding or genetic engineering, Thus, herbicide-tolerant plants of the invention can be made resistant to multiple classes of herbicides through multiple genetic modifications, such as resistance to both glyphosate and glufosinate or to both glyphosate and a herbicide from another class such as HPPD inhibitors, AHAS inhibitors, or ACCase inhibitors. These herbicide resistance technologies are, for example, described in Pest Management Science (at volume, year, page): 61, 2005, 246; 61, 2005, 258; 61, 2005, 277; 61, 2005, 269; 61, 2005, 286; 64, 2008, 326; 64, 2008, 332; Weed Science 57, 2009, 108; Australian Journal of Agricultural Research 58, 2007, 708; Science 316, 2007, 1185; and references quoted therein. For example, herbicide-tolerant plants of the invention, in some embodiments, may be tolerant to ACCase inhibitors, such as “dims” (e.g., cycloxydim, sethoxydim, clethodim, or tepraloxydim), “fops” (e.g., clodinafop, diclofop, fluazifop, haloxyfop, or quizalofop), and “dens” (such as pinoxaden); to auxinic herbicides, such as dicamba; to EPSPS inhibitors, such as glyphosate; to cellulose biosynthesis inhibitors; and to GS inhibitors, such as glufosinate.

In addition to these classes of inhibitors, herbicide-tolerant plants of the invention may also be tolerant to herbicides having other modes of action, for example, chlorophyll/carotenoid pigment inhibitors, cell membrane disrupters, photosynthesis inhibitors, cell division inhibitors, root inhibitors, shoot inhibitors, and combinations thereof.

Such tolerance traits may be expressed, e.g.: as mutant or wildtype HPPD proteins, as mutant or wildtype PPO proteins, as mutant AHASL proteins, mutant ACCase proteins, mutant EPSPS proteins, or mutant glutamine synthetase proteins; or as mutant native, inbred, or transgenic aryloxyalkanoate dioxygenase (AAD or DHT), haloarylnitrilase (BXN), 2,2-dichloropropionic acid dehalogenase (DEH), glyphosate-N-acetyltransferase (GAT), glyphosate decarboxylase (GDC), glyphosate oxidoreductase (GOX), glutathione-S-transferase (GST), phosphinothricin acetyltransferase (PAT or bar), or CYP450s proteins having an herbicide-degrading activity. Herbicide-tolerant plants hereof can also be stacked with other traits including, but not limited to, pesticidal traits such as Bt Cry and other proteins having pesticidal activity toward coleopteran, lepidopteran, nematode, or other pests; nutrition or nutraceutical traits such as modified oil content or oil profile traits, high protein or high amino acid concentration traits, and other trait types known in the art.

Furthermore, in other embodiments, herbicide-tolerant plants are also covered which are, by the use of recombinant DNA techniques and/or by breeding and/or otherwise selected for such characteristics, rendered able to synthesize one or more insecticidal proteins, especially those known from the bacterial genus Bacillus, particularly from Bacillus thuringiensis, such as [delta]-endotoxins, e.g. CryIA(b), CryIA(c), CryIF, CryIF(a2), CryIIA(b), CryIIIA, CryIIIB(bI) or Cry9c; vegetative insecticidal proteins (VIP), e.g. VIP1, VIP2, VIP3 or VIP3A; insecticidal proteins of bacteria colonizing nematodes, e.g. Photorhabdus spp. or Xenorhabdus spp.; toxins produced by animals, such as scorpion toxins, arachnid toxins, wasp toxins, or other insect-specific neurotoxins; toxins produced by fungi, such streptomycete toxins; plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxy-steroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e.g. WO 02/015701). Further examples of such toxins or genetically modified plants capable of synthesizing such toxins are disclosed, e.g., in EP-A 374 753, WO 93/007278, WO 95/34656, EP-A 427 529, EP-A 451 878, WO 03/18810 und WO 03/52073. The methods for producing such genetically modified plants are generally known to the person skilled in the art and are described, e.g. in the publications mentioned above. These insecticidal proteins contained in the genetically modified plants impart to the plants producing these proteins tolerance to harmful pests from all taxonomic groups of arthropods, especially to beetles (Coeloptera), two-winged insects (Diptera), and moths (Lepidoptera) and to nematodes (Nematoda).

In some embodiments, expression of one or more protein toxins (e.g., insecticidal proteins) in the herbicide-tolerant plants is effective for controlling organisms that include, for example, members of the classes and orders: Coleoptera such as the American bean weevil Acanthoscelides obtectus; the leaf beetle Agelastica alni; click beetles (Agriotes lineatus, Agriotes obscurus, Agriotes bicolor); the grain beetle Ahasverus advena; the summer schafer Amphimallon solstitialis; the furniture beetle Anobium punctatum; Anthonomus spp. (weevils); the Pygmy mangold beetle Atomaria linearis; carpet beetles (Anthrenus spp., Attagenus spp.); the cowpea weevil Callosobruchus maculates; the fried fruit beetle Carpophilus hemipterus; the cabbage seedpod weevil Ceutorhynchus assimilis; the rape winter stem weevil Ceutorhynchus picitarsis; the wireworms Conoderus vespertinus and Conoderus falli; the banana weevil Cosmopolites sordidus; the New Zealand grass grub Costelytra zealandica; the June beetle Cotinis nitida; the sunflower stem weevil Cylindrocopturus adspersus; the larder beetle Dermestes lardarius; the corn rootworms Diabrotica virgifera, Diabrotica virgifera virgifera, and Diabrotica barberi; the Mexican bean beetle Epilachna varivestis; the old house borer Hylotropes bajulus; the lucerne weevil Hypera postica; the shiny spider beetle Gibbium psylloides; the cigarette beetle Lasioderma serricorne; the Colorado potato beetle Leptinotarsa decemlineata; Lyctus beetles {Lyctus spp., the pollen beetle Meligethes aeneus; the common cockshafer Melolontha melolontha; the American spider beetle Mezium americanum; the golden spider beetle Niptus hololeuc s; the grain beetles Oryzaephilus surinamensis and Oryzaephilus mercator; the black vine weevil Otiorhynchus sulcatus; the mustard beetle Phaedon cochleariae, the crucifer flea beetle Phyllotreta cruciferae; the striped flea beetle Phyllotreta striolata; the cabbage steam flea beetle Psylliodes chrysocephala; Ptinus spp. (spider beetles); the lesser grain borer Rhizopertha dominica; the pea and been weevil Sitona lineatus; the rice and granary beetles Sitophilus oryzae and Sitophilus granaries; the red sunflower seed weevil Smicronyx fulvus; the drugstore beetle Stegobium paniceum; the yellow mealworm beetle Tenebrio molitor, the flour beetles Tribolium castaneum and Tribolium confusum; warehouse and cabinet beetles {Trogoderma spp.); the sunflower beetle Zygogramma exclamationis; Dermaptera (earwigs) such as the European earwig Forficula auricularia and the striped earwig Labidura riparia; Dictyoptera such as the oriental cockroach Blatta orientalis; the greenhouse millipede Oxidus gracilis; the beet fly Pegomyia betae; the frit fly Oscinella frit; fruit flies (Dacus spp., Drosophila spp.); Isoptera (termites) including species from the familes Hodotermitidae, Kalotermitidae, Mastotermitidae, Rhinotermitidae, Serritermitidae, Termitidae, Termopsidae; the tarnished plant bug Lygus lineolaris; the black bean aphid Aphis fabae; the cotton or melon aphid Aphis gossypii; the green apple aphid Aphis pomi; the citrus spiny whitefly Aleurocanthus spiniferus; the sweet potato whitefly Bemesia tabaci; the cabbage aphid Brevicoryne brassicae; the pear psylla Cacopsylla pyricola; the currant aphid Cryptomyzus ribis; the grape phylloxera Daktulosphaira vitifoliae; the citrus psylla Diaphorina citri; the potato leafhopper Empoasca fabae; the bean leafhopper Empoasca solana; the vine leafhopper Empoasca vitis; the woolly aphid Eriosoma lanigerum; the European fruit scale Eulecanium corni; the mealy plum aphid Hyalopterus arundinis; the small brown planthopper Laodelphax striatellus; the potato aphid Macrosiphum euphorbiae; the green peach aphid Myzus persicae; the green rice leafhopper Nephotettix cinticeps; the brown planthopper Nilaparvata lugens; the hop aphid Phorodon humuli; the bird-cherry aphid Rhopalosiphum padi; the grain aphid Sitobion avenae; Lepidoptera such as Adoxophyes orana (summer fruit tortrix moth); Archips podana (fruit tree tortrix moth); Bucculatrix pyrivorella (pear leafminer); Bucculatrix thurberiella (cotton leaf perforator); Bupalus piniarius (pine looper); Carpocapsa pomonella (codling moth); Chilo suppressalis (striped rice borer); Choristoneura fumiferana (eastern spruce budworm); Cochylis hospes (banded sunflower moth); Diatraea grandiosella (southwestern corn borer); Eupoecilia ambiguella (European grape berry moth); Helicoverpa armigera (cotton bollworm); Helicoverpa zea (cotton bollworm); Heliothis vires cens (tobacco budworm), Homeosoma electellum (sunflower moth); Homona magnanima (oriental tea tree tortrix moth); Lithocolletis blancardella (spotted tentiform leafminer); Lymantria dispar (gypsy moth); Malacosoma neustria (tent caterpillar); Mamestra brassicae (cabbage armyworm); Mamestra configurata (Bertha armyworm); Operophtera brumata (winter moth); Ostrinia nubilalis (European corn borer), Panolis flammea (pine beauty moth), Phyllocnistis citrella (citrus leafminer); Pieris brassicae (cabbage white butterfly); Rachiplusia ni (soybean looper); Spodoptera exigua (beet armywonn); Spodoptera littoralis (cotton leafworm); Sylepta derogata (cotton leaf roller); Trichoplusia ni (cabbage looper); Orthoptera such as the common cricket Acheta domesticus, tree locusts (Anacridium spp.), the migratory locust Locusta migratoria, the twostriped grasshopper Melanoplus bivittatus, the differential grasshopper Melanoplus differ entialis, the redlegged grasshopper Melanoplus femurrubrum, the migratory grasshopper Melanoplus sanguinipes, the northern mole cricket Neocurtilla hexadectyla, the red locust Nomadacris septemfasciata, the shortwinged mole cricket Scapteriscus abbreviatus, the southern mole cricket Scapteriscus borellii, the tawny mole cricket Scapteriscus vicinus, and the desert locust Schistocerca gregaria; Symphyla such as the garden symphylan Scutigerella immaculata; Thysanoptera such as the tobacco thrips Frankliniella fusca, the flower thrips Frankliniella intonsa, the western flower thrips Frankliniella occidentalism the cotton bud thrips Frankliniella schultzei, the banded greenhouse thrips Hercinothrips femoralis, the soybean thrips Neohydatothrips variabilis, Kelly's citrus thrips Pezothrips kellyanus, the avocado thrips Scirtothrips perseae, the melon thrips Thrips palmi, and the onion thrips Thrips tabaci; and the like, and combinations comprising one or more of the foregoing organisms.

In some embodiments, expression of one or more protein toxins (e.g., insecticidal proteins) in the herbicide-tolerant plants is effective for controlling flea beetles, i.e. members of the flea beetle tribe of family Chrysomelidae, preferably against Phyllotreta spp., such as Phyllotreta cruciferae and/or Phyllotreta triolata. In other embodiments, expression of one or more protein toxins (e.g., insecticidal proteins) in the herbicide-tolerant plants is effective for controlling cabbage seedpod weevil, the Bertha armyworm, Lygus bugs, or the diamondback moth.

Furthermore, in one embodiment, herbicide-tolerant plants are also covered which are, e.g. by the use of recombinant DNA techniques and/or by breeding and/or otherwise selected for such traits, rendered able to synthesize one or more proteins to increase the resistance or tolerance of those plants to bacterial, viral or fungal pathogens. The methods for producing such genetically modified plants are generally known to the person skilled in the art.

Furthermore, in another embodiment, herbicide-tolerant plants are also covered which are, e.g. by the use of recombinant DNA techniques and/or by breeding and/or otherwise selected for such traits, rendered able to synthesize one or more proteins to increase the productivity (e.g. oil content), tolerance to drought, salinity or other growth-limiting environmental factors or tolerance to pests and fungal, bacterial or viral pathogens of those plants.

Furthermore, in other embodiments, herbicide-tolerant plants are also covered which are, e.g. by the use of recombinant DNA techniques and/or by breeding and/or otherwise selected for such traits, altered to contain a modified amount of one or more substances or new substances, for example, to improve human or animal nutrition, e.g. oil crops that produce health-promoting long-chain omega-3 fatty acids or unsaturated omega-9 fatty acids (e.g. Nexera® rape, Dow Agro Sciences, Canada).

Furthermore, in some embodiments, herbicide-tolerant plants are also covered which are, e.g. by the use of recombinant DNA techniques and/or by breeding and/or otherwise selected for such traits, altered to contain increased amounts of vitamins and/or minerals, and/or improved profiles of nutraceutical compounds.

In one embodiment, herbicide-tolerant plants of the present invention, relative to a wild-type plant, comprise an increased amount of, or an improved profile of, a compound selected from the group consisting of: glucosinolates (e.g., glucoraphanin (4-methylsulfinylbutyl-glucosinolate), sulforaphane, 3-indolylmethyl-glucosinolate(glucobrassicin), I-methoxy-3-indolylmethyl-glucosinolate (neoglucobrassicin)); phenolics (e.g., flavonoids (e.g., quercetin, kaempferol), hydroxycinnamoyl derivatives (e.g., 1,2,2′-trisinapoylgentiobiose, 1,2-diferuloylgentiobiose, I,2′-disinapoyl-2-feruloylgentiobiose, 3-0-caffeoyl-quinic (neochlorogenic acid)); and vitamins and minerals (e.g., vitamin C, vitamin E, carotene, folic acid, niacin, riboflavin, thiamine, calcium, iron, magnesium, potassium, selenium, and zinc).

In another embodiment, herbicide-tolerant plants of the present invention, relative to a wild-type plant, comprise an increased amount of, or an improved profile of, a compound selected from the group consisting of: progoitrin; isothiocyanates; indoles (products of glucosinolate hydrolysis); glutathione; carotenoids such as beta-carotene, lycopene, and the xanthophyll carotenoids such as lutein and zeaxanthin; phenolics comprising the flavonoids such as the flavonols (e.g. quercetin, rutin), the flavans/tannins (such as the procyanidins comprising coumarin, proanthocyanidins, catechins, and anthocyanins); flavones; phytoestrogens such as coumestans, lignans, resveratrol, isoflavones e.g. genistein, daidzein, and glycitein; resorcyclic acid lactones; organosulphur compounds; phytosterols; terpenoids such as carnosol, rosmarinic acid, glycyrrhizin and saponins; chlorophyll; chlorphyllin, sugars, anthocyanins, and vanilla.

In other embodiments, herbicide-tolerant plants of the present invention, relative to a wild-type plant, comprise an increased amount of, or an improved profile of, a compound selected from the group consisting of: vincristine, vinblastine, taxanes (e.g., taxol (paclitaxel), baccatin III, 10-desacetylbaccatin III, 10-desacetyl taxol, xylosyl taxol, 7-epitaxol, 7-epibaccatin III, 10-desacetylcephalomannine, 7-epicephalomannine, taxotere, cephalomannine, xylosyl cephalomannine, taxagifine, 8-benxoyloxy taxagifine, 9-acetyloxy taxusin, 9-hydroxy taxusin, taiwanxam, taxane Ia, taxane Ib, taxane Ic, taxane Id, GMP paclitaxel, 9-dihydro 13-acetylbaccatin III, 10-desacetyl-7-epitaxol, tetrahydrocannabinol (THC), cannabidiol (CBD), genistein, diadzein, codeine, morphine, quinine, shikonin, ajmalacine, serpentine, and the like.

In other aspects, a method for treating a plant of the present invention is provided.

In some embodiments, the method comprises contacting the plant with an agronomically acceptable composition. In one embodiment, the agronomically acceptable composition comprises an auxinic herbicide A. I.

In another aspect, the present invention provides a method for preparing a descendent seed. The method comprises planting a seed of or capable of producing a plant of the present invention. In one embodiment, the method further comprises growing a descendent plant from the seed; and harvesting a descendant seed from the descendent plant. In other embodiments, the method further comprises applying a herbicides herbicidal composition to the descendent plant.

In another embodiment, the invention refers to harvestable parts of the transgenic plant according to the present invention. Preferably, the harvestable parts comprise the PPO nucleic acid or PPO protein of the present invention. The harvestable parts may be seeds, roots, leaves and/or flowers comprising the PPO nucleic acid or PPO protein or parts thereof. Preferred parts of soy plants are soy beans comprising the PPO nucleic acid or PPO protein.

In another embodiment, the invention refers to products derived from a transgenic plant according to the present invention, parts thereof or harvestable parts thereof. A preferred plant product is fodder, seed meal, oil, or seed-treatment-coated seeds.

Preferably, the meal and/or oil comprise the PPO nucleic acids or PPO proteins.

In another embodiment, the invention refers to a method for the production of a product, which method comprises
a) growing the plants of the invention or obtainable by the methods of invention and
b) producing said product from or by the plants of the invention and/or parts, e.g. seeds, of these plants.

In a further embodiment the method comprises the steps
a) growing the plants of the invention,
b) removing the harvestable parts as defined above from the plants and
c) producing said product from or by the harvestable parts of the invention.

The product may be produced at the site where the plant has been grown, the plants and/or parts thereof may be removed from the site where the plants have been grown to produce the product. Typically, the plant is grown, the desired harvestable parts are removed from the plant, if feasible in repeated cycles, and the product made from the harvestable parts of the plant. The step of growing the plant may be performed only once each time the methods of the invention is performed, while allowing repeated times the steps of product production e.g. by repeated removal of harvestable parts of the plants of the invention and if necessary further processing of these parts to arrive at the product. It is also possible that the step of growing the plants of the invention is repeated and plants or harvestable parts are stored until the production of the product is then performed once for the accumulated plants or plant parts. Also, the steps of growing the plants and producing the product may be performed with an overlap in time, even simultaneously to a large extend or sequentially. Generally the plants are grown for some time before the product is produced.

In one embodiment the products produced by said methods of the invention are plant products such as, but not limited to, a foodstuff, feedstuff, a food supplement, feed supplement, fiber, cosmetic and/or pharmaceutical. Foodstuffs are regarded as compositions used for nutrition and/or for supplementing nutrition. Animal feedstuffs and animal feed supplements, in particular, are regarded as foodstuffs.

In another embodiment the inventive methods for the production are used to make agricultural products such as, but not limited to, plant extracts, proteins, amino acids, carbohydrates, fats, oils, polymers, vitamins, and the like.

It is possible that a plant product consists of one or more agricultural products to a large extent.

### Herbicides

As described above, the present invention provides nucleic acids, polypeptides, conferring tolerance of plants to herbicides.

Generally, the term “herbicide” is used herein to mean an active ingredient that kills, controls or otherwise adversely modifies the growth of plants. The preferred amount or concentration of the herbicide is an “effective amount” or “effective concentration.” By “effective amount” and “effective concentration” is intended an amount and concentration, respectively, that is sufficient to kill or inhibit the growth of a similar, wild-type, plant, plant tissue, plant cell, or host cell, but that said amount does not kill or inhibit as severely the growth of the herbicide-resistant plants, plant tissues, plant cells, and host cells of the present invention. Typically, the effective amount of a herbicide is an amount that is routinely used in agricultural production systems to kill weeds of interest. Such an amount is known to those of ordinary skill in the art. Herbicidal activity is exhibited by herbicides useful for the the present invention when they are applied directly to the plant or to the locus of the plant at any stage of growth or before planting or emergence. The effect observed depends upon the plant species to be controlled, the stage of growth of the plant, the application parameters of dilution and spray drop size, the particle size of solid components, the environmental conditions at the time of use, the specific compound employed, the specific adjuvants and carriers employed, the soil type, and the like, as well as the amount of chemical applied. These and other factors can be adjusted as is known in the art to promote non-selective or selective herbicidal action. Generally, it is preferred to apply the herbicide postemergence to relatively immature undesirable vegetation to achieve the maximum control of weeds.

By a “herbicide-tolerant” or “herbicide-resistant” plant, it is intended that a plant that is tolerant or resistant to at least one herbicide at a level that would normally kill, or inhibit the growth of, a normal or wild-type plant. By “herbicide-tolerant wildtype or mutated PPO protein” or “herbicide-resistant wildtype or mutated PPO protein”, it is intended that such a PPO protein displays higher PPO activity, relative to the PPO activity of a wild-type PPO protein, when in the presence of at least one herbicide that is known to interfere with PPO activity and at a concentration or level of the herbicide that is known to inhibit the PPO activity of the wild-type mutated PPO protein. Furthermore, the PPO activity of such a herbicide-tolerant or herbicide-resistant mutated PPO protein may be referred to herein as “herbicide-tolerant” or “herbicide-resistant” PPO activity

Examples of herbicides which can be used according to the present invention, i.e. to which the plants according to the present invention are tolerant/resistant to, are compounds known to the skilled artisan as PPO inhibiting herbicides. Examples of PPO inhibiting herbicides are described in detail hereinafter.

Generally, if the PPO-inhibiting herbicides (also referred to as compounds A) and/or the herbicidal compounds B as described herein, which can be employed in the context of the present invention, are capable of forming geometrical isomers, for example E/Z isomers, it is possible to use both, the pure isomers and mixtures thereof, in the compositions useful for the present the invention. If the PPO-inhibiting herbicides A and/or the herbicidal compounds B as described herein have one or more centers of chirality and, as a consequence, are present as enantiomers or diastereomers, it is possible to use both, the pure enantiomers and diastereomers and their mixtures, in the compositions according to the invention. If the PPO-inhibiting herbicides A and/or the herbicidal compounds B as described herein have ionizable functional groups, they can also be employed in the form of their agriculturally acceptable salts. Suitable are, in general, the salts of those cations and the acid addition salts of those acids whose cations and anions, respectively, have no adverse effect on the activity of the active compounds. Preferred cations are the ions of the alkali metals, preferably of lithium, sodium and potassium, of the alkaline earth metals, preferably of calcium and magnesium, and of the transition metals, preferably of manganese, copper, zinc and iron, further ammonium and substituted ammonium in which one to four hydrogen atoms are replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl or benzyl, preferably ammonium, methylammonium, isopropylammonium, dimethylammonium, diisopropylammonium, trimethylammonium, heptylammonium, dodecylammonium, tetradecylammonium, tetramethylammonium, tetraethylammonium, tetrabutylammonium, 2-hydroxyethyl-ammonium (olamine salt), 2-(2-hydroxyeth-1-oxy)eth-1-ylammonium (diglycolamine salt), di(2-hydroxyeth-1-yl)ammonium (diolamine salt), tris(2-hydroxyethyl)ammonium (trolamine salt), tris(2-hydroxypropyl)ammonium, benzyltrimethylammonium, benzyltriethylammonium, N,N,N-trimethylethanolammonium (choline salt), furthermore phosphonium ions, sulfonium ions, preferably tri(C₁-C₄-alkyl)sulfonium, such as trimethylsulfonium, and sulfoxonium ions, preferably tri(C₁-C₄-alkyl)sulfoxonium, and finally the salts of polybasic amines such as N,N-bis-(3-aminopropyl)methylamine and diethylenetriamine. Anions of useful acid addition salts are primarily chloride, bromide, fluoride, iodide, hydrogensulfate, methylsulfate, sulfate, dihydrogenphosphate, hydrogenphosphate, nitrate, bicarbonate, carbonate, hexafluorosilicate, hexafluorophosphate, benzoate and also the anions of C₁-C₄-alkanoic acids, preferably formate, acetate, propionate and butyrate.

The PPO-inhibiting herbicides A and/or the herbicidal compounds B as described herein having a carboxyl group can be employed in the form of the acid, in the form of an agriculturally suitable salt as mentioned above or else in the form of an agriculturally acceptable derivative, for example as amides, such as mono- and di-C₁-C₆-alkylamides or arylamides, as esters, for example as allyl esters, propargyl esters, C₁-C₁₀-alkyl esters, alkoxyalkyl esters, tefuryl ((tetrahydrofuran-2-yl)methyl) esters and also as thioesters, for example as C₁-C₁₀-alkylthio esters. Preferred mono- and di-C₁-C₆-alkylamides are the methyl and the dimethylamides. Preferred arylamides are, for example, the anilides and the 2-chloroanilides. Preferred alkyl esters are, for example, the methyl, ethyl, propyl, isopropyl, butyl, isobutyl, pentyl, mexyl (1-methylhexyl), meptyl (1-methylheptyl), heptyl, octyl or isooctyl (2-ethylhexyl) esters. Preferred C₁-C₄-alkoxy-C₁-C₄-alkyl esters are the straight-chain or branched C₁-C₄-alkoxy ethyl esters, for example the 2-methoxyethyl, 2-ethoxyethyl, 2-butoxyethyl (butotyl), 2-butoxypropyl or 3-butoxypropyl ester. An example of a straight-chain or branched C₁-C₁₀-alkylthio ester is the ethylthio ester.

Examples of PPO inhibiting herbicides which can be used according to the present invention are acifluorfen, acifluorfen-sodium, aclonifen, azafenidin, bencarbazone, benzfendizone, bifenox, butafenacil, carfentrazone, carfentrazone-ethyl, chlomethoxyfen, cinidon-ethyl, fluazolate, flufenpyr, flufenpyr-ethyl, flumiclorac, flumiclorac-pentyl, flumioxazin, fluoroglycofen, fluoroglycofen-ethyl, fluthiacet, fluthiacet-methyl, fomesafen, halosafen, lactofen, oxadiargyl, oxadiazon, oxyfluorfen, pentoxazone, profluazol, pyraclonil, pyraflufen, pyraflufen-ethyl, saflufenacil, sulfentrazone, thidiazimin, tiafenacil, chlornitrofen, flumipropyn, fluoronitrofen, flupropacil, furyloxyfen, nitrofluorfen, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), N-ethyl-3-2,6-dichloro-4-trifluoro-methylphenoxy)-5-methyl-1H-pyrazole-1-carboxamide (CAS 452098-92-9), N-tetrahydrofurfuryl-3-(2,6-dichloro-4-trifluoromethylphenoxy)-5-methyl-1H-pyrazole-1-carboxamide (CAS 915396-43-9), N-ethyl-3-(2-chloro-6-fluoro-4-trifluoromethylphenoxy)-5-methyl-1H-pyrazole-1-carboxamide (CAS 452099-05-7), N-tetrahydrofurfuryl-3-(2-chloro-6-fluoro-4-trifluoromethylphenoxy)-5-methyl-1H-pyrazole-1-carboxamide (CAS 452100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione (CAS 451484-50-7), 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), 2-(2,2,7-Trifluoro-3-oxo-4-prop-2-ynyl-3,4-dihydro-2H-benzo[1,4]oxazin-6-yl)-4,5,6,7-tetrahydro-isoindole-1,3-dione (CAS 1300118-96-0), 1-Methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione, methyl (E)-4-[2-chloro-5-[4-chloro-5-(difluoromethoxy)-1H-methyl-pyrazol-3-yl]-4-fluoro-phenoxy]-3-methoxy-but-2-enoate [CAS 948893-00-3], 3-[7-Chloro-5-fluoro-2-(trifluoromethyl)-1H-benzimidazol-4-yl]-1-methyl-6-(trifluoromethyl)-1H-pyrimidine-2,4-dione (CAS 212754-02-4), and uracils of formula III

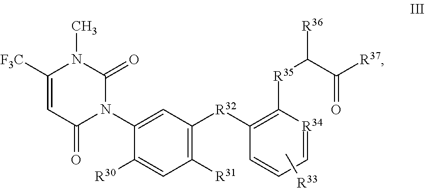

wherein
R³⁰ and R³¹ independently of one another are F, CI or CN;
R³² is O or S;
R³³ is H, F, CI, CH₃ or OCH₃;
R³⁴ is CH or N;
R³⁵ is O or S;
R³⁶ is H, CN, CH₃, CF₃, OCH₃, OC₂H5, SCH₃, SC₂H5, (CO)OC₂H₅ or CH₂R³⁸, wherein R³⁸ is F, Cl, OCH₃, SCH₃, SC₂H5, CH₂F, CH₂Br or CH₂OH;
and
R³⁷ is (C₁-C₆-alkyl)amino, (C₁-C₆-dialkyl)amino, (NH)OR³⁹, OH, OR⁴⁰ or SR⁴⁰ wherein R³¹ is CH₃, C₂H₅ or phenyl; and
   R⁴⁰ is independently of one another C₁-C₆-alkyl, C₂-C₆-alkenyl, C₃-C₆-alkynyl, C₁-C₆-haloalkyl, C₁-C₆-alkoxy-C₁-C₆-alkyl, C₁-C₆-alkoxy-C₁-C₆-alkoxy-C₁-C₆-alkyl, C₂-C₆-cyanoalkyl, C₁-C₄-alkoxy-carbonyl-C₁-C₄-alkyl, C₁-C₄-alkyl-carbonyl-amino, C₁-C₆-alkylsulfinyl-C₁-C₆-alkyl, C₁-C₆-alkyl-sulfonyl-C₁-C₆-alkyl, C₁-C₆-dialkoxy-C₁-C₆-alkyl, C₁-C₆-alkyl-carbonyloxy-C₁-C₆-alkyl, phenyl-carbonyl-C₁-C₆-alkyl, tri(C₁-C₃-alkyl)-silyl-C₁-C₆-alkyl, tri(C₁-C₃-alkyl)-silyl-C₁-C₆-alkenyl, tri(C₁-C₃-alkyl)-silyl-C₁-C₆-alkynyl, tri(C₁-C₃-alkyl)-silyl-C₁-C₆-alkoxy-C₁-C₆-alkyl, dimethylamino, tetrahydropyranyl, tetra-hydrofuranyl-C₁-C₃-alkyl, phenyl-C₁-C₆-alkoxy-C₁-C₆-alkyl, phenyl-C₁-C₃-alkyl, pyridyl-C₁-C₃-alkyl, pyridyl, phenyl,
      which pyridyls and phenyls independently of one another are substituted by one to five substituents selected from the group consisting of halogen, C₁-C₃-alkyl or C₁-C₂-haloalkyl;
   C₃-C₆-cycloalkyl or C₃-C₆-cycloalkyl-C₁-C₄-alkyl,
      which cycloalkyls independently of one another are unsubstituted or substituted by one to five substituents selected from the group consisting of halogen, C₁-C₃-alkyl and C₁-C₂-haloalkyl;
including their agriculturally acceptable alkali metal salts or ammonium salts.

Preferred PPO-inhibiting herbicides that can be used according to the present invention are:

Acifluorfen, acifluorfen-sodium, azafenidin, bencarbazone, benzfendizone, butafenacil, carfentrazone-ethyl, cinidon-ethyl, flufenpyr-ethyl, flumiclorac-pentyl, flumioxazin, fluoroglycofen-ethyl, fluthiacet-methyl, fomesafen, lactofen, oxadiargyl, oxadiazon, oxyfluorfen, pentoxazone, pyraflufen-ethyl, saflufenacil, sulfentrazone, tiafenacil, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), N-ethyl-3-(2,6-dichloro-4-trifluoromethylphenoxy)-5-methyl-1H-pyrazole-1-carboxamide (CAS 452098-92-9), N-tetrahydrofurfuryl-3-(2,6-dichloro-4-trifluoromethylphenoxy)-5-methyl-1H-pyrazole-1-carboxamide (CAS 915396-43-9), N-ethyl-3-(2-chloro-6-fluoro-4-trifluoromethylphenoxy)-5-methyl-1H-pyrazole-1-carboxamide (CAS 452099-05-7), N-tetrahydrofurfuryl-3-(2-chloro-6-fluoro-4-trifluoromethylphenoxy)-5-methyl-1H-pyrazole-1-carboxamide (CAS 452100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione (CAS 451484-50-7), 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), 2-(2,2,7-Trifluoro-3-oxo-4-prop-2-ynyl-3,4-dihydro-2H-benzo[1,4]oxazin-6-yl)-4,5,6,7-tetrahydro-isoindole-1,3-dione (CAS 1300118-96-0); 1-Methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione (CAS 1304113-05-0), 3-[7-Chloro-5-fluoro-2-(trifluoromethyl)-1H-benzimidazol-4-yl]-1-methyl-6-(trifluoromethyl)-1H-pyrimidine-2,4-dione (CAS 212754-02-4)

uracils of formula III.1 (corresponding to uracils of formula III, wherein R³⁰ is F, R³¹ is Cl, R³² is O; R³³ is H; R³⁴ is CH; R³⁵ is O and R³⁷ is OR⁴⁰)

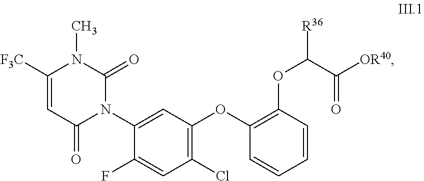

wherein
R³⁶ is OCH₃, OC₂H, SCH₃ or SC₂H₅;
and
R⁴⁰ is C₁-C₆-alkyl, C₂-C₆-alkenyl, C₃-C₆-alkynyl, C₁-C₆-haloalkyl, C₁-C₆-alkoxy-C₁-C₆-alkyl, C₁-C₆-alkoxy-C₁-C₆-alkoxy-C₁-C₆-alkyl, C₁-C₃-cyanoalkyl, phenyl-C₁-C₃-alkyl, pyridyl-C₁-C₃-alkyl, C₃-C₆-cycloalkyl or C₃-C₆-cycloalkyl-C₁-C₄-alkyl,
   which cycloalkyls are unsubstituted or substituted by one to five substituents selected from the group consisting of halogen, C₁-C₃-alkyl and C₁-C₂-haloalkyl;

and

uracils of formula III.2 (corresponding to uracils of formula III, wherein R³⁰ is F; R³¹ is Cl; R³² is O; R³³ is H; R³⁴ is N; R³⁵ is O and R³¹ is OR⁴⁰ with R⁴⁰ is C₁-C₆-alkyl)

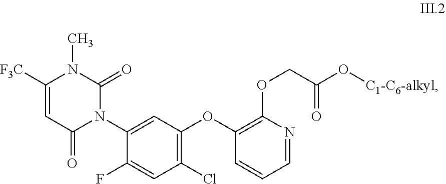

Particularly preferred PPO-inhibiting herbicides that can be used according to the present invention are:

acifluorfen, acifluorfen-sodium, butafenacil, carfentrazone-ethyl, cinidon-ethyl, flumioxazin, fluthiacet-methyl, fomesafen, lactofen, oxadiargyl, oxyfluorfen, saflufenacil, sulfentrazone, tiafenacil, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione (CAS 451484-50-7), 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), and 2-(2,2,7-Trifluoro-3-oxo-4-prop-2-ynyl-3,4-dihydro-2H-benzo[1,4]oxazin-6-yl)-4,5,6,7-tetrahydro-isoindole-1,3-dione (CAS 1300118-96-0), 1-Methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione (CAS 1304113-05-0),

uracils of formula III.1.1 (corresponding to uracils of formula III, wherein R³⁰ is F, R³¹ is Cl, R³² is O; R³³ is H; R³⁴ is CH; R³⁵ is O, R³⁶ is OCH₃ and R³¹ is OR⁴⁰)

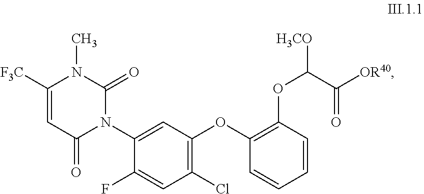

wherein
R⁴⁰ is C₁-C₆-alkyl, C₂-C₆-alkenyl, C₃-C₆-alkynyl, C₁-C₆-haloalkyl, C₁-C₆-alkoxy-C₁-C₆-alkyl, C₁-C₆-alkoxy-C₁-C₆-alkoxy-C₁-C₆-alkyl, C₁-C₃-cyanoalkyl, phenyl-C₁-C₃-alkyl, pyridyl-C₁-C₃-alkyl, C₃-C₆-cycloalkyl or C₃-C₆-cycloalkyl-C₁-C₄-alkyl,
   which cycloalkyls are unsubstituted or substituted by one to five substituents selected from the group consisting of halogen, C₁-C₃-alkyl and C₁-C₂-haloalkyl;
is preferably CH₃, CH₂CH₂OC₂H₅, CH₂CHF₂, cyclohexyl, (1-methylcyclopropyl)methyl or CH₂(pyridine-4-yl);

uracils of formula III.2.1 (corresponding to uracils of formula III, wherein R³⁰ is F; R³¹ is Cl; R³² is O; R³³ is H; R³⁴ is N; R³⁵ is O and R³¹ is OR⁴⁰ with R⁴⁰ is CH₃)

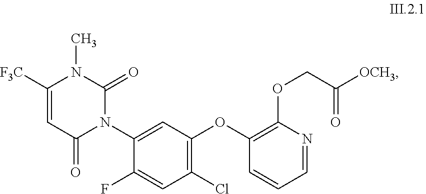 [SMILES: COC(=O)COc1ncccc1Oc1cc(-n2c(=O)cc(C(F)(F)F)n(C)c2=O)c(F)cc1Cl]

and

uracils of formula III.2.2 (corresponding to uracils of formula III, wherein R³⁰ is F; R³¹ is Cl; R³² is O; R³³ is H; R³⁴ is N; R³⁵ is O and R³⁷ is OR⁴⁰ with R⁴⁰ is C₂H₅)

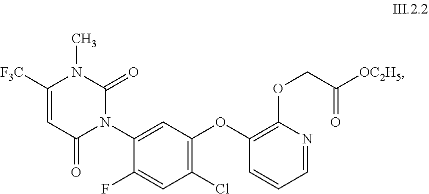 [SMILES: CCOC(=O)COc1ncccc1Oc1cc(-n2c(=O)cc(C(F)(F)F)n(C)c2=O)c(F)cc1Cl]

Especially preferred PPO-inhibiting herbicides are the PPO-inhibiting herbicides.1 to A.14 listed below in table A:

TABLE A

| | |
|---|---|
| B.1 | acifluorfen |
| B.2 | butafenacil |
| B.3 | carfentrazone-ethyl |
| B.4 | cinidon-ethyl |
| B.5 | flumioxazin |
| B.6 | fluthiacet-methyl |
| B.7 | fomesafen |
| B.8 | lactofen |
| B.9 | oxadiargyl |
| B.10 | oxyfluorfen |
| B.11 | saflufenacil |
| B.12 | sulfentrazone |
| B.13 | ethyl [3-[2-chloro-4-fluoro-5-(1-methyl-6-trifluoromethyl- |
| | 2,4-dioxo-1,2,3,4-tetrahydropyrimidin-3-yl)phenoxy]-2- |
| | pyridyloxy]acetate (CAS 353292-31-6) |
| B.14 | 1,5-dimethyl-6-thioxo-3-(2,2,7-trifluoro-3-oxo-4-(prop-2-ynyl)- |
| | 3,4-dihydro-2H-benzo[b][1,4]oxazin-6-yl)-1,3,5- |
| | triazinane-2,4-dione (CAS 1258836-72-4/trifludimoxazin) |
| B.15 | Tiafenacil |

Other preferred PPO-inhibiting herbicides include those disclosed in WO2016/120116, the content of which is herein incorporated by reference in its entirety.

Other preferred PPO-inhibiting herbicide include those disclosed in WO2017/202768, the content of which is herein incorporated by reference in its entirety.

More specifically, said PPO-inhibiting herbicide disclosed in WO2017/202768 refer to a a uracilpyridine of formula (I)

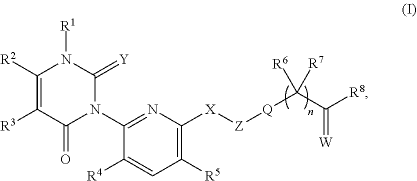

wherein the substituents have the following meanings:
R¹ hydrogen, NH₂, C₁-C₆-alkyl or C₃-C₆-alkynyl;
R² hydrogen, C₁-C₆-alkyl or C₁-C₆-haloalkyl;
R³ hydrogen or C₁-C₆-alkyl;
R⁴ H or halogen;
R⁵ halogen, CN, NO₂, NH₂, CF₃ or C(═S)NH₂;
R⁶ H, halogen, CN, C₁-C₃-alkyl, C₁-C₃-haloalkyl, C₁-C₃-alkoxy, C₁-C₃-haloalkoxy, C₁-C₃-alkylthio, (C₁-C₃-alkyl)amino, di(C₁-C₃-alkyl)amino, C₁-C₃-alkoxy-C₁-C₃-alkyl, C₁-C₃-alkoxycarbonyl;
R⁷ H, halogen, C₁-C₃-alkyl, C₁-C₃-alkoxy;
R⁸ OR⁹, SR⁹, NR¹⁰R¹¹, NR⁹OR⁹, NR⁹S(O)₂R¹⁰ or NR⁹S(O)₂NR¹⁰R¹¹, wherein
   R⁹ is hydrogen, C₁-C₆-alkyl, C₃-C₆-alkenyl, C₃-C₆-alkynyl, C₁-C₆-haloalkyl, C₃-C₆-haloalkenyl, C₃-C₆-haloalkynyl, C₁-C₆-cyanoalkyl, C₁-C₆-alkoxy-C₁-C₆-alkyl, C₁-C₆-alkoxy-C₁-C₆-alkoxy-C₁-C₆-alkyl, di(C₁-C₆-alkoxy)C₁-C₆-alkyl, C₁-C₆-haloalkoxy-C₁-C₆-alkyl, C₃-C₆-alkenyloxy-C₁-C₆-alkyl, C₃-C₆-haloalkenyloxy-C₁-C₆-alkyl, C₃-C₆-alkenyloxy-C₁-C₆-alkoxy-C₁-C₆-alkyl, C₁-C₆-alkylthio-C₁-C₆-alkyl, C₁-C₆-alkylsulfinyl-C₁-C₆-alkyl, C₁-C₆-alkylsulfonyl-C₁-C₆-alkyl, C₁-C₆-alkylcarbonyl-C₁-C₆-alkyl, C₁-C₆-alkoxycarbonyl-C₁-C₆-alkyl, C₁-C₆-haloalkoxycarbonyl-C₁-C₆-alkyl, C₃-C₆-alkenyloxycarbonyl-C₁-C₆-alkyl, C₃-C₆-alkynyloxycarbonyl-C₁-C₆-alkyl, amino, (C₁-C₆-alkyl)amino, di(C₁-C₆-alkyl)amino, (C₁-C₆-alkylcarbonyl)amino, amino-C₁-C₆-alkyl, (C₁-C₆-alkyl)amino-C₁-C₆-alkyl, di(C₁-C₆-alkyl)amino-C₁-C₆-alkyl, aminocarbonyl-C₁-C₆-alkyl, (C₁-C₆-alkyl)aminocarbonyl-C₁-C₆-alkyl, di(C₁-C₆-alkyl)aminocarbonyl-C₁-C₆-alkyl,
      —N═CR¹²R¹³, wherein R¹² and R¹³ independently of one another are H, C₁-C₄-alkyl or phenyl;
      C₃-C₆-cycloalkyl, C₃-C₆-cycloalkyl-C₁-C₆-alkyl, C₃-C₆-heterocyclyl, C₃-C₆-heterocyclyl-C₁-C₆-alkyl, phenyl, phenyl-C₁-C₄-alkyl or a 5- or 6 membered heteroaryl,
         wherein each cycloalkyl, heterocyclyl, phenyl or heteroaryl ring can be substituted by one to four substituents selected from R¹⁴ or a 3- to 7-membered carbocyclus,
         which carbocyclus optionally has in addition to carbon atoms one or two ring members selected from the group consisting of
         —N(R¹²)—, —N═N—, —C(═O)—, —O— and —S—, and which carbocyclus is optionally substituted with one to four substituents selected from R¹⁴;
         wherein R¹⁴ is halogen, NO₂, CN, C₁-C₄-alkyl, C₁-C₄-haloalkyl, C₁-C₄-alkoxy or C₁-C₄-alkoxy-carbonyl;
   R¹⁰, R¹¹ independently of one another are R⁹, or together form a 3- to 7-membered carbocyclus,
      which carbocyclus optionally has in addition to carbon atoms one or two ring members selected from the group consisting of —N(R¹²)—, —N═N—, —C(═O)—, —O— and —S—, and
      which carbocyclus is optionally substituted with one to four substituents selected from R¹⁴;
n 1 to 3;
Q CH₂, O, S, SO, SO₂, NH or (C₁-C₃-alkyl)N;
W O or S;
X NH, NCH₃, O or S;
Y O or S;
Z phenyl, pyridyl, pyridazinyl, pyrimidinyl or pyrazinyl,
   each of which is optionally substituted by 1 to 4 substituents selected from the group consisting of halogen, CN, C₁-C₆-alkyl, C₁-C₆-haloalkyl, C₁-C₆-alkoxy, C₁-C₆-haloalkoxy;
including their agriculturally acceptable salts or derivatives, provided the compounds of formula (I) have a carboxyl group.

The PPO-inhibiting herbicides described above that are useful to carry out the present invention are often best applied in conjunction with one or more other herbicides to obtain control of a wider variety of undesirable vegetation. For example, PPO-inhibiting herbicides may further be used in conjunction with additional herbicides to which the crop plant is naturally tolerant, or to which it is resistant via expression of one or more additional transgenes as mentioned supra, or to which it is resistant via mutagenesis and breeding methods as described. When used in conjunction with other targeting herbicides, the PPO-inhibiting herbicides, to which the plant of the present invention had been made resistant or tolerant, can be formulated with the other herbicide or herbicides, tank mixed with the other herbicide or herbicides, or applied sequentially with the other herbicide or herbicides.

Suitable components for mixtures are, for example, selected from the herbicides of class b1) to b15)
B) herbicides of class b1) to b15):
   b1) lipid biosynthesis inhibitors;
   b2) acetolactate synthase inhibitors (ALS inhibitors);
   b3) photosynthesis inhibitors;
   b4) protoporphyrinogen-IX oxidase inhibitors,
   b5) bleacher herbicides;
   b6) enolpyruvyl shikimate 3-phosphate synthase inhibitors (EPSP inhibitors);
   b7) glutamine synthetase inhibitors;
   b8) 7,8-dihydropteroate synthase inhibitors (DHP inhibitors);
   b9) mitosis inhibitors;
   b10) inhibitors of the synthesis of very long chain fatty acids (VLCFA inhibitors);
   b11) cellulose biosynthesis inhibitors;
   b12) decoupler herbicides;
   b13) auxinic herbicides;
   b14) auxin transport inhibitors; and
   b15) other herbicides selected from the group consisting of bromobutide, chlorflurenol, chlorflurenol-methyl, cinmethylin, cumyluron, dalapon, dazomet, difenzoquat, difenzoquat-metilsulfate, dimethipin, DSMA, dymron, endothal and its salts, etobenzanid, flamprop, flamprop-isopropyl, flamprop-methyl, flamprop-M-isopropyl, flamprop-M-methyl, flurenol, flurenol-butyl, flurprimidol, fosamine, fosamine-ammonium, indanofan, indaziflam, maleic hydrazide, mefluidide, metam, methiozolin (CAS 403640-27-7), methyl azide, methyl bromide, methyl-dymron, methyl iodide, MSMA, oleic acid, oxaziclomefone, pelargonic acid, pyributicarb, quinoclamine, triaziflam, tridiphane and 6-chloro-3-(2-cyclopropyl-6-methylphenoxy)-4-pyridazinol (CAS 499223-49-3) and its salts and esters;

including their agriculturally acceptable salts or derivatives.

Examples of herbicides B which can be used in combination with the PPO-inhibiting herbicides according to the present invention are:
b1) from the group of the lipid biosynthesis inhibitors:

ACC-herbicides such as alloxydim, alloxydim-sodium, butroxydim, clethodim, clodinafop, clodinafop-propargyl, cycloxydim, cyhalofop, cyhalofop-butyl, diclofop, diclofop-methyl, fenoxaprop, fenoxaprop-ethyl, fenoxaprop-P, fenoxaprop-P-ethyl, fluazifop, fluazifop-butyl, fluazifop-P, fluazifop-P-butyl, haloxyfop, haloxyfop-methyl, haloxyfop-P, haloxyfop-P-methyl, metamifop, pinoxaden, profoxydim, propaquizafop, quizalofop, quizalofop-ethyl, quizalofop-tefuryl, quizalofop-P, quizalofop-P-ethyl, quizalofop-P-tefuryl, sethoxydim, tepraloxydim, tralkoxydim, 4-(4′-Chloro-4-cyclopropyl-2′-fluoro[1,1′-biphenyl]-3-yl)-5-hydroxy-2,2,6,6-tetramethyl-2H-pyran-3(6H)-one (CAS 1312337-72-6); 4-(2′,4′-Dichloro-4-cyclopropyl[1,1′-biphenyl]-3-yl)-5-hydroxy-2,2,6,6-tetramethyl-2H-pyran-3(6H)-one (CAS 1312337-45-3); 4-(4′-Chloro-4-ethyl-2′-fluoro[1,1′-biphenyl]-3-yl)-5-hydroxy-2,2,6,6-tetramethyl-2H-pyran-3(6H)-one (CAS 1033757-93-5); 4-(2′,4′-Dichloro-4-ethyl[1,1′-biphenyl]-3-yl)-2,2,6,6-tetramethyl-2H-pyran-3,5(4H,6H)-dione (CAS 1312340-84-3); 5-(Acetyloxy)-4-(4′-chloro-4-cyclopropyl-2′-fluoro[1,1′-biphenyl]-3-yl)-3,6-dihydro-2,2,6,6-tetramethyl-2H-pyran-3-one (CAS 1312337-48-6); 5-(Acetyloxy)-4-(2′,4′-dichloro-4-cyclopropyl-[1,1′-biphenyl]-3-yl)-3,6-dihydro-2,2,6,6-tetramethyl-2H-pyran-3-one; 5-(Acetyloxy)-4-(4′-chloro-4-ethyl-2′-fluoro[1,1′-biphenyl]-3-yl)-3,6-dihydro-2,2,6,6-tetramethyl-2H-pyran-3-one (CAS 1312340-82-1); 5-(Acetyloxy)-4-(2′,4′-dichloro-4-ethyl[1,1′-biphenyl]-3-yl)-3,6-dihydro-2,2,6,6-tetramethyl-2H-pyran-3-one (CAS 1033760-55-2); 4-(4′-Chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester (CAS 1312337-51-1); 4-(2′,4′-Dichloro-4-cyclopropyl-[1,1′-biphenyl]-3-yl)-5,6-dihydro-2,2,6,6-tetramethyl-5-oxo-2H-pyran-3-yl carbonic acid methyl ester; 4-(4′-Chloro-4-ethyl-2′-fluoro[1,1′-biphenyl]-3-yl)-5,6-dihydro-2,2,6,6-tetramethyl-5-oxo-2H-pyran-3-yl carbonic acid methyl ester (CAS 1312340-83-2); 4-(2′,4′-Dichloro-4-ethyl[1,1′-biphenyl]-3-yl)-5,6-dihydro-2,2,6,6-tetramethyl-5-oxo-2H-pyran-3-yl carbonic acid methyl ester (CAS 1033760-58-5); and non ACC herbicides such as benfuresate, butylate, cycloate, dalapon, dimepiperate, EPTC, esprocarb, ethofumesate, flupropanate, molinate, orbencarb, pebulate, prosulfocarb, TCA, thiobencarb, tiocarbazil, triallate and vernolate;
b2) from the group of the ALS inhibitors:

sulfonylureas such as amidosulfuron, azimsulfuron, bensulfuron, bensulfuron-methyl, chlorimuron, chlorimuron-ethyl, chlorsulfuron, cinosulfuron, cyclosulfamuron, ethametsulfuron, ethametsulfuron-methyl, ethoxysulfuron, flazasulfuron, flucetosulfuron, flupyrsulfuron, flupyrsulfuron-methyl-sodium, foramsulfuron, halosulfuron, halosulfuron-methyl, imazosulfuron, iodosulfuron, iodosulfuron-methyl-sodium, iofensulfuron, iofensulfuron-sodium, mesosulfuron, metazosulfuron, metsulfuron, metsulfuron-methyl, nicosulfuron, orthosulfamuron, oxasulfuron, primisulfuron, primisulfuron-methyl, propyrisulfuron, prosulfuron, pyrazosulfuron, pyrazosulfuron-ethyl, rimsulfuron, sulfometuron, sulfometuron-methyl, sulfosulfuron, thifensulfuron, thifensulfuron-methyl, triasulfuron, tribenuron, tribenuron-methyl, trifloxysulfuron, triflusulfuron, triflusulfuron-methyl and tritosulfuron,

imidazolinones such as imazamethabenz, imazamethabenz-methyl, imazamox, imazapic, imazapyr, imazaquin and imazethapyr, triazolopyrimidine herbicides and sulfonanilides such as cloransulam, cloransulam-methyl, diclosulam, flumetsulam, florasulam, metosulam, penoxsulam, pyrimisulfan and pyroxsulam,

pyrimidinylbenzoates such as bispyribac, bispyribac-sodium, pyribenzoxim, pyriftalid, pyriminobac, pyriminobac-methyl, pyrithiobac, pyrithiobac-sodium, 4-[[[2-[(4,6-dimethoxy-2-pyrimidinyl)oxy]phenyl]methyl]amino]-benzoic acid-1-methylethyl ester (CAS 420138-41-6), 4-[[[2-[(4,6-dimethoxy-2-pyrimidinyl)oxy]phenyl]methyl]amino]-benzoic acid propyl ester (CAS 420138-40-5), N-(4-bromophenyl)-2-[(4,6-dimethoxy-2-pyrimidinyl)oxy]benzenemethanamine (CAS 420138-01-8),

sulfonylaminocarbonyl-triazolinone herbicides such as flucarbazone, flucarbazone-sodium, propoxycarbazone, propoxycarbazone-sodium, thiencarbazone and thiencarbazone-methy|; and triafamone;

among these, a preferred embodiment of the invention relates to those compositions comprising at least one imidazolinone herbicide;
b3) from the group of the photosynthesis inhibitors:

amicarbazone, inhibitors of the photosystem II, e.g. triazine herbicides, including of chlorotriazine, triazinones, triazindiones, methylthiotriazines and pyridazinones such as ametryn, atrazine, chloridazone, cyanazine, desmetryn, dimethametryn, hexazinone, metribuzin, prometon, prometryn, propazine, simazine, simetryn, terbumeton, terbuthylazin, terbutryn and trietazin, aryl urea such as chlorobromuron, chlorotoluron, chloroxuron, dimefuron, diuron, fluometuron, isoproturon, isouron, linuron, metamitron, methabenzthiazuron, metobenzuron, metoxuron, monolinuron, neburon, siduron, tebuthiuron and thiadiazuron, phenyl carbamates such as desmedipham, karbutilat, phenmedipham, phenmedipham-ethyl, nitrile herbicides such as bromofenoxim, bromoxynil and its salts and esters, ioxynil and its salts and esters, uraciles such as bromacil, lenacil and terbacil, and bentazon and bentazon-sodium, pyridate, pyridafol, pentanochlor and propanil and inhibitors of the photosystem I such as diquat, diquat-dibromide, paraquat, paraquat-dichloride and paraquat-dimetilsulfate. Among these, a preferred embodiment of the invention relates to those compositions comprising at least one aryl urea herbicide. Among these, likewise a preferred embodiment of the invention relates to those compositions comprising at least one triazine herbicide. Among these, likewise a preferred embodiment of the invention relates to those compositions comprising at least one nitrile herbicide;
b4) from the group of the protoporphyrinogen-IX oxidase inhibitors:

acifluorfen, acifluorfen-sodium, azafenidin, bencarbazone, benzfendizone, bifenox, butafenacil, carfentrazone, carfentrazone-ethyl, chlomethoxyfen, cinidon-ethyl, fluazolate, flufenpyr, flufenpyr-ethyl, flumiclorac, flumiclorac-pentyl, flumioxazin, fluoroglycofen, fluoroglycofen-ethyl, fluthiacet, fluthiacet-methyl, fomesafen, halosafen, lactofen, oxadiargyl, oxadiazon, oxyfluorfen, pentoxazone, profluazol, pyraclonil, pyraflufen, pyraflufen-ethyl, saflufenacil, sulfentrazone, thidiazimin, tiafenacil, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100, N-ethyl-3-(2,6-dichloro-4-trifluoro-methylphenoxy)-5-methyl-1H-pyrazole-1-carboxamide (CAS 452098-92-9), N-tetrahydrofurfuryl-3-(2,6-dichloro-4-trifluoromethylphenoxy)-5-methyl-1H-pyrazole-1-carboxamide (CAS 915396-43-9), N-ethyl-3-(2-chloro-6-fluoro-4-trifluoromethylphenoxy)-5-methyl-1H-pyrazole-1-carboxamide (CAS 452099-05-7), N-tetrahydrofurfuryl-3-(2-chloro-6-fluoro-4-trifluoromethylphenoxy)-5-methyl-1H-pyrazole-1-carboxamide (CAS 452100-03-7), 3-[7-fluoro-3-oxo-4-(prop-2-ynyvl-3,4-dihydro-2H-benzo[1,4]oxazin-6-yl]-1,5-dimethyl-6-thioxo-[1,3,5]triazinan-2,4-dione, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), 2-(2,2,7-Trifluoro-3-oxo-4-prop-2-ynyl-3,4-dihydro-2H-benzo[1,4]oxazin-6-yl)-4,5,6,7-tetrahydro-isoindole-1,3-dione, 1-Methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione (CAS 1304113-05-0), methyl (E)-4-[2-chloro-5-[4-chloro-5-(difluoromethoxy)-1H-methyl-pyrazol-3-yl]-4-fluoro-phenoxy]-3-methoxy-but-2-enoate [CAS 948893-00-3], and 3-[7-Chloro-5-fluoro-2-(trifluoromethyl)-1H-benzimidazol-4-yl]-1-methyl-6-(trifluoromethyl)-1H-pyrimidine-2,4-dione (CAS 212754-02-4);
b5) from the group of the bleacher herbicides:

PDS inhibitors: beflubutamid, diflufenican, fluridone, flurochloridone, flurtamone, norflurazon, picolinafen, and 4-(3-trifluoromethylphenoxy)-2-(4-trifluoromethylphenyl)-pyrimidine (CAS 180608-33-7), HPPD inhibitors: benzobicyclon, benzofenap, clomazone, isoxaflutole, mesotrione, pyrasulfotole, pyrazolynate, pyrazoxyfen, sulcotrione, tefuryltrione, tembotrione, topramezone and bicyclopyrone, bleacher, unknown target: aclonifen, amitrole and flumeturon;
b6) from the group of the EPSP synthase inhibitors:

glyphosate, glyphosate-isopropylammonium, glyposate-potassium and glyphosate-trimesium (sulfosate);
b7) from the group of the glutamine synthase inhibitors:

bilanaphos (bialaphos), bilanaphos-sodium, glufosinate, glufosinate-P and glufosinate-ammonium;
b8) from the group of the DHP synthase inhibitors:

asulam;
b9) from the group of the mitosis inhibitors:

compounds of group K1: dinitroanilines such as benfluralin, butralin, dinitramine, ethalfluralin, fluchloralin, oryzalin, pendimethalin, prodiamine and trifluralin, phosphoramidates such as amiprophos, amiprophos-methyl, and butamiphos, benzoic acid herbicides such as chlorthal, chlorthal-dimethyl, pyridines such as dithiopyr and thiazopyr, benzamides such as propyzamide and tebutam; compounds of group K2: chlorpropham, propham and carbetamide, among these, compounds of group K1, in particular dinitroanilines are preferred;
b10) from the group of the VLCFA inhibitors:

chloroacetamides such as acetochlor, alachlor, butachlor, dimethachlor, dimethenamid, dimethenamid-P, metazachlor, metolachlor, metolachlor-S, pethoxamid, pretilachlor, propachlor, propisochlor and thenylchlor, oxyacetanilides such as flufenacet and mefenacet, acetanilides such as diphenamid, naproanilide and napropamide, tetrazolinones such fentrazamide, and other herbicides such as anilofos, cafenstrole, fenoxasulfone, ipfencarbazone, piperophos, pyroxasulfone and isoxazoline compounds of the formulae II.1, II.2, II.3, II.4, II.5, II.6, II.7, II.8 and II.9

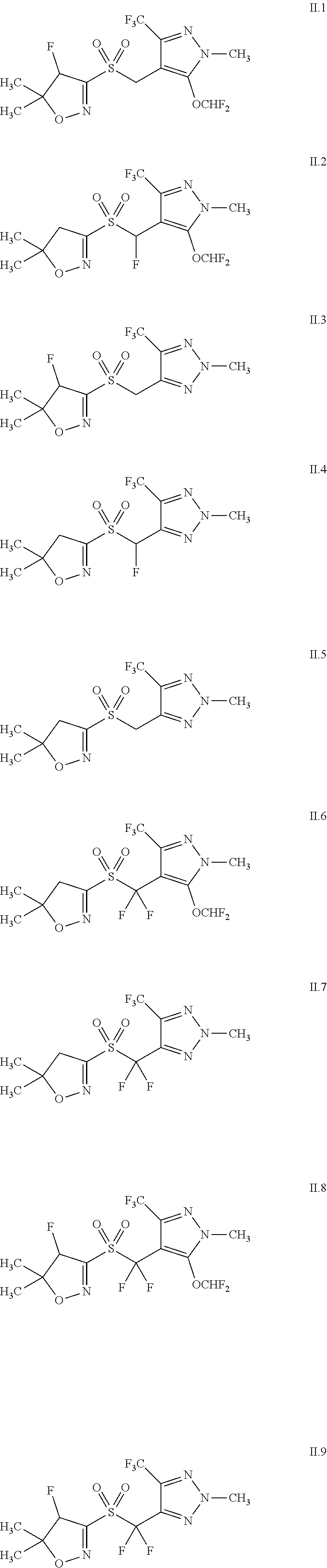

the isoxazoline compounds of the formula (I)l are known in the art, e.g. from WO 2006/024820, WO 2006/037945, WO 2007/071900 and WO 2007/096576;

among the VLCFA inhibitors, preference is given to chloroacetamides and oxyacetamides;
b11) from the group of the cellulose biosynthesis inhibitors:

chlorthiamid, dichlobenil, flupoxam, indaziflam, triaziflam, isoxaben and 1-Cyclohexyl-5-pentafluorphenyloxy-1⁴-[1,2,4,6]thiatriazin-3-ylamine;
b12) from the group of the decoupler herbicides:

dinoseb, dinoterb and DNOC and its salts;
b13) from the group of the auxinic herbicides:

2,4-D and its salts and esters such as clacyfos, 2,4-DB and its salts and esters, aminocyclopyrachlor and its salts and esters, aminopyralid and its salts such as aminopyralid-tris(2-hydroxypropyl)ammonium and its esters, benazolin, benazolin-ethyl, chloramben and its salts and esters, clomeprop, clopyralid and its salts and esters, dicamba and its salts and esters, dichlorprop and its salts and esters, dichlorprop-P and its salts and esters, fluroxypyr, fluroxypyr-butometyl, fluroxypyr-meptyl, halauxifen and its salts and esters (CAS 943832-60-8); MCPA and its salts and esters, MCPA-thioethyl, MCPB and its salts and esters, mecoprop and its salts and esters, mecoprop-P and its salts and esters, picloram and its salts and esters, quinclorac, quinmerac, TBA (2,3,6) and its salts and esters and triclopyr and its salts and esters;
b14) from the group of the auxin transport inhibitors: diflufenzopyr, diflufenzopyr-sodium, naptalam and naptalam-sodium;
b15) from the group of the other herbicides: bromobutide, chlorflurenol, chlorflurenol-methyl, cinmethylin, cumyluron, cyclopyrimorate (CAS 499223-49-3) and its salts and esters, dalapon, dazomet, difenzoquat, difenzoquat-metilsulfate, dimethipin, DSMA, dymron, endothal and its salts, etobenzanid, flamprop, flamprop-isopropyl, flamprop-methyl, flamprop-M-isopropyl, flamprop-M-methyl, flurenol, flurenol-butyl, flurprimidol, fosamine, fosamine-ammonium, indanofan, indaziflam, maleic hydrazide, mefluidide, metam, methiozolin (CAS 403640-27-7), methyl azide, methyl bromide, methyl-dymron, methyl iodide, MSMA, oleic acid, oxaziclomefone, pelargonic acid, pyributicarb, quinoclamine, triaziflam and tridiphane.

Preferred herbicides B that can be used in combination with the PPO-inhibiting herbicides according to the present invention are:
b1) from the group of the lipid biosynthesis inhibitors:

clethodim, clodinafop-propargyl, cycloxydim, cyhalofop-butyl, diclofop-methyl, fenoxaprop-P-ethyl, fluazifop-P-butyl, haloxyfop-P-methyl, metamifop, pinoxaden, profoxydim, propaquizafop, quizalofop-P-ethyl, quizalofop-P-tefuryl, sethoxydim, tepraloxydim, tralkoxydim, 4-(4′-Chloro-4-cyclopropyl-2′-fluoro[1,1′-biphenyl]-3-yl)-5-hydroxy-2,2,6,6-tetramethyl-2H-pyran-3(6H)-one (CAS 1312337-72-6); 4-(2′,4′-Dichloro-4-cyclopropyl[1,1′-biphenyl]-3-yl)-5-hydroxy-2,2,6,6-tetramethyl-2H-pyran-3(6H)-one (CAS 1312337-45-3); 4-(4′-Chloro-4-ethyl-2′-fluoro[1,1′-biphenyl]-3-yl)-5-hydroxy-2,2,6,6-tetramethyl-2H-pyran-3(6H)-one (CAS 1033757-93-5); 4-(2′,4′-Dichloro-4-ethyl[1,1′-biphenyl]-3-yl)-2,2,6,6-tetramethyl-2H-pyran-3,5(4H,6H)-dione (CAS 1312340-84-3); 5-(Acetyloxy)-4-(4′-chloro-4-cyclopropyl-2′-fluoro[1,1′-biphenyl]-3-yl)-3,6-dihydro-2,2,6,6-tetramethyl-2H-pyran-3-one (CAS 1312337-48-6); 5-(Acetyloxy)-4-(2′,4′-dichloro-4-cyclopropyl-[1,1′-biphenyl]-3-yl)-3,6-dihydro-2,2,6,6-tetramethyl-2H-pyran-3-one; 5-(Acetyloxy)-4-(4′-chloro-4-ethyl-2′-fluoro[1,1′-biphenyl]-3-yl)-3,6-dihydro-2,2,6,6-tetramethyl-2H-pyran-3-one (CAS 1312340-82-1); 5-(Acetyloxy)-4-(2′,4′-dichloro-4-ethyl[1,1′-biphenyl]-3-yl)-3,6-dihydro-2,2,6,6-tetramethyl-2H-pyran-3-one (CAS 1033760-55-2); 4-(4′-Chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester (CAS 1312337-51-1); 4-(2′,4′-Dichloro-4-cyclopropyl-[1,1′-biphenyl]-3-yl)-5,6-dihydro-2,2,6,6-tetramethyl-5-oxo-2H-pyran-3-yl carbonic acid methyl ester; 4-(4′-Chloro-4-ethyl-2′-fluoro[1,1′-biphenyll-3-yl)-5,6-dihydro-2,2,6,6-tetramethyl-5-oxo-2H-pyran-3-yl carbonic acid methyl ester (CAS 1312340-83-2); 4-(2′,4′-Dichloro-4-ethyl[1,1′-biphenyl]-3-yl)-5,6-dihydro-2,2,6,6-tetramethyl-5-oxo-2H-pyran-3-yl carbonic acid methyl ester (CAS 1033760-58-5); benfuresate, dimepiperate, EPTC, esprocarb, ethofumesate, molinate, orbencarb, prosulfocarb, thiobencarb and triallate;
b2) from the group of the ALS inhibitors:

amidosulfuron, azimsulfuron, bensulfuron-methyl, bispyribac-sodium, chlorimuron-ethyl, chlorsulfuron, cloransulam-methyl, cyclosulfamuron, diclosulam, ethametsulfuron-methyl, ethoxysulfuron, flazasulfuron, florasulam, flucarbazone-sodium, flucetosulfuron, flumetsulam, flupyrsulfuron-methyl-sodium, foramsulfuron, halosulfuron-methyl, imazamethabenz-methyl, imazamox, imazapic, imazapyr, imazaquin, imazethapyr, imazosulfuron, iodosulfuron, iodosulfuron-methyl-sodium, iofensulfuron, iofensulfuron-sodium, mesosulfuron, metazosulfuron, metosulam, metsulfuron-methyl, nicosulfuron, orthosulfamuron, oxasulfuron, penoxsulam, primisulfuron-methyl, propoxycarbazon-sodium, propyrisulfuron, prosulfuron, pyrazosulfuron-ethyl, pyribenzoxim, pyrimisulfan, pyriftalid, pyriminobac-methyl, pyrithiobac-sodium, pyroxsulam, rimsulfuron, sulfometuron-methyl, sulfosulfuron, thiencarbazone-methyl, thifensulfuron-methyl, triasulfuron, tribenuron-methyl, trifloxysulfuron, triflusulfuron-methyl, tritosulfuron and triafamone;
b3) from the group of the photosynthesis inhibitors:

ametryn, amicarbazone, atrazine, bentazone, bentazone-sodium, bromoxynil and its salts and esters, chloridazone, chlorotoluron, cyanazine, desmedipham, diquat-dibromide, diuron, fluometuron, hexazinone, ioxynil and its salts and esters, isoproturon, lenacil, linuron, metamitron, methabenzthiazuron, metribuzin, paraquat, paraquat-dichloride, phenmedipham, propanil, pyridate, simazine, terbutryn, terbuthylazine and thidiazuron;
b4) from the group of the protoporphyrinogen-IX oxidase inhibitors:

acifluorfen, acifluorfen-sodium, azafenidin, bencarbazone, benzfendizone, butafenacil, carfentrazone-ethyl, cinidon-ethyl, flufenpyr-ethyl, flumiclorac-pentyl, flumioxazin, fluoroglycofen-ethyl, fluthiacet-methyl, fomesafen, lactofen, oxadiargyl, oxadiazon, oxyfluorfen, pentoxazone, pyraflufen-ethyl, saflufenacil, sulfentrazone, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), N-ethyl-3-(2,6-dichloro-4-trifluoromethyl-phenoxy)-5-methyl-1H-pyrazole-1-carboxamide (CAS 452098-92-9), N-tetrahydrofurfuryl-3-(2,6-dichloro-4-trifluoromethylphenoxy)-5-methyl-1H-pyrazole-1-carboxamide (CAS 915396-43-9), N-ethyl-3-(2-chloro-6-fluoro-4-trifluoromethylphenoxy)-5-methyl-1H-pyrazole-1-carboxamide (CAS 452099-05-7), N-tetrahydrofurfuryl-3-(2-chloro-6-fluoro-4-trifluoromethylphenoxy)-5-methyl-1H-pyrazole-1-carboxamide (CAS 452100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), 2-(2,2,7-Trifluoro-3-oxo-4-prop-2-ynyl-3,4-dihydro-2H-benzo[1,4]oxazin-6-yl)-4,5,6,7-tetrahydro-isoindole-1,3-dione; 1-Methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione, and 3-[7-Chloro-5-fluoro-2-(trifluoromethyl)-1H-benzimidazol-4-yl]-1-methyl-6-(trifluoromethyl)-1H-pyrimidine-2,4-dione (CAS 212754-02-4);
b5) from the group of the bleacher herbicides:

aclonifen, beflubutamid, benzobicyclon, clomazone, diflufenican, flurochloridone, flurtamone, isoxaflutole, mesotrione, norflurazon, picolinafen, pyrasulfotole, pyrazolynate, sulcotrione, tefuryltrione, tembotrione, topramezone, bicyclopyrone, 4-(3-trifluoromethyl-phenoxy)-2-(4-trifluoromethylphenyl)pyrimidine (CAS 180608-33-7), amitrole and flumeturon;
b6) from the group of the EPSP synthase inhibitors:

glyphosate, glyphosate-isopropylammonium, glyphosate-potassium and glyphosate-trimesium (sulfosate);
b7) from the group of the glutamine synthase inhibitors:

glufosinate, glufosinate-P, glufosinate-ammonium;
b8) from the group of the DHP synthase inhibitors: asulam;
b9) from the group of the mitosis inhibitors:

benfluralin, dithiopyr, ethalfluralin, oryzalin, pendimethalin, thiazopyr and trifluralin;
b10) from the group of the VLCFA inhibitors:

acetochlor, alachlor, anilofos, butachlor, cafenstrole, dimethenamid, dimethenamid-P, fentrazamide, flufenacet, mefenacet, metazachlor, metolachlor, S-metolachlor, naproanilide, napropamide, pretilachlor, fenoxasulfone, ipfencarbazone, pyroxasulfone thenylchlor and isoxazoline-compounds of the formulae II.1, II.2, II.3, II.4, II.5, II.6, II.7, II.8 and II.9 as mentioned above;
b11) from the group of the cellulose biosynthesis inhibitors: dichlobenil, flupoxam, isoxaben and 1-Cyclohexyl-5-pentafluorphenyloxy-14-[1,2,4,6]thiatriazin-3-ylamine;
b13) from the group of the auxinic herbicides:

2,4-D and its salts and esters, aminocyclopyrachlor and its salts and esters, aminopyralid and its salts such as aminopyralid-tris(2-hydroxypropyl)ammonium and its esters, clopyralid and its salts and esters, dicamba and its salts and esters, dichlorprop-P and its salts and esters, fluroxypyr-meptyl, halauxifen and its salts and esters (CAS 943832-60-8), MCPA and its salts and esters, MCPB and its salts and esters, mecoprop-P and its salts and esters, picloram and its salts and esters, quinclorac, quinmerac and triclopyr and its salts and esters;
b14) from the group of the auxin transport inhibitors: diflufenzopyr and diflufenzopyr-sodium;
b15) from the group of the other herbicides: bromobutide, cinmethylin, cumyluron, cyclopyrimorate (CAS 499223-49-3) and its salts and esters, dalapon, difenzoquat, difenzoquat-metilsulfate, DSMA, dymron (=daimuron), flamprop, flamprop-isopropyl, flamprop-methyl, flamprop-M-isopropyl, flamprop-M-methyl, indanofan, indaziflam, metam, methylbromide, MSMA, oxaziclomefone, pyributicarb, triaziflam and tridiphane.

Particularly preferred herbicides B that can be used in combination with the PPO-inhibiting herbicides according to the present invention are:
b1) from the group of the lipid biosynthesis inhibitors: clodinafop-propargyl, cycloxydim, cyhalofop-butyl, fenoxaprop-P-ethyl, pinoxaden, profoxydim, tepraloxydim, tralkoxydim, 4-(4′-Chloro-4-cyclopropyl-2′-fluoro[1,1′-biphenyl]-3-yl)-5-hydroxy-2,2,6,6-tetramethyl-2H-pyran-3(6H)-one (CAS 1312337-72-6); 4-(2′,4′-Dichloro-4-cyclopropyl[1,1′-biphenyl]-3-yl)-5-hydroxy-2,2,6,6-tetramethyl-2H-pyran-3(6H)-one (CAS 1312337-45-3); 4-(4′-Chloro-4-ethyl-2′-fluoro[1,1′-biphenyl]-3-yl)-5-hydroxy-2,2,6,6-tetramethyl-2H-pyran-3(6H)-one (CAS 1033757-93-5); 4-(2′,4′-Dichloro-4-ethyl[1,1′-biphenyl]-3-yl)-2,2,6,6-tetramethyl-2H-pyran-3,5(4H,6H)-dione (CAS 1312340-84-3); 5-(Acetyloxy)-4-(4′-chloro-4-cyclopropyl-2′-fluoro[1,1′-biphenyl]-3-yl)-3,6-dihydro-2,2,6,6-tetramethyl-2H-pyran-3-one (CAS 1312337-48-6); 5-(Acetyloxy)-4-(2′,4′-dichloro-4-cyclopropyl-[1,1′-biphenyl]-3-yl)-3,6-dihydro-2,2,6,6-tetramethyl-2H-pyran-3-one; 5-(Acetyloxy)-4-(4′-chloro-4-ethyl-2′-fluoro[1,1′-biphenyl]-3-yl)-3,6-dihydro-2,2,6,6-tetramethyl-2H-pyran-3-one (CAS 1312340-82-1); 5-(Acetyloxy)-4-(2′,4′-dichloro-4-ethyl[1,1′-biphenyl]-3-yl)-3,6-dihydro-2,2,6,6-tetramethyl-2H-pyran-3-one (CAS 1033760-55-2); 4-(4′-Chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester (CAS 1312337-51-1); 4-(2′,4′-Dichloro-4-cyclopropyl-[1,1′-biphenyl]-3-yl)-5,6-dihydro-2,2,6,6-tetramethyl-5-oxo-2H-pyran-3-yl carbonic acid methyl ester; 4-(4′-Chloro-4-ethyl-2′-fluoro[1,1′-biphenyl]-3-yl)-5,6-dihydro-2,2,6,6-tetramethyl-5-oxo-2H-pyran-3-yl carbonic acid methyl ester (CAS 1312340-83-2); 4-(2′,4′-Dichloro-4-ethyl[1,1′-biphenyl]-3-yl)-5,6-dihydro-2,2,6,6-tetramethyl-5-oxo-2H-pyran-3-yl carbonic acid methyl ester (CAS 1033760-58-5); esprocarb, prosulfocarb, thiobencarb and triallate;
b2) from the group of the ALS inhibitors: bensulfuron-methyl, bispyribac-sodium, cyclosulfamuron, diclosulam, flumetsulam, flupyrsulfuron-methyl-sodium, foramsulfuron, imazamox, imazapic, imazapyr, imazaquin, imazethapyr, imazosulfuron, iodosulfuron, iodosulfuron-methyl-sodium, iofensulfuron, iofensulfuron-sodium, mesosulfuron, metazosulfuron, nicosulfuron, penoxsulam, propoxycarbazon-sodium, propyrisulfuron, pyrazosulfuron-ethyl, pyroxsulam, rimsulfuron, sulfosulfuron, thiencarbazon-methyl, tritosulfuron and triafamone;
b3) from the group of the photosynthesis inhibitors: ametryn, atrazine, diuron, fluometuron, hexazinone, isoproturon, linuron, metribuzin, paraquat, paraquat-dichloride, propanil, terbutryn and terbuthylazine;
b4) from the group of the protoporphyrinogen-IX oxidase inhibitors: acifluorfen, acifluorfen-sodium, butafenacil, cinidon-ethyl, carfentrazone-ethyl, flumioxazin, fluthiacet-methyl, fomesafen, lactofen, oxadiargyl. oxyfluorfen, saflufenacil. sulfentrazone, ethyl [3-[2-chloro-4-fluoro-5-(1-methyl-6-trifluoromethyl-2,4-dioxo-1,2,3,4-tetrahydropyrimidin-3-yl)-phenoxy]-2-pyridyloxy]acetate (CAS 353292-31-6; S-3100), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), and 2-(2,2,7-Trifluoro-3-oxo-4-prop-2-ynyl-3,4-dihydro-2H-benzo[1,4]oxazin-6-yl)-4,5,6,7-tetrahydro-isoindole-1,3-dione, and 1-Methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione;
b5) from the group of the bleacher herbicides: clomazone, diflufenican, flurochloridone, isoxaflutole, mesotrione, picolinafen, sulcotrione, tefuryltrione, tembotrione, topramezone, bicyclopyrone, amitrole and flumeturon;
b6) from the group of the EPSP synthase inhibitors: glyphosate, glyphosate-isopropylammonium and glyphosate-trimesium (sulfosate);
b7) from the group of the glutamine synthase inhibitors: glufosinate, glufosinate-P and glufosinate-ammonium;
b9) from the group of the mitosis inhibitors: pendimethalin and trifluralin;
b10) from the group of the VLCFA inhibitors: acetochlor, cafenstrole, dimethenamid-P, fentrazamide, flufenacet, mefenacet, metazachlor, metolachlor, S-metolachlor, fenoxasulfone, ipfencarbazone and pyroxasulfone; likewise, preference is given to isoxazoline compounds of the formulae II.1, II.2, II.3, II.4, II.5, II.6, II.7, II.8 and II.9 as mentioned above;
b11) from the group of the cellulose biosynthesis inhibitors: isoxaben;
b13) from the group of the auxinic herbicides: 2,4-D and its salts and esters such as clacyfos, and aminocyclopyrachlor and its salts and esters, aminopyralid and its salts and its esters, clopyralid and its salts and esters, dicamba and its salts and esters, fluroxypyr-meptyl, quinclorac and quinmerac;
b14) from the group of the auxin transport inhibitors: diflufenzopyr and diflufenzopyr-sodium,
b15) from the group of the other herbicides: dymron (=daimuron), indanofan, indaziflam, oxaziclomefone and triaziflam.

Moreover, it may be useful to apply the PPO-inhibiting herbicides, when used in combination with a compound B described SUPRA, in combination with safeners. Safeners are chemical compounds which prevent or reduce damage on useful plants without having a major impact on the herbicidal action of herbicides towards unwanted plants. They can be applied either before sowings (e.g. on seed treatments, shoots or seedlings) or in the pre-emergence application or post-emergence application of the useful plant.

Furthermore, the safeners C, the PPO-inhibiting herbicides and/or the herbicides B can be applied simultaneously or in succession.

Suitable safeners are e.g. (quinolin-8-oxy)acetic acids, 1-phenyl-5-haloalkyl-1H-1,2,4-triazol-3-carboxylic acids, 1-phenyl-4,5-dihydro-5-alkyl-1H-pyrazol-3,5-dicarboxylic acids, 4,5-dihydro-5,5-diaryl-3-isoxazol carboxylic acids, dichloroacetamides, alpha-oximinophenylacetonitriles, acetophenonoximes, 4,6-dihalo-2-phenylpyrimidines, N-[[4-(aminocarbonyl)phenyl]sulfonyl]-2-benzoic amides, 1,8-naphthalic anhydride, 2-halo-4-(haloalkyl)-5-thiazol carboxylic acids, phosphorthiolates and N-alkyl-O-phenyl-carbamates and their agriculturally acceptable salts and their agriculturally acceptable derivatives such amides, esters, and thioesters, provided they have an acid group.

Examples of preferred safeners C are benoxacor, cloquintocet, cyometrinil, cyprosulfamide, dichlormid, dicyclonon, dietholate, fenchlorazole, fenclorim, flurazole, fluxofenim, furilazole, isoxadifen, mefenpyr, mephenate, naphthalic anhydride, oxabetrinil, 4-(dichloroacetyl)-1-oxa-4-azaspiro[4.5]decane (MON4660, CAS 71526-07-3) and 2,2,5-trimethyl-3-(dichloroacetyl)-1,3-oxazolidine (R-29148, CAS 52836-31-4).

Especially preferred safeners C are benoxacor, cloquintocet, cyprosulfamide, dichlormid, fenchlorazole, fenclorim, flurazole, fluxofenim, furilazole, isoxadifen, mefenpyr, naphthalic anhydride, oxabetrinil, 4-(dichloroacetyl)-1-oxa-4-azaspiro[4.5]decane (MON4660, CAS 71526-07-3) and 2,2,5-trimethyl-3-(dichloroacetyl)-1,3-oxazolidine (R-29148, CAS 52836-31-4).

Particularly preferred safeners C are benoxacor, cloquintocet, cyprosulfamide, dichlormid, fenchlorazole, fenclorim, furilazole, isoxadifen, mefenpyr, naphtalic anhydride, 4-(dichloroacetyl)-1-oxa-4-azaspiro[4.5]decane (MON4660, CAS 71526-07-3), and 2,2,5-trimethyl-3-(dichloroacetyl)-1,3-oxazolidine (R-29148, CAS 52836-31-4).

Also preferred safeners C are benoxacor, cloquintocet, cyprosulfamide, dichlormid, fenchlorazole, fenclorim, furilazole, isoxadifen, mefenpyr, 4-(dichloroacetyl)-1-oxa-4-azaspiro[4.5]decane (MON4660, CAS 71526-07-3) and 2,2,5-trimethyl-3-(dichloroacetyl)-1,3-oxazolidine (R-29148, CAS 52836-31-4).

Particularly preferred safeners C, which, as component C, are constituent of the composition according to the invention are the safeners C as defined above; in particular the safeners C.1-C.12 listed below in table C:

TABLE C
Safener C

C.1 benoxacor
C.2 cloquintocet
C.3 cyprosulfamide
C.4 dichlormid
C.5 fenchlorazole
C.6 fenclorim
C.7 furilazole
C.8 isoxadifen
C.9 mefenpyr
C.10 naphtalic acid anhydride
C.11 4-(dichloroacetyl)-1-oxa-4-azaspiro[4.5]decane
(MON4660, CAS 71526-07-3)
C.12 2,2,5-trimethyl-3-(dichloro-acetyl)-1,3-oxazolidine
(R-29148, CAS 52836-31-4)

The PPO-inhibiting herbicides (compounds A) and the active compounds B of groups b1) to b15) and the active compounds C are known herbicides and safeners, see, for example, The Compendium of Pesticide Common Names (www.alanwood.net/pesticides/); Farm Chemicals Handbook 2000 volume 86, Meister Publishing Company, 2000; B. Hock, C. Fedtke, R. R. Schmidt, Herbizide [Herbicides], Georg Thieme Verlag, Stuttgart 1995; W. H. Ahrens, Herbicide Handbook, 7th edition, Weed Science Society of America, 1994; and K. K. Hatzios, Herbicide Handbook, Supplement for the 7th edition, Weed Science Society of America, 1998. 2,2,5-Trimethyl-3-(dichloroacetyl)-1,3-oxazolidine [CAS No. 52836-31-4] is also referred to as R-29148. 4-(Dichloroacetyl)-1-oxa-4-azaspiro[4.5]decane [CAS No. 71526-07-3] is also referred to as AD-67 and MON 4660.

The assignment of the active compounds to the respective mechanisms of action is based on current knowledge. If several mechanisms of action apply to one active compound, this substance was only assigned to one mechanism of action.

Active compounds B and C having a carboxyl group can be employed in the form of the acid, in the form of an agriculturally suitable salt as mentioned above or else in the form of an agriculturally acceptable derivative in the compositions according to the invention.

In the case of dicamba, suitable salts include those, where the counterion is an agri-culturally acceptable cation. For example, suitable salts of dicamba are dicamba-sodium, dicamba-potassium, dicamba-methylammonium, dicamba-dimethylammonium, dicamba-isopropylammonium, dicamba-diglycolamine, dicamba-olamine, dicamba-diolamine, dicamba-trolamine, dicamba-N,N-bis-(3-aminopropyl)methylamine and dicamba-diethylenetriamine. Examples of a suitable ester are dicamba-methyl and dicamba-butotyl.

Suitable salts of 2,4-D are 2,4-D-ammonium, 2,4-D-dimethylammonium, 2,4-D-diethylammonium, 2,4-D-diethanolammonium (2,4-D-diolamine), 2,4-D-triethanol-ammonium, 2,4-D-isopropylammonium, 2,4-D-triisopropanolammonium, 2,4-D-heptylammonium, 2,4-D-dodecylammonium, 2,4-D-tetradecylammonium, 2,4-D-triethylammonium, 2,4-D-tris(2-hydroxypropyl)ammonium, 2,4-D-tris(isopropyl)-ammonium, 2,4-D-trolamine, 2,4-D-lithium, 2,4-D-sodium. Examples of suitable esters of 2,4-D are 2,4-D-butotyl, 2,4-D-2-butoxypropyl, 2,4-D-3-butoxypropyl, 2,4-D-butyl, 2,4-D-ethyl, 2,4-D-ethylhexyl, 2,4-D-isobutyl, 2,4-D-isooctyl, 2,4-D-isopropyl, 2,4-D-meptyl, 2,4-D-methyl, 2,4-D-octyl, 2,4-D-pentyl, 2,4-D-propyl, 2,4-D-tefuryl and clacyfos.

Suitable salts of 2,4-DB are for example 2,4-DB-sodium, 2,4-DB-potassium and 2,4-DB-dimethylammonium. Suitable esters of 2,4-DB are for example 2,4-DB-butyl and 2,4-DB-isoctyl.

Suitable salts of dichlorprop are for example dichlorprop-sodium, dichlorprop-potassium and dichlorprop-dimethylammonium. Examples of suitable esters of dichlorprop are dichlorprop-butotyl and dichlorprop-isoctyl.

Suitable salts and esters of MCPA include MCPA-butotyl, MCPA-butyl, MCPA-dimethyl-ammonium, MCPA-diolamine, MCPA-ethyl, MCPA-thioethyl, MCPA-2-ethylhexyl, MCPA-isobutyl, MCPA-isoctyl, MCPA-isopropyl, MCPA-isopropylammonium, MCPA-methyl, MCPA-olamine, MCPA-potassium, MCPA-sodium and MCPA-trolamine. A suitable salt of MCPB is MCPB sodium. A suitable ester of MCPB is MCPB-ethyl. Suitable salts of clopyralid are clopyralid-potassium, clopyralid-olamine and clopyralid-tris-(2-hydroxypropyl)ammonium. Example of suitable esters of clopyralid is clopyralid-methyl.

Examples of a suitable ester of fluroxypyr are fluroxypyr-meptyl and fluroxypyr-2-butoxy-1-methylethyl, wherein fluroxypyr-meptyl is preferred.

Suitable salts of picloram are picloram-dimethylammonium, picloram-potassium, picloram-triisopropanolammonium, picloram-triisopropylammonium and picloram-trolamine. A suitable ester of picloram is picloram-isoctyl.

A suitable salt of triclopyr is triclopyr-triethylammonium. Suitable esters of triclopyr are for example triclopyr-ethyl and triclopyr-butotyl.

Suitable salts and esters of chloramben include chloramben-ammonium, chloramben-diolamine, chloramben-methyl, chloramben-methylammonium and chloramben-sodium.

Suitable salts and esters of 2,3,6-TBA include 2,3,6-TBA-dimethylammonium, 2,3,6-TBA-lithium, 2,3,6-TBA-potassium and 2,3,6-TBA-sodium.

Suitable salts and esters of aminopyralid include aminopyralid-potassium and amino-pyralid-tris(2-hydroxypropyl)ammonium.

Suitable salts of glyphosate are for example glyphosate-ammonium, glyphosate-diammonium, glyphoste-dimethylammonium, glyphosate-isopropylammonium, glyphosate-potassium, glyphosate-sodium, glyphosate-trimesium as well as the ethanolamine and diethanolamine salts, preferably glyphosate-diammonium, glyphosate-isopropylammonium and glyphosate-trimesium (sulfosate).

A suitable salt of glufosinate is for example glufosinate-ammonium.

A suitable salt of glufosinate-P is for example glufosinate-P-ammonium.

Suitable salts and esters of bromoxynil are for example bromoxynil-butyrate, bromoxynil-heptanoate, bromoxynil-octanoate, bromoxynil-potassium and bromoxynil-sodium.

Suitable salts and esters of ioxonil are for example ioxonil-octanoate, ioxonil-potassium and ioxonil-sodium.

Suitable salts and esters of mecoprop include mecoprop-butotyl, mecoprop-dimethylammonium, mecoprop-diolamine, mecoprop-ethadyl, mecoprop-2-ethylhexyl, mecoprop-isoctyl, mecoprop-methyl, mecoprop-potassium, mecoprop-sodium and mecoprop-trolamine.

Suitable salts of mecoprop-P are for example mecoprop-P-butotyl, mecoprop-P-dimethylammonium, mecoprop-P-2-ethylhexyl, mecoprop-P-isobutyl, mecoprop-P-potassium and mecoprop-P-sodium.

A suitable salt of diflufenzopyr is for example diflufenzopyr-sodium.

A suitable salt of naptalam is for example naptalam-sodium.

Suitable salts and esters of aminocyclopyrachlor are for example aminocyclopyrachlor-dimethylammonium, aminocyclopyrachlor-methyl, aminocyclopyrachlor-triisopropanolammonium, aminocyclopyrachlor-sodium and aminocyclopyrachlor-potassium.

A suitable salt of quinclorac is for example quinclorac-dimethylammonium.

A suitable salt of quinmerac is for example quinclorac-dimethylammonium.

A suitable salt of imazamox is for example imazamox-ammonium.

Suitable salts of imazapic are for example imazapic-ammonium and imazapic-isopropylammonium.

Suitable salts of imazapyr are for example imazapyr-ammonium and imazapyr-isopropylammonium.

A suitable salt of imazaquin is for example imazaquin-ammonium.

Suitable salts of imazethapyr are for example imazethapyr-ammonium and imazethapyr-isopropylammonium.

A suitable salt of topramezone is for example topramezone-sodium.

The preferred embodiments of the invention mentioned herein below have to be understood as being preferred either independently from each other or in combination with one another.

According to a preferred embodiment of the invention, the composition comprises as component B at least one, preferably exactly one herbicide B.

According to another preferred embodiment of the invention, the composition comprises at least two, preferably exactly two, herbicides B different from each other.

According to another preferred embodiment of the invention, the composition comprises at least three, preferably exactly three, herbicides B different from each other.

According to another preferred embodiment of the invention, the composition comprises as component A at least one, preferably exactly one PPO-inhibiting herbicide, preferably acifluorfen, acifluorfen-sodium, butafenacil, cinidon-ethyl, carfentrazone-ethyl, flumioxazin, fluthiacet-methyl, fomesafen, lactofen, oxadiargyl, oxyfluorfen, saflufenacil, sulfentrazone, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100; 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), especially preferred saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), and as component B at least one, preferably exactly one, herbicide B.

According to another preferred embodiment of the invention, the composition comprises as component A at least one, preferably exactly preferably exactly one PPO-inhibiting herbicide, preferably acifluorfen, acifluorfen-sodium, butafenacil, cinidon-ethyl, carfentrazone-ethyl, flumioxazin, fluthiacet-methyl, fomesafen, lactofen, oxadiargyl, oxyfluorfen, saflufenacil, sulfentrazone, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), especially preferred saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), and at least two, preferably exactly two, herbicides B different from each other.

According to another preferred embodiment of the invention, the composition comprises as component A at least one, preferably exactly preferably exactly one PPO-inhibiting herbicide, preferably acifluorfen, acifluorfen-sodium, butafenacil, cinidon-ethyl, carfentrazone-ethyl, flumioxazin, fluthiacet-methyl, fomesafen, lactofen, oxadiargyl, oxyfluorfen, saflufenacil, sulfentrazone, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), especially preferred saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin) and at least three, preferably exactly three, herbicides B different from each other.

According to another preferred embodiment of the invention, the composition comprises, in addition to a PPO-inhibiting herbicide, preferably acifluorfen, acifluorfen-sodium, butafenacil, cinidon-ethyl, carfentrazone-ethyl, flumioxazin, fluthiacet-methyl, fomesafen, lactofen, oxadiargyl, oxyfluorfen, saflufenacil, sulfentrazone, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), especially preferred saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), at least one and especially exactly one herbicidally active compound from group b1), in particular selected from the group consisting of clethodim, clodinafop-propargyl, cycloxydim, cyhalofop-butyl, fenoxaprop-P-ethyl, fluazifop, pinoxaden, profoxydim, quizalofop, sethoxydim, tepraloxydim, tralkoxydim, esprocarb, prosulfocarb, thiobencarb and triallate.

According to another preferred embodiment of the invention, the composition comprises, in addition to a PPO-inhibiting herbicide, preferably acifluorfen, acifluorfen-sodium, butafenacil, cinidon-ethyl, carfentrazone-ethyl, flumioxazin, fluthiacet-methyl, fomesafen, lactofen, oxadiargyl, oxyfluorfen, saflufenacil, sulfentrazone, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin) especially preferred saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), at least one and especially exactly one herbicidally active compound from group b2), in particular selected from the group consisting of bensulfuron-methyl, bispyribac-sodium, cloransulam-methyl, cyclosulfamuron, diclosulam, flumetsulam, flupyrsulfuron-methyl-sodium, foramsulfuron, halosulfuron-methyl, imazamox, imazapic, imazapyr, imazaquin, imazethapyr, imazosulfuron, iodosulfuron, iodosulfuron-methyl-sodium, mesosulfuron-methyl, metazosulfuron, nicosulfuron, penoxsulam, propoxycarbazon-sodium, pyrazosulfuron-ethyl, pyrithiobac-sodium, pyroxsulam, rimsulfuron, sulfosulfuron, thiencarbazon-methyl, thifensulfuron-methyl, trifloxysulfuron and tritosulfuron.

According to another preferred embodiment of the invention, the composition comprises, in addition to a a PPO-inhibiting herbicide, preferably acifluorfen, acifluorfen-sodium, butafenacil, cinidon-ethyl, carfentrazone-ethyl, flumioxazin, fluthiacet-methyl, fomesafen, lactofen, oxadiargyl, oxyfluorfen, saflufenacil, sulfentrazone, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), especially preferred saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), at least one and especially exactly one herbicidally active compound from group b3), in particular selected from the group consisting of ametryn, atrazine, bentazon, bromoxynil, diuron, fluometuron, hexazinone, isoproturon, linuron, metribuzin, paraquat, paraquat-dichloride, prometryne, propanil, terbutryn and terbuthylazine.

According to another preferred embodiment of the invention, the composition comprises, in addition to a a PPO-inhibiting herbicide, preferably acifluorfen, acifluorfen-sodium, butafenacil, cinidon-ethyl, carfentrazone-ethyl, flumioxazin, fluthiacet-methyl, fomesafen, lactofen, oxadiargyl, oxyfluorfen, saflufenacil, sulfentrazone, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), especially preferred saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), at least one and especially exactly one herbicidally active compound from group b4), in particular selected from the group consisting of acifluorfen, acifluorfen-sodium, azafenidin, bencarbazone, benzfendizone, bifenox, butafenacil, carfentrazone, carfentrazone-ethyl, chlomethoxyfen, cinidon-ethyl, fluazolate, flufenpyr, flufenpyr-ethyl, flumiclorac, flumiclorac-pentyl, flumioxazin, fluoroglycofen, fluoroglycofen-ethyl, fluthiacet, fluthiacet-methyl, fomesafen, halosafen, lactofen, oxadiargyl, oxadiazon, oxyfluorfen, pentoxazone, profluazol, pyraclonil, pyraflufen, pyraflufen-ethyl, saflufenacil, sulfentrazone, thidiazimin, tiafenacil, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), N-ethyl-3-(2,6-dichloro-4-trifluoromethylphenoxy)-5-methyl-1H-pyrazole-1-carboxamide (CAS 452098-92-9), N-tetrahydrofurfuryl-3-(2,6-dichloro-4-trifluoromethylphenoxy)-5-methyl-1H-pyrazole-1-carboxamide (CAS 915396-43-9), N-ethyl-3-(2-chloro-6-fluoro-4-trifluoromethylphenoxy)-5-methyl-1H-pyrazole-1-carboxamide (CAS 452099-05-7), N-tetrahydrofurfuryl-3-(2-chloro-6-fluoro-4-trifluoromethylphenoxy)-5-methyl-1H-pyrazole-1-carboxamide (CAS 452100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), 2-(2,2,7-Trifluoro-3-oxo-4-prop-2-ynyl-3,4-dihydro-2H-benzo[1,4]oxazin-6-yl)-4,5,6,7-tetrahydro-isoindole-1,3-dione, 1-Methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione, methyl (E)-4-[2-chloro-5-[4-chloro-5-(difluoromethoxy)-1H-methyl-pyrazol-3-yl]-4-fluoro-phenoxy]-3-methoxy-but-2-enoate [CAS 948893-00-3], 3-[7-Chloro-5-fluoro-2-(trifluoromethyl)-1H-benzimidazol-4-yl]-1-methyl-6-(trifluoromethyl)-1H-pyrimidine-2,4-dione (CAS 212754-02-4).

According to another preferred embodiment of the invention, the composition comprises, in addition to a a PPO-inhibiting herbicide, preferably acifluorfen, acifluorfen-sodium, butafenacil, cinidon-ethyl, carfentrazone-ethyl, flumioxazin, fluthiacet-methyl, fomesafen, lactofen, oxadiargyl, oxyfluorfen, saflufenacil, sulfentrazone, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), especially preferred saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), at least one and especially exactly one herbicidally active compound from group b5), in particular selected from the group consisting of clomazone, diflufenican, flurochloridone, isoxaflutole, mesotrione, picolinafen, sulcotrione, tefuryltrione, tembotrione, topramezone, bicyclopyrone, amitrole and flumeturon.

According to another preferred embodiment of the invention, the composition comprises, in addition to a a PPO-inhibiting herbicide, preferably acifluorfen, acifluorfen-sodium, butafenacil, cinidon-ethyl, carfentrazone-ethyl, flumioxazin, fluthiacet-methyl, fomesafen, lactofen, oxadiargyl, oxyfluorfen, saflufenacil, sulfentrazone, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), especially preferred saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), at least one and especially exactly one herbicidally active compound from group b6), in particular selected from the group consisting of glyphosate, glyphosate-isopropylammonium and glyphosate-trimesium (sulfosate).

According to another preferred embodiment of the invention, the composition comprises, in addition to a a PPO-inhibiting herbicide, preferably acifluorfen, acifluorfen-sodium, butafenacil, cinidon-ethyl, carfentrazone-ethyl, flumioxazin, fluthiacet-methyl, fomesafen, lactofen, oxadiargyl, oxyfluorfen, saflufenacil, sulfentrazone, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), especially preferred saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), at least one and especially exactly one herbicidally active compound from group b7), in particular selected from the group consisting of glufosinate, glufosinate-P and glufosinate-ammonium.

According to another preferred embodiment of the invention, the composition comprises, in addition to a a PPO-inhibiting herbicide, preferably acifluorfen, acifluorfen-sodium, butafenacil, cinidon-ethyl, carfentrazone-ethyl, flumioxazin, fluthiacet-methyl, fomesafen, lactofen, oxadiargyl, oxyfluorfen, saflufenacil, sulfentrazone, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin) especially preferred saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), at least one and especially exactly one herbicidally active compound from group b9), in particular selected from the group consisting of pendimethalin and trifluralin.

According to another preferred embodiment of the invention, the composition comprises, in addition to a PPO-inhibiting herbicide, preferably acifluorfen, acifluorfen-sodium, butafenacil, cinidon-ethyl, carfentrazone-ethyl, flumioxazin, fluthiacet-methyl, fomesafen, lactofen, oxadiargyl, oxyfluorfen, saflufenacil, sulfentrazone, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin)), especially preferred saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), at least one and especially exactly one herbicidally active compound from group b10), in particular selected from the group consisting of acetochlor, cafenstrole, dimethenamid-P, fentrazamide, flufenacet, mefenacet, metazachlor, metolachlor, S-metolachlor, fenoxasulfone and pyroxasulfone. Likewise, preference is given to compositions comprising in addition to a a PPO-inhibiting herbicide, preferably acifluorfen, acifluorfen-sodium, butafenacil, cinidon-ethyl, carfentrazone-ethyl, flumioxazin, fluthiacet-methyl, fomesafen, lactofen, oxadiargyl, oxyfluorfen, saflufenacil, sulfentrazone, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), especially preferred saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), at least one and especially exactly one herbicidally active compound from group b10), in particular selected from the group consisting of isoxazoline compounds of the formulae II.1, II.2, II.3, II.4, II.5, II.6, II.7, II.8 and II.9, as defined above.

According to another preferred embodiment of the invention, the composition comprises, in addition to a PPO-inhibiting herbicide, preferably acifluorfen, acifluorfen-sodium, butafenacil, cinidon-ethyl, carfentrazone-ethyl, flumioxazin, fluthiacet-methyl, fomesafen, lactofen, oxadiargyl, oxyfluorfen, saflufenacil, sulfentrazone, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), especially preferred saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), at least one and especially exactly one herbicidally active compound from group b13), in particular selected from the group consisting of 2,4-D and its salts and esters, aminocyclopyrachlor and its salts and esters, aminopyralid and its salts such as aminopyralid-tris(2-hydroxypropyl)ammonium and its esters, clopyralid and its salts and esters, dicamba and its salts and esters, fluroxypyr-meptyl, quinclorac and quinmerac.

According to another preferred embodiment of the invention, the composition comprises, in addition to a PPO-inhibiting herbicide, preferably acifluorfen, acifluorfen-sodium, butafenacil, cinidon-ethyl, carfentrazone-ethyl, flumioxazin, fluthiacet-methyl, fomesafen, lactofen, oxadiargyl, oxyfluorfen, saflufenacil, sulfentrazone, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), especially preferred saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), at least one and especially exactly one herbicidally active compound from group b14), in particular selected from the group consisting of diflufenzopyr and diflufenzopyr-sodium.

According to another preferred embodiment of the invention, the composition comprises, in addition to a PPO-inhibiting herbicide, preferably acifluorfen, acifluorfen-sodium, butafenacil, cinidon-ethyl, carfentrazone-ethyl, flumioxazin, fluthiacet-methyl, fomesafen, lactofen, oxadiargyl, oxyfluorfen, saflufenacil, sulfentrazone, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), especially preferred saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), at least one and especially exactly one herbicidally active compound from group b15), in particular selected from the group consisting of dymron (=daimuron), indanofan, indaziflam, oxaziclomefone and triaziflam.

Here and below, the term “binary compositions” includes compositions comprising one or more, for example 1, 2 or 3, active compounds of the PPO-inhibiting herbicide and either one or more, for example 1, 2 or 3, herbicides B.

In binary compositions comprising at least one PPO-inhibiting herbicide as component A and at least one herbicide B, the weight ratio of the active compounds A:B is generally in the range of from 1:1000 to 1000:1, preferably in the range of from 1:500 to 500:1, in particular in the range of from 1:250 to 250:1 and particularly preferably in the range of from 1:75 to 75:1.

Particularly preferred herbicides B are the herbicides B as defined above; in particular the herbicides B.1-B.229 listed below in table B:

TABLE B

| | Herbicide B |
|---|---|
| B.1 | clethodim |
| B.2 | clodinafop-propargyl |
| B.3 | cycloxydim |
| B.4 | cyhalofop-butyl |
| B.5 | fenoxaprop-ethyl |
| B.6 | fenoxaprop-P-ethyl |
| B.7 | fluazifop |
| B.8 | metamifop |
| B.9 | pinoxaden |
| B.10 | profoxydim |
| B.11 | quizalofop |
| B.12 | sethoxydim |
| B.13 | tepraloxydim |
| B.14 | tralkoxydim |
| B.15 | esprocarb |
| B.16 | ethofumesate |
| B.17 | molinate |
| B.18 | prosulfocarb |
| B.19 | thiobencarb |
| B.20 | triallate |
| B.21 | bensulfuron-methyl |
| B.22 | bispyribac-sodium |
| B.23 | cloransulam-methyl |
| B.24 | chlorsulfuron |
| B.25 | clorimuron |
| B.26 | cyclosulfamuron |
| B.27 | diclosulam |
| B.28 | florasulam |
| B.29 | flumetsulam |
| B.30 | flupyrsulfuron-methyl-sodium |
| B.31 | foramsulfuron |
| B.32 | halosulfuron-methyl |
| B.33 | imazamox |
| B.34 | imazamox-ammonium |
| B.35 | imazapic |
| B.36 | imazapic-ammonium |
| B.37 | imazapic-isopropylammonium |
| B.38 | imazapyr |
| B.39 | imazapyr-ammonium |
| B.40 | imazapyr-isopropylammonium |
| B.41 | imazaquin |
| B.42 | imazaquin-ammonium |
| B.43 | imazethapyr |
| B.44 | imazethapyr-ammonium |
| B.45 | imazethapyr-isopropylammonium |
| B.46 | imazosulfuron |
| B.47 | iodosulfuron-methyl-sodium |
| B.48 | iofensulfuron |
| B.49 | iofensulfuron-sodium |
| B.50 | mesosulfuron-methyl |
| B.51 | metazosulfuron |
| B.52 | metsulfuron-methyl |
| B.53 | metosulam |
| B.54 | nicosulfuron |
| B.55 | penoxsulam |
| B.56 | propoxycarbazon-sodium |
| B.57 | pyrazosulfuron-ethyl |
| B.58 | pyribenzoxim |
| B.59 | pyriftalid |
| B.60 | pyrithiobac-sodium |
| B.61 | pyroxsulam |
| B.62 | propyrisulfuron |
| B.63 | rimsulfuron |
| B.64 | sulfosulfuron |
| B.65 | thiencarbazone-methyl |
| B.66 | thifensulfuron-methyl |
| B.67 | tribenuron-methyl |
| B.68 | trifloxysulfuron |
| B.69 | tritosulfuron |
| B.70 | triafamone |
| B.71 | ametryne |
| B.72 | atrazine |
| B.73 | bentazon |
| B.74 | bromoxynil |
| B.75 | bromoxynil-octanoate |
| B.76 | bromoxynil-heptanoate |
| B.77 | bromoxynil-potassium |
| B.78 | diuron |
| B.79 | fluometuron |
| B.80 | hexazinone |
| B.81 | isoproturon |
| B.82 | linuron |
| B.83 | metamitron |
| B.84 | metribuzin |
| B.85 | prometryne |
| B.86 | propanil |
| B.87 | simazin |
| B.88 | terbuthylazine |
| B.89 | terbutryn |
| B.90 | paraquat-dichloride |
| B.91 | acifluorfen |
| B.92 | acifluorfen-sodium |
| B.93 | azafenidin |
| B.94 | bencarbazone |
| B.95 | benzfendizone |
| B.96 | bifenox |
| B.97 | butafenacil |
| B.98 | carfentrazone |
| B.99 | carfentrazone-ethyl |
| B.100 | chlomethoxyfen |
| B.101 | cinidon-ethyl |
| B.102 | fluazolate |
| B.103 | flufenpyr |
| B.104 | flufenpyr-ethyl |
| B.105 | flumiclorac |
| B.106 | flumiclorac-pentyl |
| B.107 | flumioxazin |
| B.108 | fluoroglycofen |
| B.109 | fluoroglycofen-ethyl |
| B.110 | fluthiacet |
| B.111 | fluthiacet-methyl |
| B.112 | fomesafen |
| B.113 | halosafen |
| B.114 | lactofen |
| B.115 | oxadiargyl |
| B.116 | oxadiazon |
| B.117 | oxyfluorfen |
| B.118 | pentoxazone |
| B.119 | profluazol |
| B.120 | pyraclonil |
| B.121 | pyraflufen |
| B.122 | pyraflufen-ethyl |
| B.123 | saflufenacil |
| B.124 | sulfentrazone |
| B.125 | thidiazimin |
| B.126 | tiafenacil |
| B.127 | ethyl [3-[2-chloro-4-fluoro-5- |
| | (1-methyl-6-trifluoromethyl- |
| | 2,4-dioxo-1,2,3,4-tetrahydro- |
| | pyrimidin-3-yl)phenoxy]-2- |
| | pyridyloxy]acetate (CAS |
| | 353292-31-6) |
| B.128 | 1,5-dimethyl-6-thioxo-3-(2,2,7- |
| | trifluoro-3-oxo-4-(prop-2- |
| | ynyl)-3,4-dihydro-2H- |
| | benzo[b][1,4]oxazin-6-yl)-1,3,5- |
| | triazinane-2,4-dione (CAS |
| | 1258836-72-4/ |
| | trifludimoxazin) |
| B.129 | N-ethyl-3-(2,6-dichloro-4- |
| | trifluoromethylphenoxy)-5- |
| | methyl-1H-pyrazole-1- |
| | carboxamide (CAS 452098-92-9) |
| B.130 | N-tetrahydrofurfuryl-3-(2,6- |
| | dichloro-4- |
| | trifluoromethylphenoxy)-5- |
| | methyl-1H-pyrazole-1- |
| | carboxamide (CAS 915396-43-9) |
| B.131 | N-ethyl-3-(2-chloro-6-fluoro- |
| | 4-trifluoromethylphenoxy)-5- |
| | methyl-1H-pyrazole-1- |
| | carboxamide (CAS 452099-05-7) |
| B.132 | N-tetrahydrofurfuryl-3-(2- |
| | chloro-6-fluoro-4-trifluoro- |
| | methylphenoxy)-5-methyl-1H- |
| | pyrazole-1-carboxamide (CAS |
| | 452100-03-7) |
| B.133 | 3-[7-fluoro-3-oxo-4-(prop-2- |
| | ynyl)-3,4-dihydro-2H- |
| | benzo[1,4]oxazin-6-yl]-1,5- |
| | dimethyl-6-thioxo- |
| | [1,3,5]triazinan-2,4-dione |
| B.134 | 2-(2,2,7-Trifluoro-3-oxo-4- |
| | prop-2-ynyl-3,4-dihydro-2H- |
| | benzo[1,4]oxazin-6-yl)-4,5,6,7- |
| | tetrahydro-isoindole-1,3-dione |
| B.135 | 1-Methyl-6-trifluoromethyl-3- |
| | (2,2,7-trifluoro-3-oxo-4-prop- |
| | 2-ynyl-3,4-dihydro-2H- |
| | benzo[1,4]oxazin-6-yl)-1H- |
| | pyrimidine-2,4-dione |
| B.136 | methyl (E)-4-[2-chloro-5-[4- |
| | chloro-5-(difluoromethoxy)- |
| | 1H-methyl-pyrazol-3-yl]-4- |
| | fluoro-phenoxy]-3-methoxy- |
| | but-2-enoate [CAS 948893- |
| | 00-3] |
| B.137 | 3-[7-Chloro-5-fluoro-2- |
| | (trifluoromethyl)-1H- |
| | benzimidazol-4-yl]-1-methyl- |
| | 6-(trifluoromethyl)-1H- |
| | pyrimidine-2,4-dione (CAS |
| | 212754-02-4) |
| B.138 | benzobicyclon |
| B.139 | clomazone |
| B.140 | diflufenican |
| B.141 | flurochloridone |
| B.142 | isoxaflutole |
| B.143 | mesotrione |
| B.144 | norflurazone |
| B.145 | picolinafen |
| B.146 | sulcotrione |
| B.147 | tefuryltrione |
| B.148 | tembotrione |
| B.149 | topramezone |
| B.150 | topramezone-sodium |
| B.151 | bicyclopyrone |
| B.152 | amitrole |
| B.153 | fluometuron |
| B.154 | glyphosate |
| B.155 | glyphosate-ammonium |
| B.156 | glyphosate-dimethylammonium |
| B.157 | glyphosate-isopropylammonium |
| B.158 | glyphosate-trimesium (sulfosate) |
| B.159 | glyphosate-potassium |
| B.160 | glufosinate |
| B.161 | glufosinate-ammonium |
| B.162 | glufosinate-P |
| B.163 | glufosinate-P-ammonium |
| B.164 | pendimethalin |
| B.165 | trifluralin |
| B.166 | acetochlor |
| B.167 | butachlor |
| B.168 | cafenstrole |
| B.169 | dimethenamid-P |
| B.170 | fentrazamide |
| B.171 | flufenacet |
| B.172 | mefenacet |
| B.173 | metazachlor |
| B.174 | metolachlor |
| B.175 | S-metolachlor |
| B.176 | pretilachlor |
| B.177 | fenoxasulfone |
| B.178 | isoxaben |
| B.179 | ipfencarbazone |
| B.180 | pyroxasulfone |
| B.181 | 2,4-D |
| B.182 | 2,4-D-isobutyl |
| B.183 | 2,4-D-dimethylammonium |
| B.184 | 2,4-D-N,N,N-trimethylethanolammonium |
| B.185 | aminopyralid |
| B.186 | aminopyralid-methyl |
| B.187 | aminopyralid-tris(2-hydroxypropyl)ammonium |
| B.188 | clopyralid |
| B.189 | clopyralid-methyl |
| B.190 | clopyralid-olamine |
| B.191 | dicamba |
| B.192 | dicamba-butotyl |
| B.193 | dicamba-diglycolamine |
| B.194 | dicamba-dimethylammonium |
| B.195 | dicamba-diolamine |
| B.196 | dicamba-isopropylammonium |
| B.197 | dicamba-potassium |
| B.198 | dicamba-sodium |
| B.199 | dicamba-trolamine |
| B.200 | dicamba-N,N-bis-(3-aminopropyl)methylamine |
| B.201 | dicamba-diethylenetriamine |
| B.202 | fluroxypyr |
| B.203 | fluroxypyr-meptyl |
| B.204 | MCPA |
| B.205 | MCPA-2-ethylhexyl |
| B.206 | MCPA-dimethylammonium |
| B.207 | quinclorac |
| B.208 | quinclorac-dimethylammonium |
| B.209 | quinmerac |
| B.210 | quinmerac-dimethylammonium |
| B.211 | aminocyclopyrachlor |
| B.212 | aminocyclopyrachlor-potassium |
| B.213 | aminocyclopyrachlor-methyl |
| B.214 | diflufenzopyr |
| B.215 | diflufenzopyr-sodium |
| B.216 | dymron |
| B.217 | indanofan |
| B.218 | indaziflam |
| B.219 | oxaziclomefone |
| B.220 | triaziflam |
| B.221 | II.1 |
| B.222 | II.2 |
| B.223 | II.3 |
| B.224 | II.4 |
| B.225 | II.5 |
| B.226 | II.6 |
| B.227 | II.7 |
| B.228 | II.8 |
| B.229 | II.9 |

Particularly preferred are compositions 1.1 to 1.229, comprising acifluorfen and the substance(s) as defined in the respective row of table B-1:

TABLE B-1
(compositions 1.1 to 1.229):

| comp. no. | herbicide B |
|---|---|
| 1.1 | B.1 |
| 1.2 | B.2 |
| 1.3 | B.3 |
| 1.4 | B.4 |
| 1.5 | B.5 |
| 1.6 | B.6 |
| 1.7 | B.7 |
| 1.8 | B.8 |
| 1.9 | B.9 |
| 1.10 | B.10 |
| 1.11 | B.11 |
| 1.12 | B.12 |
| 1.13 | B.13 |
| 1.14 | B.14 |
| 1.15 | B.15 |
| 1.16 | B.16 |
| 1.17 | B.17 |
| 1.18 | B.18 |
| 1.19 | B.19 |
| 1.20 | B.20 |
| 1.21 | B.21 |
| 1.22 | B.22 |
| 1.23 | B.23 |
| 1.24 | B.24 |
| 1.25 | B.25 |
| 1.26 | B.26 |
| 1.27 | B.27 |
| 1.28 | B.28 |
| 1.29 | B.29 |
| 1.30 | B.30 |
| 1.31 | B.31 |
| 1.32 | B.32 |
| 1.33 | B.33 |
| 1.34 | B.34 |
| 1.35 | B.35 |
| 1.36 | B.36 |
| 1.37 | B.37 |
| 1.38 | B.38 |
| 1.39 | B.39 |
| 1.40 | B.40 |
| 1.41 | B.41 |
| 1.42 | B.42 |
| 1.43 | B.43 |
| 1.44 | B.44 |
| 1.45 | B.45 |
| 1.46 | B.46 |
| 1.47 | B.47 |
| 1.48 | B.48 |
| 1.49 | B.49 |
| 1.50 | B.50 |
| 1.51 | B.51 |
| 1.52 | B.52 |
| 1.53 | B.53 |
| 1.54 | B.54 |
| 1.55 | B.55 |
| 1.56 | B.56 |
| 1.57 | B.57 |
| 1.58 | B.58. |
| 1.59 | B.59 |
| 1.60 | B.60 |
| 1.61 | B.61 |
| 1.62 | B.62 |
| 1.63 | B.63 |
| 1.64 | B.64 |
| 1.65 | B.65 |
| 1.66 | B.66 |
| 1.67 | B.67 |
| 1.68 | B.68 |
| 1.69 | B.69 |
| 1.70 | B.70 |
| 1.71 | B.71 |
| 1.72 | B.72 |
| 1.73 | B.73 |
| 1.74 | B.74 |
| 1.75 | B.75 |
| 1.76 | B.76 |
| 1.77 | B.77 |
| 1.78 | B.78 |
| 1.79 | B.79 |
| 1.80 | B.80 |
| 1.81 | B.81 |
| 1.82 | B.82 |
| 1.83 | B.83 |
| 1.84 | B.84 |
| 1.85 | B.85 |
| 1.86 | B.86 |
| 1.87 | B.87 |
| 1.88 | B.88 |
| 1.89 | B.89 |
| 1.90 | B.90 |
| 1.91 | B.91 |
| 1.92 | B.92 |
| 1.93 | B.93 |
| 1.94 | B.94 |
| 1.95 | B.95 |
| 1.96 | B.96 |
| 1.97 | B.97 |
| 1.98 | B.98 |
| 1.99 | B.99 |
| 1.100 | B.100 |
| 1.101 | B.101 |
| 1.102 | B.102 |
| 1.103 | B.103 |
| 1.104 | B.104 |
| 1.105 | B.105 |
| 1.106 | B.106 |
| 1.107 | B.107 |
| 1.108 | B.108 |
| 1.109 | B.109 |
| 1.110 | B.110 |
| 1.111 | B.111 |
| 1.112 | B.112 |
| 1.113 | B.113 |
| 1.114 | B.114 |
| 1.115 | B.115 |
| 1.116 | B.116 |
| 1.117 | B.117 |
| 1.118 | B.118 |
| 1.119 | B.119 |
| 1.120 | B.120 |
| 1.121 | B.121 |
| 1.122 | B.122 |
| 1.123 | B.123 |
| 1.124 | B.124 |
| 1.125 | B.125 |
| 1.126 | B.126 |
| 1.127 | B.127 |
| 1.128 | B.128 |
| 1.129 | B.129 |
| 1.130 | B.130 |
| 1.131 | B.131 |
| 1.132 | B.132 |
| 1.133 | B.133 |
| 1.134 | B.134 |
| 1.135 | B.135 |
| 1.136 | B.136 |
| 1.137 | B.137 |
| 1.138 | B.138 |
| 1.139 | B.139 |
| 1.140 | B.140 |
| 1.141 | B.141 |
| 1.142 | B.142 |
| 1.143 | B.143 |
| 1.144 | B.144 |
| 1.145 | B.145 |
| 1.146 | B.146 |
| 1.147 | B.147 |
| 1.148 | B.148 |
| 1.149 | B.149 |
| 1.150 | B.150 |
| 1.151 | B.151 |
| 1.152 | B.152 |
| 1.153 | B.153 |
| 1.154 | B.154 |
| 1.155 | B.155 |
| 1.156 | B.156 |
| 1.157 | B.157 |
| 1.158 | B.158 |
| 1.159 | B.159 |
| 1.160 | B.160 |
| 1.161 | B.161 |
| 1.162 | B.162 |
| 1.163 | B.163 |
| 1.164 | B.164 |
| 1.165 | B.165 |
| 1.166 | B.166 |
| 1.167 | B.167 |
| 1.168 | B.168 |
| 1.169 | B.169 |
| 1.170 | B.170 |
| 1.171 | B.171 |
| 1.172 | B.172 |
| 1.173 | B.173 |
| 1.174 | B.174 |
| 1.175 | B.175 |
| 1.176 | B.176 |
| 1.177 | B.177 |
| 1.178 | B.178 |
| 1.179 | B.179 |
| 1.180 | B.180 |
| 1.181 | B.181 |
| 1.182 | B.182 |
| 1.183 | B.183 |
| 1.184 | B.184 |
| 1.185 | B.185 |
| 1.186 | B.186 |
| 1.187 | B.187 |
| 1.188 | B.188 |
| 1.189 | B.189 |
| 1.190 | B.190 |
| 1.191 | B.191 |
| 1.192 | B.192 |
| 1.193 | B.193 |
| 1.194 | B.194 |
| 1.195 | B.195 |
| 1.196 | B.196 |
| 1.197 | B.197 |
| 1.198 | B.198 |
| 1.199 | B.199 |
| 1.200 | B.200 |
| 1.201 | B.201 |
| 1.202 | B.202 |
| 1.203 | B.203 |
| 1.204 | B.204 |
| 1.205 | B.205 |
| 1.206 | B.206 |
| 1.207 | B.207 |
| 1.208 | B.208 |
| 1.209 | B.209 |
| 1.210 | B.210 |
| 1.211 | B.211 |
| 1.212 | B.212 |
| 1.213 | B.213 |
| 1.214 | B.214 |
| 1.215 | B.215 |
| 1.216 | B.216 |
| 1.217 | B.217 |
| 1.218 | B.218 |
| 1.219 | B.219 |
| 1.220 | B.220 |
| 1.221 | B.221 |
| 1.222 | B.222 |
| 1.223 | B.223 |
| 1.224 | B.224 |
| 1.225 | B.225 |
| 1.226 | B.226 |
| 1.227 | B.227 |
| 1.228 | B.228 |
| 1.229 | B.229 |

Also especially preferred are compositions 2.1. to 2.229 which differ from the corresponding compositions 1.1 to 1.229 only in that they comprise as component A acifluorfen-sodium.

Also especially preferred are compositions 3.1. to 3.229 which differ from the corresponding compositions 1.1 to 1.229 only in that they comprise as component A azafenidin.

Also especially preferred are compositions 4.1. to 4.229 which differ from the corresponding compositions 1.1 to 1.229 only in that they comprise as component A bencarbazone.

Also especially preferred are compositions 5.1. to 5.229 which differ from the corresponding compositions 1.1 to 1.229 only in that they comprise as component A benzfendizone.

Also especially preferred are compositions 6.1. to 6.229 which differ from the corresponding compositions 1.1 to 1.229 only in that they comprise as component A bifenox.

Also especially preferred are compositions 7.1. to 7.229 which differ from the corresponding compositions 1.1 to 1.227 only in that they comprise as component A butafenacil.

Also especially preferred are compositions 8.1. to 8.229 which differ from the corresponding compositions 1.1 to 1.229 only in that they comprise as component A carfentrazone.

Also especially preferred are compositions 9.1. to 9.229 which differ from the corresponding compositions 1.1 to 1.229 only in that they comprise as component A carfentrazone-ethyl.

Also especially preferred are compositions 10.1. to 10.229 which differ from the corresponding compositions 1.1 to 1.229 only in that they comprise as component A chlomethoxyfen.

Also especially preferred are compositions 11.1. to 11.229 which differ from the corresponding compositions 1.1 to 1.229 only in that they comprise as component A cinidon-ethyl.

Also especially preferred are compositions 12.1. to 12.229 which differ from the corresponding compositions 1.1 to 1.229 only in that they comprise as component A fluazolate.

Also especially preferred are compositions 13.1. to 13.229 which differ from the corresponding compositions 1.1 to 1.229 only in that they comprise as component A flufenpyr.

Also especially preferred are compositions 14.1. to 14.229 which differ from the corresponding compositions 1.1 to 1.229 only in that they comprise as component A flufenpyr-ethyl.

Also especially preferred are compositions 15.1. to 15.229 which differ from the corresponding compositions 1.1 to 1.229 only in that they comprise as component A flumiclorac.

Also especially preferred are compositions 16.1. to 16.229 which differ from the corresponding compositions 1.1 to 1.229 only in that they comprise as component A flumiclorac-pentyl.

Also especially preferred are compositions 17.1. to 17.229 which differ from the corresponding compositions 1.1 to 1.229 only in that they comprise as component A flumioxazin.

Also especially preferred are compositions 18.1. to 18.229 which differ from the corresponding compositions 1.1 to 1.229 only in that they comprise as component A fluoroglycofen.

Also especially preferred are compositions 19.1. to 19.229 which differ from the corresponding compositions 1.1 to 1.229 only in that they comprise as component A fluoroglycofen-ethyl.

Also especially preferred are compositions 20.1. to 20.229 which differ from the corresponding compositions 1.1 to 1.229 only in that they comprise as component A fluthiacet.

Also especially preferred are compositions 21.1. to 21.229 which differ from the corresponding compositions 1.1 to 1.229 only in that they comprise as component A fluthiacet-methyl.

Also especially preferred are compositions 22.1. to 22.229 which differ from the corresponding compositions 1.1 to 1.229 only in that they comprise as component A fomesafen.

Also especially preferred are compositions 23.1. to 23.229 which differ from the corresponding compositions 1.1 to 1.229 only in that they comprise as component A halosafen.

Also especially preferred are compositions 24.1. to 24.229 which differ from the corresponding compositions 1.1 to 1.229 only in that they comprise as component A lactofen.

Also especially preferred are compositions 25.1. to 25.229 which differ from the corresponding compositions 1.1 to 1.229 only in that they comprise as component A oxadiargyl.

Also especially preferred are compositions 26.1. to 26.229 which differ from the corresponding compositions 1.1 to 1.229 only in that they comprise as component A oxadiazon.

Also especially preferred are compositions 27.1. to 27.229 which differ from the corresponding compositions 1.1 to 1.229 only in that they comprise as component A oxyfluorfen.

Also especially preferred are compositions 28.1. to 28.229 which differ from the corresponding compositions 1.1 to 1.229 only in that they comprise as component A pentoxazone.

Also especially preferred are compositions 29.1. to 29.229 which differ from the corresponding compositions 1.1 to 1.229 only in that they comprise as component A profluazol.

Also especially preferred are compositions 30.1. to 30.229 which differ from the corresponding compositions 1.1 to 1.229 only in that they comprise as component A pyraclonil.

Also especially preferred are compositions 31.1. to 31.229 which differ from the corresponding compositions 1.1 to 1.229 only in that they comprise as component A pyraflufen.

Also especially preferred are compositions 32.1. to 32.229 which differ from the corresponding compositions 1.1 to 1.229 only in that they comprise as component A pyraflufen-ethyl.

Also especially preferred are compositions 33.1. to 33.229 which differ from the corresponding compositions 1.1 to 1.229 only in that they comprise as component A saflufenacil.

Also especially preferred are compositions 34.1. to 34.229 which differ from the corresponding compositions 1.1 to 1.229 only in that they comprise as component A sulfentrazone.

Also especially preferred are compositions 35.1. to 35.229 which differ from the corresponding compositions 1.1 to 1.229 only in that they comprise as component A thidiazimin.

Also especially preferred are compositions 36.1. to 36.229 which differ from the corresponding compositions 1.1 to 1.229 only in that they comprise as component A tiafenacil.

Also especially preferred are compositions 37.1. to 37.229 which differ from the corresponding compositions 1.1 to 1.229 only in that they comprise as component A ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100).

Also especially preferred are compositions 38.1. to 38.229 which differ from the corresponding compositions 1.1 to 1.229 only in that they comprise as component A 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin) Also especially preferred are compositions 39.1. to 39.229 which differ from the corresponding compositions 1.1 to 1.229 only in that they comprise as component A N-ethyl-3-(2,6-dichloro-4-trifluoromethylphenoxy)-5-methyl-1H-pyrazole-1-carboxamide (CAS 452098-92-9).

Also especially preferred are compositions 40.1. to 40.229 which differ from the corresponding compositions 1.1 to 1.229 only in that they comprise as component A N-tetrahydrofurfuryl-3-(2,6-dichloro-4-trifluoromethylphenoxy)-5-methyl-1H-pyrazole-1-carboxamide (CAS 915396-43-9).

Also especially preferred are compositions 41.1. to 41.229 which differ from the corresponding compositions 1.1 to 1.229 only in that they comprise as component A N-ethyl-3-(2-chloro-6-fluoro-4-trifluoromethylphenoxy)-5-methyl-1H-pyrazole-1-carboxamide (CAS 452099-05-7).

Also especially preferred are compositions 42.1. to 42.229 which differ from the corresponding compositions 1.1 to 1.229 only in that they comprise as component A N-tetrahydrofurfuryl-3-(2-chloro-6-fluoro-4-trifluoromethylphenoxy)-5-methyl-1H-pyrazole-1-carboxamide (CAS 452100-03-7).

Also especially preferred are compositions 43.1. to 43.229 which differ from the corresponding compositions 1.1 to 1.229 only in that they comprise as component A 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione.

Also especially preferred are compositions 44.1. to 44.229 which differ from the corresponding compositions 1.1 to 1.229 only in that they comprise as component A methyl (E)-4-[2-chloro-5-[4-chloro-5-(difluoromethoxy)-1H-methyl-pyrazol-3-yl]-4-fluoro-phenoxy]-3-methoxy-but-2-enoate (CAS 948893-00-3).

Also especially preferred are compositions 45.1. to 45.229 which differ from the corresponding compositions 1.1 to 1.229 only in that they comprise as component A 3-[7-Chloro-5-fluoro-2-(trifluoromethyl)-1H-benzimidazol-4-yl]-1-methyl-6-(trifluoromethyl)-1H-pyrimidine-2,4-dione (CAS 212754-02-4).

Also especially preferred are compositions 46.1. to 46.229 which differ from the corresponding compositions 1.1 to 1.229 only in that they comprise as component A 2-(2,2,7-Trifluoro-3-oxo-4-prop-2-ynyl-3,4-dihydro-2H-benzo[1,4]oxazin-6-yl)-4,5,6,7-tetrahydro-isoindole-1,3-dione.

Also especially preferred are compositions 47.1. to 47.229 which differ from the corresponding compositions 1.1 to 1.229 only in that they comprise as component A 1-Methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione Also especially preferred are compositions 48.1. to 48.229 which differ from the corresponding compositions 1.1 to 1.229 only in that they additionally comprise benoxacor as safener C.

Also especially preferred are compositions 49.1. to 49.229 which differ from the corresponding compositions 1.1 to 1.229 only in that they additionally comprise cloquintocet as safener C.

Also especially preferred are compositions 50.1. to 50.229 which differ from the corresponding compositions 1.1 to 1.229 only in that they additionally comprise cyprosulfamide as safener C.

Also especially preferred are compositions 51.1. to 51.229 which differ from the corresponding compositions 1.1 to 1.229 only in that they additionally comprise dichlormid as safener C.

Also especially preferred are compositions 52.1. to 52.229 which differ from the corresponding compositions 1.1 to 1.229 only in that they additionally comprise fenchlorazole as safener C.

Also especially preferred are compositions 53.1. to 53.229 which differ from the corresponding compositions 1.1 to 1.229 only in that they additionally comprise fenclorim as safener C.

Also especially preferred are compositions 54.1. to 54.229 which differ from the corresponding compositions 1.1 to 1.229 only in that they additionally comprise furilazole as safener C.

Also especially preferred are compositions 55.1. to 55.229 which differ from the corresponding compositions 1.1 to 1.229 only in that they additionally comprise isoxadifen as safener C.

Also especially preferred are compositions 56.1. to 56.229 which differ from the corresponding compositions 1.1 to 1.229 only in that they additionally comprise mefenpyr as safener C.

Also especially preferred are compositions 57.1. to 57.229 which differ from the corresponding compositions 1.1 to 1.229 only in that they additionally comprise 4-(dichloroacetyl)-1-oxa-4-azaspiro[4.5]decane (MON4660, CAS 71526-07-3) as safener C.

Also especially preferred are compositions 58.1. to 58.229 which differ from the corresponding compositions 1.1 to 1.229 only in that they additionally comprise 2,2,5-trimethyl-3-(dichloroacetyl)-1,3-oxazolidine (R-29148, CAS 52836-31-4) as safener C.

It is generally preferred to use the compounds of the invention in combination with herbicides that are selective for the crop being treated and which complement the spectrum of weeds controlled by these compounds at the application rate employed. It is further generally preferred to apply the compounds of the invention and other complementary herbicides at the same time, either as a combination formulation or as a tank mix.

In another embodiment, the present invention refers to a method for identifying a herbicide by using a mutated PPO encoded by a nucleic acid which comprises the nucleotide sequence of SEQ ID NO: 118, 119, 120, 121, 122, 123, 124, 125, 126, 127, 128, or 130, or a variant or derivative thereof.

Said method comprises the steps of:
a) generating a transgenic cell or plant comprising a nucleic acid encoding a mutated PPO, wherein the mutated PPO is expressed;
b) applying a herbicide to the transgenic cell or plant of a) and to a control cell or plant of the same variety;
c) determining the growth or the viability of the transgenic cell or plant and the control cell or plant after application of said herbicide, and
d) selecting “herbicides” which confer reduced growth to the control cell or plant as compared to the growth of the transgenic cell or plant.

As described above, the present invention teaches compositions and methods for increasing the tolerance of a crop plant or seed as compared to a wild-type variety of the plant or seed. In a preferred embodiment, the tolerance of a crop plant or seed is increased such that the plant or seed can withstand a herbicide application of preferably approximately 1-1000 g ai ha⁻¹, more preferably 1-200 g ai ha⁻¹, even more preferably 5-150 g ai ha⁻¹, and most preferably 10-100 g ai ha⁻¹. As used herein, to “withstand” a herbicide application means that the plant is either not killed or only moderately injured by such application. It will be understood by the person skilled in the art that the application rates may vary, depending on the environmental conditions such as temperature or humidity, and depending on the chosen kind of herbicide (active ingredient ai).

Post-emergent weed control methods useful in various embodiments hereof utilize about >0.3× application rates of herbicides; in some embodiments, this can be about, for example, >0.3×, >0.4×, >0.5×, >0.6×, >0.7×, >0.8×, >0.9×, or >l× of herbicides. In one embodiment, herbicide-tolerant plants of the present invention have tolerance to a post-emergant application of a herbicides at an amount of about 25 to about 200 g ai/ha. In some embodiments, wherein the herbicide-tolerant plant is a dicot (e.g., soy, cotton), the post-emergant application of the herbicides is at an amount of about 50 g ai/ha. In another embodiment, wherein the herbicide-tolerant plant is a monocot (e.g., maize, rice, sorghum), the post-emergant application of the herbicides is at an amount of about 200 g ai/ha. In other embodiments, wherein the herbicide-tolerant plant is a Brassica (e.g., canola), the post-emergant application of the herbicides is at an amount of about 25 g ai/ha. In post-emergent weed control methods hereof, in some embodiments, the method can utilize herbicides application rates at about 7 to 10 days post-emergent. In another embodiment, the application rate can exceed lx herbicides; in some embodiments, the rate can be up to 4× herbicides, though more typically it will be about 2.5× or less, or about 2× or less, or about 1× or less.

Furthermore, the present invention provides methods that involve the use of at least one herbicide, optionally in combination with one or more herbicidal compounds B, and, optionally, a safener C, as described in detail supra.

In these methods, the herbicide can be applied by any method known in the art including, but not limited to, seed treatment, soil treatment, and foliar treatment. Prior to application, the herbicide can be converted into the customary formulations, for example solutions, emulsions, suspensions, dusts, powders, pastes and granules. The use form depends on the particular intended purpose; in each case, it should ensure a fine and even distribution of the compound according to the invention.

By providing plants having increased tolerance to herbicide, a wide variety of formulations can be employed for protecting plants from weeds, so as to enhance plant growth and reduce competition for nutrients. A herbicide can be used by itself for pre-emergence, post-emergence, pre-planting, and at-planting control of weeds in areas surrounding the crop plants described herein, or a herbicide formulation can be used that contains other additives. The herbicide can also be used as a seed treatment. Additives found in a herbicide formulation include other herbicides, detergents, adjuvants, spreading agents, sticking agents, stabilizing agents, or the like. The herbicide formulation can be a wet or dry preparation and can include, but is not limited to, flowable powders, emulsifiable concentrates, and liquid concentrates. The herbicide and herbicide formulations can be applied in accordance with conventional methods, for example, by spraying, irrigation, dusting, or the like.

Suitable formulations are described in detail in PCT/EP2009/063387 and PCT/EP2009/063386, which are incorporated herein by reference.

As disclosed herein, the PPO nucleic acids of the invention find use in enhancing the herbicide tolerance of plants that comprise in their genomes a gene encoding a herbicide-tolerant mutated PPO protein. Such a gene may be an endogenous gene or a transgene, as described above. Additionally, in certain embodiments, the nucleic acids of the present invention can be stacked with any combination of polynucleotide sequences of interest in order to create plants with a desired phenotype. For example, the nucleic acids of the present invention may be stacked with any other polynucleotides encoding polypeptides having pesticidal and/or insecticidal activity, such as, for example, the Bacillus thuringiensis toxin proteins (described in U.S. Pat. Nos. 5,366,892; 5,747,450; 5,737,514; 5,723,756; 5,593,881; and Geiser et al (1986) Gene 48: 109), 5-enolpyruvylshikimate-3-phosphate synthase (EPSPS), Glyphosate acetyl transferase (GAT), cytochrome P450 monooxygenase, phosphinothricin acetyltransferase (PAT), Acetohydroxyacid synthase (AHAS; EC 4.1.3.18, also known as acetolactate synthase or ALS), hydroxyphenyl pyruvate dioxygenase (HPPD), Phytoene desaturase (PD), Protoporphyrinogen oxidase (PPO) and dicamba degrading enzymes as disclosed in WO 02/068607, or phenoxyaceticacid- and phenoxypropionicacid-derivative degrading enzymes as disclosed in WO 2008141154 or WO 2005107437. The combinations generated can also include multiple copies of any one of the polynucleotides of interest.

Consequently, Herbicide-tolerant plants of the invention can be used in conjunction with an herbicide to which they are tolerant. Herbicides can be applied to the plants of the invention using any techniques known to those skilled in the art. Herbicides can be applied at any point in the plant cultivation process. For example, herbicides can be applied pre-planting, at planting, pre-emergence, post-emergence or combinations thereof. Herbicides may be applied to seeds and dried to form a layer on the seeds.

In some embodiments, seeds are treated with a safener, followed by a post-emergent application of a herbicides. In one embodiment, the post-emergent application of the herbicides is about 7 to 10 days following planting of safener-treated seeds. In some embodiments, the safener is cloquintocet, dichlormid, fluxofenim, or combinations thereof.

### Methods of Controlling Weeds or Undesired Vegetation

In other aspects, the present invention provides a method for controlling weeds at a locus for growth of a plant or plant part thereof, the method comprising: applying a composition comprising a herbicides to the locus.

In some aspects, the present invention provides a method for controlling weeds at a locus for growth of a plant, the method comprising: applying an herbicide composition comprising herbicides to the locus; wherein said locus is: (a) a locus that contains: a plant or a seed capable of producing said plant; or (b) a locus that is to be after said applying is made to contain the plant or the seed; wherein the plant or the seed comprises in at least some of its cells a polynucleotide operably linked to a promoter operable in plant cells, the promoter capable of expressing a mutated PPO polypeptide encoded by the polynucleotide, the expression of the mutated PPO polypeptide conferring to the plant tolerance to herbicides.

Herbicide compositions hereof can be applied, e.g., as foliar treatments, soil treatments, seed treatments, or soil drenches. Application can be made, e.g., by spraying, dusting, broadcasting, or any other mode known useful in the art.

In one embodiment, herbicides can be used to control the growth of weeds that may be found growing in the vicinity of the herbicide-tolerant plants invention. In embodiments of this type, an herbicide can be applied to a plot in which herbicide-tolerant plants of the invention are growing in vicinity to weeds. An herbicide to which the herbicide-tolerant plant of the invention is tolerant can then be applied to the plot at a concentration sufficient to kill or inhibit the growth of the weed. Concentrations of herbicide sufficient to kill or inhibit the growth of weeds are known in the art and are disclosed above.

In other embodiments, the present invention provides a method for controlling weeds in the vicinity of a herbicide-tolerant plant of the invention. The method comprises applying an effective amount of a herbicides to the weeds and to the auxinic herbicide-tolerant plant, wherein the plant has increased tolerance to auxinic herbicide when compared to a wild-type plant. In some embodiments, the herbicide-tolerant plants of the invention are preferably crop plants, including, but not limited to, sunflower, alfalfa, Brassica sp., soybean, cotton, safflower, peanut, tobacco, tomato, potato, wheat, rice, maize, sorghum, barley, rye, millet, and sorghum.

In other aspects, herbicide(s) (e.g., herbicides) can also be used as a seed treatment. In some embodiments, an effective concentration or an effective amount of herbicide(s), or a composition comprising an effective concentration or an effective amount of herbicide(s) can be applied directly to the seeds prior to or during the sowing of the seeds. Seed Treatment formulations may additionally comprise binders and optionally colorants.

Binders can be added to improve the adhesion of the active materials on the seeds after treatment. In one embodiments, suitable binders are block copolymers EO/PO surfactants but also polyvinylalcoholsl, polyvinylpyrrolidones, polyacrylates, polymethacrylates, polybutenes, polyisobutylenes, polystyrene, polyethyleneamines, polyethyleneamides, polyethyleneimines (Lupasol®, Polymin®), polyethers, polyurethans, polyvinylacetate, tylose and copolymers derived from these polymers. Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 1 12, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

The term seed treatment comprises all suitable seed treatment techniques known in the art, such as seed dressing, seed coating, seed dusting, seed soaking, and seed pelleting. In one embodiment, the present invention provides a method of treating soil by the application, in particular into the seed drill: either of a granular formulation containing the herbicides as a composition/formulation (e.g., a granular formulation), with optionally one or more solid or liquid, agriculturally acceptable carriers and/or optionally with one or more agriculturally acceptable surfactants. This method is advantageously employed, for example, in seedbeds of cereals, maize, cotton, and sunflower.

The present invention also comprises seeds coated with or containing with a seed treatment formulation comprising herbicides and at least one other herbicide such as, e.g., an AHAS-inhibitor selected from the group consisting of amidosulfuron, azimsulfuron, bensulfuron, chlorimuron, chlorsulfuron, cinosulfuron, cyclosulfamuron, ethametsulfuron, ethoxysulfuron, flazasulfuron, flupyrsulfuron, foramsulfuron, halosulfuron, imazosulfuron, iodosulfuron, mesosulfuron, metsulfuron, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, imazamethabenz, imazamox, imazapic, imazapyr, imazaquin, imazethapyr, cloransulam, diclosulam, florasulam, flumetsulam, metosulam, penoxsulam, bispyribac, pyriminobac, propoxycarbazone, flucarbazone, pyribenzoxim, pyriftalid and pyrithiobac.

The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

In some embodiments, the seed treatment application with herbicides or with a formulation comprising the herbicides is carried out by spraying or dusting the seeds before sowing of the plants and before emergence of the plants.

In other embodiments, in the treatment of seeds, the corresponding formulations are applied by treating the seeds with an effective amount of herbicides or a formulation comprising the herbicides.

In other aspects, the present invention provides a method for combating undesired vegetation or controlling weeds comprising contacting the seeds of the herbicide-tolerant plants of the present invention before sowing and/or after pregermination with herbicides. The method can further comprise sowing the seeds, for example, in soil in a field or in a potting medium in greenhouse. The method finds particular use in combating undesired vegetation or controlling weeds in the immediate vicinity of the seed. The control of undesired vegetation is understood as the killing of weeds and/or otherwise retarding or inhibiting the normal growth of the weeds. Weeds, in the broadest sense, are understood as meaning all those plants which grow in locations where they are undesired.

The weeds of the present invention include, for example, dicotyledonous and monocotyledonous weeds. Dicotyledonous weeds include, but are not limited to, weeds of the genera: Sinapis, Lepiclium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solarium, Rorippa, Rotala, Lindernia, Lamium, Veronica, Abutilon, Emex, Datura, Viola, Galeopsis, Papaver, Centaurea, Trifolium, Ranunculus, and Taraxacum. Monocotyledonous weeds include, but are not limited to, weeds of the genera: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, and Apera.

In addition, the weeds of the present invention can include, for example, crop plants that are growing in an undesired location. For example, a volunteer maize plant that is in a field that predominantly comprises soybean plants can be considered a weed, if the maize plant is undesired in the field of soybean plants.

In other embodiments, in the treatment of seeds, the corresponding formulations are applied by treating the seeds with an effective amount of herbicides or a formulation comprising the herbicides.

In still further aspects, treatment of loci, plants, plant parts, or seeds of the present invention comprises application of an agronomically acceptable composition that does not contain an A.I. In one embodiment, the treatment comprises application of an agronomically acceptable composition that does not contain a herbicides A.I. In some embodiments, the treatment comprises application of an agronomically acceptable composition that does not contain a herbicides A.L, wherein the composition comprises one or more of agronomically-acceptable carriers, diluents, excipients, plant growth regulators, and the like. In other embodiments, the treatment comprises application of an agronomically acceptable composition that does not contain a herbicides A.I., wherein the composition comprises an adjuvant. In one embodiment, the adjuvant is a surfactant, a spreader, a sticker, a penetrant, a drift-control agent, a crop oil, an emulsifier, a compatibility agent, or combinations thereof.

It should also be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims.

### EXAMPLES

Legend to PPO-inhibitors referring to Uracilpyridines which can be used in the following examples:

| | |
|---|---|
| Uracilpyridine 1 | ethyl 2-[[3-[2-chloro-4-fluoro-5-[3-methyl-2,6-dioxo- |
| | 4-(trifluoromethyl)pyrimidin-1-yl]phenoxy]-2- |
| | pyridyl]oxy]acetate |
| Uracilpyridine 2 | ethyl 2-[[3-[[3-chloro-5-fluoro-6-[3-methyl-2,6-dioxo- |
| | 4-(trifluoromethyl)pyrimidin-1-yl]-2-pyridyl]oxy]-2- |
| | pyridyl]oxy]acetate |
| Uracilpyridine 3 | 2-[[3-[[3-chloro-5-fluoro-6-[3-methyl-2,6-dioxo- |
| | 4-(trifluoromethyl)pyrimidin-1-yl]-2-pyridyl]oxy]-2- |
| | pyridyl]oxy]acetic acid |
| Uracilpyridine 4 | ethyl 2-[2-[[3-chloro-5-fluoro-6-[3-methyl-2,6-dioxo- |
| | 4-(trifluoromethyl)pyrimidin-1-yl]-2- |
| | pyridyl]oxy]phenoxy]acetate |
| Uracilpyridine 5 | 2-[2-[[3-chloro-5-fluoro-6-[3-methyl-2,6-dioxo- |
| | 4-(trifluoromethyl)pyrimidin-1-yl]-2- |
| | pyridyl]oxy]phenoxy]acetic acid |
| Uracilpyridine 6 | ethyl 2-[2-[[3-bromo-6-[3-methyl-2,6-dioxo- |
| | 4-(trifluoromethyl)pyrimidin-1-yl]-2- |
| | pyridyl]oxy]phenoxy]acetate |
| Uracilpyridine 7 | ethyl 2-[2-[[3-chloro-5-fluoro-6-[3-methyl-2,6-dioxo- |
| | 4-(trifluoromethyl)pyrimidin-1-yl]-2- |
| | pyridyl]oxy]-4-fluoro-phenoxy]acetate |
| Uracilpyridine 8 | ethyl 2-[2-[[3,5-difluoro-6-[3-methyl-2,6-dioxo- |
| | 4-(trifluoromethyl)pyrimidin-1-yl]-2- |
| | pyridyl]oxy]phenoxy]acetate |
| Uracilpyridine 9 | 2-[2-[[3,5-difluoro-6-[3-methyl-2,6-dioxo- |
| | 4-(trifluoromethyl)pyrimidin-1-yl]-2- |
| | pyridyl]oxy]phenoxy]acetic acid |
| Uracilpyridine 10 | 2-[2-[[3-chloro-5-fluoro-6-[3-methyl-2,6-dioxo- |
| | 4-(trifluoromethyl)pyrimidin-1-yl]-2- |
| | pyridyl]oxy]phenoxy]-N-methylsulfonyl-acetamide |
| Uracilpyridine 11 | ethyl 2-[[3-[[3-chloro-6-[3,5-dimethyl-2,6-dioxo- |
| | 4-(trifluoromethyl)pyrimidin-1-yl]-5-fluoro-2- |
| | pyridyl]oxy]-2-pyridyl]oxy]acetate |
| Uracilpyridine 12 | ethyl 2-[2-[[3-chloro-6-[3,5-dimethyl-2,6-dioxo- |
| | 4-(trifluoromethyl)pyrimidin-1-yl]-5-fluoro-2- |
| | pyridyl]oxy]phenoxy]acetate |
| Uracilpyridine 13 | allyl 2-[[3-[[3-chloro-5-fluoro-6-[3-methyl-2,6-dioxo- |
| | 4-(trifluoromethyl)pyrimidin-1-yl]-2-pyridyl]oxy]-2- |
| | pyridyl]oxy]acetate |
| Uracilpyridine 14 | prop-2-ynyl 2-[[3-[[3-chloro-5-fluoro-6-[3- |
| | methyl-2,6-dioxo-4-(trifluoromethyl)pyrimidin-1- |
| | yl]-2-pyridyl]oxy]-2-pyridyl]oxy]acetate |
| Uracilpyridine 15 | cyclopropylmethyl 2-[[3-[[3-chloro-5-fluoro-6-[3- |
| | methyl-2,6-dioxo-4-(trifluoromethyl)pyrimidin-1- |
| | yl]-2-pyridyl]oxy]-2-pyridyl]oxy]acetate |
| Uracilpyridine 16 | 2,2-difluoroethyl 2-[[3-[[3-chloro-5-fluoro-6-[3- |
| | methyl-2,6-dioxo-4-(trifluoromethyl)pyrimidin-1-yl]-2- |
| | pyridyl]oxy]-2-pyridyl]oxy]acetate |
| Uracilpyridine 17 | isobutyl 2-[[3-[[3-chloro-5-fluoro-6-[3-methyl-2,6- |
| | dioxo-4-(trifluoromethyl)pyrimidin-1-yl]-2- |
| | pyridyl]oxy]-2-pyridyl]oxy]acetate |
| Uracilpyridine 18 | (2-ethoxy-2-oxo-ethyl) 2-[[3-[[3-chloro-5-fluoro-6-[3- |
| | methyl-2,6-dioxo-4-(trifluoromethyl)pyrimidin-1- |
| | yl]-2-pyridyl]oxy]-2-pyridyl]oxy]acetate |
| Uracilpyridine 19 | 2-methoxyethyl 2-[[3-[[3-chloro-5-fluoro-6-[3-methyl- |
| | 2,6-dioxo-4-(trifluoromethyl)pyrimidin-1-yl]-2- |
| | pyridyl]oxy]-2-pyridyl]oxy]acetate |

### Example 1: Site-Directed Mutagenesis PPO

All nucleic acid coding sequence and all single and double mutants encoding a polypeptide comprising SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129, are synthesized and cloned by Geneart (Geneart AG, Regensburg, Germany). Rational design mutants are synthesized by Geneart. Random PPO gene libraries are synthesized by Geneart. Plasmids are isolated from E. coli TOP10 by performing a plasmid minpreparation and confirmed by DNA sequencing.

### Example 2: Expression and Purification of Recombinant Wildtype and Mutant PPO

(Taken from: Franck E. Dayan, Pankaj R. Daga, Stephen O. Duke, Ryan M. Lee, Patrick J. Tranel, Robert J. Doerksen. Biochemical and structural consequences of a glycine deletion in the α-8 helix of protoporphyrinogen oxidase. Biochimica et Biophysica Acta 1804 (2010), 1548-56) Clones in pRSET vector are transformed into BL21(DE3)-pLysS strain of E. coli. Cells are grown in 250 mL of LB with 100 μgmL-1 of carbenicillin, shaking overnight at 37° C. Cultures are diluted in 1 L of LB with antibiotic and grown at 37° C. shaking for 2 h, induced with 1 mM IPTG and grown at 25° C. shaking for 5 more hours. The cells are harvested by centrifugation at 1600×g, washed with 0.09% NaCl, and stored at −80° C. Cells are lysed using a French press at 140 MPa in 50 mM sodium phosphate pH 7.5, 1 M NaCl, 5 mM imidazole, 5% glycerol, and 1 μg mL-1 leupeptin. Following lysis, 0.5 U of benzonase (Novagen, EMD Chemicals, Inc., Gibbstown, NJ) and PMSF (final concentration of 1 mM) are added. Cell debris is removed by centrifugation at 3000×g. His-tagged PPO proteins are purified on a nickel activated Hitrap Chelating HP column (GE Healthcare Bio-Sciences Corp., Piscataway, NJ) equilibrated with 20 mM sodium phosphate pH 8.0, 50 mM NaCl, 5 mM imidazole, 5 mM MgCl2, 0.1 mM EDTA, and 17% glycerol. PPO is eluted with 250 mM imidazole. The active protein is desalted on a PD-10 column (GE Healthcare Bio-Sciences Corp., Piscataway, NJ) equilibrated with a 20 mM sodium phosphate buffer, pH 7.5, 5 mM MgCl2, 1 mM EDTA and 17% glycerol. Each litre of culture provided approximately 10 mg of pure PPO, which is stored at −20° C. until being used in assays.

### Example 3: PPO Enzyme Assay (Non-Recombinant)

PPO protein (EC 1.3.3.4) is extracted from coleoptiles or shoots (150 g fresh weight) of dark-grown corn, black nightshade, morning glory, and velvetleaf seedlings as described previously (Grossmann et al. 2010). Before harvesting, the seedlings are allowed to green for 2 hours in the light in order to achieve the highest specific enzyme activities in the thylakoid fractions at low chlorophyll concentrations. At high chlorophyll concentrations significant quenching of fluorescence occurs, which limits the amount of green thylakoids that can be used in the test. Plant materials are homogenized in the cold with a Braun blender using a fresh-weight-to-volume ratio of 1:4. Homogenization buffer consisted of tris(hydroxymethyl)aminomethane (Tris)-HCl (50 mM; pH 7.3), sucrose (0.5 M), magnesium chloride (1 mM), ethylenediaminetetraacetic acid (EDTA) (1 mM) and bovine serum albumin (2 g L⁻¹). After filtration through four layers of Miracloth, crude plastid preparations are obtained after centrifugation at 10 000×g for 5 min and resuspension in homogenization buffer before centrifugation at 150×g for 2 min to remove crude cell debris. The supernatant is centrifuged at 4000×g for 15 min and the pellet fraction is resuspended in 1 ml of a buffer containing Tris-HCl (50 mM; pH 7.3), EDTA (2 mM), leupeptin (2 μM), pepstatin (2 μM) and glycerol (200 ml L⁻¹) and stored at −80° C. until use. Protein is determined in the enzyme extract with bovine serum albumin as a standard. PPO activity is assayed fluorometrically by monitoring the rate of Proto formation from chemically reduced protoporphyrinogen IX under initial velocity conditions. The assay mixture consisted of Tris-HCl (100 mM; pH 7.3), EDTA (1 mM), dithiothreitol (5 mM), Tween 80 (0.085%), protoporphyrinogen IX (2 PM), and 40 μg extracted protein in a total volume of 200 μl. The reaction is initiated by addition of substrate protoporphyrinogen IX at 22° C. saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), flumioxazin, butafenacil, acifluorfen, lactofen, bifenox, sulfentrazone, and photosynthesis inhibitor diuron as negative control are prepared in dimethyl sulfoxide (DMSO) solution (0.1 mM concentration of DMSO in the assay) and added to the assay mixture in concentrations of 0.005 pM to 5 μM before incubation. Fluorescence is monitored directly from the assay mixture using a POLARstar Optima/Galaxy (BMG) with excitation at 405 nm and emission monitored at 630 nm. Non-enzymatic activity in the presence of heat-inactivated extract is negligible. Inhibition of enzyme activity induced by the herbicide is expressed as percentage inhibition relative to untreated controls. Molar concentrations of compound required for 50% enzyme inhibition (IC₅₀ values) are calculated by fitting the values to the dose-response equation using non-linear regression analysis.

### Example 4: PPO Enzyme Assay (Recombinant)

Proto is purchased from Sigma-Aldrich (Milwaukee, WI). Protogen is prepared according to Jacobs and Jacobs (N. J. Jacobs, J. M. Jacobs, Assay for enzymatic protoporphyrinogen oxidation, a late step in heme synthesis, Enzyme 28 (1982) 206-219). Assays were conducted in 100 mM sodium phosphate pH 7.4 with 0.1 mM EDTA, 0.1% Tween 20, 5 p M FAD, and 500 mM imidazole. Dose-response curves with the PPO inhibitors saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), flumioxazin, butafenacil, acifluorfen, lactofen, bifenox, sulfentrazone, and photosynthesis inhibitor diuron as negative control, and MC-15608 are obtained in the presence of 150 μM Protogen. The excitation and emission bandwidths are set at 1.5 and 30 nm, respectively. All assays are made in duplicates or triplicates and measured using a POLARstar Optima/Galaxy (BMG) with excitation at 405 nm and emission monitored at 630 nm. Molar concentrations of compound required for 50% enzyme inhibition (IC₅₀ values) were calculated by fitting the values to the dose-response equation using non-linear regression analysis. The results are shown in the following table.

| | | Normalized | | | | |
|---|---|---|---|---|---|---|
| | SEQ. | Activity | | | | |
| | ID | (FU · | Saflufenacil | Trifludimoxazine | Saflufenacil | Trifludimoxazine |
| Amino Acid Substitution | NO. | min⁻¹ · ng⁻¹) | IC50 (M) | | Tolerance Factor | |
| PPO herbicide sensitive | 1 | 40.00 | 1.86E−09 | 5.17E−10 | 1.00 | 1.00 |
| PPO2 WC | | | | | | |
| PPO herbicide sensitive | | | | | | |
| PPO2 AC | 2 | 32.00 | 1.78E−10 | 5.96E−11 | 0.10 | 0.12 |
| dG210 | 3 & 4 | 3.20 | 1.60E−06 | 2.12E−09 | 860.26 | 4.09 |
| R128L | 1 & 2 | 28.00 | 2.22E−07 | 7.73E−10 | 119.60 | 1.50 |
| R128A, G211N, F420M | 1 & 2 | 0.33 | 1.00E−05 | 2.91E−07 | 5376.63 | 562.54 |
| G211D, F420M | 1 & 2 | 0.40 | 3.38E−07 | 1.32E−07 | 181.98 | 254.58 |
| R128A, G211V, F420M | 1 & 2 | 0.49 | 1.00E−05 | 1.15E−07 | 5376.63 | 222.66 |
| R128A, G211A, F420V | 1 & 2 | 0.62 | 1.00E−05 | 9.31E−08 | 5376.63 | 180.06 |
| R128A, G2105, F420M | 1 & 2 | 0.40 | 1.00E−05 | 6.83E−08 | 5376.63 | 132.20 |
| G211N, F420M | 1 & 2 | 0.56 | 1.00E−05 | 3.55E−08 | 5376.63 | 68.62 |
| R128A, S412N, F420M | 1 & 2 | 42.00 | 1.00E−05 | 2.43E−08 | 5376.63 | 46.93 |
| Q119L, R128A, F420M | 1 & 2 | 25.83 | 1.00E−05 | 2.06E−08 | 5376.63 | 39.94 |
| R128A, G211C, F420M | 1 & 2 | 12.49 | 1.00E−05 | 1.97E−08 | 5376.63 | 38.16 |
| R128A, G211A, F420M | 1 & 2 | 6.53 | 1.00E−05 | 1.52E−08 | 5376.63 | 29.49 |
| G211A, L397Q, F420M | 1 & 2 | 2.60 | 1.00E−05 | 1.09E−08 | 5376.63 | 21.13 |
| G211C, F420V | 1 & 2 | 4.23 | 7.67E−06 | 6.28E−09 | 4125.38 | 12.14 |
| G211A, F420V | 1 & 2 | 3.65 | 4.12E−06 | 5.46E−09 | 2215.82 | 10.57 |
| R128V, G211V | 1 & 2 | 0.58 | 6.60E−06 | 2.91E−09 | 3546.09 | 5.64 |
| G210S, F420M | 1 & 2 | 2.22 | 1.00E−05 | 2.03E−09 | 5376.63 | 3.93 |
| G210S, F420M | 1 & 2 | 2.13 | 1.00E−05 | 2.03E−09 | 5376.63 | 3.93 |
| G211A, F420M | 1 & 2 | 35.95 | 2.76E−06 | 1.51E−09 | 1481.85 | 2.93 |
| G210C, F420M | 1 & 2 | 5.66 | 2.58E−06 | 1.44E−09 | 1385.04 | 2.78 |
| R128A, G210S | 1 & 2 | 0.56 | 1.00E−05 | 1.34E−09 | 5376.63 | 2.60 |
| G211R | 1 & 2 | 1.20 | 1.13E−08 | 1.22E−09 | 6.09 | 2.36 |
| G211V, F420M | 1 & 2 | 5.09 | 7.89E−06 | 1.21E−09 | 4240.14 | 2.33 |
| G211W | 1 & 2 | 0.76 | 2.68E−08 | 1.10E−09 | 14.41 | 2.13 |
| M414Q, F420M | 1 & 2 | 17.20 | 2.80E−06 | 1.09E−09 | 1504.86 | 2.10 |
| G210T | 1 & 2 | 1.24 | 1.06E−06 | 1.00E−09 | 567.63 | 1.94 |
| Q119L, F420M | 1 & 2 | 46.86 | 1.20E−06 | 9.78E−10 | 644.80 | 1.89 |
| G211C, F420M | 1 & 2 | 9.30 | 2.32E−06 | 8.62E−10 | 1246.07 | 1.67 |
| F420M, d, V527 | 1 & 2 | 18.89 | 1.19E−06 | 8.15E−10 | 638.14 | 1.58 |
| G211D | 1 & 2 | 0.80 | 2.54E−08 | 7.53E−10 | 13.66 | 1.46 |
| G211L | 1 & 2 | 0.66 | 4.27E−07 | 7.42E−10 | 229.47 | 1.44 |
| d_K528 | 1 & 2 | 73.43 | | 7.33E−10 | | 1.42 |
| G211P | 1 & 2 | 0.62 | 1.41E−08 | 7.01E−10 | 7.58 | 1.36 |
| G211A | 1 & 2 | 16.81 | 3.86E−09 | 6.87E−10 | 2.08 | 1.33 |
| G210S | 1 & 2 | 5.90 | 5.18E−08 | 6.39E−10 | 27.87 | 1.24 |
| d_M529 | 1 & 2 | 70.49 | | 6.31E−10 | | 1.22 |
| S387Y, F420M | 1 & 2 | 9.28 | 9.92E−07 | 6.26E−10 | 533.18 | 1.21 |
| D372E, F420M | 1 & 2 | 20.82 | 2.40E−06 | 6.21E−10 | 1287.85 | 1.20 |
| G211N | 1 & 2 | 6.00 | 7.47E−09 | 6.05E−10 | 4.02 | 1.17 |
| S412N, F420M | 1 & 2 | 36.54 | 1.51E−06 | 6.01E−10 | 813.36 | 1.16 |
| G211K | 1 & 2 | 6.25 | 2.43E−09 | 5.91E−10 | 1.30 | 1.14 |
| E436I, F420M | 1 & 2 | 33.28 | 3.06E−06 | 5.78E−10 | 1644.41 | 1.12 |
| E436I, F420M | 1 & 2 | 33.29 | 3.06E−06 | 5.78E−10 | 1644.41 | 1.12 |
| M343T, F420M | 1 & 2 | 25.24 | 2.77E−06 | 5.52E−10 | 1489.90 | 1.07 |
| R128A, G211A | 1 & 2 | 11.92 | 3.71E−07 | 5.39E−10 | 199.39 | 1.04 |
| d_K532 | 1 & 2 | 76.24 | | 5.33E−10 | | 1.03 |
| G211F | 1 & 2 | 0.81 | 1.79E−08 | 5.00E−10 | 9.64 | 0.97 |
| Q447K | 1 & 2 | 21.00 | 8.82E−10 | 4.93E−10 | 0.47 | 0.95 |
| d_V527 | 1 & 2 | 74.52 | | 4.40E−10 | | 0.85 |
| G211I | 1 & 2 | 0.95 | 1.05E−08 | 4.35E−10 | 5.63 | 0.84 |
| Q119L, R128A, Q323H, | 1 & 2 | 86.12 | 2.12E−07 | 4.00E−10 | 114.19 | 0.77 |
| S412N | | | | | | |
| G211V | 1 & 2 | 11.69 | 4.23E−09 | 3.99E−10 | 2.28 | 0.77 |
| G211S | 1 & 2 | 2.86 | 2.58E−08 | 3.94E−10 | 13.87 | 0.76 |
| G211A, L397Q | 1 & 2 | 2.33 | 5.63E−07 | 3.88E−10 | 302.70 | 0.75 |
| d_D530 | 1 & 2 | 69.56 | | 3.61E−10 | | 0.70 |
| d_E531 | 1 & 2 | 77.64 | | 3.52E−10 | | 0.68 |
| S387L | 1 & 2 | 7.38 | 6.78E−09 | 3.03E−10 | 3.64 | 0.59 |
| S412T | 1 & 2 | 33.24 | 6.44E−10 | 2.89E−10 | 0.35 | 0.56 |
| G210S, G211A | 1 & 2 | 3.25 | 4.36E−08 | 2.70E−10 | 23.43 | 0.52 |
| G211T | 1 & 2 | 1.63 | 9.98E−08 | 2.67E−10 | 53.65 | 0.52 |
| S387K | 1 & 2 | 11.21 | 6.21E−09 | 2.57E−10 | 3.34 | 0.50 |
| Q119L, Q323H, S412N, | 1 & 2 | 51.44 | 1.41E−06 | 2.55E−10 | 759.42 | 0.49 |
| F420M | | | | | | |
| R128A, G211C | 1 & 2 | 4.28 | 3.57E−07 | 2.49E−10 | 191.74 | 0.48 |
| d_T533 | 1 & 2 | 76.72 | | 2.39E−10 | | 0.46 |
| d_A534 | 1 & 2 | 64.01 | | 2.34E−10 | | 0.45 |
| S412L | 1 & 2 | 51.35 | 4.54E−10 | 2.27E−10 | 0.24 | 0.44 |
| S412M | 1 & 2 | 78.96 | 4.46E−10 | 2.25E−10 | 0.24 | 0.43 |
| G211M | 1 & 2 | 5.25 | 3.11E−09 | 2.22E−10 | 1.67 | 0.43 |
| S412V | 1 & 2 | 50.22 | 7.60E−10 | 2.16E−10 | 0.41 | 0.42 |
| Q119L, Q323H, S412N | 1 & 2 | 52.00 | 8.24E−10 | 2.05E−10 | 0.44 | 0.40 |
| Q323H, S412N | 1 & 2 | 58.70 | 4.79E−10 | 1.96E−10 | 0.26 | 0.38 |
| S412Y | 1 & 2 | 43.26 | 5.39E−10 | 1.91E−10 | 0.29 | 0.37 |
| S412D | 1 & 2 | 70.61 | 5.92E−10 | 1.85E−10 | 0.32 | 0.36 |
| S412P | 1 & 2 | 40.06 | 6.66E−10 | 1.82E−10 | 0.36 | 0.35 |
| d_S523 | 1 & 2 | 56.26 | | 1.73E−10 | | 0.33 |
| S412Q | 1 & 2 | 28.47 | 5.25E−10 | 1.72E−10 | 0.28 | 0.33 |
| S412K | 1 & 2 | 95.26 | 3.10E−10 | 1.64E−10 | 0.17 | 0.32 |
| M343T | 1 & 2 | 73.85 | 1.23E−09 | 1.64E−10 | 0.66 | 0.32 |
| d_I525 | 1 & 2 | 58.17 | | 1.59E−10 | | 0.31 |
| T451V | 1 & 2 | 72.24 | 1.01E−09 | 1.59E−10 | 0.54 | 0.31 |
| G211H | 1 & 2 | 1.78 | 2.94E−09 | 1.57E−10 | 1.58 | 0.30 |
| S412A | 1 & 2 | 27.00 | 4.53E−10 | 1.53E−10 | 0.24 | 0.30 |
| Q119L, R128A | 1 & 2 | 77.43 | 1.82E−07 | 1.52E−10 | 97.59 | 0.29 |
| S412E | 1 & 2 | 65.94 | 5.92E−10 | 1.51E−10 | 0.32 | 0.29 |
| N431G | 1 & 2 | 53.81 | 7.68E−10 | 1.47E−10 | 0.41 | 0.28 |
| E436I | 1 & 2 | 49.37 | 1.13E−09 | 1.47E−10 | 0.61 | 0.28 |
| S412R | 1 & 2 | 30.41 | 6.97E−10 | 1.44E−10 | 0.38 | 0.28 |
| Q446G | 1 & 2 | 71.53 | 7.40E−10 | 1.40E−10 | 0.40 | 0.27 |
| S412I | 1 & 2 | 89.83 | 5.16E−10 | 1.40E−10 | 0.28 | 0.27 |
| d_Y526 | 1 & 2 | 40.19 | | 1.39E−10 | | 0.27 |
| d_H524 | 1 & 2 | 66.29 | | 1.34E−10 | | 0.26 |
| S412H | 1 & 2 | 83.20 | 4.98E−10 | 1.33E−10 | 0.27 | 0.26 |
| R335S | 1 & 2 | 58.65 | 8.02E−10 | 1.21E−10 | 0.43 | 0.23 |
| G210C, G211A | 1 & 2 | 2.78 | 5.80E−08 | 1.16E−10 | 31.16 | 0.22 |
| S433P | 1 & 2 | 58.76 | 6.87E−10 | 1.14E−10 | 0.37 | 0.22 |
| F345G | 1 & 2 | 43.65 | 7.17E−10 | 1.14E−10 | 0.39 | 0.22 |
| d_D522 | 1 & 2 | 29.16 | | 1.13E−10 | | 0.22 |
| D435S | 1 & 2 | 39.66 | 8.39E−10 | 1.13E−10 | 0.45 | 0.22 |
| K428D | 1 & 2 | 65.87 | 6.17E−10 | 1.11E−10 | 0.33 | 0.22 |
| G210C, G211C | 1 & 2 | 1.07 | 4.42E−08 | 1.10E−10 | 23.75 | 0.21 |
| M414Q | 1 & 2 | 42.49 | 1.15E−09 | 1.04E−10 | 0.62 | 0.20 |
| G210C | 1 & 2 | 2.08 | 4.74E−08 | 9.93E−11 | 25.50 | 0.19 |
| S412W | 1 & 2 | 21.68 | 5.82E−10 | 9.86E−11 | 0.31 | 0.19 |
| S412F | 1 & 2 | 33.89 | 3.21E−10 | 8.76E−11 | 0.17 | 0.17 |
| T358D | 1 & 2 | 54.96 | 9.66E−10 | 8.33E−11 | 0.52 | 0.16 |
| G210T, G211A | 1 & 2 | 0.61 | 3.67E−06 | 8.33E−11 | 1970.69 | 0.16 |
| S464R | 1 & 2 | 49.36 | 5.35E−10 | 8.11E−11 | 0.29 | 0.16 |
| D372E | 1 & 2 | 46.86 | 1.02E−09 | 8.02E−11 | 0.55 | 0.16 |
| S412G | 1 & 2 | 49.90 | 4.15E−10 | 7.95E−11 | 0.22 | 0.15 |
| R128A, S412N | 1 & 2 | 55.34 | 1.89E−07 | 7.45E−11 | 101.43 | 0.14 |
| R425H | 1 & 2 | 21.44 | 7.71E−10 | 7.15E−11 | 0.41 | 0.14 |
| R128A, G120C | 1 & 2 | 3.77 | 2.85E−06 | 7.15E−11 | 1532.53 | 0.14 |
| G210S, G211C | 1 & 2 | 0.86 | 2.09E−08 | 6.99E−11 | 11.21 | 0.14 |
| S412C | 1 & 2 | 19.62 | 2.69E−10 | 6.70E−11 | 0.14 | 0.13 |
| S412N | 1 & 2 | 15.00 | 1.01E−08 | 6.69E−11 | 5.43 | 0.13 |
| Q119L, S412N | 1 & 2 | 48.64 | 6.43E−10 | 6.13E−11 | 0.35 | 0.12 |
| K373D | 1 & 2 | 26.17 | 8.76E−10 | 4.64E−11 | 0.47 | 0.09 |
| S387Y | 1 & 2 | 17.02 | 1.27E−09 | 4.51E−11 | 0.68 | 0.09 |
| E356K | 1 & 2 | 19.90 | 7.12E−10 | 4.40E−11 | 0.38 | 0.09 |
| i_Q391 | 1 & 2 | 14.30 | 7.99E−10 | 3.32E−11 | 0.43 | 0.06 |
| D372E, K373D | 1 & 2 | 14.21 | 5.78E−10 | 2.92E−11 | 0.31 | 0.06 |
| D372E, K373D | 1 & 2 | 40.00 | 5.78E−10 | 2.92E−11 | 0.31 | 0.06 |
| C415H | 1 & 2 | 20.35 | 8.15E−10 | 2.52E−11 | 0.44 | 0.05 |
| M414Q, E436I | 1 & 2 | 32.72 | 1.19E−09 | | 0.64 | 0.00 |
| R128A, G211A, S387L, F420M | 1 & 2 | 51.20 | 1.00E−05 | | 5376.63 | |
| G210T, G211C | 1 & 2 | 0.50 | 8.97E−06 | | 4823.04 | |
| Q119T, G211A, F420M | 1 & 2 | 38.68 | 3.49E−06 | | 1878.54 | |
| S387L, F420M | 1 & 2 | 10.44 | 2.95E−06 | | 1584.85 | |
| G210C, L397Q | 1 & 2 | 0.22 | 2.47E−06 | | 1326.31 | |
| G210C, G211V | 1 & 2 | 0.18 | 2.13E−06 | | 1144.05 | |
| G211A, S387L, F420M | 1 & 2 | 115.08 | 1.99E−06 | | 1069.44 | |
| Q119T, F420M | 1 & 2 | 56.44 | 7.90E−07 | | 424.49 | |
| R128A, S387L | 1 & 2 | 53.92 | 3.25E−07 | | 174.56 | |
| K127N, R128C | 1 & 2 | 12.16 | 3.09E−08 | | 16.63 | |
| S387V | 1 & 2 | 3.84 | 1.76E−08 | 2.06E−10 | 9.48 | |
| S387V, R425N | 1 & 2 | 5.00 | 1.54E−08 | | 8.29 | |
| Y129N | 1 & 2 | 6.16 | 7.20E−09 | | 3.87 | |
| Y129F | 1 & 2 | 45.06 | 5.57E−09 | | 2.99 | |
| Q119T | 1 & 2 | 12.52 | 5.46E−09 | | 2.93 | |
| Q119I | 1 & 2 | 6.61 | 4.68E−09 | | 2.52 | |
| M343I | 1 & 2 | 21.00 | 4.29E−09 | | 2.31 | |
| S387T | 1 & 2 | 18.25 | 4.22E−09 | | 2.27 | |
| S387I | 1 & 2 | 5.33 | 4.11E−09 | | 2.21 | |
| M414W | 1 & 2 | 9.56 | 4.09E−09 | | 2.20 | |
| D372S | 1 & 2 | 10.39 | 4.07E−09 | | 2.19 | |
| d_P40 | 1 & 2 | 38.64 | 3.93E−09 | | 2.11 | |
| G211A, S387L | 1 & 2 | 38.68 | 3.58E−09 | | 1.92 | |
| D372N | 1 & 2 | 18.56 | 3.53E−09 | | 1.90 | |
| G264A | 1 & 2 | 23.60 | 3.42E−09 | | 1.84 | |
| E436M | 1 & 2 | 6.85 | 3.28E−09 | | 1.76 | |
| Q323P | 1 & 2 | 42.67 | 3.25E−09 | | 1.75 | |
| M414D | 1 & 2 | 12.70 | 3.20E−09 | | 1.72 | |
| S387L, M414Q | 1 & 2 | 0.00 | 3.13E−09 | | 1.68 | |
| Q119T, M414Q | 1 & 2 | 17.52 | 3.06E−09 | | 1.65 | |
| S387D | 1 & 2 | 33.37 | 3.00E−09 | | 1.61 | |
| Q119H | 1 & 2 | 4.52 | 2.98E−09 | | 1.60 | |
| M414R | 1 & 2 | 7.32 | 2.91E−09 | | 1.56 | |
| M414A | 1 & 2 | 3.16 | 2.83E−09 | | 1.52 | |
| D286G | 1 & 2 | 27.60 | 2.79E−09 | | 1.50 | |
| i_Q391, H392K, N393H | 1 & 2 | 36.90 | 2.78E−09 | | 1.49 | |
| D372K | 1 & 2 | 22.19 | 2.77E−09 | | 1.49 | |
| E436A | 1 & 2 | 21.31 | 2.73E−09 | | 1.47 | |
| M343K | 1 & 2 | 23.30 | 2.66E−09 | | 1.43 | |
| S387N | 1 & 2 | 4.57 | 2.65E−09 | | 1.42 | |
| K248G | 1 & 2 | 52.40 | 2.65E−09 | | 1.42 | |
| D372F | 1 & 2 | 15.16 | 2.58E−09 | | 1.39 | |
| S387F | 1 & 2 | 9.56 | 2.51E−09 | | 1.35 | |
| Q119A | 1 & 2 | 8.60 | 2.51E−09 | | 1.35 | |
| M343A | 1 & 2 | 46.40 | 2.49E−09 | | 1.34 | |
| M414V | 1 & 2 | 24.60 | 2.44E−09 | | 1.31 | |
| E436W | 1 & 2 | 14.87 | 2.43E−09 | | 1.31 | |
| M343F | 1 & 2 | 64.97 | 2.41E−09 | | 1.30 | |
| S387C | 1 & 2 | 28.97 | 2.38E−09 | | 1.28 | |
| M343R | 1 & 2 | 38.24 | 2.38E−09 | | 1.28 | |
| E436Y | 1 & 2 | 22.32 | 2.34E−09 | | 1.26 | |
| D372V | 1 & 2 | 22.28 | 2.25E−09 | | 1.21 | |
| S387L, S412N, E436I | 1 & 2 | 10.80 | 2.25E−09 | | 1.21 | |
| D372M | 1 & 2 | 11.96 | 2.23E−09 | | 1.20 | |
| S412N, E436I | 1 & 2 | 55.60 | 2.19E−09 | | 1.18 | |
| D372Y | 1 & 2 | 29.55 | 2.19E−09 | | 1.18 | |
| M414Y | 1 & 2 | 41.86 | 2.18E−09 | | 1.17 | |
| M343D | 1 & 2 | 109.09 | 2.17E−09 | | 1.16 | |
| S387M | 1 & 2 | 17.37 | 2.15E−09 | | 1.16 | |
| N126H | 1 & 2 | 43.52 | 2.11E−09 | | 1.13 | |
| M414S | 1 & 2 | 9.98 | 2.10E−09 | | 1.13 | |
| M414H | 1 & 2 | 18.84 | 2.09E−09 | | 1.13 | |
| M343G | 1 & 2 | 57.77 | 2.07E−09 | | 1.11 | |
| D372Q | 1 & 2 | 27.27 | 2.07E−09 | | 1.11 | |
| M343L | 1 & 2 | 16.36 | 2.06E−09 | | 1.11 | |
| E436V | 1 & 2 | 25.92 | 2.06E−09 | | 1.11 | |
| Q323K | 1 & 2 | 11.36 | 2.06E−09 | | 1.11 | |
| P260R | 1 & 2 | 38.88 | 2.04E−09 | | 1.10 | |
| D372P | 1 & 2 | 18.90 | 2.04E−09 | | 1.10 | |
| M414N | 1 & 2 | 5.76 | 2.02E−09 | | 1.09 | |
| M343S | 1 & 2 | 40.54 | 1.96E−09 | | 1.05 | |
| E436F | 1 & 2 | 10.80 | 1.95E−09 | | 1.05 | |
| E436G | 1 & 2 | 10.02 | 1.94E−09 | | 1.04 | |
| Q323V | 1 & 2 | 37.40 | 1.94E−09 | | 1.04 | |
| Q323Y | 1 & 2 | 16.92 | 1.94E−09 | | 1.04 | |
| D372T | 1 & 2 | 32.59 | 1.94E−09 | | 1.04 | |
| S387H | 1 & 2 | 24.32 | 1.93E−09 | | 1.04 | |
| L400F | 1 & 2 | 42.92 | 1.93E−09 | | 1.04 | |
| Q323N | 1 & 2 | 17.72 | 1.91E−09 | | 1.03 | |
| M414K | 1 & 2 | 10.50 | 1.88E−09 | | 1.01 | |
| M414I | 1 & 2 | 7.40 | 1.88E−09 | | 1.01 | |
| E436S | 1 & 2 | 19.88 | 1.88E−09 | | 1.01 | |
| A104L | 1 & 2 | 37.88 | 1.86E−09 | | 1.00 | |
| Q119W | 1 & 2 | 2.84 | 1.86E−09 | | 1.00 | |
| S108R | 1 & 2 | 41.22 | 1.85E−09 | | 0.99 | |
| V53I | 1 & 2 | 28.30 | 1.83E−09 | | 0.99 | |
| M343V | 1 & 2 | 24.66 | 1.83E−09 | | 0.98 | |
| H65S | 1 & 2 | 25.72 | 1.79E−09 | | 0.96 | |
| Q323S | 1 & 2 | 15.60 | 1.79E−09 | | 0.96 | |
| Q323D | 1 & 2 | 97.37 | 1.78E−09 | | 0.96 | |
| D372G | 1 & 2 | 25.12 | 1.75E−09 | | 0.94 | |
| E436K | 1 & 2 | 12.71 | 1.72E−09 | | 0.93 | |
| S387G | 1 & 2 | 12.56 | 1.72E−09 | | 0.93 | |
| S387A | 1 & 2 | 20.13 | 1.68E−09 | | 0.90 | |
| G308D | 1 & 2 | 16.52 | 1.65E−09 | | 0.89 | |
| E436D | 1 & 2 | 48.58 | 1.64E−09 | | 0.88 | |
| Q323M | 1 & 2 | 10.27 | 1.64E−09 | | 0.88 | |
| L139I | 1 & 2 | 38.44 | 1.64E−09 | | 0.88 | |
| G211A, M414Q | 1 & 2 | 60.72 | 1.61E−09 | | 0.86 | |
| Q323I | 1 & 2 | 37.28 | 1.60E−09 | | 0.86 | |
| K61R | 1 & 2 | 40.92 | 1.59E−09 | | 0.86 | |
| Q323E | 1 & 2 | 20.85 | 1.53E−09 | | 0.82 | |
| G210A | 1 & 2 | 1.96 | 1.51E−09 | 3.34E−11 | 0.81 | |
| E249D | 1 & 2 | 32.36 | 1.51E−09 | | 0.81 | |
| Q119V | 1 & 2 | 24.43 | 1.49E−09 | | 0.80 | |
| Q119S | 1 & 2 | 3.24 | 1.49E−09 | | 0.80 | |
| K87D | 1 & 2 | 26.40 | 1.48E−09 | | 0.79 | |
| L71V | 1 & 2 | 29.80 | 1.47E−09 | | 0.79 | |
| S64K | 1 & 2 | 36.44 | 1.46E−09 | | 0.78 | |
| D372A | 1 & 2 | 23.84 | 1.44E−09 | | 0.78 | |
| D372R | 1 & 2 | 34.71 | 1.44E−09 | | 0.77 | |
| R116E | 1 & 2 | 8.74 | 1.42E−09 | | 0.76 | |
| S387L, E436I | 1 & 2 | 11.12 | 1.41E−09 | | 0.76 | |
| V262K | 1 & 2 | 14.76 | 1.41E−09 | | 0.76 | |
| Q323T | 1 & 2 | 33.63 | 1.40E−09 | | 0.75 | |
| Q323W | 1 & 2 | 26.80 | 1.39E−09 | | 0.75 | |
| Q323G | 1 & 2 | 33.71 | 1.38E−09 | | 0.74 | |
| Q119C | 1 & 2 | 16.32 | 1.38E−09 | | 0.74 | |
| S412N, M414Q | 1 & 2 | 53.92 | 1.37E−09 | | 0.74 | |
| E436N | 1 & 2 | 23.80 | 1.36E−09 | | 0.73 | |
| G32A | 1 & 2 | 37.88 | 1.35E−09 | | 0.72 | |
| d_K250 | 1 & 2 | 49.52 | 1.35E−09 | | 0.72 | |
| M343Q | 1 & 2 | 33.34 | 1.32E−09 | | 0.71 | |
| Q119T, M414Q, E436I | 1 & 2 | 16.44 | 1.31E−09 | | 0.70 | |
| D372I | 1 & 2 | 15.58 | 1.29E−09 | | 0.69 | |
| Q119T, G211A, S387L | 1 & 2 | 9.84 | 1.29E−09 | | 0.69 | |
| G252K | 1 & 2 | 20.80 | 1.28E−09 | | 0.69 | |
| I257G | 1 & 2 | 39.08 | 1.28E−09 | | 0.69 | |
| D372L | 1 & 2 | 7.59 | 1.27E−09 | | 0.68 | |
| D372H | 1 & 2 | 20.08 | 1.27E−09 | | 0.68 | |
| A255V | 1 & 2 | 53.62 | 1.26E−09 | | 0.68 | |
| D453G | 1 & 2 | 56.62 | 1.26E−09 | | 0.68 | |
| I91L | 1 & 2 | 35.48 | 1.24E−09 | | 0.66 | |
| L245T | 1 & 2 | 39.72 | 1.22E−09 | | 0.66 | |
| D482G | 1 & 2 | 23.58 | 1.21E−09 | | 0.65 | |
| S387L, S412N | 1 & 2 | 6.72 | 1.20E−09 | | 0.65 | |
| Q323C | 1 & 2 | 19.95 | 1.20E−09 | | 0.65 | |
| M414L | 1 & 2 | 17.36 | 1.20E−09 | | 0.64 | |
| Q119R | 1 & 2 | 4.29 | 1.20E−09 | | 0.64 | |
| M434C | 1 & 2 | 33.31 | 1.19E−09 | | 0.64 | |
| M343W | 1 & 2 | 30.46 | 1.19E−09 | | 0.64 | |
| E436H | 1 & 2 | 24.43 | 1.18E−09 | | 0.64 | |
| S387W | 1 & 2 | 3.31 | 1.16E−09 | | 0.62 | |
| N309G | 1 & 2 | 40.04 | 1.15E−09 | | 0.62 | |
| K259G | 1 & 2 | 45.70 | 1.14E−09 | | 0.61 | |
| E436L | 1 & 2 | 9.44 | 1.13E−09 | | 0.61 | |
| S246A | 1 & 2 | 40.08 | 1.12E−09 | | 0.60 | |
| K127F | 1 & 2 | 11.84 | 1.12E−09 | | 0.60 | |
| Q119T, S412N, E436I | 1 & 2 | 30.88 | 1.12E−09 | | 0.60 | |
| Q119T, S412N | 1 & 2 | 41.72 | 1.11E−09 | | 0.60 | |
| Q119M | 1 & 2 | 41.27 | 1.11E−09 | | 0.60 | |
| E253G | 1 & 2 | 37.22 | 1.10E−09 | | 0.59 | |
| S387E | 1 & 2 | 58.75 | 1.10E−09 | | 0.59 | |
| Q119T, S387L, E436I | 1 & 2 | 4.68 | 1.10E−09 | | 0.59 | |
| D88S | 1 & 2 | 24.50 | 1.10E−09 | | 0.59 | |
| Q119T, E436I | 1 & 2 | 64.80 | 1.09E−09 | | 0.59 | |
| L67V | 1 & 2 | 34.12 | 1.07E−09 | | 0.57 | |
| N254S | 1 & 2 | 59.68 | 1.06E−09 | | 0.57 | |
| V85N | 1 & 2 | 26.94 | 1.05E−09 | | 0.57 | |
| Q119Y | 1 & 2 | 8.07 | 1.05E−09 | | 0.57 | |
| P305S | 1 & 2 | 50.58 | 1.05E−09 | | 0.56 | |
| E436R | 1 & 2 | 21.81 | 1.03E−09 | | 0.55 | |
| M414F | 1 & 2 | 30.06 | 1.02E−09 | | 0.55 | |
| M343H | 1 & 2 | 30.84 | 1.02E−09 | | 0.55 | |
| K83R | 1 & 2 | 17.24 | 1.01E−09 | | 0.54 | |
| D372W | 1 & 2 | 17.84 | 1.00E−09 | | 0.54 | |
| M343Y | 1 & 2 | 9.02 | 9.87E−10 | | 0.53 | |
| E436C | 1 & 2 | 19.62 | 9.74E−10 | | 0.52 | |
| Q323F | 1 & 2 | 22.61 | 9.52E−10 | | 0.51 | |
| K127N | 1 & 2 | 8.20 | 9.37E−10 | | 0.50 | |
| E436T | 1 & 2 | 15.79 | 9.35E−10 | | 0.50 | |
| M343N | 1 & 2 | 38.49 | 9.29E−10 | | 0.50 | |
| D372C | 1 & 2 | 25.31 | 9.14E−10 | | 0.49 | |
| E103A | 1 & 2 | 26.74 | 9.00E−10 | | 0.48 | |
| K127H | 1 & 2 | 7.84 | 8.91E−10 | | 0.48 | |
| Q323A | 1 & 2 | 32.81 | 8.75E−10 | | 0.47 | |
| Q119F | 1 & 2 | 7.96 | 8.68E−10 | | 0.47 | |
| Q291G | 1 & 2 | 24.16 | 8.65E−10 | | 0.47 | |
| L82I | 1 & 2 | 18.72 | 8.57E−10 | | 0.46 | |
| H391K | 1 & 2 | 39.76 | 8.27E−10 | | 0.44 | |
| M414T | 1 & 2 | 19.07 | 8.25E−10 | | 0.44 | |
| N392H | 1 & 2 | 18.32 | 8.16E−10 | | 0.44 | |
| K127R | 1 & 2 | 19.72 | 8.09E−10 | | 0.44 | |
| S76D | 1 & 2 | 25.46 | 8.04E−10 | | 0.43 | |
| Q119E | 1 & 2 | 3.76 | 8.02E−10 | | 0.43 | |
| K127P | 1 & 2 | 15.16 | 8.01E−10 | | 0.43 | |
| Q323R | 1 & 2 | 44.35 | 7.73E−10 | | 0.42 | |
| K86S | 1 & 2 | 22.38 | 7.65E−10 | | 0.41 | |
| M414C | 1 & 2 | 22.85 | 7.56E−10 | | 0.41 | |
| K63S | 1 & 2 | 25.74 | 7.44E−10 | | 0.40 | |
| A57V | 1 & 2 | 26.82 | 7.31E−10 | | 0.39 | |
| E224G | 1 & 2 | 19.68 | 7.28E−10 | | 0.39 | |
| K127M | 1 & 2 | 25.96 | 7.08E−10 | | 0.38 | |
| S387Q | 1 & 2 | 13.81 | 6.91E−10 | | 0.37 | |
| Q119K | 1 & 2 | 7.03 | 6.84E−10 | | 0.37 | |
| E436Q | 1 & 2 | 13.28 | 6.66E−10 | | 0.36 | |
| K127L | 1 & 2 | 16.56 | 6.36E−10 | | 0.34 | |
| K127Q | 1 & 2 | 13.44 | 6.27E−10 | | 0.34 | |
| M414E | 1 & 2 | 29.35 | 6.23E−10 | | 0.33 | |
| Q323L | 1 & 2 | 21.44 | 6.19E−10 | | 0.33 | |
| K127A | 1 & 2 | 8.08 | 6.14E−10 | | 0.33 | |
| Q159K | 1 & 2 | 66.10 | 5.94E−10 | | 0.32 | |
| K127V | 1 & 2 | 32.84 | 5.12E−10 | | 0.28 | |
| K127G | 1 & 2 | 6.92 | 5.09E−10 | | 0.27 | |
| K127Y | 1 & 2 | 14.04 | 4.98E−10 | | 0.27 | |
| K127S | 1 & 2 | 5.64 | 4.97E−10 | | 0.27 | |
| K127D | 1 & 2 | 80.64 | 4.91E−10 | | 0.26 | |
| Q119L, Q323H | 1 & 2 | 114.45 | 4.82E−10 | | 0.26 | |
| Y129W | 1 & 2 | 270.88 | 4.45E−10 | | 0.24 | |
| K127I | 1 & 2 | 31.28 | 4.42E−10 | | 0.24 | |
| K127W | 1 & 2 | 9.16 | 4.40E−10 | | 0.24 | |
| V106A | 1 & 2 | 26.68 | 4.28E−10 | | 0.23 | |
| K127T | 1 & 2 | 21.00 | 3.54E−10 | | 0.19 | |
| K127C | 1 & 2 | 10.76 | 3.42E−10 | | 0.18 | |
| K127E | 1 & 2 | 6.60 | 1.63E−10 | | 0.09 | |

### Example 5: Engineering PPO-Derivative Herbicide Tolerant Plants Having Wildtype or Mutated PPO Sequences

PPO-derivative herbicide tolerant soybean (Glyceine max), corn (Zea mays), and Canola (Brassica napus or Brassica Rapa var. or Brassica campestris L.) plants are produced by a method as described by Olhoft et al. (US patent 2009/0049567). For transformation of soybean or Arabidlopsis thaliana, Wildtype or Mutated PPO sequences encoding mutated PPO polypeptides comprising SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 129, are cloned with standard cloning techniques as described in Sambrook et al. (Molecular cloning (2001) Cold Spring Harbor Laboratory Press) in a binary vector containing resistance marker gene cassette (AHAS) and mutated PPO sequence (marked as GOI) in between ubiquitin promoter (PcUbi) and nopaline synthase terminator (NOS) sequence. For corn transformation, Wildtype or Mutated PPO sequences are cloned with standard cloning techniques as described in Sambrook et al. (Molecular cloning (2001) Cold Spring Harbor Laboratory Press) in a binary vector containing resistance marker gene cassette (AHAS) and mutated PPO sequence (marked as GOI) in between corn ubiquitin promoter (ZmUbi) and nopaline synthase terminator (NOS) sequence. Binary plasmids are introduced to Agrobacterium tumefaciens for plant transformation. Plasmid constructs are introduced into soybean's axillary meristem cells at the primary node of seedling explants via Agrobacterium-mediated transformation. After inoculation and co-cultivation with Agrobacteria, the explants are transferred to shoot introduction media without selection for one week. The explants are subsequently transferred to a shoot induction medium with 1-3 μM imazapyr (Arsenal) for 3 weeks to select for transformed cells. Explants with healthy callus/shoot pads at the primary node are then transferred to shoot elongation medium containing 1-3 μM imazapyr until a shoot elongated or the explant died. Transgenic plantlets are rooted, subjected to TaqMan analysis for the presence of the transgene, transferred to soil and grown to maturity in greenhouse. Transformation of corn plants are done by a method described by McElver and Singh (WO 2008/124495). Plant transformation vector constructs containing mutated PPO sequences are introduced into maize immature embryos via Agrobacterium-mediated transformation.

Transformed cells are selected in selection media supplemented with 0.5-1.5 μM imazethapyr for 3-4 weeks. Transgenic plantlets are regenerated on plant regeneration media and rooted afterwards. Transgenic plantlets are subjected to TaqMan analysis for the presence of the transgene before being transplanted to potting mixture and grown to maturity in greenhouse. Arabidopsis thaliana are transformed with wildtype or mutated PPO sequences by floral dip method as described by McElver and Singh (WO 2008/124495). Transgenic Arabidopsis plants are subjected to TaqMan analysis for analysis of the number of integration loci. Transformation of Oryza sativa (rice) are done by protoplast transformation as described by Peng et al. (U.S. Pat. No. 6,653,529) T0 or T1 transgenic plant of soybean, corn, and rice containing mutated PPO sequences are tested for improved tolerance to PPO-derived herbicides in greenhouse studies and mini-plot studies with the following PPO-inhibiting herbicides: saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), flumioxazin, butafenacil, acifluorfen, lactofen, bifenox, sulfentrazone, and photosynthesis inhibitor diuron as negative control.

Transgenic Arabidopsis thaliana plants are assayed for improved tolerance to saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), flumioxazin, butafenacil, acifluorfen, lactofen, bifenox, sulfentrazone, and photosynthesis inhibitor diuron as negative control in 48-well plates. Therefore, T2 seeds are surface sterilized by stirring for 5 min in ethanol+water (70+30 by volume), rinsing one time with ethanol+water (70+30 by volume) and two times with sterile, deionized water. The seeds are resuspended in 0.1% agar dissolved in water (w/v) Four to five seeds per well are plated on solid nutrient medium consisting of half-strength murashige skoog nutrient solution, pH 5.8 (Murashige and Skoog (1962) Physiologia Plantarum 15: 473-497). Compounds are dissolved in dimethylsulfoxid (DMSO) and added to the medium prior solidification (final DMSO concentration 0.1%). Multi well plates are incubated in a growth chamber at 220 C, 75% relative humidity and 110 μmol Phot*m⁻²*s⁻¹ with 14:10 h light:dark photoperiod. Growth inhibition is evaluated seven to ten days after seeding in comparison to wild type plants.

Additionally, transgenic T1 Arabidopsis plants are tested for improved tolerance to PPO-inhibiting herbicides in greenhouse studies with the following PPO-inhibiting herbicides: saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), flumioxazin, butafenacil, acifluorfen, lactofen, bifenox, sulfentrazone, and photosynthesis inhibitor diuron as negative control.

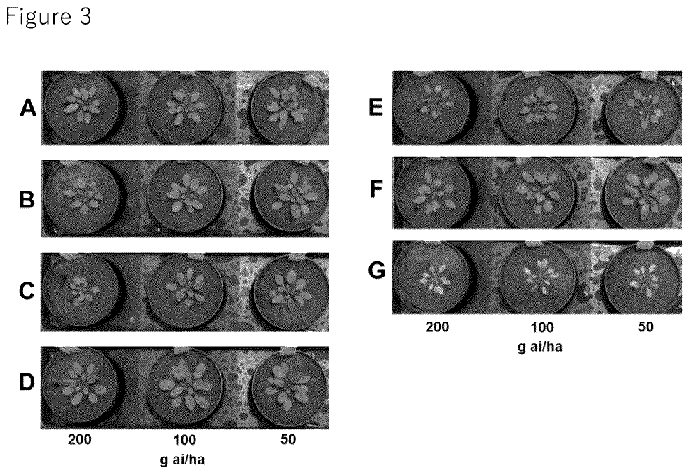

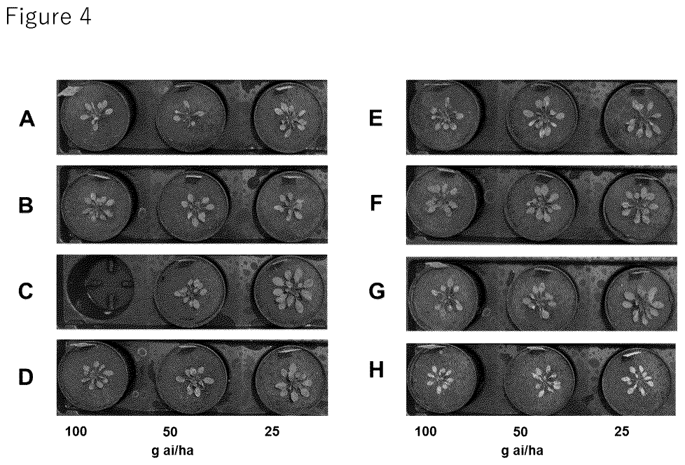

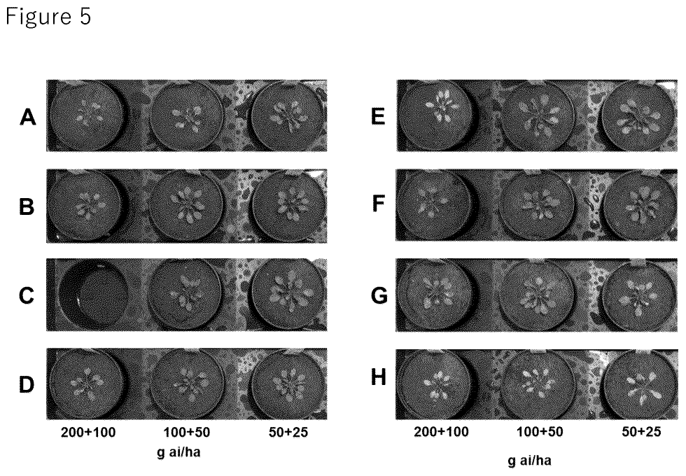

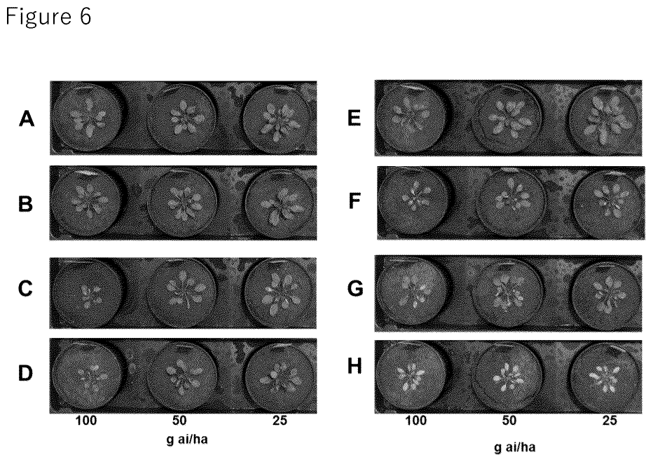

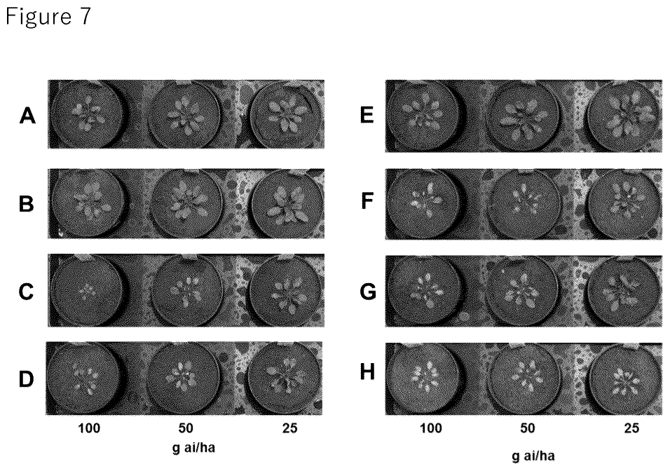

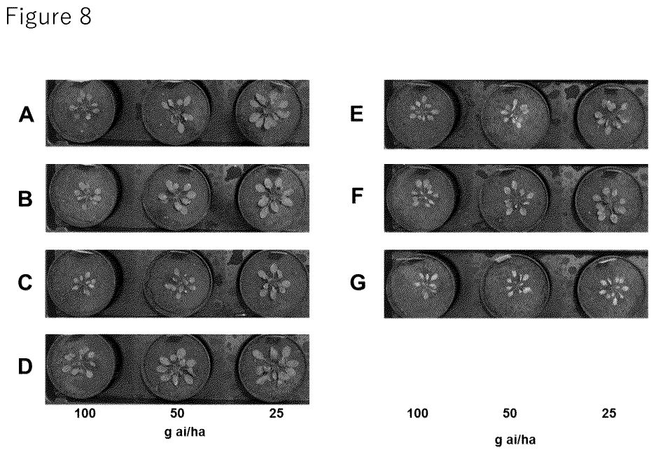

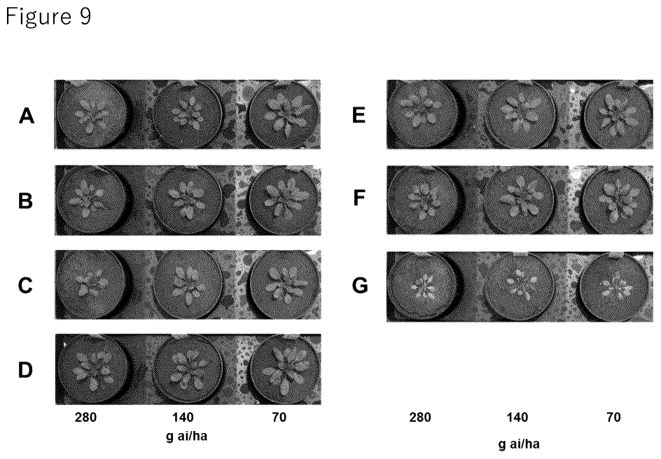

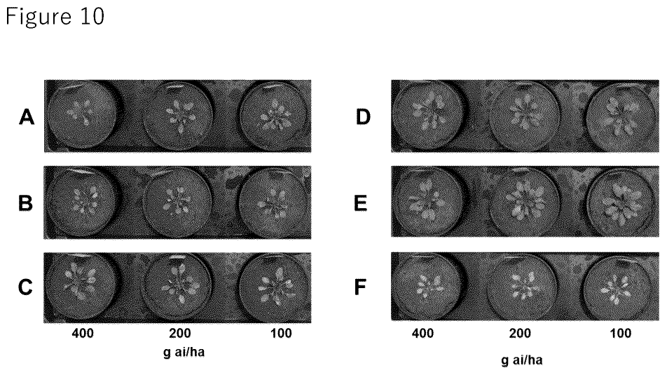

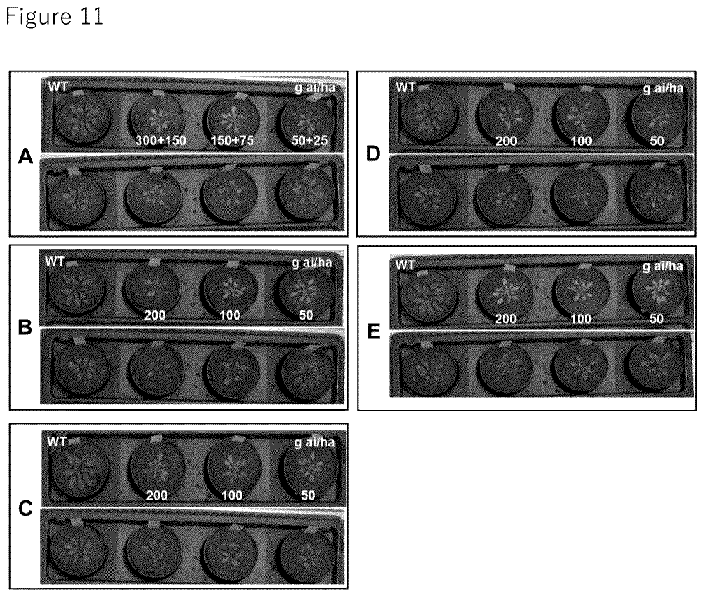

Additionally, transgenic T2 Arabidopsis plants were tested for improved tolerance to PPO-inhibiting herbicides. Plants were treated with the following herbicides in the greenhouse: Saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), Uracilpyridine 2, Uracilpyridine 4, Uracilpyridine 1, Sulfentrazone, and Flumioxazin+1% MSO. Results are shown in FIGS. 3 through 11.

### Example 6: Tissue Culture Conditions

An in vitro tissue culture mutagenesis assay has been developed to isolate and characterize plant tissue (e.g., maize, rice tissue) that is tolerant to protoporphyrinogen oxidase inhibiting herbicides, (saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), flumioxazin, butafenacil, acifluorfen, lactofen, bifenox, sulfentrazone, and photosynthesis inhibitor diuron as negative control). The assay utilizes the somaclonal variation that is found in in vitro tissue culture. Spontaneous mutations derived from somaclonal variation can be enhanced by chemical mutagenesis and subsequent selection in a stepwise manner, on increasing concentrations of herbicide.

The present invention provides tissue culture conditions for encouraging growth of friable, embryogenic maize or rice callus that is regenerable. Calli are initiated from 4 different maize or rice cultivars encompassing Zea mays and Japonica (Taipei 309, Nipponbare, Koshihikari) and Indica (Indica 1) varieties, respectively. Seeds are surface sterilized in 70% ethanol for approximately 1 min followed by 20% commercial Clorox bleach for 20 minutes. Seeds are rinsed with sterile water and plated on callus induction media. Various callus induction media are tested. The ingredient lists for the media tested are presented in Table y.

TABLE Y

| Ingredient | Supplier | R001M | | R025M | | R026M | | R327M | | R008M | | MS711R | |
|---|---|---|---|---|---|---|---|---|---|---|---|---|---|
| B5 Vitamins | Sigma | | | | | | | | | 1.0 | X | | |
| MS salts | Sigma | | | | | 1.0 | X | 1.0 | X | 1.0 | X | 1.0 | X |
| MS Vitamins | Sigma | | | | | 1.0 | X | 1.0 | X | | | | |
| N6 salts | Phytotech | 4.0 | g/L | 4.0 | g/L | | | | | | | | |
| N6 vitamins | Phytotech | 1.0 | X | 1.0 | X | | | | | | | | |
| L-Proline | Sigma | 2.9 | g/L | 0.5 | g/L | | | | | | | 1.2 | g/L |
| Casannino | BD | 0.3 | g/L | 0.3 | g/L | 2 | g/L | | | | | | |
| Acids | | | | | | | | | | | | | |
| Casein | Sigma | | | | | | | | | | | 1.0 | g/L |
| Hydrolysate | | | | | | | | | | | | | |
| L-Asp | Phytotech | | | | | | | | | | | 150 | mg/L |
| Monohydrate | | | | | | | | | | | | | |
| Nicotinic Acid | Sigma | | | | | | | | | | | 0.5 | mg/L |
| Pyridoxine HCl | Sigma | | | | | | | | | | | 0.5 | mg/L |
| Thiamine HCl | Sigma | | | | | | | | | | | 1.0 | mg/L |
| Myo-inositol | Sigma | | | | | | | | | | | 100 | mg/L |
| MES | Sigma | 500 | mg/L | 500 | mg/L | 500 | mg/L | 500 | mg/L | 500 | mg/L | 500 | mg/L |
| Maltose | VWR | 30 | g/L | 30 | g/L | 30 | g/L | 30 | g/L | | | | |
| Sorbitol | Duchefa | | | | | 30 | g/L | | | | | | |
| Sucrose | VWR | | | | | | | | | 10 | g/L | 30 | g/L |
| NAA | Duchefa | | | | | | | | | 50 | μg/L | | |
| 2,4-D | Sigma | 2.0 | mg/L | | | | | | | | | 1.0 | mg/L |
| MgCl₂•6H₂O | VWR | | | | | | | | | 750 | mg/L | | |
| →pH | | 5.8 | | 5.8 | | 5.8 | | 5.8 | | 5.8 | | 5.7 | |
| Gelrite | Duchefa | 4.0 | g/L | | | | | | | 2.5 | g/L | | |
| Agarose Type1 | Sigma | | | 7.0 | g/L | 10 | g/L | 10 | g/L | | | | |
| →Autoclave | | 15 | min | 15 | min | 15 | min | 15 | min | 15 | min | 20 | min |
| Kinetin | Sigma | | | 2.0 | mg/L | 2.0 | mg/L | | | | | | |
| NAA | Duchefa | | | 1.0 | mg/L | 1.0 | mg/L | | | | | | |
| ABA | Sigma | | | 5.0 | mg/L | | | | | | | | |
| Cefotaxinne | Duchefa | | | 0.1 | g/L | 0.1 | g/L | 0.1 | g/L | | | | |
| Vancomycin | Duchefa | | | 0.1 | g/L | 0.1 | g/L | 0.1 | g/L | | | | |
| G418 Disulfate | Sigma | | | 20 | mg/L | 20 | mg/L | 20 | mg/L | | | | |

R001M callus induction media is selected after testing numerous variations. Cultures are kept in the dark at 30° C. Embryogenic callus is subcultured to fresh media after 10-14 days.

### Example 7: Selection of Herbicide-Tolerant Calli

Once tissue culture conditions are determined, further establishment of selection conditions are established through the analysis of tissue survival in kill curves with saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), flumioxazin, butafenacil, acifluorfen, lactofen, bifenox, sulfentrazone, and photosynthesis inhibitor diuron as negative control. Careful consideration of accumulation of the herbicide in the tissue, as well as its persistence and stability in the cells and the culture media is performed. Through these experiments, a sub-lethal dose has been established for the initial selection of mutated material. After the establishment of the starting dose of saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), flumioxazin, butafenacil, acifluorfen, lactofen, bifenox, sulfentrazone, and photosynthesis inhibitor diuron as negative control in selection media, the tissues are selected in a step-wise fashion by increasing the concentration of the PPO inhibitor with each transfer until cells are recovered that grew vigorously in the presence of toxic doses. The resulting calli are further subcultured every 3-4 weeks to R001M with selective agent. Over 26,000 calli are subjected to selection for 4-5 subcultures until the selective pressure is above toxic levels as determined by kill curves and observations of continued culture. Alternatively, liquid cultures initiated from calli in MS711R with slow shaking and weekly subcultures. Once liquid cultures are established, selection agent is added directly to the flask at each subculture. Following 2-4 rounds of liquid selection, cultures are transferred to filters on solid R001M media for further growth.

### Example 8: Regeneration of Plants

Tolerant tissue is regenerated and characterized molecularly for PPO gene sequence mutations and/or biochemically for altered PPO activity in the presence of the selective agent. In addition, genes involved directly and/or indirectly in tetrapyrrole biosynthesis and/or metabolism pathways are also sequenced to characterize mutations. Finally, enzymes that change the fate (e.g. metabolism, translocation, transportation) are also sequence to characterized mutations. Following herbicide selection, calli are regenerated using a media regime of R025M for 10-14 days, R026M for ca. 2 weeks, R327M until well formed shoots are developed, and R008S until shoots are well rooted for transfer to the greenhouse. Regeneration is carried out in the light. No selection agent is included during regeneration. Once strong roots are established, MO regenerants are transplant to the greenhouse in square or round pots. Transplants are maintained under a clear plastic cup until they are adapted to greenhouse conditions. The greenhouse is set to a day/night cycle of 27° C./21° C. (80° F./70° F.) with 600 W high pressure sodium lights supplementing light to maintain a 14-hour day length. Plants are watered according to need, depending in the weather and fertilized daily.

### Example 9: Sequence Analysis

Leaf tissue is collected from clonal plants separated for transplanting and analyzed as individuals. Genomic DNA is extracted using a Wizard® 96 Magnetic DNA Plant System kit (Promega, U.S. Pat. Nos. 6,027,945 & 6,368,800) as directed by the manufacturer. Isolated DNA is PCR amplified using the appropriate forward and reverse primer.

PCR amplification is performed using Hotstar Taq DNA Polymerase (Qiagen) using touchdown thermocycling program as follows: 96° C. for 15 min, followed by 35 cycles (96° C., 30 sec; 58° C.-0.2° C. per cycle, 30 sec; 72° C., 3 min and 30 sec), 10 min at 72° C. PCR products are verified for concentration and fragment size via agarose gel electrophoresis. Dephosphorylated PCR products are analyzed by direct sequence using the PCR primers (DNA Landmarks, or Entelechon). Chromatogram trace files (.scf) are analyzed for mutation relative to the wild-type gene using Vector NTI Advance 10™ (Invitrogen). Based on sequence information, mutations are identified in several individuals. Sequence analysis is performed on the representative chromatograms and corresponding AlignX alignment with default settings and edited to call secondary peaks.

### Example 10: Demonstration of Herbicide-Tolerance

T0 or T1 transgenic plant of soybean, corn, Canola varieties and rice containing PPO1 and or PPO2 sequences are tested for improved tolerance to herbicides in greenhouse studies and mini-plot studies with the following herbicides: saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), flumioxazin, butafenacil, acifluorfen, lactofen, bifenox, sulfentrazone, and photosynthesis inhibitor diuron as negative control. For the pre-emergence treatment, the herbicides are applied directly after sowing by means of finely distributing nozzles. The containers are irrigated gently to promote germination and growth and subsequently covered with transparent plastic hoods until the plants have rooted. This cover causes uniform germination of the test plants, unless this has been impaired by the herbicides. For post emergence treatment, the test plants are first grown to a height of 3 to 15 cm, depending on the plant habit, and only then treated with the herbicides. For this purpose, the test plants are either sown directly, and grown in the same containers or they are first grown separately and transplanted into the test containers a few days prior to treatment.

For testing of TO plants, cuttings can be used. In the case of soybean plants, an optimal shoot for cutting is about 7.5 to 10 cm tall, with at least two nodes present. Each cutting is taken from the original transformant (mother plant) and dipped into rooting hormone powder (indole-3-butyric acid, IBA). The cutting is then placed in oasis wedges inside a bio-dome. Wild type cuttings are also taken simultaneously to serve as controls. The cuttings are kept in the bio-dome for 5-7 days and then transplanted to pots and then acclimated in the growth chamber for two more days. Subsequently, the cuttings are transferred to the greenhouse, acclimated for approximately 4 days, and then subjected to spray tests as indicated. Depending on the species, the plants are kept at 10-25° C. or 20-35° C. The test period extends over 3 weeks. During this time, the plants are tended and their response to the individual treatments is evaluated. Herbicide injury evaluations are taken at 2 and 3 weeks after treatment. Plant injury is rated on a scale of 0% to 100%, 0% being no injury and 100% being complete death.

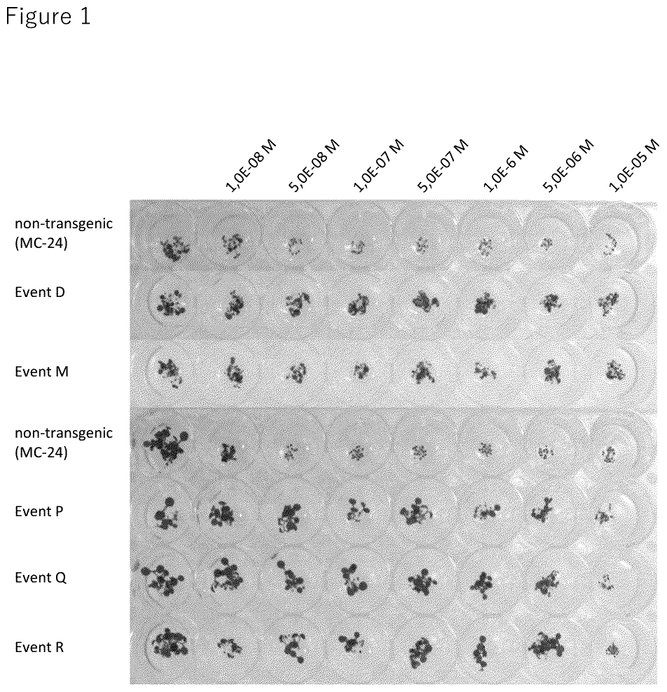

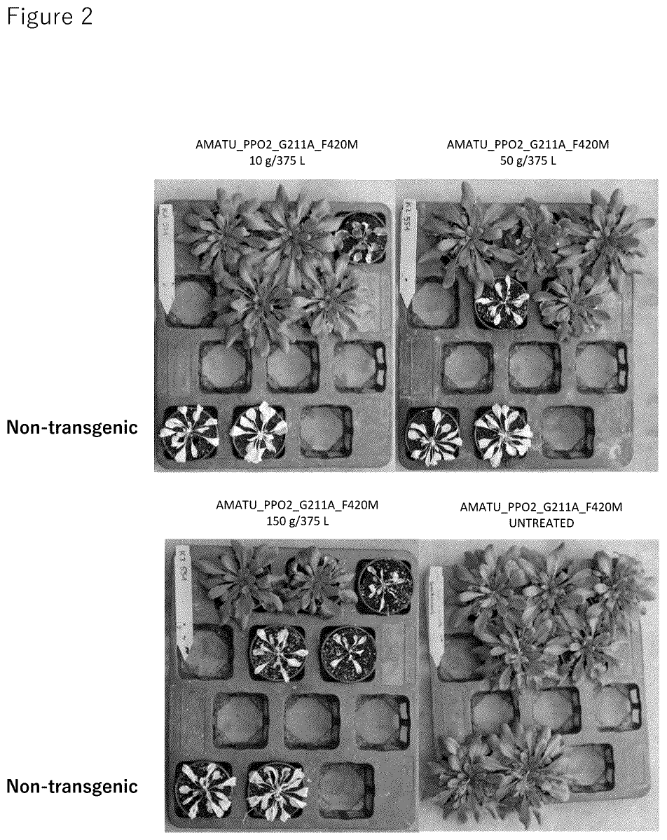

Transgenic Arabidopsis thaliana plants are assayed for improved tolerance to saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), flumioxazin, butafenacil, acifluorfen, lactofen, bifenox, sulfentrazone, and photosynthesis inhibitor diuron as negative control, in 48-well plates. Therefore, T2 seeds are surface sterilized by stirring for 5 min in ethanol+water (70+30 by volume), rinsing one time with ethanol+water (70+30 by volume) and two times with sterile, deionized water. The seeds are resuspended in 0.1% agar dissolved in water (w/v) Four to five seeds per well are plated on solid nutrient medium consisting of half-strength murashige skoog nutrient solution, pH 5.8 (Murashige and Skoog (1962) Physiologia Plantarum 15: 473-497). Compounds are dissolved in dimethylsulfoxid (DMSO) and added to the medium prior solidification (final DMSO concentration 0.1%). Multi well plates are incubated in a growth chamber at 220 C, 75% relative humidity and 110 μmol Phot*m⁻²*s⁻¹ with 14:10 h light:dark photoperiod. Growth inhibition was evaluated seven to ten days after seeding in comparison to wild type plants. Additionally, transgenic T1 Arabidopsis plants were tested for improved tolerance to herbicides in greenhouse studies with the following herbicides: saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), flumioxazin, butafenacil, acifluorfen, lactofen, bifenox, sulfentrazone, and photosynthesis inhibitor diuron as negative control. The results are shown in the following table and in FIGS. 1 and 2.

Germination Assay - trangenic ARBTH germinating
in medium containing selection agent

| | | | | Tolerance Factor |
|---|---|---|---|---|
| Event | Species_gene_substitution | Compound | IC 50 (M) | (non transgeneic = 1.0) |
| | non-transgenic (MC-24) | Saflufenacil | 2.50E−08 | 1 |
| | AMATU_PPO2_wild_type | Saflufenacil | 1.06E−07 | 10 |
| D | AMATU_PPO2_G211A_F420M | Saflufenacil | 2.50E−05 | 1000 |
| M | AMATU_PPO2_G211A_F420M | Saflufenacil | 2.50E−05 | 1000 |
| P | AMATU_PPO2_G211A_F420M | Saflufenacil | 7.50E−06 | 300 |
| Q | AMATU_PPO2_G211A_F420M | Saflufenacil | 7.50E−06 | 300 |
| R | AMATU_PPO2_G211A_F420M | Saflufenacil | 7.50E−06 | 300 |

### Example 11: Herbicide Selection Using Tissue Culture

Media is selected for use and kill curves developed as specified above. For selection, different techniques are utilized. Either a step wise selection is applied, or an immediate lethal level of herbicide is applied. In either case, all of the calli are transferred for each new round of selection. Selection is 4-5 cycles of culture with 3-5 weeks for each cycle. Cali are placed onto nylon membranes to facilitate transfer (200 micron pore sheets, Biodesign, Saco, Maine). Membranes are cut to fit 100×20 mm Petri dishes and are autoclaved prior to use 25-35 calli (average weight/calli being 22 mg) are utilized in every plate. In addition, one set of calli are subjected to selection in liquid culture media with weekly subcultures followed by further selection on semi-solid media. Mutant lines are selected using saflufenacil, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin), flumioxazin, butafenacil, acifluorfen, lactofen, bifenox, sulfentrazone, and photosynthesis inhibitor diuron as negative control. Efficiencies of obtaining mutants is high either based on a percentage of calli that gave rise to a regenerable, mutant line or the number of lines as determined by the gram of tissue utilized.

### Example 12: Maize Whole Plant Transformation and PPO Inhibitor Tolerance Testing

Immature embryos are transformed according to the procedure outlined in Peng et al. (WO2006/136596). Plants are tested for the presence of the T-DNA by Taqman analysis with the target being the nos terminator which is present in all constructs. Healthy looking plants are sent to the greenhouse for hardening and subsequent spray testing. The plants are individually transplanted into MetroMix 360 soil in 4″ pots. Once in the greenhouse (day/night cycle of 27° C./21° C. with 14 hour day length supported by 600 W high pressure sodium lights), they are allowed to grow for 14 days. They are then sprayed with a treatment of 25 to 200 g ai/ha saflufenacil+1.0% v/v methylated seed oil (MSO) and/or 25-200 g ai/ha 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin) plus 1% MSO. Other PPO inhibiting herbicides are also tested in a similar fashion for confirming cross resistance: flumioxazin, butafenacil, acifluorfen, lactofen, bifenox, sulfentrazone, and photosynthesis inhibitor diuron as negative control. Herbicide injury evaluations are taken at 7, 14 and 21 days after treatment. Herbicide injury evaluations are taken 2, 7, 14 and 21 days post-spray to look for injury to new growth points and overall plant health. The top survivors are transplanted into gallon pots filled with MetroMix 360 for seed production.

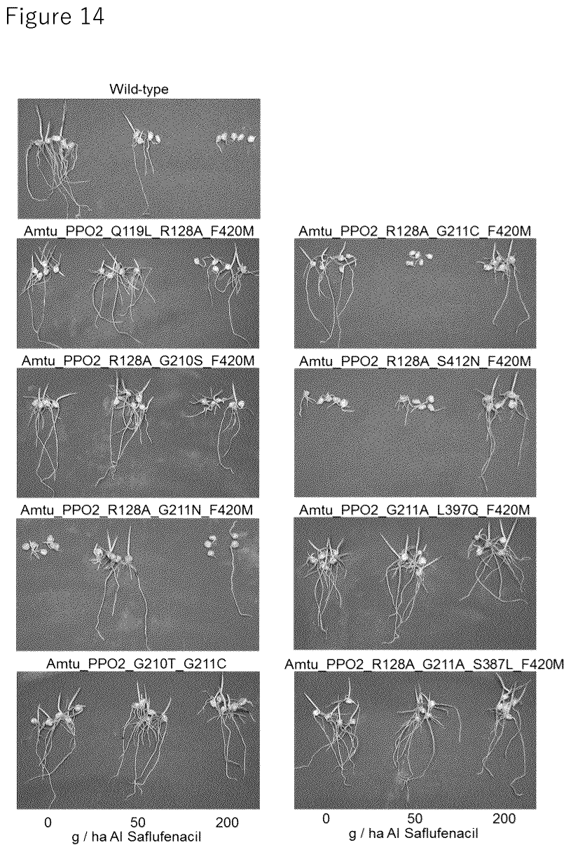

In addition, T1 transgenic maize harboring Amtu_PPO2 variants was tested for tolerance for pre-emergence application of Saflufenacil. Seeds were planted in a mixture of sand an Metromix and then immediately treated with 50 and 200 g ai/ha Saflufenacil. Seeds and plantlets were assessed for germination and root length. Results are shown in FIG. 14.

### Example 13: Soybean Transformation and PPO Inhibitor Tolerance Testing

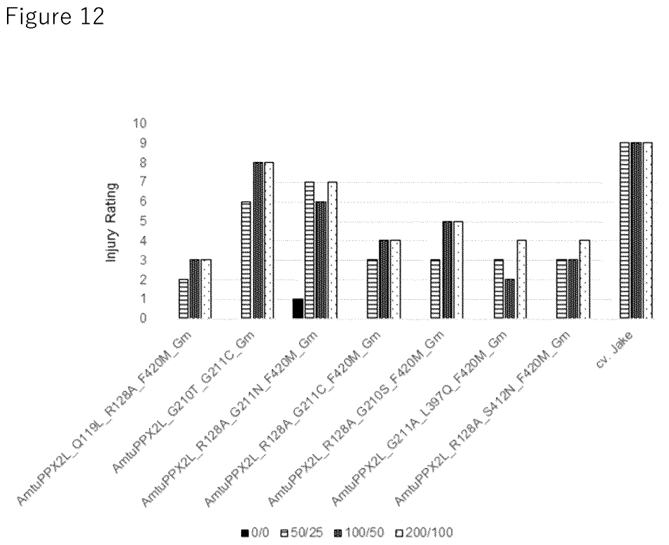

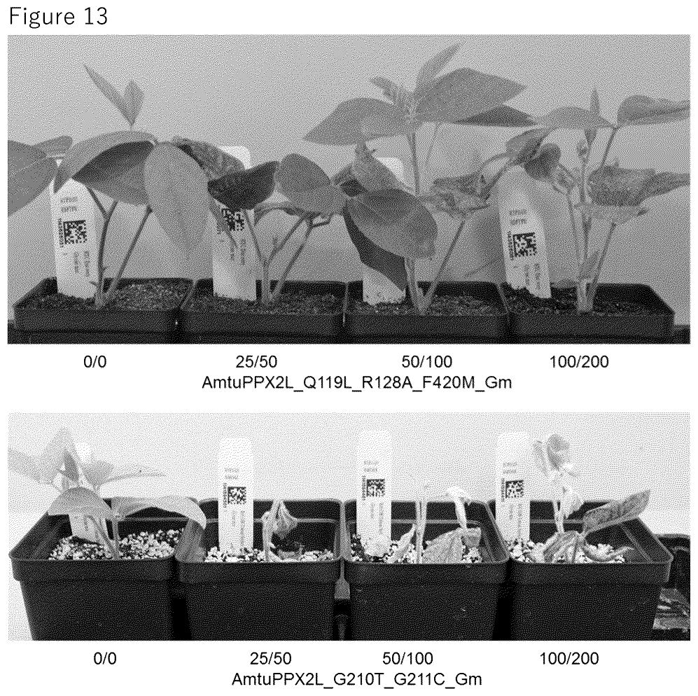

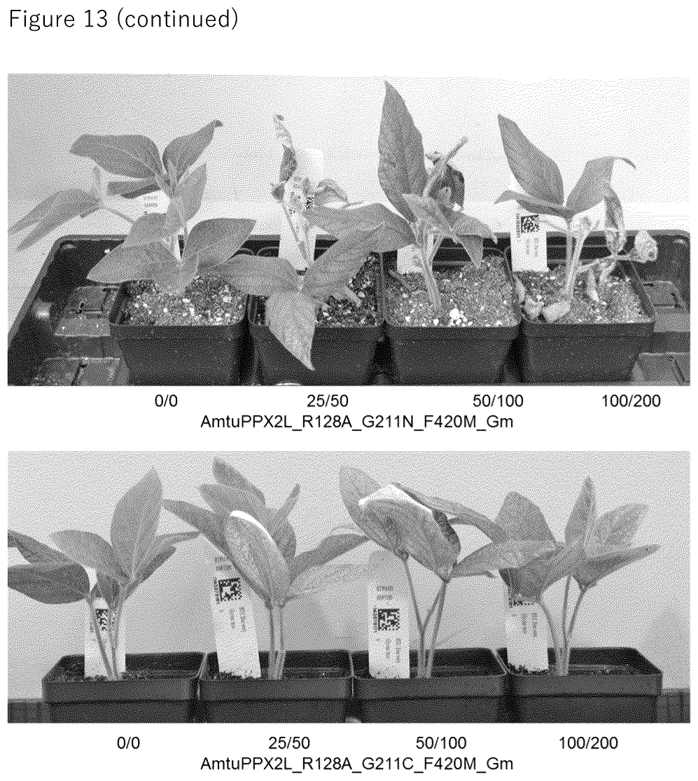

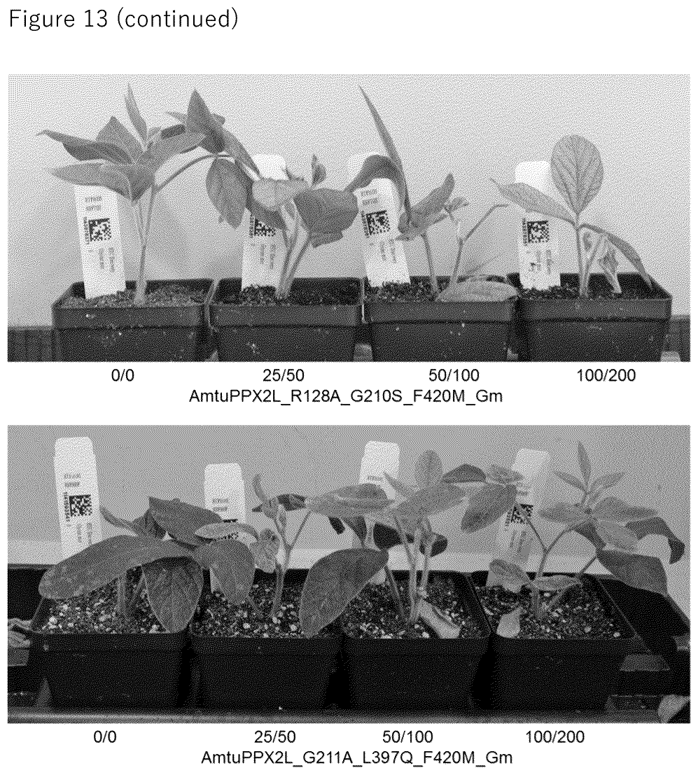

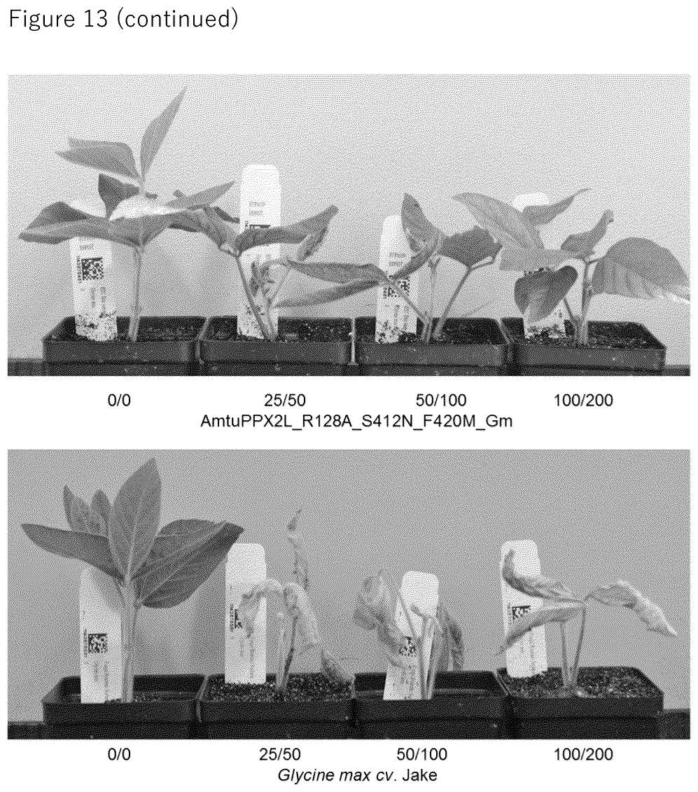

Soybean cv Jake is transformed as previously described by Siminszky et al., Phytochem Rev. 5:445-458 (2006). After regeneration, transformants are transplanted to soil in small pots, placed in growth chambers (16 hr day/8 hr night; 25° C. day/23° C. night; 65% relative humidity; 130-150 microE m-2 s-1) and subsequently tested for the presence of the T-DNA via Taqman analysis. After a few weeks, healthy, transgenic positive, single copy events are transplanted to larger pots and allowed to grow in the growth chamber. An optimal shoot for cutting is about 3-4 inches tall, with at least two nodes present. Each cutting is taken from the original transformant (mother plant) and dipped into rooting hormone powder (indole-3-butyric acid, IBA). The cutting is then placed in oasis wedges inside a bio-dome. The mother plant is taken to maturity in the greenhouse and harvested for seed. Wild type cuttings are also taken simultaneously to serve as negative controls. The cuttings are kept in the bio-dome for 5-7 days and then transplanted to 3 inch pots and then acclimated in the growth chamber for two more days. Subsequently, the cuttings were transferred to the greenhouse, acclimated for approximately 4 days, and then sprayed with a treatment of 0-200 g ai/ha saflufenacil plus 1% MSO and/or 25-200 g ai/ha 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4/trifludimoxazin) plus 1% MSO. Other PPO inhibiting herbicides are also tested in a similar fashion for confirming cross resistance: flumioxazin, butafenacil, acifluorfen, lactofen, bifenox, sulfentrazone, and photosynthesis inhibitor diuron as negative control. Herbicide injury evaluations are taken at 7 days after treatment. Results are shown in FIGS. 12 and 13.

The following gives a definition of the injury scores measured above:

Score Description of Injury
0 No Injury
1 Minimal injury, only a few patches of leaf injury or chlorosis.
2 Minimal injury with slightly stronger chlorosis. Overall growth points remain undamaged.
3 Slightly stronger injury on secondary leaf tissue, but primary leaf and growth points are still undamaged.
4 Overall plant morphology is slightly different, some chlorosis and necrosis in secondary growth points and leaf tissue. Stems are intact. Regrowth is highly probable within 1 week.
5 Overall plant morphology is clearly different, some chlorosis and necrosis on a few leaves and growth points, but primary growth point is intact. Stem tissue is still green. Regrowth is highly probably within 1 week.
6 Strong injury can be seen on the new leaflet growth. Plant has a high probability to survive only through regrowth at different growth points. Most of the leaves are chlorotic/necrotic but stem tissue is still green. May have regrowth but with noticeable injured appearance.
7 Most of the active growth points are necrotic. There may be a single growth point that could survive and may be partially chlorotic or green and partially necrotic. Two leaves may still be chlorotic with some green; the rest of the plant including stem is necrotic.
8 Plant will likely die, and all growth points are necrotic. One leaf may still be chlorotic with some green. The remainder of the plant is necrotic.
9 Plant is dead.
*Not tested

### Example 14: Transient Protein Expression in Tobacco Leafs

Transient expression of mutated PPO sequences as wildtype or with respective mutations were done as described previously (Voinnet O., et al., 2003, The Plant Journal 33, 949-956). In brief, cloning of GOI and Agrobacterium transformation (strain: GV2260) were done as described in EXAMPLE 5. Young leaves of Nicotiana benthamiana were infiltrated with transgenic Agrobacterium suspension (OD⁶⁰⁰ of 1.0) harboring binary vector constructs containing a GOI gene controlled by a promoter and terminator sequence. 48 h to 72 h after infiltration punches of leave discs (0.75 cm in diameter) were transferred to 6-well plates with medium (half strength Linsmaier-Skoog (Linsmaier and Skoog (1965) Physiol. Plant. 18: 100-127) nutrient solution or water) containing herbicide of interest in different concentrations. Multi well plates were incubated in a growth chamber at 220 C, 75% relative humidity and 110 μmol Phot*m⁻²*s⁻¹ with 14:10 h light:dark photoperiod.

### Example 15: Demonstration of Herbicide Tolerance of Transiently Transformed Tobacco Leaf Discs

Leaf discs, generated as described in EXAMPLE 14, expressing a protein encoded by GOI, were subjected to analysis on improved tolerance to herbicide treatment. For analysis of herbicide damage, chlorophyll fluorescence were identified as indicative marker (Dayan and Zaccaro (2012) Pest. Biochem. Physiol. 102: 189-197). In addition to monitor herbicide effect by visual inspection the photosynthetic yield of photosystem II were done with a MAXI imaging PAM machine (IMAGINE-PAM M-Series, Walz, Effeltrich, Germany) 48 h after starting herbicide treatment. PSII yield were measured as per manufacturer instructions. Tolerance factors were calculated based on IC₅₀ values of PSII yield inhibition of transformed versus empty vector-transformed leaf discs. IC₅₀ of PSII yield inhibition in empty vector-transformed leaf discs treated with Saflufenacil or 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione for 48 h was measured with 1.1*10⁻⁷ M or 1.1*10⁻⁸ M, respectively.

1. A plant or plant part comprising a polynucleotide encoding a mutated PPO polypeptide, the expression of said polynucleotide confers to the plant or plant part tolerance to herbicides, wherein said mutated PPO polypeptide comprises one or more of the following motifs.
1)
Motif 1:
GT[C/S]GGDP
Wherein the glycine at position 4, and/or 5 within said motif of the corresponding wildtype sequence is substituted by any other amino acid
2)
Motif 2:
[A/S/C]PS[D/N][X][X]L
Wherein the serine at position 3 within said motif of the corresponding wildtype sequence is substituted by any other amino acid
3)
Motif 3:
[R/Q][E/D]KQQ[L/Y]P
Wherein the glutamine at position 4 within said motif of the corresponding wildtype sequence is substituted by any other amino acid
4)
Motif 4:
L[I/V]PSKE
wherein the serine at position 4 within said motif of the corresponding wildtype sequence is substituted by any other amino acid.

2. The plant or plant part of claim 1, wherein the mutated PPO polypeptide in addition comprises one or more of the following motifs
a. Motif 5: SQ[N/K/H]KRYI, wherein the Arg at position 5 within said motif is substituted by any other amino acid;
b. Motif 6: TLGTLFSS, wherein the Leu at position 2 within said motif is substituted by by any other amino acid;
c. Motif 7: [F/Y]TTF[V/I]GG, wherein the Phe at position 4 within said motif is substituted by by any other amino acid.

3. The plant or plant part of claim 1, wherein said mutated PPO polypeptide comprises a combination of Motifs, said combination is selected from the group consisting of: Motif 5-Motif 1-Motif-2-Motif 7; Motif 5-Motif 1-Motif 7, Motif 5-Motif 4-Motif 7; Motif 3-Motif 5-Motif 7; Motif 1-Motif 7; and Motif 1-Motif 6-Motif 7.

4. The plant or plant part of claim 1, wherein the polynucleotide encoding the mutated PPO polypeptide comprises the nucleic acid sequence set forth in SEQ ID NO: 118, 119, 120, 121, 122, 123, 124, 125, 126, 127, 128 or 130, or a homologue, variant or derivative thereof.

5. The plant or plant part of claim 1, wherein the mutated PPO polypeptide is a functional variant having, over the full-length of the variant, at least about 60%, illustratively, at least about 80%, 90%, 95%, 98%, 99% or more amino acid sequence identity to SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104,105, 106, 107, 108, 109, 110, 111, 112, 113, 114, 115, 116, 117 or 129.

6. The plant or plant part of claim 1, wherein the mutated PPO refers to a PPO polypeptide the amino acid sequence of which differs from the respective wildtype amino acid sequence at one or more positions corresponding to positions 32, 53, 57, 61, 63, 64, 65, 67, 71, 76, 82, 83, 85, 86, 87, 88, 91, 103, 104, 106, 108, 116,119, 126, 127, 129, 139, 159, 210, 211, 224, 245, 246, 248, 249, 252, 253, 254, 255, 257, 259, 260, 262, 264, 286, 291, 305, 308, 309, 323, 335, 343, 345, 358, 372, 373, 387, 391, 392, 400, 412, 414, 415, 425, 428, 431, 433, 434, 435, 436, 447, 451, 453, 464, 466, 482 of SEQ ID NO: 1 or 2, wherein said difference refers to a substitution of the amino acid at that positions by any other amino acid.

7. A seed capable of germination into a plant comprising in at least some of its cells a polynucleotide operably linked to a promoter operable in plant cells, the promoter capable of expressing a mutated PPO polypeptide encoded by the polynucleotide as defined in claim 1, the expression of the mutated PPO polypeptide conferring to the plant tolerance to herbicides.

8. A plant cell of or capable of regenerating a plant comprising in at least some of its cells a polynucleotide operably linked to a promoter operable in plant cells, the promoter capable of expressing a mutated PPO polypeptide encoded by the polynucleotide as defined in claim 1, the expression of the mutated PPO polypeptide conferring to the plant tolerance to herbicides, wherein the plant cell comprises the polynucleotide operably linked to a promoter.

9. A plant cell comprising a polynucleotide operably linked to a promoter operable in a cell, the promoter capable of expressing a mutated PPO polypeptide encoded by the polynucleotide as defined in claim 1, the expression of the mutated PPO polypeptide conferring to the plant tolerance to herbicides.

10. A plant product prepared from a plant or plant part comprising in at least some of its cells a polynucleotide operably linked to a promoter operable in plant cells, the promoter capable of expressing a mutated PPO polypeptide encoded by the polynucleotide as defined in claim 1, the expression of the mutated PPO polypeptide conferring to the plant tolerance to herbicides.

11. A progeny or descendant plant derived from a plant comprising in at least some of its cells a polynucleotide operably linked to a promoter operable in plant cells, the promoter capable of expressing a mutated PPO polypeptide encoded by the polynucleotide as defined in claim 1, wherein the progeny or descendant plant comprises in at least some of its cells the polynucleotide operably linked to the promoter, the expression of the mutated PPO polypeptide conferring to the progeny or descendant plant tolerance to the herbicides.

12. A method for controlling weeds at a locus for growth of a plant, the method comprising: (a) applying an herbicide composition comprising herbicides to the locus; and (b) planting a seed at the locus, wherein the seed is capable of producing a plant that comprises in at least some of its cells a polynucleotide operably linked to a promoter operable in plant cells, the promoter capable of expressing a mutated PPO polypeptide encoded by the polynucleotide as defined in claim 1, the expression of the mutated PPO polypeptide conferring to the plant tolerance to herbicides.

13. The method of claim 12, wherein herbicide composition is applied to the weeds and to the plant produced by the seed.

14. A method of producing a plant having tolerance to herbicide, the method comprising regenerating a plant from a plant cell transformed with a recombinant polynucleotide operably linked to a promoter operable in plant cells, the promoter capable of expressing a mutated PPO polypeptide encoded by the polynucleotide as defined in claim 1, the expression of the mutated PPO polypeptide conferring to the plant tolerance to herbicides.

15. A method of producing a progeny plant having tolerance to herbicide, the method comprising: crossing a first herbicide-tolerant plant with a second plant to produce a herbicide-tolerant progeny plant, wherein the first plant and the progeny plant comprise in at least some of their cells a polynucleotide operably linked to a promoter operable in plant cells, the promoter capable of expressing a mutated PPO polypeptide encoded by the polynucleotide as defined in claim 1, the expression of the mutated PPO polypeptide conferring to the plant tolerance to herbicides.

16. A plant or plant part comprising in at least some of its cells a polynucleotide operably linked to a promoter operable in plant cells, the promoter capable of expressing a mutated PPO polypeptide encoded by the polynucleotide as defined in claim 1, the expression of the mutated PPO polypeptide conferring to the plant tolerance to herbicides, wherein the plant or plant part further exhibits a second or third herbicide-tolerant trait.

17. An isolated and/or recombinantly produced and/or synthetic nucleic acid molecule comprising a nucleic acid molecule encoding a mutated PPO polypeptide selected from the group consisting of:
(a) a nucleic acid molecule encoding a mutated PPO polypeptide comprising the sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105,106, 107, 108, 109, 110, 111, 112, 113, 114, 115, 116, 117 or 129, or a variant, paralogue, orthologue or homolog thereof;
(b) a nucleic acid molecule, which, as a result of the degeneracy of the genetic code, can be derived from a nucleic acid molecule encoding a mutated PPO polypeptide comprising the sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117 or 129, or a variant, paralogue, orthologue or homolog thereof, and confers increased herbicide tolerance or resistance, as compared to a corresponding, non-transformed, wild type plant cell, a plant or a part thereof;
(c) a nucleic acid molecule encoding a mutated PPO polypeptide having 30% or more identity with the amino acid sequence of the PPO polypeptide sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117 or 129, and confers increased herbicide tolerance or resistance, as compared to a corresponding, non-transformed, wild type plant cell, a plant or a part thereof;
(d) nucleic acid molecule which hybridizes with a nucleic acid molecule of (a), (b), or (c), under stringent hybridization conditions and confers increased herbicide tolerance or resistance, as compared to a corresponding, e.g. non-transformed, wild type plant cell, a plant or a part thereof;
wherein the amino acid sequence of the encoded mutated PPO polypeptide differs from the wildtype amino acid sequence of a PPO polypeptide at one or more positions corresponding to positions 32, 53, 57, 61, 63, 64, 65, 67, 71, 76, 82, 83, 85, 86, 87, 88, 91, 103, 104, 106, 108, 116,119, 126, 127, 129, 139, 159, 210, 211, 224, 245, 246, 248, 249, 252, 253, 254, 255, 257, 259, 260, 262, 264, 286, 291, 305, 308, 309, 323, 335, 343, 345, 358, 372, 373, 387, 391, 392, 400, 412, 414, 415, 425, 428, 431, 433, 434, 435, 436, 447, 451, 453, 464, 466, 482 of SEQ ID NO: 1 or 2, wherein said difference refers to a substitution of the amino acid at that positions by any other amino acid.

18. The nucleic acid molecule of claim 17, wherein the the amino acid sequence of the encoded mutated PPO polypeptide additionally differs from the wildtype amino acid sequence of a PPO polypeptide at one or more positions corresponding to positions 128, 397, or 420 of SEQ ID NO: 1 or 2, wherein said difference refers to a substitution of the amino acid at that positions by any other amino acid.

19. The nucleic acid of claim 17, wherein the amino acids at positions corresponding to positions 128, 211, 387, and 420 of SEQ ID NO: 1 or 2 are substituted by any other amino acid.

20. The nucleic acid of claim 17, wherein the amino acids at positions corresponding to positions 128, 211, and 420 of SEQ ID NO: 1 or 2 are substituted by any other amino acid.

21. The nucleic acid of claim 17, wherein the amino acids at positions corresponding to positions 128, 210, and 420 of SEQ ID NO: 1 or 2 are substituted by any other amino acid.

22. The nucleic acid of claim 17, wherein the amino acids at positions corresponding to positions 128, 412, and 420 of SEQ ID NO: 1 or 2 are substituted by any other amino acid.

23. The nucleic acid of claim 17, wherein the amino acids at positions corresponding to positions 119, 128, and 420 of SEQ ID NO: 1 or 2 are substituted by any other amino acid.

24. The nucleic acid of claim 17, wherein the amino acids at positions corresponding to positions 211, 397 and 420 of SEQ ID NO: 1 or 2 are substituted by any other amino acid.

25. The nucleic acid of claim 17, wherein the amino acids at positions corresponding to positions 211, and 420 of SEQ ID NO: 1 or 2 are substituted by any other amino acid.

26. An expression cassette comprising the nucleic acid molecule as defined in claim 17, and a promoter operable in plant cells.

27. A vector comprising the nucleic acid molecule as defined in claim 17.

28. An isolated, recombinant and/or chemically synthesized mutated PPO polypeptide encoded by the nucleic acid molecule as claimed in claim 17 or a polypeptide having at least 80%, 90%, 95%, 98%, 99% identity to the sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117 or 129, or a variant, paralogue, orthologue or homolog thereof, wherein the amino acid sequence of the mutated PPO polypeptide differs from the wildtype amino acid sequence of a PPO polypeptide at one or more positions corresponding to positions 32, 53, 57, 61, 63, 64, 65, 67, 71, 76, 82, 83, 85, 86, 87, 88, 91, 103, 104, 106, 108, 116, 119, 126, 127,129, 139, 159, 210, 211, 224, 245, 246, 248, 249, 252, 253, 254, 255, 257, 259, 260, 262, 264, 286, 291, 305, 308, 309, 323, 335, 343, 345, 358, 372, 373, 387, 391, 392, 400, 412, 414, 415, 425, 428, 431, 433, 434, 435, 436, 447, 451, 453, 464, 466, 482 of SEQ ID NO: 1 or 2, wherein said difference refers to a substitution of the amino acid at that positions by any other amino acid.

29. A method for producing a plant product from the plant of claim 1, the method comprising processing the plant or a plant part thereof to obtain the plant product.

30. The method of claim 29, wherein the plant product is fodder, seed meal, oil, or seed-treatment-coated seeds.

31. (canceled)

32. (canceled)
